SEQUENCE LISTING (11 sequences)

SEQ ID NO: 1 - protein, 707 aa, Homo sapiens [sequence omitted]

SEQ ID NO: 2 - protein, 427 aa, Homo sapiens
MAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGP

SEQ ID NO: 3 - protein, 417 aa, Homo sapiens
MVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYG

SEQ ID NO: 4 - protein, 242 aa, Homo sapiens
MVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGQGYSLFLVAAHEFGHALGLDHSSVPEALMYPMYRFTEGPPLHKDDVNGIRHLYG

SEQ ID NO: 5 - protein, 204 aa, Homo sapiens
MLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGQGYSLFLVAAHEFGHALGLDHSSVPEALMYPMYRFTEGPPLHKDDVNGIRHLYG

SEQ ID NO: 6 - protein, 251 aa, Homo sapiens
MAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGQGYSLFLVAAHEFGHALGLDHSSVPEALMYPMYRFTEGPPLHKDDVNGIRHLYG

SEQ ID NO: 7 - protein, 268 aa, Homo sapiens
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGP

SEQ ID NO: 8 - protein, 167 aa, Homo sapiens
MFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGP

SEQ ID NO: 9 - protein, 7 aa, Homo sapiens
FQTFEGD

SEQ ID NO: 10 - protein, 15 aa, Homo sapiens
CGVPDLGRFQTFEGD

SEQ ID NO: 11 - protein, 708 aa, Rattus norvegicus
MSPWQPLLLVLLALGYSFAAPHQRQPTYVVFPRDLKTSNLTDTQLAEDYLYRYGYTRAAQMMGEKQSLRPALLMLQKQLSLPQTGELDSETLKAIRSPRCGVPDVGKFQTFDGDLKWHHHNITYWIQSYTEDLPRDVIDDSFARAFAVWSAVTPLTFTRVYGLEADIVIQFGVAEHGDGYPFDGKDGLLAHAFPPGPGIQGDAHFDDDELWSLGKGAVVPTYFGNANGAPCHFPFTFEGRSYLSCTTDGRNDGKPWCGTTADYDTDRKYGFCPSENLYTEHGNGDGKPCVFPFIFEGHSYSACTTKGRSDGYRWCATTANYDQDKADGFCPTRADVTVTGGNSAGEMCVFPFVFLGKQYSTCTSEGRSDGRLWCATTSNFDADKKWGFCPDQGYSLFLVAAHEFGHALGLDHSSVPEALMYPMYHYHEDSPLHEDDIKGIHHLYGRGSKPDPRPPATTAAEPQPTAPPTMCSTAPPMAYPTGGPTVAPTGAPSPGPTGPPTAGPSEAPTESSTPDDNPCNVDVFDAIADIQGALHFFKDGRYWKFSNHGGNQLQGPFLIARTWPAFPSKLNSAFEDPQPKKIFFFLWAQMWVYTGQSVLGPRSLDKLGLGSEVTLVTGLLPRRGGKALLISRERIWKFDLKSQKVDPQSVTRLDNEFSGVPWNSHNVFQYQDKAYFCHDKYFWRVSFHNRVNQVDHVAYVTYDLLQCP

(19) United States
(12) Patent Application Publication
(10) Pub. No.: US 2012/0129811 A1
(43) Pub. Date: May 24, 2012

(54) TRICYCLIC INHIBITORS OF PRO-MATRIX METALLOPROTEINASE ACTIVATION

(76) Inventors: Aihua WANG, Jamison, PA (US); Yan Zhang, Fort Washington, PA (US); Kristi Anne Leonard, Lansdale, PA (US); Michael Hawkins, Ambler, PA (US); Brett Andrew Tounge, Blue Bell, PA (US); Umar S.M. Maharoof, North Wales, PA (US); Joseph Kent Barbay, Flourtown, PA (US)

(21) Appl. No.: 13/297,421

(22) Filed: Nov. 16, 2011

Related U.S. Application Data
(60) Provisional application No. 61/414,972, filed on Nov. 18, 2010.

(51) Int. Cl. A61K 31/429; C07F 9/6561; A61K 31/496; A61K 31/675; A61K 31/4439; A61K 31/454; A61K 31/5377; A61K 31/437; A61P 35/00; A61P 19/02; A61P 19/08; A61P 9/00; A61P 1/04; A61P 9/12; A61P 11/00; A61P 1/00; A61P 29/00; A61P 1/02; A61P 17/02; A61P 1/16; A61P 25/00; A61P 11/06; A61P 13/12; A61P 25/06; A61P 9/10; C07D 513/04

(52) U.S. Cl. 514/81; 548/151; 544/368; 548/113; 546/270.1; 546/199; 544/135; 546/83; 514/366; 514/254.02; 514/338; 514/322; 514/232.8; 514/293

(57) ABSTRACT

This invention relates to tricycle I and its therapeutic and prophylactic uses, wherein the variables C¹, C², Z¹, Z², Q, J, R¹, and R³ are defined in the specification. Disorders treated and/or prevented include rheumatoid arthritis.

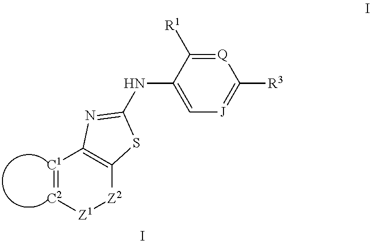

### CROSS-REFERENCE TO RELATED APPLICATIONS

The present application claims the benefits of the filing of U.S. Provisional Application No. 61/414,972 filed Nov. 18, 2010. The complete disclosures of the aforementioned related patent applications are hereby incorporated herein by reference for all purposes.

### TECHNICAL FIELD

The present invention relates to inhibitors of novel tricyclic compounds and their therapeutic and prophylactic uses. Disorders treated and/or prevented include inflammation related disorders and disorders ameliorated by inhibiting the proteolytic activation of pro-matrix metalloproteinases.

### BACKGROUND OF THE INVENTION

Matrix metalloproteinases (MMPs) are a family of structurally related zinc-dependent proteolytic enzymes that digest extracellular matrix proteins such as collagen, elastin, laminin and fibronectin. Currently, at least 28 different mammalian MMP proteins have been identified and they are grouped based on substrate specificity and domain structure. Enzymatic activities of the MMPs are precisely controlled, not only by their gene expression in various cell types, but also by activation of their inactive zymogen precursors (proMMPs) and inhibition by endogenous inhibitors and tissue inhibitors of metalloproteinases (TIMPs). The enzymes play a key role in normal homeostatic tissue remodeling events, but are also considered to play a key role in pathological destruction of the matrix in many connective tissue diseases such as arthritis, periodontitis, and tissue ulceration and also in cancer cell invasion and metastasis.

A role for MMPs in oncology is well established, as up-regulation of any number of MMPs are one mechanism by which malignant cells can overcome connective tissue barriers and metastasize (Curr Cancer Drug Targets 5(3): 203-20, 2005). MMPs also appear to have a direct role in angiogenesis, which is another reason they have been an important target for oncology indications (Int J Cancer 115(6): 849-60, 2005; J Cell Mol Med 9(2): 267-85, 2005). Several different classes of MMPs are involved in these processes, including MMP9. Other MMP mediated indications include the cartilage and bone degeneration that results in osteoarthritis and rheumatoid arthritis. The degeneration is due primarily to MMP digestion of the extracellular matrix (ECM) in bone and joints (Aging Clin Exp Res 15(5): 364-72, 2003). Various MMPs, including MMP9 and MMP13 have been found to be elevated in the tissues and body fluids surrounding the damaged areas.

Elevated MMP levels, including MMP9 and MMP13 are also believed to be involved in atherosclerotic plaque rupture, aneurysm and vascular and myocardial tissue morphogenesis (Expert Opin Investig Drugs 9(5): 993-1007, 2000; Curr Med Chem 12(8): 917-25, 2005). Elevated levels of MMPs, including MMP9 and MMP13, have often been associated with these conditions. Several other pathologies such as gastric ulcers, pulmonary hypertension, chronic obstructive pulmonary disease, inflammatory bowel disease, periodontal disease, skin ulcers, liver fibrosis, emphysema, and Marfan syndrome all appear to have an MMP component as well (Expert Opinion on Therapeutic Patents 12(5): 665-707, 2002). Within the central nervous system, altered MMP expression has been linked to several neurodegenerative disease states (Expert Opin Investig Drugs 8(3): 255-68, 1999), most notably in stroke (Glia 50(4): 329-39, 2005). MMPs, including MMP9, have been shown to have an impact in propagating the brain tissue damage that occurs following an ischemic or hemorrhagic insult. Studies in human stroke patients and in animal stroke models have demonstrated that expression levels and activity of MMPs, including MMP9, increase sharply over a 24 hour period following an ischemic event. Administration of MMP inhibitors has been shown to be protective in animal models of stroke (Expert Opin Investig Drugs 8(3): 255-68, 1999; J Neurosci 25(27): 6401-8, 2005). In addition, MMP9 knockout animals also demonstrate significant neuroprotection in similar stroke models (J Cereb Blood Flow Metab 20(12): 1681-9, 2000). In the US, stroke is the third leading cause of mortality, and the leading cause of disability. Thus stroke comprises a large unmet medical need for acute interventional therapy that could potentially be addressed with MMP inhibitors.

It has also been suggested that MMP9 may play a role in the progression of multiple sclerosis (MS). Studies have indicated that serum levels of MMP9 are elevated in active patients, and are concentrated around MS lesions (Lancet Neurol 2(12): 747-56, 2003). Increased serum MMP9 activity would promote infiltration of leukocytes into the CNS, a causal factor and one of the hallmarks of the disease. MMPs may also contribute to severity and prolongation of migraines. In animal models of migraine (cortical spreading depression), MMP9 is rapidly upregulated and activated leading to a breakdown in the BBB, which results in mild to moderate edema (J Clin Invest 113(10): 1447-55, 2004). It is this brain swelling and subsequent vasoconstriction which causes the debilitating headaches and other symptoms associated with migraine. In the cortical spreading depression model, MMP inhibitors have been shown to prevent the opening of the BBB (J Clin Invest 113(10): 1447-55, 2004). Related research has shown that MMP9 is specifically upregulated in damaged brain tissues following traumatic brain injury (J Neurotrauma 19(5): 615-25, 2002), which would be predicted to lead to further brain damage due to edema and immune cell infiltration. MMPs may also have additional roles in additional chronic CNS disorders. In an animal model of Parkinson's disease, MMP9 was found to be rapidly upregulated after striatal injection of a dopaminergic neuron poison (MPTP).

With regard to structure and activation of the inactive zymogen form, a prototypical MMP is matrix metalloproteinase 9 (MMP9). MMP9 is also known as macrophage gelatinase, gelatinase B, 92 kDa gelatinase, 92 kDa type IV collagenase, and type V collagenase. The inactive form of MMP9, proMMP9, is expressed with several different domains including a signal sequence for secretion, a propeptide domain which inhibits activity of proMMP9, a catalytic domain for protein cleavage, a fibronectin type-II (FnII) domain consisting of three fibronectin-type II repeats, and a hemopexin-like domain thought to assist in substrate docking. The hemopexin-like domain also serves as a binding domain for interaction with tissue inhibitors of metalloproteinases (TIMPs). The inactive zymogen form of MMP9, proMMP9, is maintained through a cysteine-switch mechanism, in which a Cys in the propeptide forms a complex with the catalytic zinc in the catalytic domain and occludes the active site (Proc Natl Acad Sci USA 87(14): 5578-82, 1990). Activation of proMMP9 occurs in a two-step process. A protease cleaves an initial site after Met60, disrupting the zinc coordination and destabilizing the propeptide interaction with the catalytic domain. This initial cleavage allows access to the second cleavage site at Phe107, after which the propeptide is removed and the mature active form of the enzyme is released (Biol Chem 378(3-4): 151-60, 1997). The identity of the proMMP9 activating proteases is unknown in vivo, although there is evidence that activation can occur through the actions of MMP3, chymase and trypsin (J Biol Chem 267(6): 3581-4, 1992; J Biol Chem 272(41): 25628-35, 1997; J Biol Chem 280(10): 9291-6, 2005).

Based on the demonstrated involvement in numerous pathological conditions, inhibitors of matrix metalloproteases (MMPs) have therapeutic potential in a range of disease states. However, non-selective active site MMP inhibitors have performed poorly in clinical trials. The failures have often been caused by dose-limiting toxicity and the manifestation of significant side effects, including the development of musculoskeletal syndrome (MSS). It has been suggested that development of more selective MMP inhibitors might help to overcome some of the problems that hindered clinical success in the past, but there are a number of obstacles to developing more selective MMP active site inhibitors. MMPs share a catalytically important Zn2+ ion in the active site and a highly conserved zinc-binding motif In addition, there is considerable sequence conservation across the entire catalytic domain for members of the MMP family.

A novel approach to developing more selective MMP inhibitors is to target the pro domain of the inactive zymogens, proMMPs, with allosteric small-molecule inhibitors that bind and stabilize the inactive pro form of the protein and inhibit processing to the active enzyme. There is significantly less sequence identity within the pro domains of MMP proteins, no catalytically important Zn2+ ion, and no highly conserved zinc-binding motif. Thus targeting the pro domain of proMMPs is an attractive mechanism of action for inhibiting the activity of the MMP proteins Inhibition of proMMP9 activation has been observed with a specific monoclonal antibody (Hybridoma 12(4): 349-63, 1993). The activation of proMMP9 by trypsin has also been shown to be inhibited by Bowman-Birk inhibitor proteins and derived peptide inhibitors (Biotechnol Lett 26(11): 901-5, 2004). There are no reports, however, of allosteric small-molecule inhibitors that bind the pro domain and inhibit activation of proMMP9, proMMP 13, or any other proMMP. The present invention provides tricyclic compounds as allosteric small-molecule inhibitors of the proteolytic activation of proMMP9, proMMP 13, and methods of treatment using such inhibitors.

### SUMMARY OF THE INVENTION

The invention comprises the compounds of Formula I

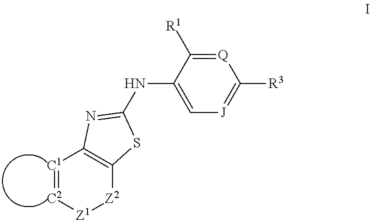

### Wherein:

R¹ is OC₍₁₋₅₎alkyl, O—C₍₃₋₅₎cycloalkyl, SCF₃, CF₃, CH₂CF₃, OCH₂—C₍₃₋₅₎cycloalkyl, OCF₃, OCH₂CF₃, SCH₂CF₃, NO₂, —CN, C₍₁₋₅₎alkyl, Cl, F, SC₍₁₋₄₎alkyl, SCH₂—C₍₃₋₅₎cycloalkyl, S—C₍₃₋₅₎cycloalkyl, or H;

### Q is N or C—R²;

R² is F, Cl, Br, CF₃, CH₃, or H; or R² and R¹ may be taken together with the ring to which they are attached, to form a fused ring system selected from the group consisting of: quinolinyl, isoquinolinyl, quinazolinyl, quinoxalinyl, benzimidazolyl, napthalyl, benzofuranyl, 2,3-dihydro-benzofuranyl, benzothiophenyl, benzothiazolyl, benzotriazolyl, indolyl, indolinyl, and indazolyl, wherein said quinolinyl, isoquinolinyl, quinazolinyl, quinoxalinyl, benzimidazolyl, benzothiazolyl, napthalyl, benzofuranyl, 2,3-dihydro-benzofuranyl, benzothiophenyl, benzotriazolyl, indolyl, indolinyl, and indazolyl are optionally substituted with one methyl group or up to two fluorine atoms;
R³ is C₁, SO₂NH₂, SO₂CH₃, CO₂H, F, CONH₂, OCH₃, NO₂, —CN, CH₃, CF₃, or H; provided that R³ is not F if R⁴ is H;

### J is N, or C—R⁴;

R⁴ is H, NH₂, NHC₍₁₋₃₎alkyl, N(C₍₁₋₃₎alkyl)₂, CONH₂, C₍₁₋₅₎alkyl, CO₂C₍₁₋₅₎alkyl, OC₍₁₋₅₎alkyl, CO₂H, SO₂NH₂, Cl, NHC(O)C₍₁₋₅₎alkyl, CF₃, CH₂CF₃, SO₂C₍₁₋₅₎alkyl, SO₂NHC₍₁₋₅₎alkyl, SO₂N(C₍₁₋₅₎alkyl)₂, SO₂NL¹L², CONL¹L², F, P(O)(OCH₂CH₃)₂, NO₂, —CN, P(O)(OH)₂, Br, —CH═CH₂, CONHC₍₁₋₃₎alkyl, CON(C₍₁₋₃₎alkyl)₂, CH₂CONH₂, —CONHCH₂CH₂-piperidinyl, —CONHCH₂Ph, or NHC(O)NH₂; provided that R⁴ is not OCH₃, if R¹ is C₍₁₋₄₎alkyl or H; or R⁴ is selected from the group consisting of: phenyl, pyridyl, pyrazolyl, imidazolyl, oxazolyl, isoxazolyl, thiazolyl, thiophenyl, pyrimidyl, pyrazyl, and furyl wherein said phenyl, pyridyl, pyrazolyl, imidazolyl, oxazolyl, isoxazolyl, thiazolyl, thiophenyl, pyrimidyl, pyrazyl, and furyl are optionally substituted with one substituent selected from the group consisting of C(O)C₍₁₋₄₎alkyl, C₍₁₋₄₎alkyl, F, Br, Cl, —CN, OC₍₁₋₄₎alkyl; or R⁴ and R³ may be taken together with the ring to which they are attached, to form the fused ring system 2,3-dihydroisoindolin-1-one;
L¹ and L² are taken together with their attached nitrogen to form a ring, selected from the group consisting of:

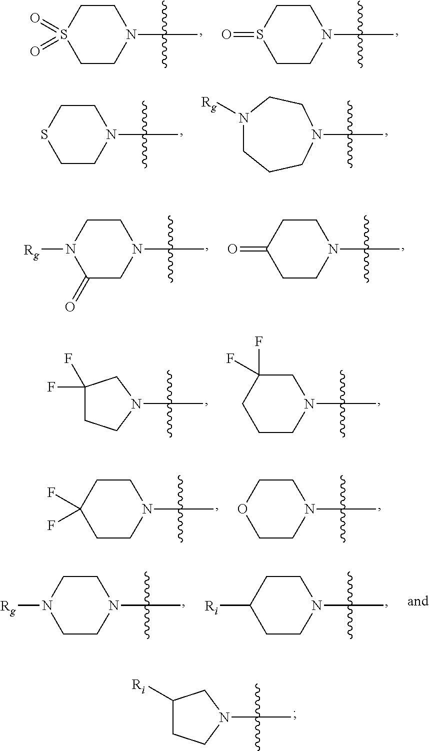

R_{g} is selected from the group consisting of H, CF₃, CH₂CF₃, CH₂CH₂CF₃, C₍₁₋₃₎alkyl, COC₍₁₋₃₎alkyl;
Rᵢ is H, CH₂OH, N(C₍₁₋₄₎alkyl)₂, C₍₁₋₆₎alkyl, CH₂OH, F, or OH;
wherein any said L¹ and L² ring may be optionally substituted with up to four methyl groups on two or more ring carbon atoms or optionally substituted with up to two CF₃ groups on any two ring carbon atoms;
Z¹—Z² is —CH═CH—, —(CH₂)₂—, or —(CH₂)₃—, provided that if Z¹—Z² is CH₂—CH₂ then R⁴ is not H;
C¹ and C² are carbon atoms which are further substituted to make a ring selected from the group consisting of:

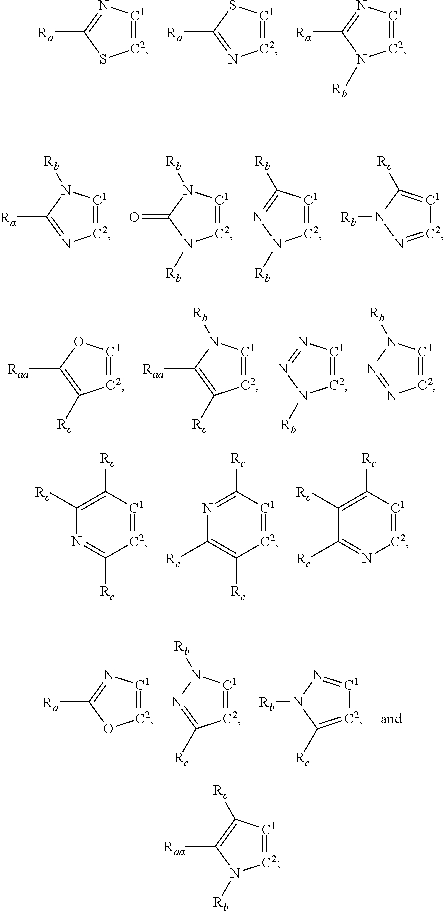

Rₐ is H, CF₃, CH₂CF₃, CH₂OH, Cl, Br, or C₍₁₋₆₎alkyl; or Rₐ may also be

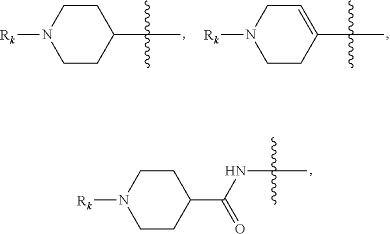

CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, pyridinyl, pyrimidinyl, pyrazinyl, NHPhOCH₂CH₃, NA¹A², C(O)NA¹A², SO₂NA¹A², SONA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², or C₍₁₋₆₎alkylNA¹A², provided that R_{b} is H, CF₃, CH₂CF₃, C₍₁₋₆₎alkyl, or C₍₁₋₆₎cycloalkyl; wherein said

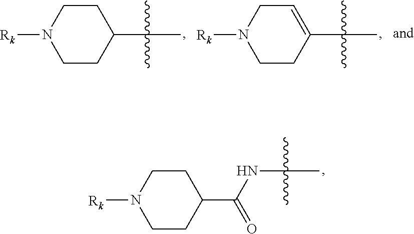

are optionally substituted with up to four methyl groups on two or more ring carbon atoms or optionally substituted with up to two CF₃ groups on any two ring carbon atoms;
A¹ is H, or C₍₁₋₃₎alkyl;
A² is H, C₍₁₋₆₎alkyl, C₍₃₋₆₎cycloalkyl,

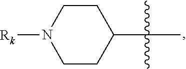

C₍₂₋₆₎alkylOH, C₍₂₋₆₎alkylOCH₃, SO₂C₍₁₋₄₎alkyl, C(O)Ph, C(O)C₍₁₋₄₎alkyl, pyrazinyl, or pyridyl, wherein said cycloalkyl, alkyl, pyrazinyl, pyridyl, or Ph groups may be optionally be substituted with two substituents selected from the group consisting of F, C₍₁₋₆₎alkyl, CF₃, pyrrolidinyl, CO₂H, C(O)NH₂, SO₂NH₂, OC₍₁₋₄₎alkyl, —CN, NO₂, OH, NH₂, NHC₍₁₋₄₎alkyl, N(C₍₁₋₄₎alkyl)₂; and said pyridyl, or Ph may be additionally be substituted with up to two halogens independently selected from the group consisting of: Cl, and Br; or A¹ and A² are taken together with their attached nitrogen to form a ring selected from the group consisting of:

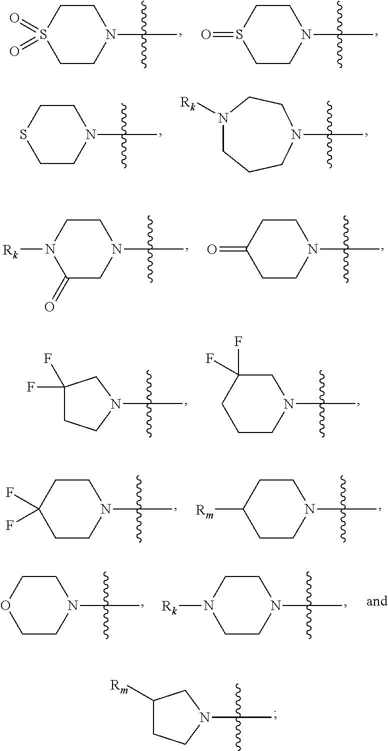

wherein any said A¹ and A² ring may be optionally substituted with up to four methyl groups on two or more ring carbon atoms or optionally substituted with up to two CF₃ groups on any two ring carbon atoms;
Rₖ is selected from the group consisting of H, CH₂CF₃, CH₂CH₂CF₃, C₍₁₋₆₎alkyl, COC₍₁₋₄₎alkyl, SO₂C₍₁₋₄₎alkyl, trifluoromethylpyridyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, C₍₁₋₆₎alkyl, F, or OH;
Rₐₐ is H, CF₃, CH₂CF₃, Cl, Br, C₍₁₋₆₎alkyl, CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, SO₂NA¹A², SONA¹A², C(O)NA¹A², C(O)N(C₍₁₋₃₎alkyl)C₍₂₋₄₎alkylNA¹A², C(O)NHC₍₂₋₄₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², or C₍₁₋₆₎alkylNA¹A²;
R_{b} is H, CF₃, CH₂CF₃, or C₍₁₋₆₎alkyl, or C₍₃₋₆₎cycloalkyl; or R_{b} may also be

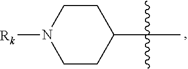

C(O)C₍₁₋₄₎alkyl, C(O)Ph, SO₂C₍₁₋₄₎alkyl, C₍₂₋₆₎alkylOC₍₁₋₆₎alkyl, C₍₂₋₆₎alkylOC₍₃₋₆₎cycloalkyl, C₍₂₋₆₎alkylOC₍₂₋₆₎alkylNA¹A², C₍₂₋₆₎alkylNHC₍₂₋₆₎alkylNA¹A², C₍₂₋₆₎alkylN(C₍₁₋₃₎alkyl)C₍₂₋₆₎alkylNA¹A², or C₍₂₋₆₎alkylNA¹A², provided that Rₐ is H, Cl, Br, CH₂OH, NH₂, CF₃, CH₂CF₃, or C₍₁₋₆₎alkyl; wherein said

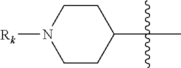

is optionally substituted with up to four methyl groups on two or more ring carbon atoms or optionally substituted with up to two CF₃ groups on any two ring carbon atoms;
Rₑ is H, C₍₁₋₃₎alkyl, or CF₃; and solvates, hydrates, tautomers, and pharmaceutically acceptable salts thereof.

### BRIEF DESCRIPTION OF THE DRAWINGS

Embodiments of the present invention will now be described, by way of an example only, with reference to the accompanying drawings wherein:

FIG. 1: Shown are western blots with two different antibodies illustrating the effects of a small molecule allosteric processing inhibitor, Compound α, on the activation of proMMP9 in synoviocytes harvested from female Lewis rats after inducing arthritis with i.p. administration of Streptococcal cell wall peptidoglycan polysaccharides. A mouse monoclonal antibody, mAb L51/82, detected pro and processed forms of MMP9. The mouse monoclonal antibody showed that Compound α caused a dose-dependent reduction in the appearance of the 80 kD active form of MMP9 and the appearance of an 86 kD form of the protein (FIG. 1A, lanes 3-6). A rabbit polyclonal antibody, pAb-1246, detected the 80 kD active form of MMP9, but did not recognize the 100 kD form of proMMP9. The rabbit polyclonal antibody showed that the small molecule allosteric processing inhibitor caused a dose-dependent reduction in the appearance of the 80 kD active form of MMP9 (FIG. 1B, lanes 2-6).

FIG. 2: Shown are western blots illustrating increased proMMP9 and increased active MMP9 in tibia-tarsus joints (ankles) from female Lewis rats after inducing arthritis with i.p. administration of Streptococcal cell wall peptidoglycan polysaccharides (SCW). In healthy ankles of rats administered saline, mAb-L51/82 detected small amounts of an approximately 100 kD proMMP9 and an approximately 80 kD form of active MMP9 (FIG. 2A, lanes 1 and 2). The amount of proMMP9 increased markedly in ankle homogenates 5 and 18 days after SCW-administration (FIG. 2A, lanes 3-5 and 6-8, respectively). The amount of active 80 kD MMP9 increased mildly 5 days after SCW-administration (FIG. 2A, lanes 3-5) and increased markedly 18 days after SCW-administration (FIG. 2A, lanes 6-8). In healthy ankles of rats administered saline, mAb-1246 detected small amounts active 80 kD MMP9 (FIG. 2B, lanes 1 and 2). The 80 kD active MMP9 increased mildly 5 days after SCW-administration (FIG. 2A, lanes 3-5) and increased markedly 18 days after SCW-administration (FIG. 2A, lanes 6-8).

FIG. 3: Shown are western blots with two different antibodies illustrating the effects of a small molecule allosteric processing inhibitor, Compound α, on the activation of proMMP9 in tibia-tarsus joints (ankles) from female Lewis rats after inducing arthritis with i.p. administration of Streptococcal cell wall peptidoglycan polysaccharides (SCW). Both proMMP9 and active MMP9 were abundantly present in ankles of SCW-induced vehicle-treated rats (FIGS. 3A and 3B, lanes 1-3). Treatment of rats with Compound α did not reduce the abundance of proMMP-9 (FIG. 3A, lanes 4-9). However, treatment of rats with Compound α resulted in a notable reduction in the active 80 kD form of MMP9 detected with pAb-1246 (FIG. 3B, lanes 4-9) and also with mAb-L51/82 (FIG. 3A, lanes 4-9).

### DETAILED DESCRIPTION OF THE INVENTION

The invention comprises the compounds of Formula I

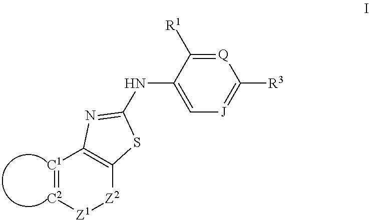

wherein:

R¹ is OC₍₁₋₅₎alkyl, O—C₍₃₋₅₎cycloalkyl, SCF₃, CF₃, CH₂CF₃, OCH₂—C₍₃₋₅₎cycloalkyl, OCF₃, OCH₂CF₃, SCH₂CF₃, NO₂, —CN, C₍₁₋₅₎alkyl, Cl, F, SC₍₁₋₄₎alkyl, SCH₂—C₍₃₋₅₎cycloalkyl, S—C₍₃₋₅₎cycloalkyl, or H;

### Q is N or C—R²;

R² is F, Cl, Br, CF₃, CH₃, or H; or R² and R¹ may be taken together with the ring to which they are attached, to form a fused ring system selected from the group consisting of: quinolinyl, isoquinolinyl, quinazolinyl, quinoxalinyl, benzimidazolyl, napthalyl, benzofuranyl, 2,3-dihydro-benzofuranyl, benzothiophenyl, benzothiazolyl, benzotriazolyl, indolyl, indolinyl, and indazolyl, wherein said quinolinyl, isoquinolinyl, quinazolinyl, quinoxalinyl, benzimidazolyl, benzothiazolyl, napthalyl, benzofuranyl, 2,3-dihydro-benzofuranyl, benzothiophenyl, benzotriazolyl, indolyl, indolinyl, and indazolyl are optionally substituted with one methyl group or up to two fluorine atoms;
R³ is C₁, SO₂NH₂, SO₂CH₃, CO₂H, F, CONH₂, OCH₃, NO₂, —CN, CH₃, CF₃, or H; provided that R³ is not F if R⁴ is H;

### J is N, or C—R⁴;

R⁴ is H, NH₂, NHC₍₁₋₃₎alkyl, N(C₍₁₋₃₎alkyl)₂, CONH₂, C₍₁₋₅₎alkyl, CO₂C₍₁₋₅₎alkyl, OC₍₁₋₅₎alkyl, CO₂H, SO₂NH₂, Cl, NHC(O)C₍₁₋₅₎alkyl, CF₃, CH₂CF₃, SO₂C₍₁₋₅₎alkyl, SO₂NHC₍₁₋₅₎alkyl, SO₂N(C₍₁₋₅₎alkyl)₂, SO₂NL¹L², CONL¹L², F, P(O)(OCH₂CH₃)₂, NO₂, —CN, P(O)(OH)₂, Br, —CH═CH₂, CONHC₍₁₋₃₎alkyl, CON(C₍₁₋₃₎alkyl)₂, CH₂CONH₂, —CONHCH₂CH₂-piperidinyl, —CONHCH₂Ph, or NHC(O)NH₂; provided that R⁴ is not OCH₃, if R¹ is C₍₁₋₄₎alkyl or H; or R⁴ is selected from the group consisting of: phenyl, pyridyl, pyrazolyl, imidazolyl, oxazolyl, isoxazolyl, thiazolyl, thiophenyl, pyrimidyl, pyrazyl, and furyl wherein said phenyl, pyridyl, pyrazolyl, imidazolyl, oxazolyl, isoxazolyl, thiazolyl, thiophenyl, pyrimidyl, pyrazyl, and furyl are optionally substituted with one substituent selected from the group consisting of C(O)C₍₁₋₄₎alkyl, C₍₁₋₄₎alkyl, F, Br, Cl, —CN, OC₍₁₋₄₎alkyl; or R⁴ and R³ may be taken together with the ring to which they are attached, to form the fused ring system 2,3-dihydroisoindolin-1-one;
L¹ and L² are taken together with their attached nitrogen to form a ring, selected from the group consisting of:

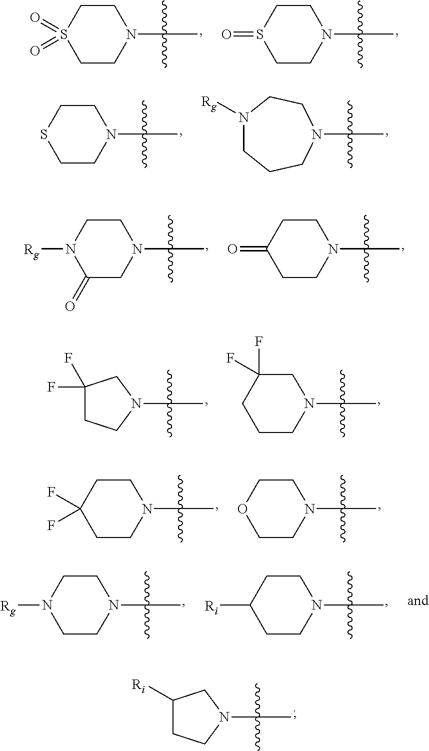

R_{g} is selected from the group consisting of H, CF₃, CH₂CF₃, CH₂CH₂CF₃, C₍₁₋₃₎alkyl, COC₍₁₋₃₎alkyl;
Rᵢ is H, CH₂OH, N(C₍₁₋₄₎alkyl)₂, C₍₁₋₆₎alkyl, CH₂OH, F, or OH;
wherein any said L¹ and L² ring may be optionally substituted with up to four methyl groups on two or more ring carbon atoms or optionally substituted with up to two CF₃ groups on any two ring carbon atoms;
Z¹—Z² is —CH═CH—, —(CH₂)₂—, or —(CH₂)₃—, provided that if Z¹—Z² is CH₂—CH₂ then R⁴ is not H;
C¹ and C² are carbon atoms which are further substituted to make a ring selected from the group consisting of:

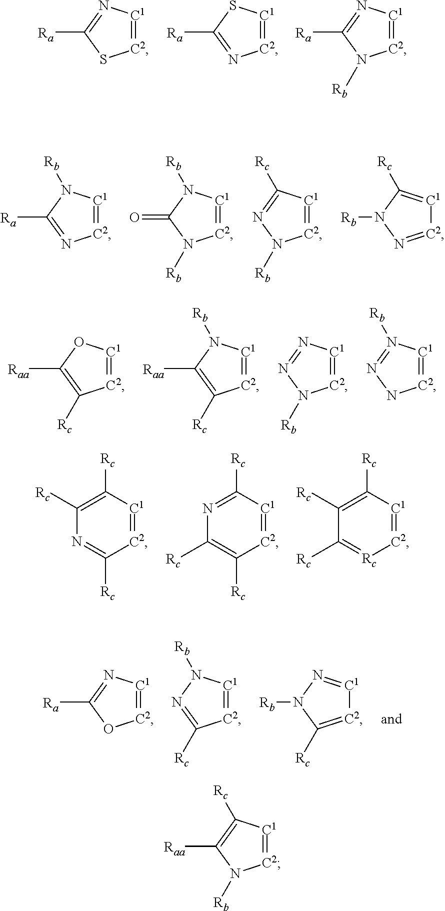

R₁ is H, CF₃, CH₂CF₃, CH₂OH, Cl, Br, or C₍₁₋₆₎alkyl; or Rₐ may also be

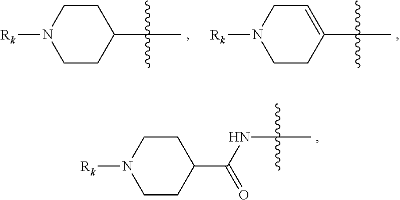

CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, pyridinyl, pyrimidinyl, pyrazinyl, NHPhOCH₂CH₃, NA¹A², C(O)NA¹A², SO₂NA¹A², SONA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎)alkylNA¹A², or C₍₁₋₆₎alkylNA¹A², provided that R_{b} is H, CF₃, CH₂CF₃, C₍₁₋₆₎alkyl, or C₍₁₋₆₎cycloalkyl; wherein said

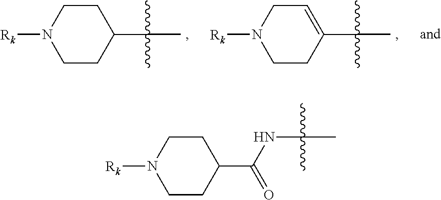

are optionally substituted with up to four methyl groups on two or more ring carbon atoms or optionally substituted with up to two CF₃ groups on any two ring carbon atoms;
A¹ is H, or C₍₁₋₃₎alkyl;
A² is H, C₍₁₋₆₎alkyl, C₍₃₋₆₎cycloalkyl

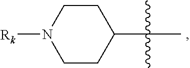

C₍₂₋₆₎alkylOH, C₍₂₋₆₎alkylOCH₃, SO₂C₍₁₋₄₎alkyl, C(O)Ph, C(O)C₍₁₋₄₎alkyl, pyrazinyl, or pyridyl, wherein said cycloalkyl, alkyl, pyrazinyl, pyridyl, or Ph groups may be optionally be substituted with two substituents selected from the group consisting of F, C₍₁₋₆₎alkyl, CF₃, pyrrolidinyl, CO₂H, C(O)NH₂, SO₂NH₂, OC₍₁₋₄₎alkyl, —CN, NO₂, OH, NH₂, NHC₍₁₋₄₎alkyl, N(C₍₁₋₄₎alkyl)₂; and said pyridyl, or Ph may be additionally be substituted with up to two halogens independently selected from the group consisting of: Cl, and Br; or A¹ and A² are taken together with their attached nitrogen to form a ring selected from the group consisting of:

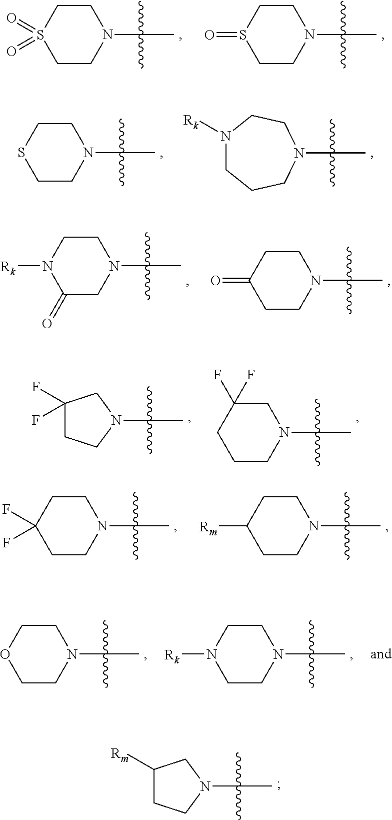

wherein any said A¹ and A² ring may be optionally substituted with up to four methyl groups on two or more ring carbon atoms or optionally substituted with up to two CF₃ groups on any two ring carbon atoms;
Rₖ is selected from the group consisting of H, CH₂CF₃, CH₂CH₂CF₃, C₍₁₋₆₎alkyl, C₍₁₋₄₎alkyl, SO₂C₍₁₋₄₎alkyl, trifluoromethylpyridyl, and C₍₃₋₆₎cycloalkyl;
Rₘ is H, OCH₃, CH₂OH, NH(C₍₁₋₄₎alkyl), N(C₍₁₋₆₎alkyl)₂, NH₂, C₍₁₋₆₎alkyl, F, or OH;
Rₐₐ is H, CF₃, CH₂CF₃, Cl, Br, C₍₁₋₆₎alkyl, CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, SO₂NA¹A², SONA¹A², C(O)NA¹A², C(O)N(C₍₁₋₃₎alkyl)C₍₂₋₄₎alkylNA¹A², C(O)NHC₍₂₋₄₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², or C₍₁₋₆₎alkylNA¹A²;
R_{b} is H, CF₃, CH₂CF₃, or C₍₁₋₆₎alkyl, or C₍₃₋₆₎cycloalkyl; or R_{b} may also be

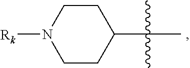

C(O)C₍₁₋₄₎alkyl, C(O)Ph, SO₂C₍₁₋₄₎alkyl, C₍₂₋₆₎alkylOC₍₁₋₆₎alkyl, C₍₂₋₆₎alkylOC₍₃₋₆₎cycloalkyl, C₍₂₋₆₎alkylOC₍₂₋₆₎alkylNA¹A², C₍₂₋₆₎alkylNHC₍₂₋₆₎alkylNA¹A², C₍₂₋₆₎alkylN(C₍₁₋₃₎alkyl)C₍₂₋₆₎alkylNA¹A², or C₍₂₋₆₎alkylNA¹A², provided that Rₐ is H, Cl, Br, CH₂OH, NH₂, CF₃, CH₂CF₃, or C₍₁₋₆₎alkyl; wherein said

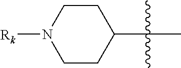

is optionally substituted with up to four methyl groups on two or more ring carbon atoms or optionally substituted with up to two CF₃ groups on any two ring carbon atoms;
R_{c} is H, C₍₁₋₃₎alkyl, or CF₃;
and solvates, hydrates, tautomers, and pharmaceutically acceptable salts thereof.
In another embodiment of the invention:
R¹ is OC₍₁₋₅₎alkyl, O—C₍₃₋₅₎cycloalkyl, CF₃, CH₂CF₃, OCH₂—C₍₃₋₅₎cycloalkyl, OCF₃, OCH₂CF₃, NO₂, C₍₁₋₅₎alkyl, Cl, F, SC₍₁₋₄₎alkyl, SCH₂-cyclopentyl, S-cyclopropyl, or H;

### Q is N or C—R²;

R² is F, Cl, Br, CF₃, CH₃, or H; or R² and R¹ may be taken together with the ring to which they are attached, to form a fused ring system selected from the group consisting of: quinolinyl, isoquinolinyl, quinazolinyl, quinoxalinyl, benzimidazolyl, benzofuranyl, 2,3-dihydro-benzofuranyl, benzothiophenyl, benzothiazolyl, and indazolyl, wherein said quinolinyl, isoquinolinyl, quinazolinyl, quinoxalinyl, benzimidazolyl, benzothiazolyl, benzofuranyl, 2,3-dihydro-benzofuranyl, benzothiophenyl, and indazolyl are optionally substituted with one methyl group or up to two fluorine atoms;
R³ is Cl, SO₂NH₂, SO₂CH₃, CO₂H, F, CONH₂, OCH₃, NO₂, —CN, CH₃, CF₃, or H; provided that R³ is not F if R⁴ is H;

### J is N, or C—R⁴;

R⁴ is H, NH₂, CONH₂, C₍₁₋₅₎alkyl, CO₂C₍₁₋₅₎alkyl, OC₍₁₋₅₎alkyl, CO₂H, SO₂NH₂, Cl, NHC(O)C₍₁₋₅₎alkyl, CF₃, CH₂CF₃, SO₂C₍₁₋₅₎alkyl, SO₂NHC₍₁₋₅₎alkyl, SO₂N(C₍₁₋₅₎alkyl)₂, SO₂NL¹L², F, P(O)(OCH₂CH₃)₂, NO₂, —CN, P(O)(OH)₂, Br, —CH═CH₂, —CONHCH₃, CH₂CONH₂, —CONHCH₂CH₂-piperidinyl, —CONHCH₂Ph, or NHC(O)NH₂; provided that R⁴ is not OCH₃, if R¹ is C₍₁₋₄₎alkyl or H; or R⁴ is selected from the group consisting of: phenyl, pyridyl, pyrazolyl, imidazolyl, oxazolyl, isoxazolyl, thiazolyl, and thiophenyl wherein said phenyl, pyridyl, pyrazolyl, imidazolyl, oxazolyl, isoxazolyl, thiazolyl, and thiophenyl are optionally substituted with one substituent selected from the group consisting of C(O)CH₃, F, Cl, —CN, OCH₃, and CH₃; L¹ and L² are taken together with their attached nitrogen to form a ring, selected from the group consisting of:

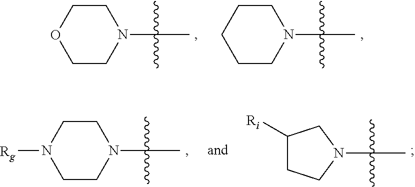

R_{g} is selected from the group consisting of H, COCH₃, and CH₃;

### Rᵢ is H, CH₃, or CH₂OH;

Z¹—Z² is —CH═CH—, —(CH₂)₂—, or —(CH₂)₃—, provided that if Z¹—Z² is CH₂—CH₂ then R⁴ is not H;
C¹ and C² are carbon atoms which are further substituted to make a ring selected from the group consisting of:

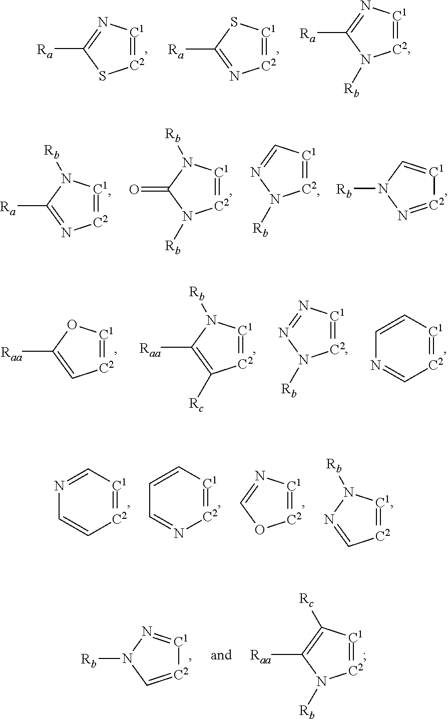

Rₐ is H, CF₃, CH₂CF₃, CH₂OH, Cl, Br, or C₍₁₋₆₎alkyl; or Rₐ may also be

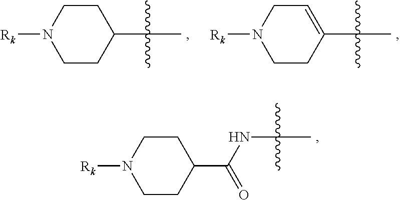

NHPhOCH₂CH₃, NA¹A², C(O)NA¹A², SO₂NA¹A², SONA¹A², C(O)N(CH₃)C₍₂₋₆₎alkylNA¹A², C(O)NHC₍₂₋₆₎alkylNA¹A², NHC(O)C₍₁₋₆₎alkylNA¹A², N(CH₃)C(O)C₍₁₋₆₎alkylNA¹A², CH₂OC₍₁₋₆₎alkyl, CH₂OC₍₃₋₆₎cycloalkyl, CH₂OC₍₂₋₆₎alkylNA¹A², CH₂NHC₍₂₋₆₎alkylNA¹A², CH₂N(CH₃)C₍₂₋₆₎alkylNA¹A², NHC₍₂₋₆₎alkylNA¹A², N(CH₃)C₍₂₋₆₎alkylNA¹A², or CH₂NA¹A², provided that R_{b} is H, CF₃, CH₂CF₃, C₍₁₋₆₎alkyl, or C₍₃₋₆₎cycloalkyl;
A¹ is H, or C₍₁₋₃₎alkyl;
A² is H, C₍₁₋₆₎alkyl, C₍₃₋₆₎cycloalkyl,

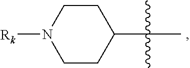

C₍₂₋₆₎alkylOH, C₍₂₋₆₎alkylOCH₃, SO₂C₍₁₋₄₎alkyl, C(O)Ph, C(O)C₍₁₋₄₎alkyl, pyrazinyl, or pyridyl; or A¹ and A² are taken together with their attached nitrogen to form a ring selected from the group consisting of:

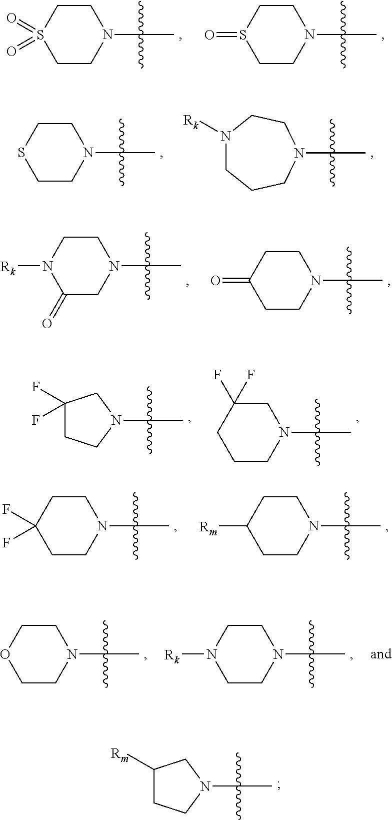

wherein any said A¹ and A² ring may be optionally substituted with up to four methyl groups on two or more ring carbon atoms or optionally substituted with up to two CF₃ groups on any two ring carbon atoms;
Rₖ is selected from the group consisting of H, CH₂CF₃, CH₂CH₂CF₃, C₍₁₋₃₎alkyl, COC₍₁₋₄₎alkyl, SO₂C₍₁₋₄₎alkyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, CH₃, F, or OH;
Rₐₐ is H, CF₃, CH₂CF₃, Cl, Br, C₍₁₋₆₎alkyl, SO₂NA¹A², SONA¹A², C(O)NA¹A², C(O)N(CH₃)C₍₂₋₄₎alkylNA¹A², C(O)NHC₍₂₋₄₎alkylNA¹A², CH₂OC₍₁₋₆₎alkyl, CH₂OC₍₃₋₆₎cycloalkyl, CH₂OC₍₂₋₆₎alkylNA¹A², CH₂NHC₍₂₋₆₎alkylNA¹A², CH₂N(CH₃)C₍₂₋₆₎alkylNA¹A², or CH₂NA¹A²;
R_{b} is H, CF₃, CH₂CF₃, or C₍₁₋₆₎alkyl, or C₍₃₋₆₎cycloalkyl; or R_{b} may also be

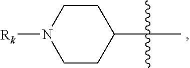

CH₂CH₂OC₍₁₋₆₎alkyl, CH₂CH₂OC₍₃₋₆₎cycloalkyl, CH₂CH₂OC₍₂₋₆₎alkylNA¹A², CH₂CH₂NHC₍₂₋₆₎alkylNA¹A², CH₂CH₂N(CH₃)C₍₂₋₆₎alkylNA¹A², or CH₂CH₂NA¹A², provided that Rₐ is H, Cl, Br, CH₂OH, NH₂, CF₃, CH₂CF₃, or C₍₁₋₆₎alkyl;
R_{c} is H, C₍₁₋₃₎alkyl, or CF₃;
and solvates, hydrates, tautomers, and pharmaceutically acceptable salts thereof.
In another embodiment of the invention:
R¹ is OC₍₁₋₅₎alkyl, O-cyclopentyl, CF₃, CH₂CF₃, SCH₃, OCH₂-cyclopropyl, OCF₃, OCH₂CF₃, NO₂, C₍₁₋₅₎alkyl or H;

### Q is N or C—R²;

R² is F, CF₃, CH₃, or H; or R² and R¹ may be taken together with the ring to which they are attached, to form a fused ring system selected from the group consisting of: benzimidazolyl, quinolinyl, benzofuranyl, and 2,3-dihydro-benzofuranyl, wherein said benzimidazolyl, quinolinyl, benzofuranyl, and 2,3-dihydro-benzofuranyl are optionally substituted with one methyl group or up to two fluorine atoms;
R³ is F, CONH₂, OCH₃, NO₂, —CN, CH₃, CF₃, or H; provided that R³ is not F if R⁴ is H;

### J is N, or C—R⁴;

R⁴ is H, NH₂, CONH₂, C₍₁₋₅₎alkyl, CO₂C₍₁₋₅₎alkyl, OC₍₁₋₅₎alkyl, CO₂H, SO₂NH₂, Cl, NHC(O)C₍₁₋₅₎alkyl, CF₃, CH₂CF₃, SO₂C₍₁₋₅₎alkyl, SO₂NHC₍₁₋₅₎alkyl, SO₂N(C₍₁₋₅₎alkyl)₂, SO₂NL¹L², F, P(O)(OCH₂CH₃)₂, NO₂, —CN, P(O)(OH)₂, Br, pyrazolyl, —CH═CH₂, —CONHCH₃, CH₂CONH₂, —CONHCH₂CH₂-piperidinyl, —CONHCH₂Ph, or NHC(O)NH₂; provided that R⁴ is not OCH₃, if R¹ is C₍₁₋₄₎alkyl or H;
L¹ and L² are taken together with their attached nitrogen to form a ring, selected from the group consisting of:

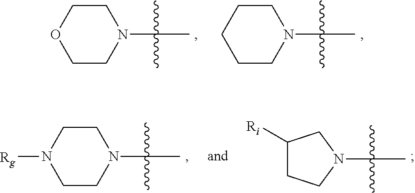

R_{g} is selected from the group consisting of H, COCH₃, and CH₃;

### Rᵢ is H, CH₃, or CH₂OH;

Z¹—Z² is —CH═CH—, —(CH₂)₂—, or —(CH₂)₃—, provided that if Z¹—Z² is CH₂—CH₂ then R⁴ is not H;
C¹ and C² are carbon atoms which are further substituted to make a ring selected from the group consisting of:

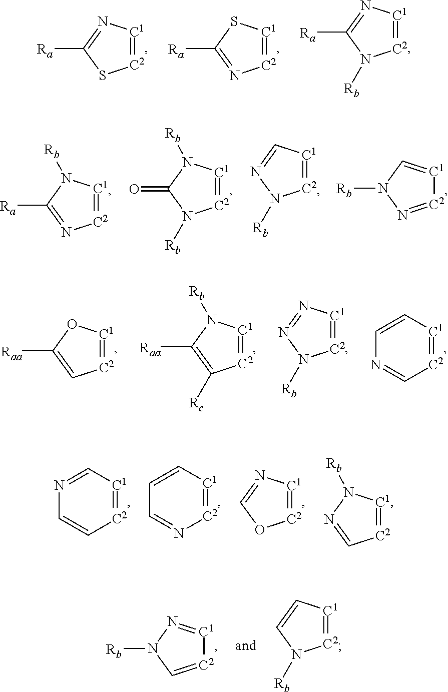

Rₐ is H, CF₃, Cl, CH₂OH, CH₂CF₃, or C₍₁₋₅₎alkyl; or Rₐ may also be

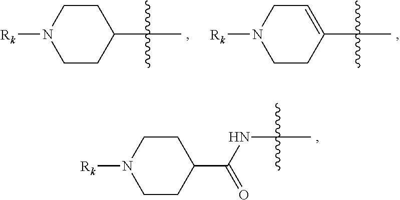

NHPhOCH₂CH₃, NA¹A², C(O)NA¹A², SO₂NA¹A², SONA¹A², C(O)N(CH₃)C₍₂₋₃₎alkylNA¹A², C(O)NHC₍₂₋₃₎alkylNA¹A², NHC(O)C₍₁₋₃₎alkylNA¹A², N(CH₃)C(O)C₍₁₋₃₎alkylNA¹A², CH₂OC₍₂₋₃₎alkylNA¹A², CH₂NHC₍₂₋₃₎alkylNA¹A², CH₂N(CH₃)C₍₂₋₃₎alkylNA¹A², NHC₍₂₋₃₎alkylNA¹A², N(CH₃)C₍₂₋₃₎alkylNA¹A², or CH₂NA¹A², provided that R_{b} is H, CF₃, CH₂CF₃, C₍₁₋₅₎alkyl, or C₍₁₋₆₎cycloalkyl;
A¹ is H, or C₍₁₋₃₎alkyl;
A² is H, C₍₁₋₃₎alkyl,

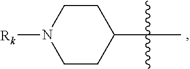

or C(O)C₍₁₋₄₎alkyl; or A¹ and A² are taken together with their attached nitrogen to form a ring selected from the group consisting of:

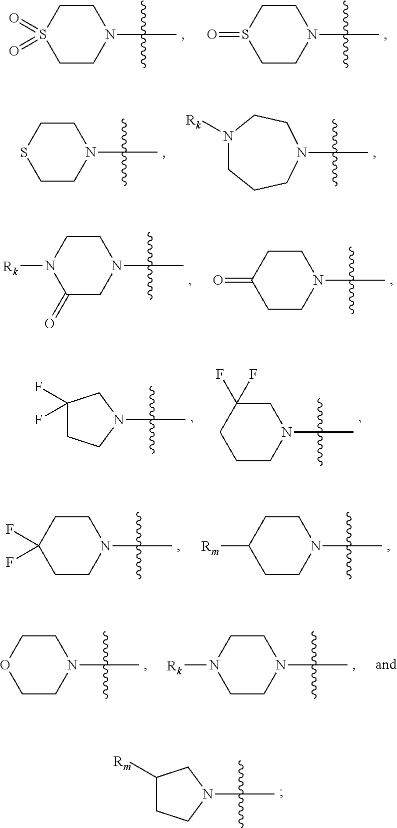

Rₖ is selected from the group consisting of H, C₍₁₋₃₎alkyl, COC₍₁₋₄₎alkyl, SO₂C₍₁₋₄₎alkyl, and C₍₃₋₆₎cycloalkyl;

### Rₘ is H, OCH₃, CH₂OH, NHCH₃, N(CH₃)₂, NH₂, F, or OH;

Rₐₐ is H, CF₃, CH₂CF₃, C₍₁₋₅₎alkyl, SO₂NA¹A², SONA¹A², C(O)NA¹A², C(O)N(CH₃)C₍₂₋₃₎alkylNA¹A², C(O)NHC₍₂₋₃₎alkylNA¹A², CH₂OC₍₂₋₃₎alkylNA¹A², CH₂NHC₍₂₋₃₎alkylNA¹A², CH₂N(CH₃)C₍₂₋₃₎alkylNA¹A², or CH₂NA¹A²;
R_{b} is H, CF₃, CH₂CF₃, or C₍₁₋₅₎alkyl, or C₍₃₋₆₎cycloalkyl; or R_{b} may also be

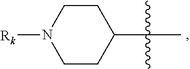

CH₂CH₂OC₍₂₋₃₎alkylNA¹A², CH₂CH₂NHC₍₂₋₃₎alkylNA¹A², CH₂CH₂N(CH₃)C₍₂₋₃₎alkylNA¹A², or CH₂CH₂NA¹A², provided that Rₐ is H, NH₂, CF₃, CH₂CF₃, Cl, CH₂OH, or C₍₁₋₅₎alkyl;
R_{c} is H, C₍₁₋₃₎alkyl, or CF₃;
and solvates, hydrates, tautomers, and pharmaceutically acceptable salts thereof.

In another embodiment of the invention:

R¹ is OC₍₁₋₄₎alkyl, O-cyclopentyl, SCH₃, OCH₂-cyclopropyl, OCF₃, OCH₂CF₃, NO₂, C₍₁₋₅₎alkyl or H;

### Q is N or C—R²;

R² is F, CF₃, CH₃, or H; or R² and R¹ may be taken together with the ring to which they are attached, to form a fused ring system selected from the group consisting of: 1-methyl benzoimidazol-4-yl;
R³ is F, CONH₂, OCH₃, NO₂, —CN, CH₃, CF₃, or H; provided that R³ is not F if R⁴ is H;

### J is N, or C—R⁴;

R⁴ is H, NH₂, CONH₂, C₍₁₋₅₎alkyl, CO₂C₍₁₋₅₎alkyl, OC₍₁₋₅₎alkyl, CO₂H, SO₂NH₂, Cl, NHC(O)C₍₁₋₅₎alkyl, CF₃, CH₂CF₃, SO₂C₍₁₋₅₎alkyl, SO₂NHC₍₁₋₅₎alkyl, SO₂N(C₍₁₋₅₎alkyl)₂, SO₂-pyrrolidinyl, SO₂-piperidinyl, SO₂-morpholinyl, SO₂-piperazinyl, F, P(O)(OCH₂CH₃)₂, NO₂, —CN, P(O)(OH)₂, Br, pyrazolyl, or NHC(O)NH₂; provided that R⁴ is not OCH₃, if R¹ is C₍₁₋₄₎alkyl or H;
Z¹—Z² is —CH═CH—, —(CH₂)₂—, or —(CH₂)₃—, provided that if Z¹—Z² is CH₂—CH₂ then R⁴ is not H;
C¹ and C² are carbon atoms which are further substituted to make a ring selected from the group consisting of:

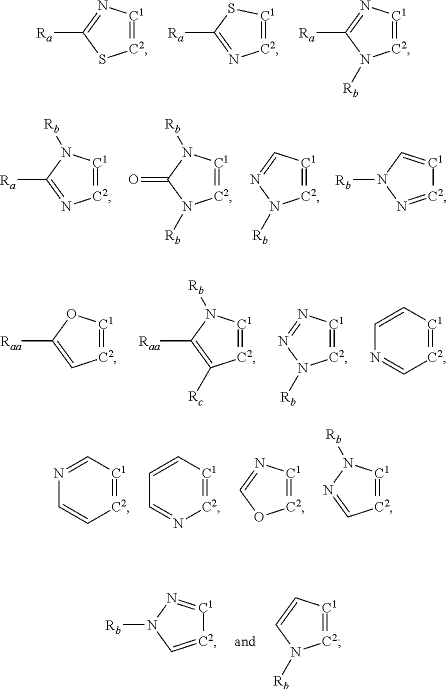

R₁ is H, CF₃, CH₂CF₃, C₍₁₋₅₎alkyl, C₁, NH₂, CH₂OH, NHC(O)C₍₁₋₄₎alkyl, NHC₍₁₋₃₎alkyl, N(C₍₁₋₃₎alkyl)₂, NHPhOCH₂CH₃, C(O)NA¹A², or NA¹A²;
A¹ and A² are taken together with their attached nitrogen to form a ring selected from the group consisting of:

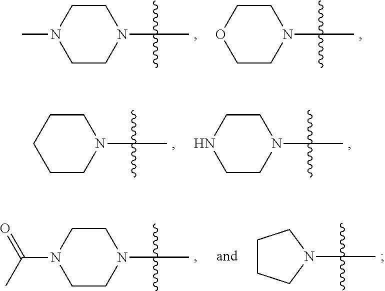

Rₐₐ is H, or C₍₁₋₅₎alkyl;
R_{b} is H, CH₂CF₃, or C₍₁₋₅₎alkyl;
R_{c} is H, or C₍₁₋₃₎alkyl;
and solvates, hydrates, tautomers, and pharmaceutically acceptable salts thereof.

In another embodiment of the invention:

R¹ is OC₍₁₋₄₎alkyl, OCF₃, OCH₂CF₃, NO₂, C₍₁₋₄₎alkyl or H;

### Q is N or C—R²;

R² is F, CF₃, or H; or R² and R¹ may be taken together with the ring to which they are attached, to form a fused ring system selected from the group consisting of: 1-methyl benzoimidazol-4-yl;
R³ is F, CONH₂, OCH₃, NO₂, —CN, CH₃, or H; provided that R³ is not F if R⁴ is H;

### J is N, or C—R⁴;

R⁴ is H, NH₂, CONH₂, CH₃, CO₂CH₃, OCH₃, CO₂H, SO₂NH₂, Cl, NHC(O)CH₃, CF₃, SO₂CH₃, SO₂CH₂CH₃, SO₂NHCH₃, SO₂N(CH₃)₂, SO₂N(CH₂CH₃)₂, SO₂-pyrrolidinyl, SO₂-piperidinyl, SO₂-morpholinyl, F, P(O)(OCH₂CH₃)₂, NO₂, —CN, P(O)(OH)₂, Br, pyrazolyl, or NHC(O)NH₂; provided that R⁴ is not OCH₃, if R¹ is C₍₁₋₄₎alkyl or H;
Z¹—Z² is —CH═CH—, —(CH₂)₂—, or —(CH₂)₃—, provided that if Z¹—Z² is CH₂—CH₂ then R⁴ is not H;
C² and C² are carbon atoms which are further substituted to make a ring selected from the group consisting of:

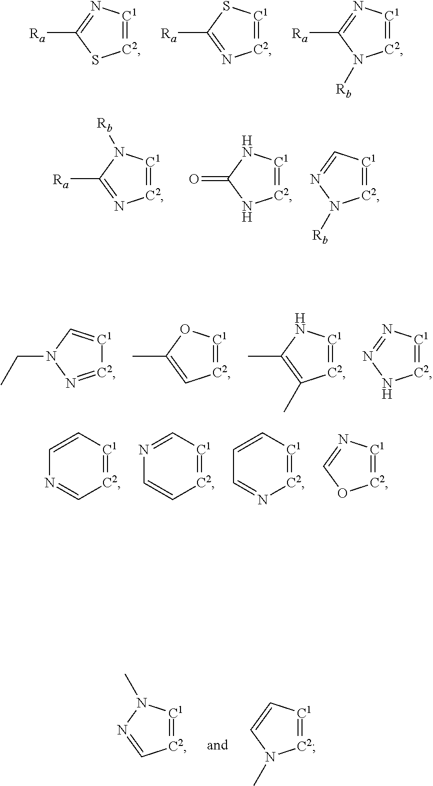

Rₐ is H, CH₃, C₁, NH₂, CH₂OH, NHC(O)CH₃, NHPhOCH₂CH₃, C(O)NA¹A², or NA¹A²;
A¹ and A² are taken together with their attached nitrogen to form a ring selected from the group consisting of:

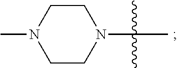

R_{b} is H, CH₂CF₃, or C₍₁₋₅₎alkyl;
and solvates, hydrates, tautomers, and pharmaceutically acceptable salts thereof. Another embodiment of the invention is a compound which is selected from the group consisting of:

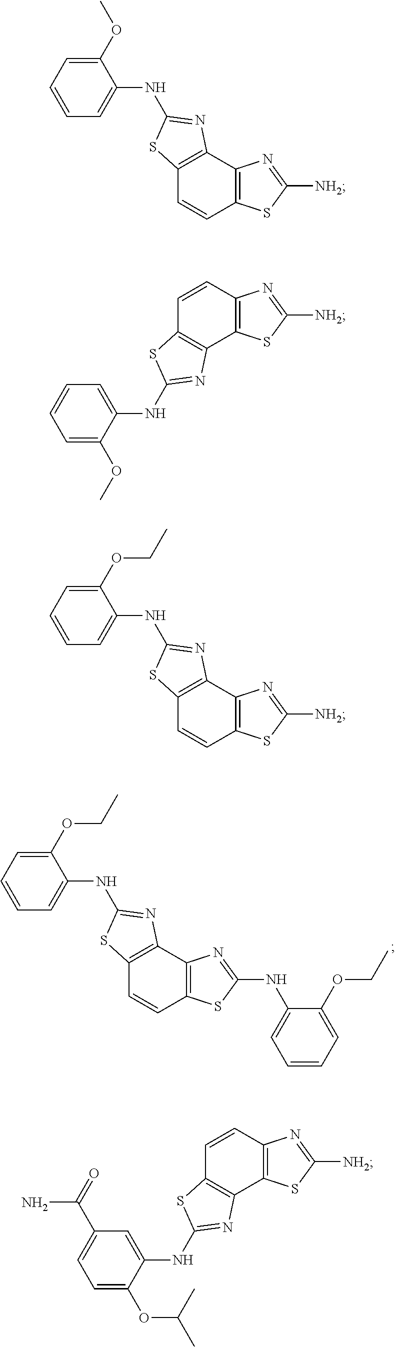

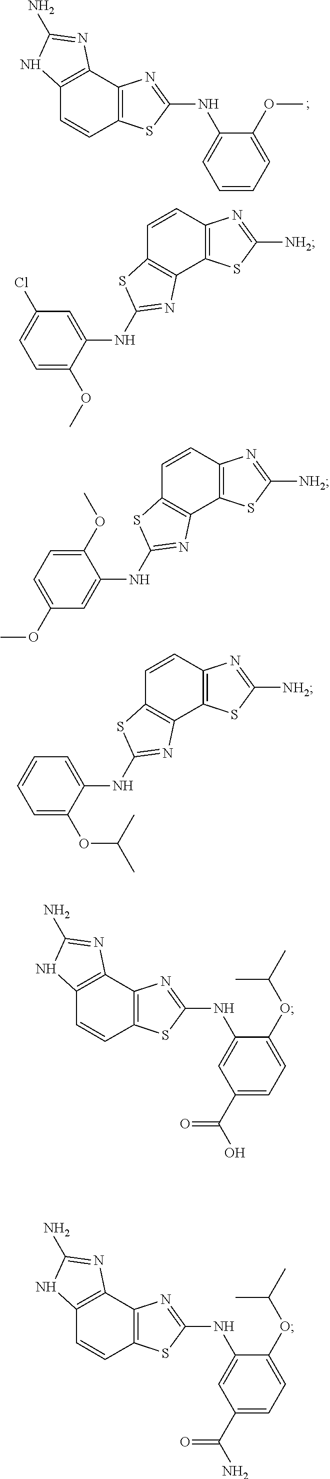

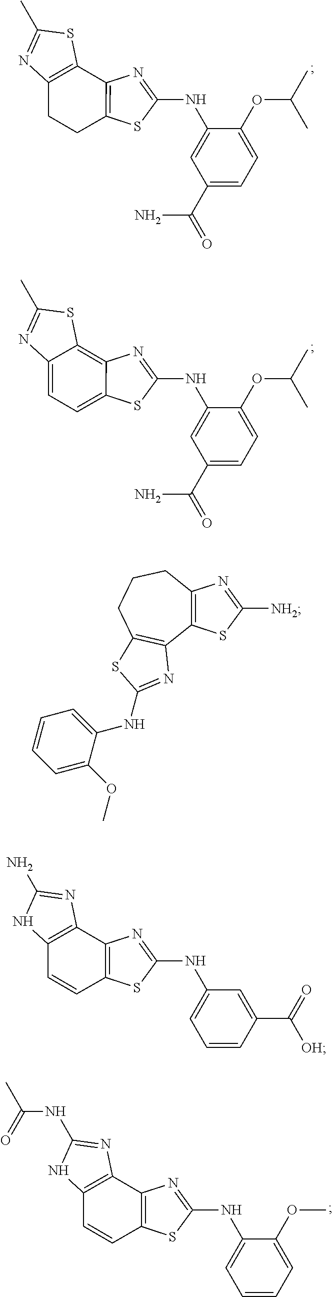

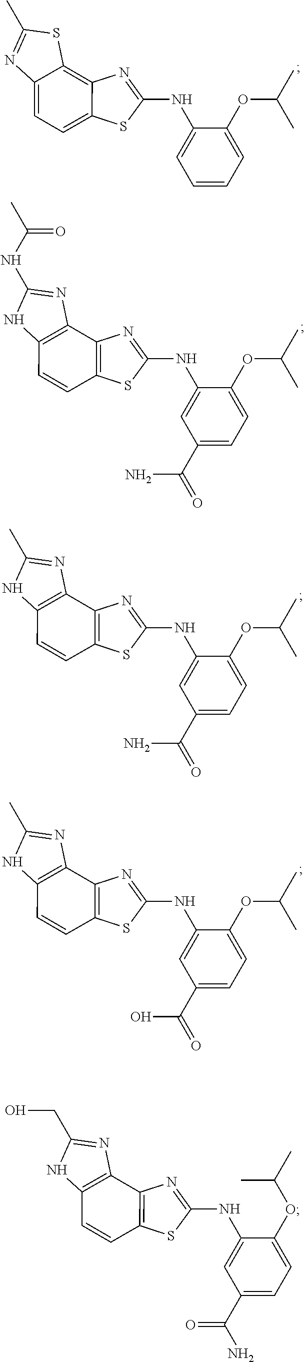

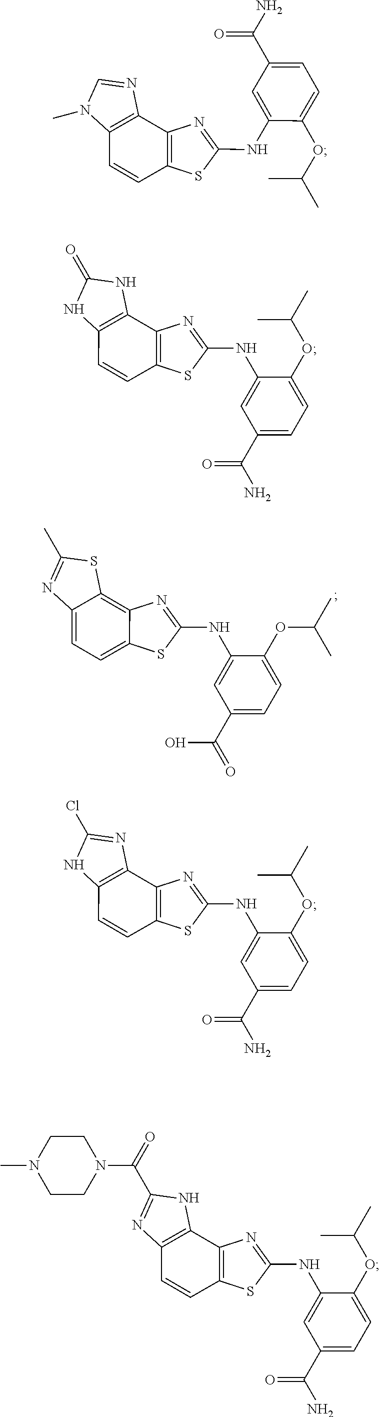

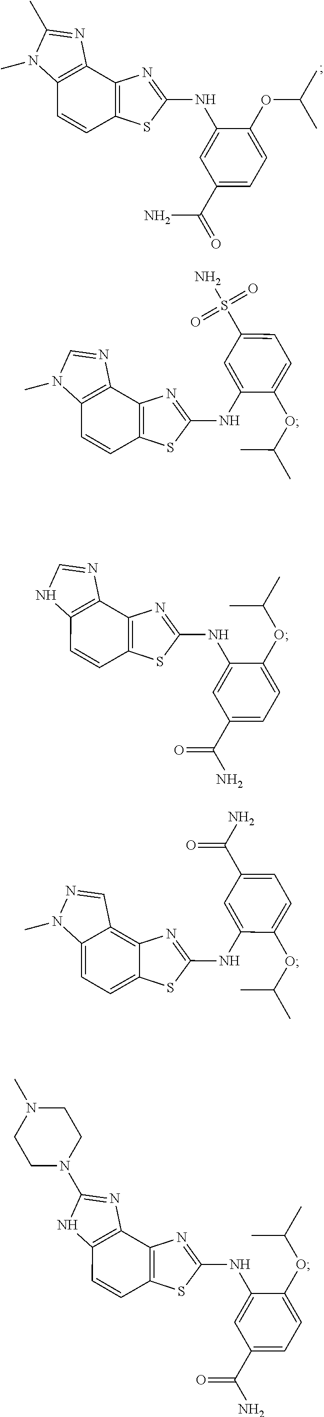

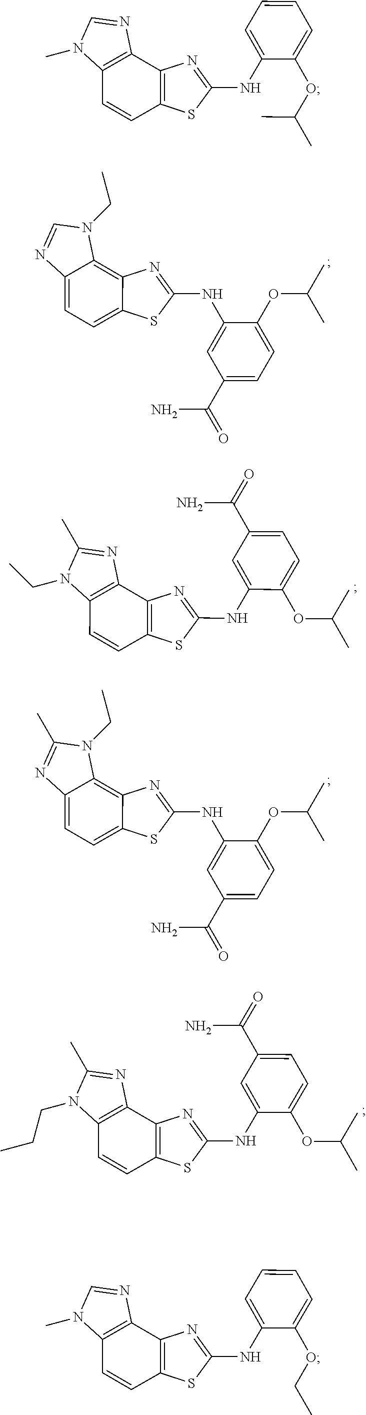

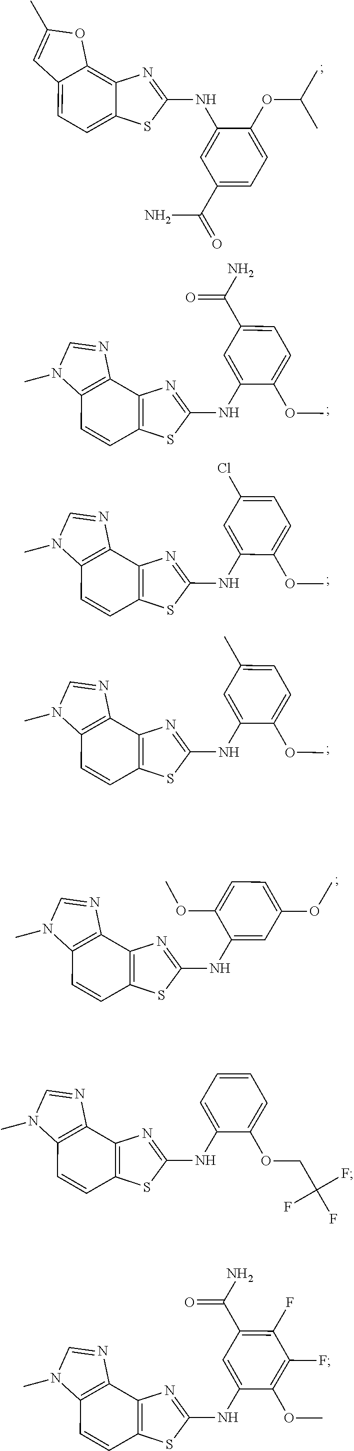

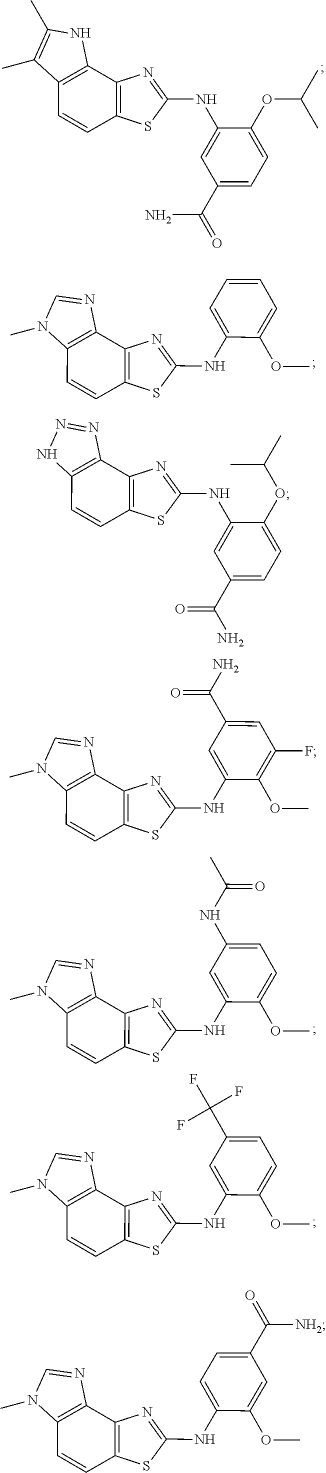

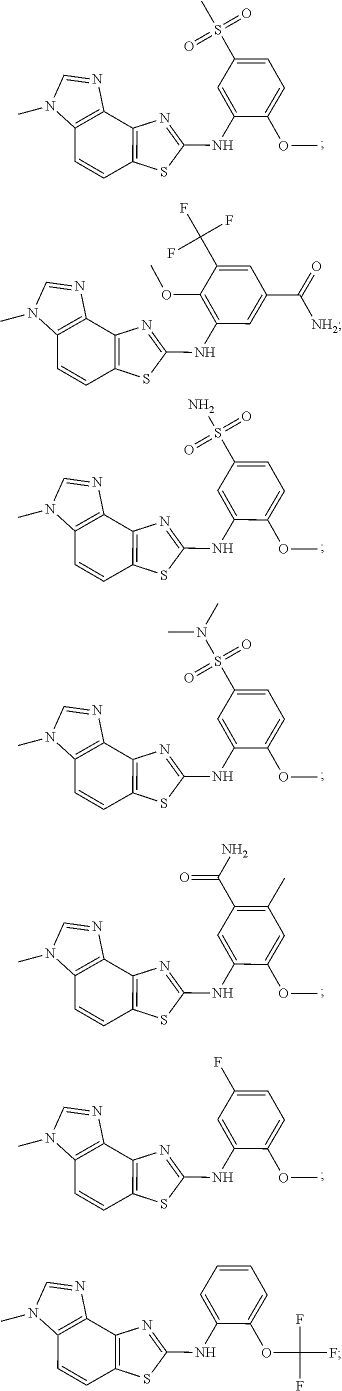

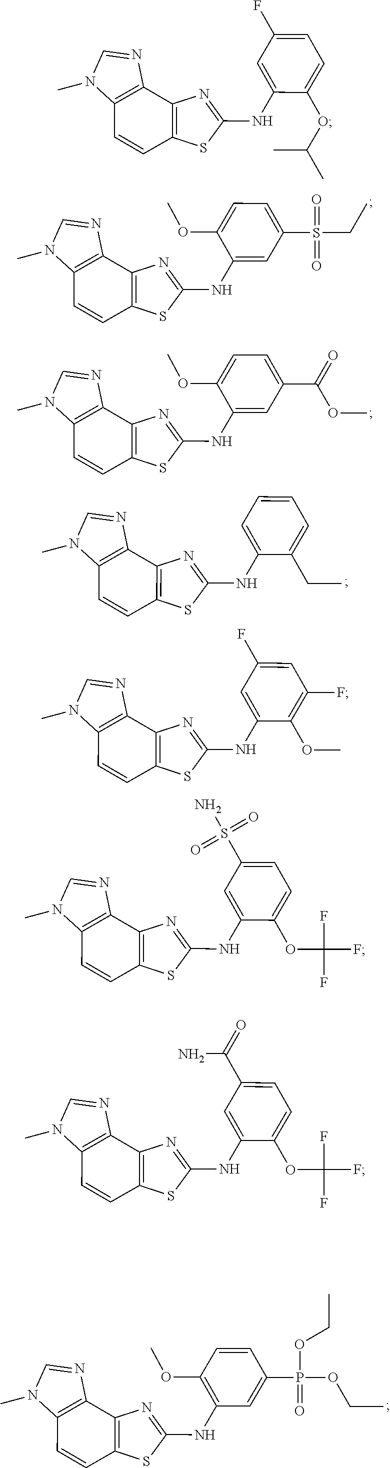

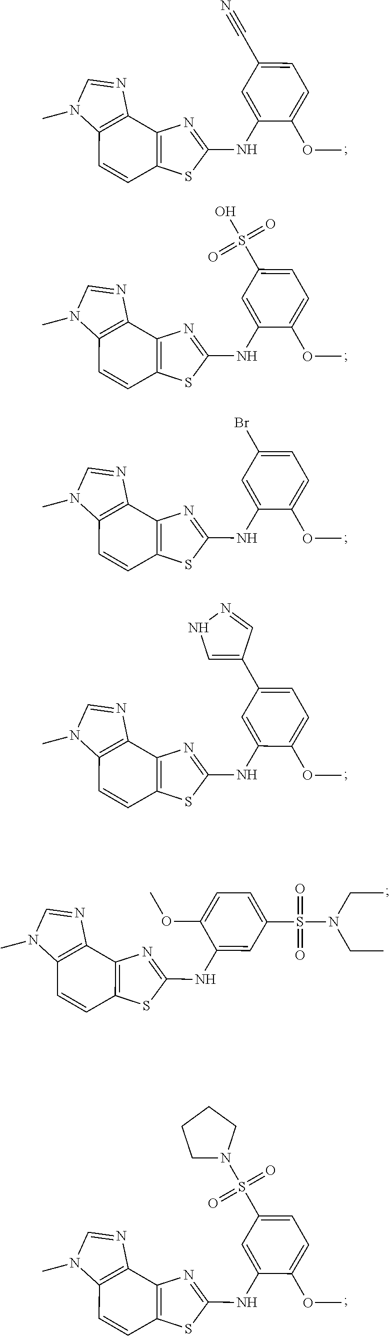

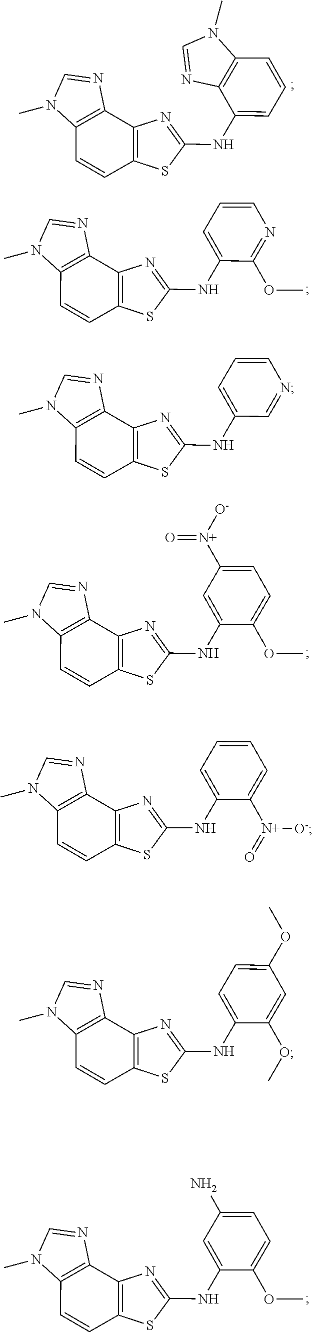

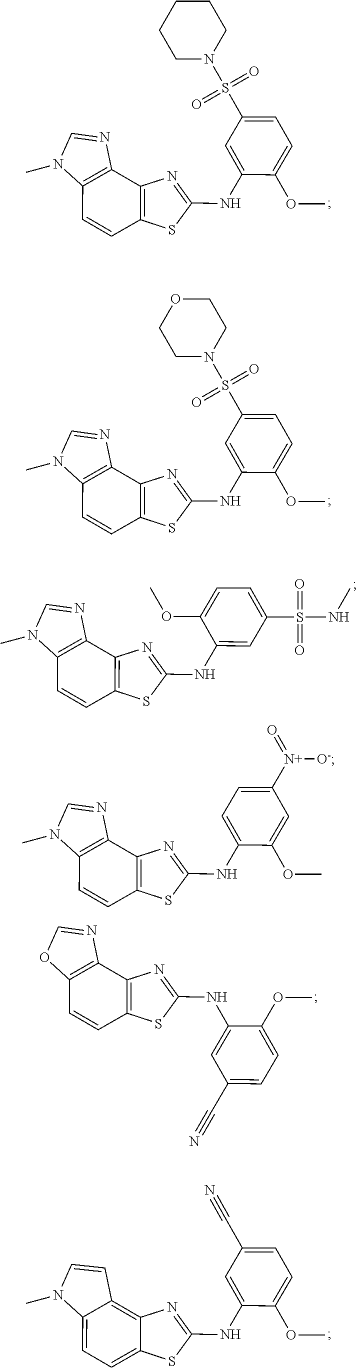

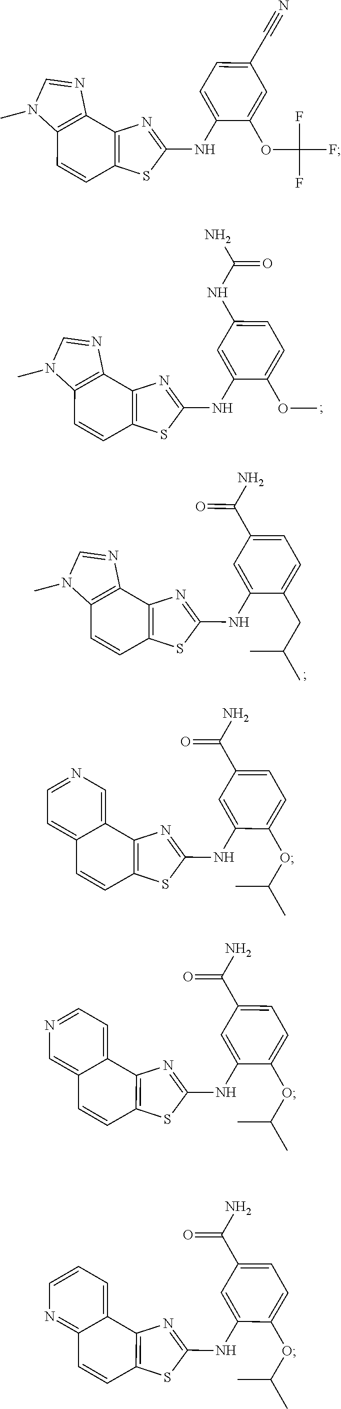

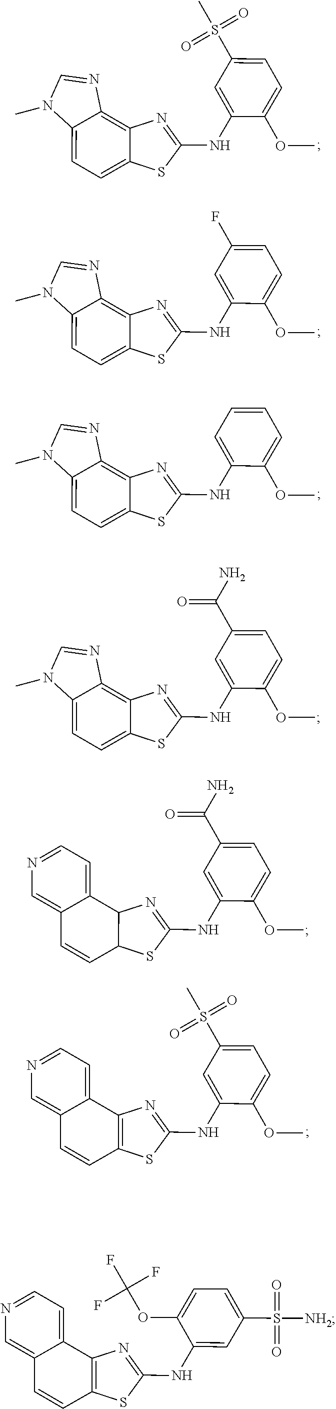

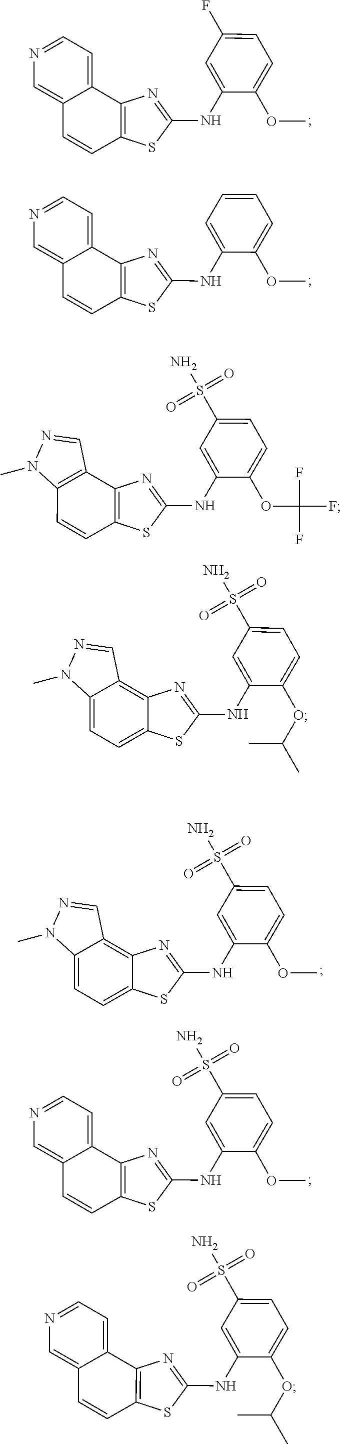

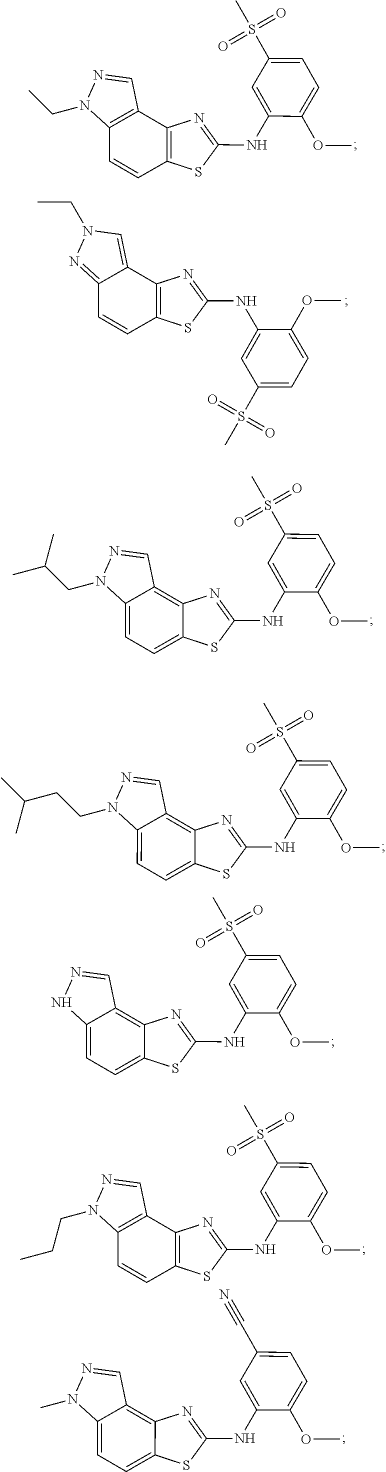

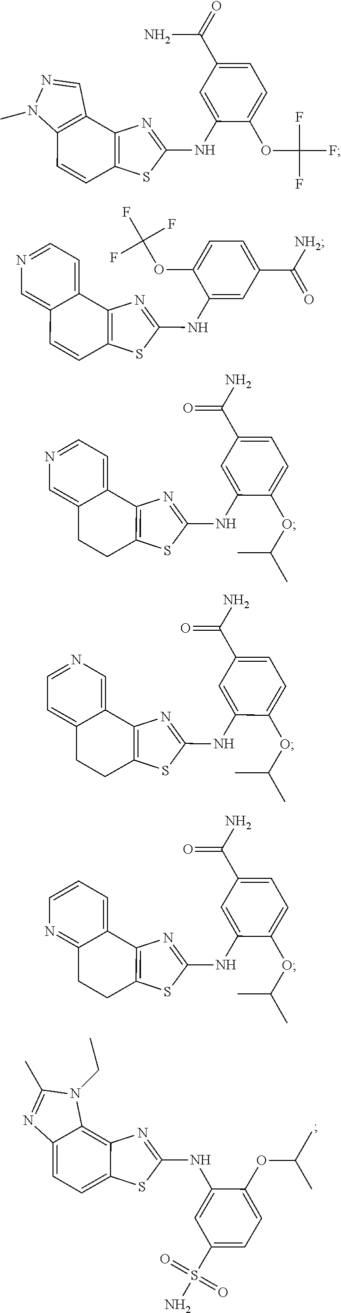

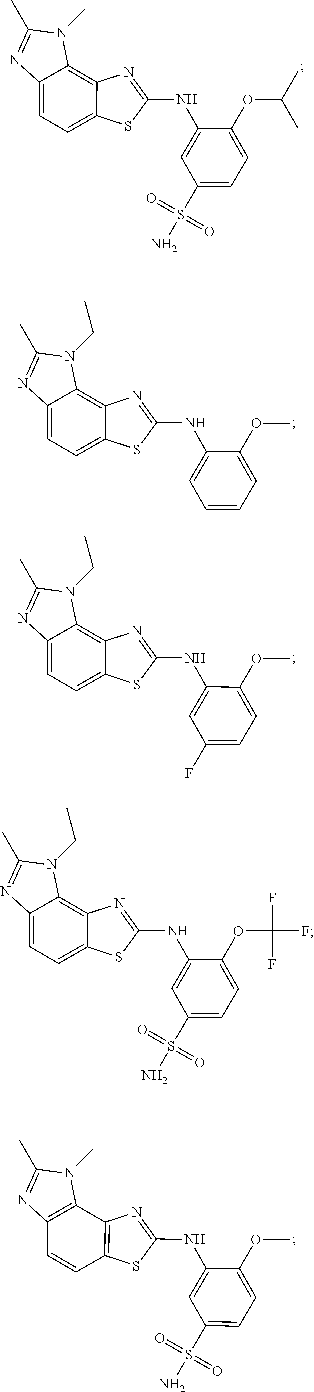

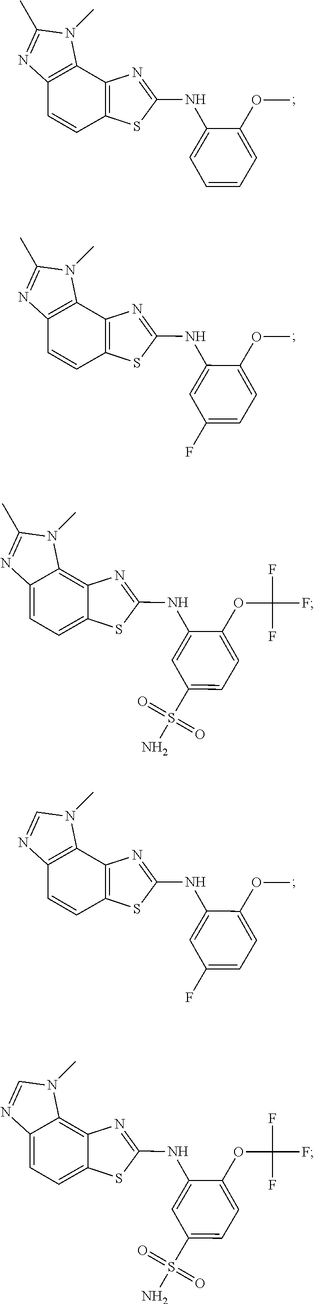

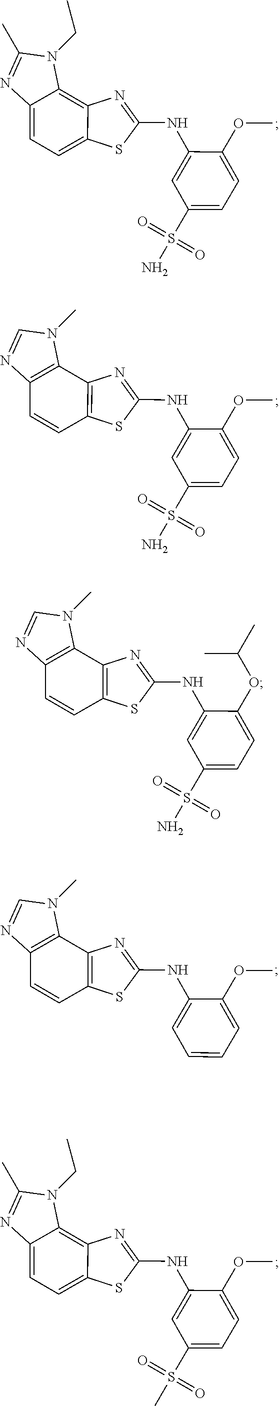

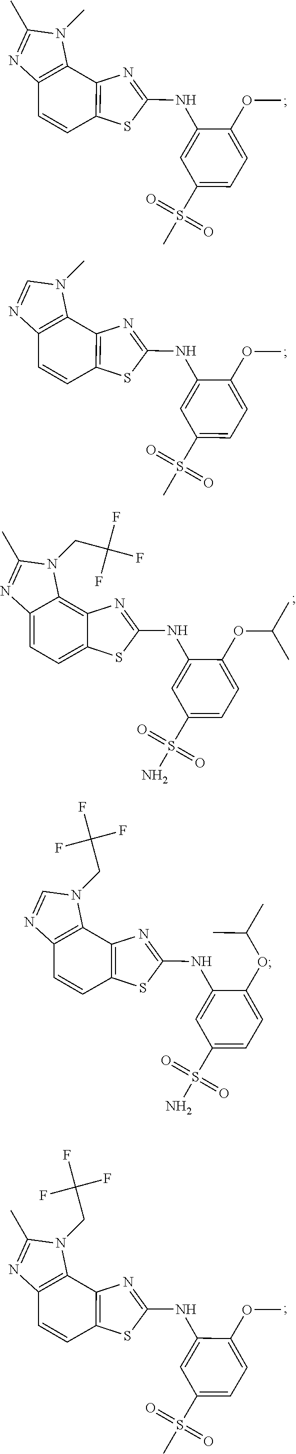

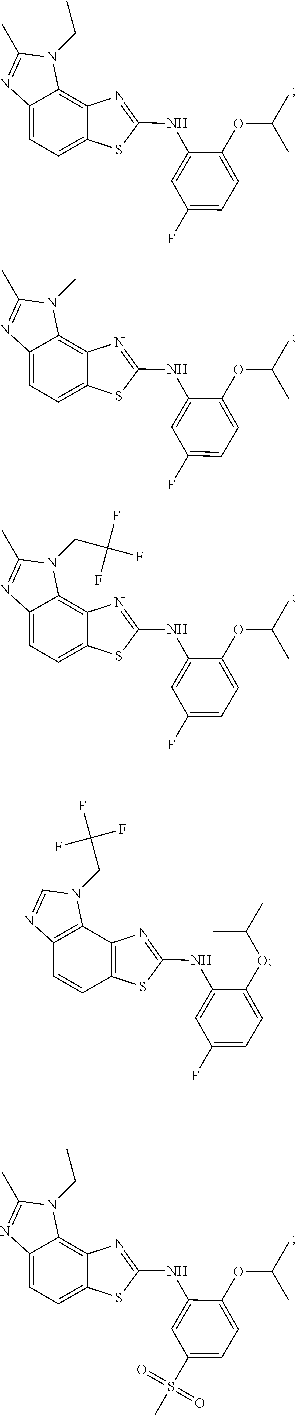

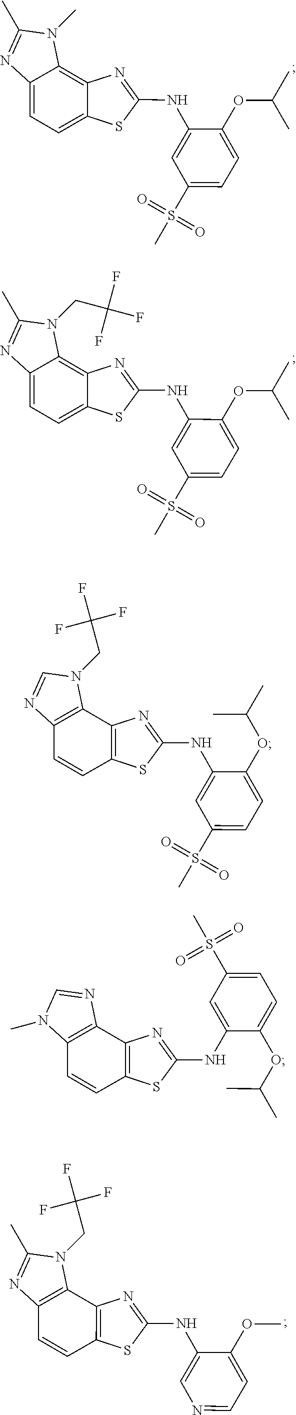

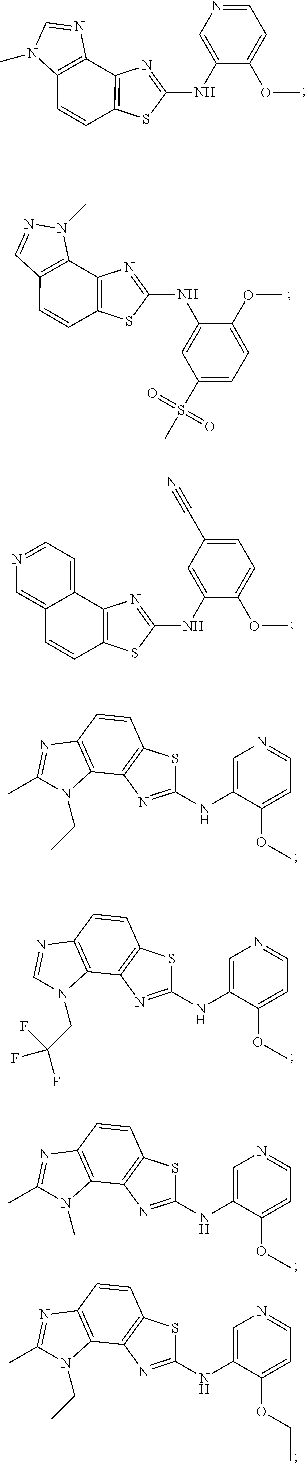

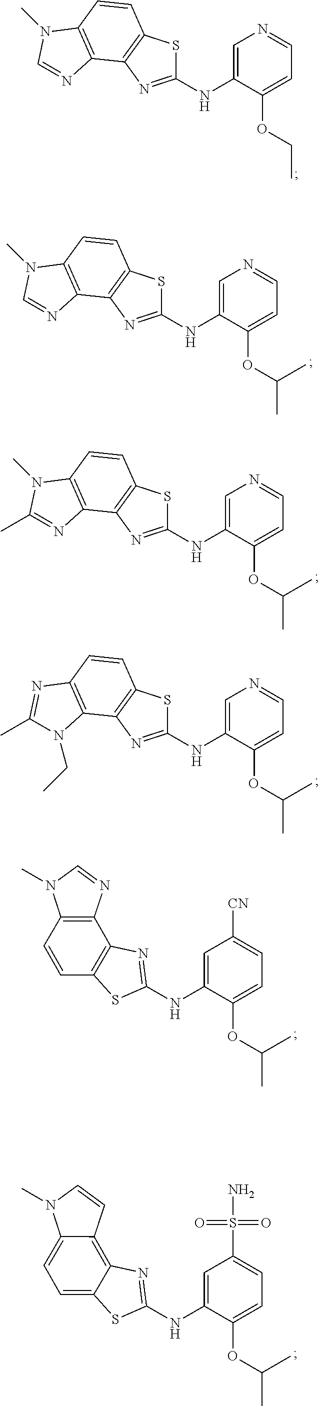

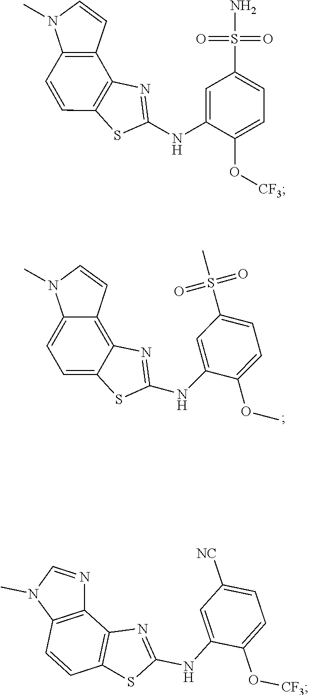

and solvates, hydrates, tautomers, and pharmaceutically acceptable salts thereof.

In another embodiment of the invention:

### R¹ is OCH(CH₃)₂;

### Q is C—R²;

### R² is H;

### R³ is H;

### J is C—R⁴;

### R⁴ is —CONH₂, —CO₂H, or —SO₂NH₂;

Z¹—Z² is —CH═CH—, —(CH₂)₂—, or —(CH₂)₃—, provided that if Z¹—Z² is CH₂—CH₂ then R⁴ is not H;
C¹ and C² are carbon atoms which are further substituted to make a ring selected from the group consisting of:

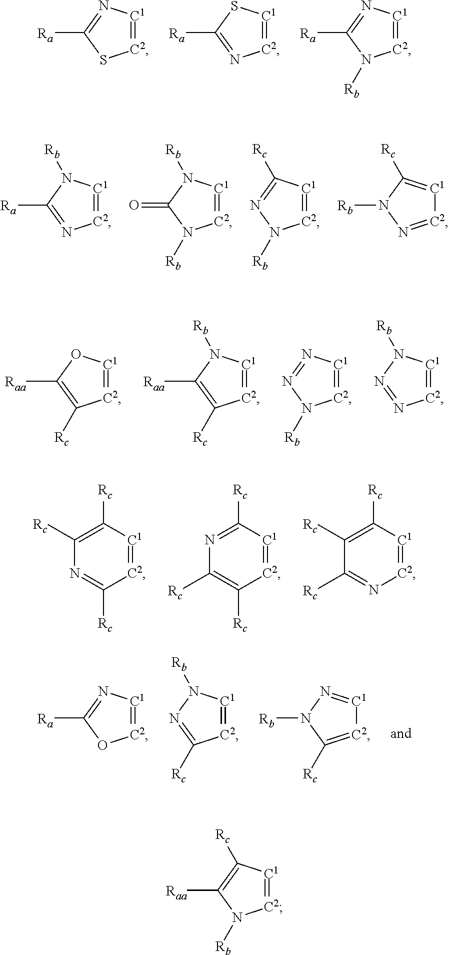

Rₐ is H, CF₃, CH₂CF₃, CH₂OH, Cl, Br, or C₍₁₋₆₎alkyl; or Rₐ may also be

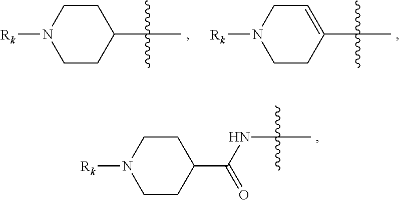

CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, pyridinyl, pyrimidinyl, pyrazinyl, NHPhOCH₂CH₃, NA¹A², C(O)NA¹A², SO₂NA¹A², SONA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍ₗ₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², or C₍₁₋₆₎alkylNA¹A², provided that R_{b} is H, CF₃, CH₂CF₃, C₍₁₋₆₎alkyl, or C₍₃₋₆₎cycloalkyl; wherein said

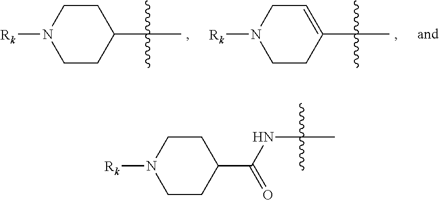

are optionally substituted with up to four methyl groups on two or more ring carbon atoms or optionally substituted with up to two CF₃ groups on any two ring carbon atoms;
A¹ is H, or C₍₁₋₃₎alkyl;
A² is H, C₍₁₋₆₎alkyl, C₍₃₋₆₎cycloalkyl,

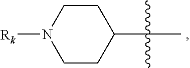

C₍₂₋₆₎alkylOH, C₍₂₋₆₎alkylOCH₃, SO₂C₍₁₋₄₎alkyl, C(O)Ph, C(O)C₍₁₋₄₎alkyl, pyrazinyl, or pyridyl, wherein said cycloalkyl, alkyl, pyrazinyl, pyridyl, or Ph groups may be optionally be substituted with two substituents selected from the group consisting of F, C₍₁₋₆₎alkyl, CF₃, pyrrolidinyl, CO₂H, C(O)NH₂, SO₂NH₂, OC₍₁₋₄₎alkyl, —CN, NO₂, OH, NH₂, NHC₍₁₋₄₎alkyl, N(C₍₁₋₄₎alkyl)₂; and said pyridyl, or Ph may be additionally be substituted with up to two halogens independently selected from the group consisting of: Cl, and Br; or A¹ and A² are taken together with their attached nitrogen to form a ring selected from the group consisting of:

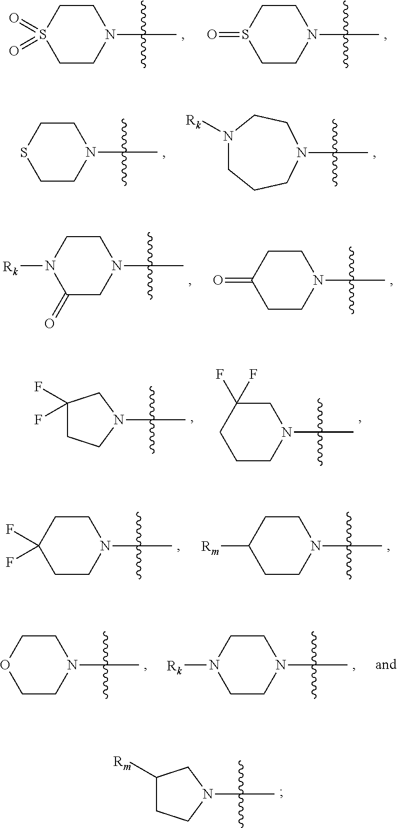

wherein any said A¹ and A² ring may be optionally substituted with up to four methyl groups on two or more ring carbon atoms or optionally substituted with up to two CF₃ groups on any two ring carbon atoms;
Rₖ is selected from the group consisting of H, CH₂CF₃, CH₂CH₂CF₃, C₍₁₋₆₎alkyl, COC₍₁₋₄₎alkyl, SO₂C₍₁₋₄₎alkyl, trifluoromethylpyridyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, C₍₁₋₆₎alkyl, F, or OH;
Rₐₐ is H, CF₃, CH₂CF₃, Cl, Br, C₍₁₋₆₎alkyl, CO₂H, CO₂C₍₁₋₄₎alkyl, C(O)C₍₁₋₄₎alkyl, C(O)Ph, SO₂C₍₁₋₄₎alkyl, SO₂NA¹A², SONA¹A², C(O)NA¹A², C(O)N(C₍₁₋₃₎alkyl)C₍₂₋₄₎alkylNA¹A², C(O)NHC₍₂₋₄₎alkylNA¹A², C₍₁₋₆₎alkylOC₍₁₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², or C₍₁₋₆₎alkylNA¹A²;
R_{b} is H, CF₃, CH₂CF₃, or C₍₁₋₆₎alkyl, or C₍₃₋₆₎cycloalkyl; or R_{b} may also be

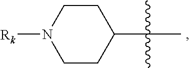

C(O)C₍₁₋₄₎alkyl, C(O)Ph, SO₂C₍₁₋₄₎alkyl, C₍₂₋₆₎alkylOC₍₁₋₆₎alkyl, C₍₂₋₆₎alkylOC₍₃₋₆₎cycloalkyl, C₍₂₋₆₎alkylOC₍₂₋₆₎alkylNA¹A², C₍₂₋₆₎alkylNHC₍₂₋₆₎alkylNA¹A², C₍₂₋₆₎alkylN(C₍₁₋₃₎alkyl)C₍₂₋₆₎alkylNA¹A², or C₍₂₋₆₎alkylNA¹A², provided that Rₐ is H, Cl, Br, CH₂OH, NH₂, CF₃, CH₂CF₃,
or C₍₁₋₆₎alkyl; wherein said

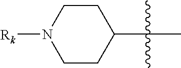

is optionally substituted with up to four methyl groups on two or more ring carbon atoms or optionally substituted with up to two CF₃ groups on any two ring carbon atoms;
R_{c} is H, C₍₁₋₃₎alkyl, or CF₃;
and solvates, hydrates, tautomers, and pharmaceutically acceptable salts thereof.

Another embodiment of the invention is a pharmaceutical composition, comprising a compound of Formula I and a pharmaceutically acceptable carrier.

Another embodiment of the invention is a pharmaceutical composition, comprising a compound listed in the Examples section of this specification and a pharmaceutically acceptable carrier.

The present invention also provides a method for preventing, treating or ameliorating an MMP9 mediated syndrome, disorder or disease comprising administering to a subject in need thereof an effective amount of a compound of Formula I or a form, composition or medicament thereof.

The present invention also provides a method for preventing, treating or ameliorating an MMP13 mediated syndrome, disorder or disease comprising administering to a subject in need thereof an effective amount of a compound of Formula I or a form, composition or medicament thereof.

The present invention also provides a method for preventing, treating or ameliorating an MMP9 mediated syndrome, disorder or disease wherein said syndrome, disorder or disease is associated with elevated MMP9 expression or MMP9 overexpression, or is a condition that accompanies syndromes, disorders or diseases associated with elevated MMP9 expression or MMP9 overexpression comprising administering to a subject in need thereof an effective amount of a compound of Formula I or a form, composition or medicament thereof.

The present invention also provides a method for preventing, treating or ameliorating an MMP13 mediated syndrome, disorder or disease wherein said syndrome, disorder or disease is associated with elevated MMP13 expression or MMP13 overexpression, or is a condition that accompanies syndromes, disorders or diseases associated with elevated MMP13 expression or MMP13 overexpression comprising administering to a subject in need thereof an effective amount of a compound of Formula I or a form, composition or medicament thereof.

The present invention provides a method of preventing, treating or ameliorating a syndrome, disorder or disease, wherein said syndrome, disorder or disease is selected from the group consisting of: neoplastic disorders, osteoarthritis, rheumatoid arthritis, cardiovascular diseases, gastric ulcer, pulmonary hypertension, chronic obstructive pulmonary disease, inflammatory bowel syndrome, periodontal disease, skin ulcers, liver fibrosis, emphysema, Marfan syndrome, stroke, multiple sclerosis, asthma, abdominal aortic aneurysm, coronary artery disease, idiopathic pulmonary fibrosis, renal fibrosis, and migraine, comprising administering to a subject in need thereof an effective amount of a compound of Formula I or a form, composition or medicament thereof.

The present invention provides a method of preventing, treating or ameliorating a neoplastic disorder, wherein said neoplastic disorder is ovarian cancer, comprising administering to a subject in need thereof an effective amount of a compound of Formula I or a form, composition or medicament thereof.

The present invention provides a method of preventing, treating or ameliorating a cardiovascular disease, wherein said cardiovascular disease is selected from the group consisting of: atherosclerotic plaque rupture, aneurysm, vascular tissue morphogenesis, coronary artery disease, and myocardial tissue morphogenesis, comprising administering to a subject in need thereof an effective amount of a compound of Formula I or a form, composition or medicament thereof.

The present invention provides a method of preventing, treating or ameliorating atherosclerotic plaque rupture, comprising administering to a subject in need thereof an effective amount of a compound of Formula I or a form, composition or medicament thereof.

The present invention provides a method of preventing, treating or ameliorating rheumatoid arthritis, comprising administering to a subject in need thereof an effective amount of a compound of Formula I or a form, composition or medicament thereof.

The present invention provides a method of preventing, treating or ameliorating asthma, comprising administering to a subject in need thereof an effective amount of a compound of Formula I or a form, composition or medicament thereof.

The present invention provides a method of preventing, treating or ameliorating chronic obstructive pulmonary disease, comprising administering to a subject in need thereof an effective amount of a compound of Formula I or a form, composition or medicament thereof.

The present invention provides a method of preventing, treating or ameliorating inflammatory bowel syndrome, comprising administering to a subject in need thereof an effective amount of a compound of Formula I or a form, composition or medicament thereof.

The present invention provides a method of preventing, treating or ameliorating abdominal aortic aneurism, comprising administering to a subject in need thereof an effective amount of a compound of Formula I or a form, composition or medicament thereof.

The present invention provides a method of preventing, treating or ameliorating osteoarthritis, comprising administering to a subject in need thereof an effective amount of a compound of Formula I or a form, composition or medicament thereof.

The present invention provides a method of preventing, treating or ameliorating idiopathic pulmonary fibrosis, comprising administering to a subject in need thereof an effective amount of a compound of Formula I or a form, composition or medicament thereof.

The invention also relates to methods of inhibiting MMP9 activity in a mammal by administration of an effective amount of at least one compound of Formula I.

The invention also relates to methods of inhibiting MMP13 activity in a mammal by administration of an effective amount of at least one compound of Formula I.

In another embodiment, the invention relates to a compound as described in the Examples section for use as a medicament, in particular, for use as a medicament for treating a MMP9 mediated syndrome, disorder or disease.

In another embodiment, the invention relates to the use of a compound as described in the Examples section for the preparation of a medicament for the treatment of a disease associated with an elevated or inappropriate MMP9 activity.

In another embodiment, the invention relates to a compound as described in the Examples section for use as a medicament, in particular, for use as a medicament for treating a MMP13 mediated syndrome, disorder or disease.

In another embodiment, the invention relates to the use of a compound as described in the Examples section for the preparation of a medicament for the treatment of a disease associated with an elevated or inappropriate MMP13 activity.

### DEFINITIONS

The term “alkyl” refers to both linear and branched chain radicals of up to 12 carbon atoms, preferably up to 6 carbon atoms, unless otherwise indicated, and includes, but is not limited to, methyl, ethyl, propyl, isopropyl, butyl, isobutyl, sec-butyl, tert-butyl, pentyl, isopentyl, hexyl, isohexyl, heptyl, octyl, 2,2,4-trimethylpentyl, nonyl, decyl, undecyl and dodecyl. Any alkyl group may be optionally substituted with one OCH₃, one OH, or up to two fluorine atoms.

The term “alkoxy” refers to a saturated branched or straight chain monovalent hydrocarbon alcohol radical derived by the removal of the hydrogen atom from the hydroxide oxygen substituent on a parent alkane. Examples include C₍₁₋₆₎alkoxy or C₍₁₋₄₎alkoxy groups. Any alkoxy group may be optionally substituted with one OCH₃, one OH, or up to two fluorine atoms.

The term “C_{(a-b)}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₍₁₋₄₎ denotes a radical containing 1, 2, 3 or 4 carbon atoms.

The term “cycloalkyl” refers to a saturated or partially unsaturated monocyclic or bicyclic hydrocarbon ring radical derived by the removal of one hydrogen atom from a single ring carbon atom. Typical cycloalkyl radicals include cyclopropyl, cyclobutyl, cyclopentyl, cyclopentenyl, cyclohexyl, cyclohexenyl, cycloheptyl and cyclooctyl. Additional examples include C₍₃₋₆₎cycloalkyl, C₍₅₋₈₎cycloalkyl, decahydronaphthalenyl, and 2,3,4,5,6,7-hexahydro-1H-indenyl. Any cycloalkyl group may be optionally substituted with one OCH₃, one OH, or up to two fluorine atoms.

### ABBREVIATIONS

Herein and throughout this application, the following abbreviations may be used.

### Ac —C(O)CH₃

aq. aqueous
conc. concentrated
DDQ 2,3-dichloro-5,6-dicyanobenzoquinone
DIPEA diisopropylethylamine
DMF dimethylformamide
DMSO dimethylsulfoxide
Et ethyl
g gram
h hours
hept heptanes
HPLC high pressure liquid chromatography

### KHMDS ((CH₃)₃Si)₂NK

M molar
Me methyl
mL milliliter
mmol millimole
mg milligram
min minutes
N normal

### NMP N-methylpyrrolidinone

NMR nuclear magnetic resonance
Pd(dppf)Cl₂ [1,1′-bis(diphenylphosphino)ferrocene]dichloropalladium(II)
Ph phenyl
iPr isopropyl
PS polystyrene
psi pounds/square inch
RP-HPLC reverse phase high pressure liquid chromatography
RT or rt room temperature
sat. saturated
TBDMS tert-butyldimethylsilyl
TFA trifluoroacetic acid
THF tetrahydrofuran
TLC thin layer chromatography
v volume

Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate (SEH), sodium hydroxide, triethanolamine or zinc.

### Methods of Use

The present invention is directed to a method for preventing, treating or ameliorating a MMP9 and/or MMP13 mediated syndrome, disorder or disease comprising administering to a subject in need thereof an effective amount of a compound of Formula I or a form, composition or medicament thereof.

Examples of a MMP9 and/or MMP13 mediated syndrome, disorder or disease for which the compounds of Formula I are useful include angiogenesis, osteoarthritis, rheumatoid arthritis, gastric ulcers, pulmonary hypertension, chronic obstructive pulmonary disorder, inflammatory bowel syndrome, periodontal disease, skin ulcers, liver fibrosis, emphysema, Marfan syndrome, stroke, multiple sclerosis, abdominal aortic aneurysm, coronary artery disease, idiopathic pulmonary fibrosis, renal fibrosis, migraine, and cardiovascular disorders including: atherosclerotic plaque, ruptive aneurysm, vascular tissue morphogenesis, and myocardial tissue morphogenesis.

The term “administering” with respect to the methods of the invention, means a method for therapeutically or prophylactically preventing, treating or ameliorating a syndrome, disorder or disease as described herein by using a compound of Formula I or a form, composition or medicament thereof. Such methods include administering an effective amount of said compound, compound form, composition or medicament at different times during the course of a therapy or concurrently in a combination form. The methods of the invention are to be understood as embracing all known therapeutic treatment regimens.

The term “subject” refers to a patient, which may be animal, typically a mammal, typically a human, which has been the object of treatment, observation or experiment. In one aspect of the invention, the subject is at risk of (or susceptible to) developing a syndrome, disorder or disease that is associated with elevated MMP9 and/or MMP13 expression or MMP9 and/or MMP13 overexpression, or a patient with an inflammatory condition that accompanies syndromes, disorders or diseases associated with elevated MMP9 and/or MMP13 expression or MMP9 and/or MMP13 overexpression.

The term “therapeutically effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

When employed as inhibitors of pro-matrix metalloproteinase activation, the compounds of the invention may be administered in an effective amount within the dosage range of about 0.5 mg to about 10 g, preferably between about 0.5 mg to about 5 g, in single or divided daily doses. The dosage administered will be affected by factors such as the route of administration, the health, weight and age of the recipient, the frequency of the treatment and the presence of concurrent and unrelated treatments.

It is also apparent to one skilled in the art that the therapeutically effective dose for compounds of the present invention or a pharmaceutical composition thereof will vary according to the desired effect. Therefore, optimal dosages to be administered may be readily determined by one skilled in the art and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this invention.

The compounds of Formula I may be formulated into pharmaceutical compositions comprising any known pharmaceutically acceptable carriers. Exemplary carriers include, but are not limited to, any suitable solvents, dispersion media, coatings, antibacterial and antifungal agents and isotonic agents. Exemplary excipients that may also be components of the formulation include fillers, binders, disintegrating agents and lubricants.

The pharmaceutically-acceptable salts of the compounds of Formula I include the conventional non-toxic salts or the quaternary ammonium salts which are formed from inorganic or organic acids or bases. Examples of such acid addition salts include acetate, adipate, benzoate, benzenesulfonate, citrate, camphorate, dodecylsulfate, hydrochloride, hydrobromide, lactate, maleate, methanesulfonate, nitrate, oxalate, pivalate, propionate, succinate, sulfate and tartrate. Base salts include ammonium salts, alkali metal salts such as sodium and potassium salts, alkaline earth metal salts such as calcium and magnesium salts, salts with organic bases such as dicyclohexylamino salts and salts with amino acids such as arginine. Also, the basic nitrogen-containing groups may be quaternized with, for example, alkyl halides.

The pharmaceutical compositions of the invention may be administered by any means that accomplish their intended purpose. Examples include administration by parenteral, subcutaneous, intravenous, intramuscular, intraperitoneal, transdermal, buccal or ocular routes. Alternatively or concurrently, administration may be by the oral route. Suitable formulations for parenteral administration include aqueous solutions of the active compounds in water-soluble form, for example, water-soluble salts, acidic solutions, alkaline solutions, dextrose-water solutions, isotonic carbohydrate solutions and cyclodextrin inclusion complexes.

The present invention also encompasses a method of making a pharmaceutical composition comprising mixing a pharmaceutically acceptable carrier with any of the compounds of the present invention. Additionally, the present invention includes pharmaceutical compositions made by mixing a pharmaceutically acceptable carrier with any of the compounds of the present invention. As used herein, the term “composition” is intended to encompass a product comprising the specified ingredients in the specified amounts, as well as any product which results, directly or indirectly, from combinations of the specified ingredients in the specified amounts.

### Polymorphs and Solvates

Furthermore, the compounds of the present invention may have one or more polymorph or amorphous crystalline forms and as such are intended to be included in the scope of the invention. In addition, the compounds may form solvates, for example with water (i.e., hydrates) or common organic solvents. As used herein, the term “solvate” means a physical association of the compounds of the present invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. The term “solvate” is intended to encompass both solution-phase and isolatable solvates. Non-limiting examples of suitable solvates include ethanolates, methanolates, and the like.

It is intended that the present invention include within its scope polymorphs and solvates of the compounds of the present invention. Thus, in the methods of treatment of the present invention, the term “administering” shall encompass the means for treating, ameliorating or preventing a syndrome, disorder or disease described herein with the compounds of the present invention or a polymorph or solvate thereof, which would obviously be included within the scope of the invention albeit not specifically disclosed.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

### General Schemes

Compounds of Formula I can be prepared by methods known to those who are skilled in the art. The following schemes are only meant to represent examples of the invention and are in no way meant to be a limit of the invention.

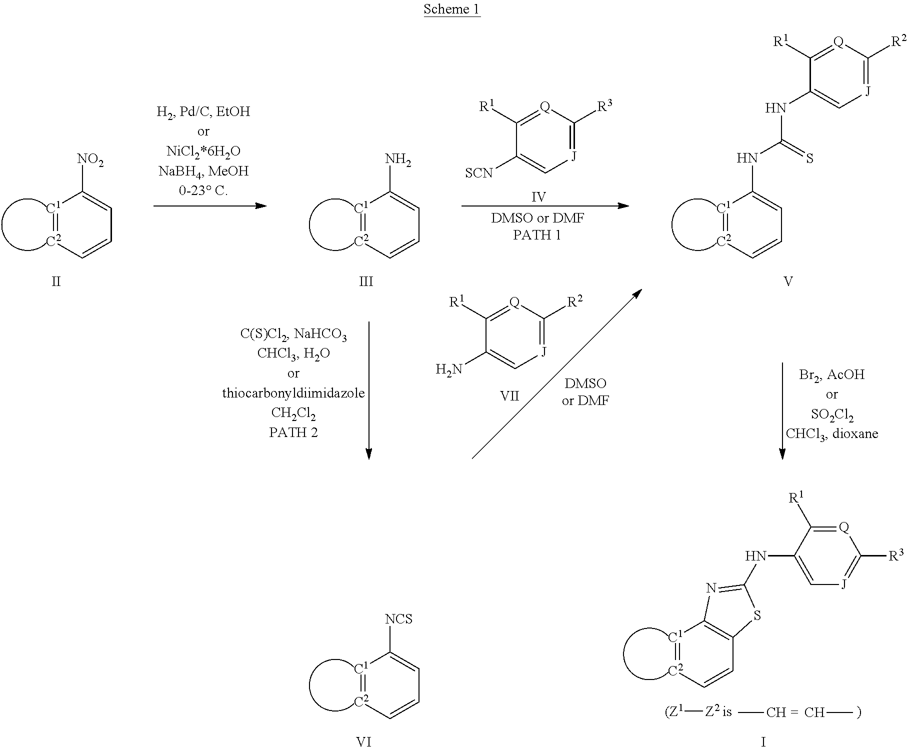

Scheme 1 illustrates synthetic routes leading to compounds of Formula I, where Z¹—Z² is —CH═CH—. Nitro-substituted heterocycle II is reduced, for instance by catalytic hydrogenation or using the reagent generated from nickel (II) chloride and sodium borohydride, to amino heterocycle III. As path 1 illustrates, compound III can be reacted with aryl isothiocyanate IV to provide thiourea V. The reaction is typically conducted in a polar aprotic solvent, such as DMSO, DMF or THF, at a temperature between 20° C. and 100° C. Thiourea V may be activated for cyclization to a compound of Formula I by treatment with a number of reagents, including bromine in acetic acid or sulfuryl chloride in chloroform, dioxane, or a mixture of the two solvents. The cyclization reaction is typically conducted at a temperature between −20° C. and 100° C. An alternative route to compounds of Formula I, where Z¹—Z² is —CH═CH—, involves conversion of amino heterocycle III to isothiocyanate VI. This transformation can be accomplished by treatment with thiophosgene and an inorganic base, such as sodium carbonate, in a biphasic mixture of chloroform and water, or by reaction with 1,1′-thiocarbonyldiimidazole in dichloromethane. Isothiocyanate VI undergoes reaction with aniline VII, typically in a polar aprotic solvent such as DMF or DMSO at a temperature between 20° C. and 100° C., to form thiourea V, which may be converted to a compound of Formula I as described above.

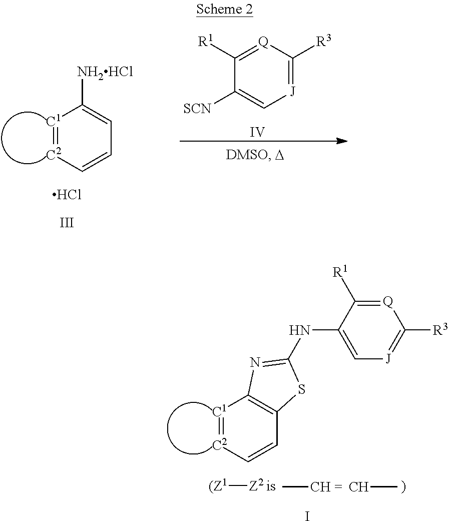

Scheme 2 depicts an alternative route to compounds of Formula I, where Z¹—Z² is —CH═CH—. The hydrochloride salt of amino heterocycle III can be converted in one step to a compound of Formula I by reaction with aryl isothiocyanate IV in DMSO. The reaction can be conducted at a temperature between 50 and 100° C.

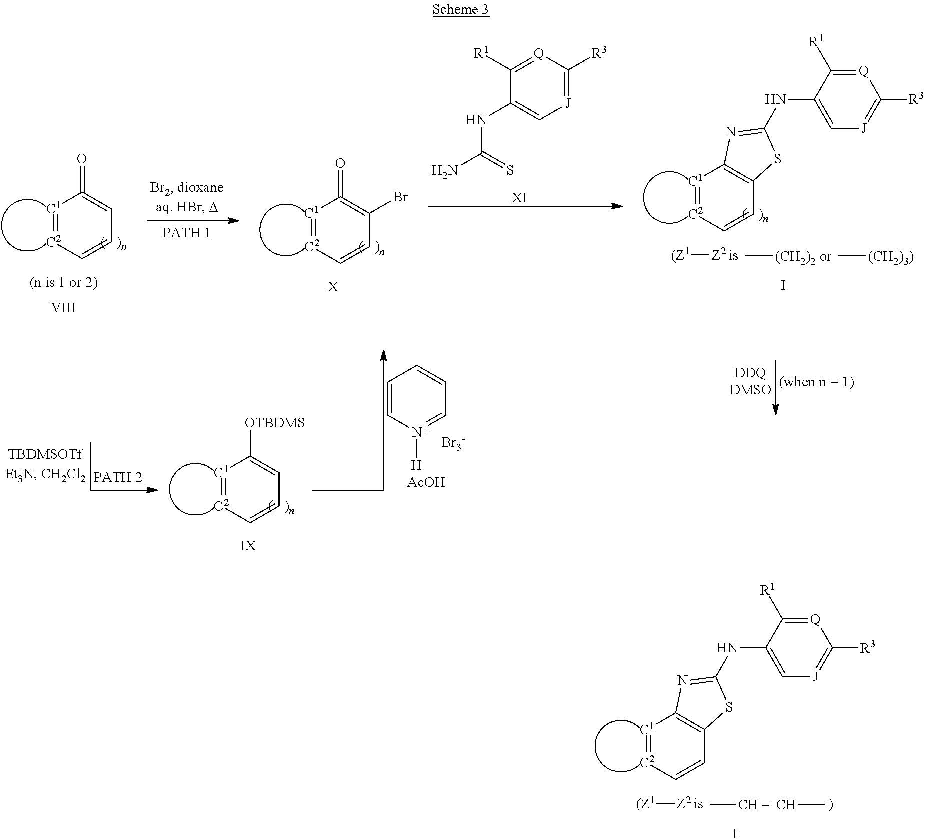

Synthetic routes to compounds of Formula I, where Z¹—Z² is —(CH₂)₂— or —(CH₂)₃— are illustrated in Scheme 3. Ketone VIII, where n is one or two, may be brominated, for instance by heating with bromine in dioxane, with or without addition of aqueous hydrobromic acid, to form bromide X, as shown in Path 1. Alternatively, according to Path 2, VIII can be converted to tert-butyldimethylsilyl enol ether IX by reaction with tert-butyldimethylsilyl triflate and a base, such as triethylamine. Enol ether IX may be brominated by treatment with pyridinium tribromide in acetic acid. Bromide X is condensed with thiourea XI to form a compound of Formula I, where Z¹—Z² is —(CH₂)₂— or —(CH₂)₃—. When n is one, the compound of Formula I, where Z¹—Z² is —(CH₂)₂—, may be treated with an oxidant, such as DDQ, to aromatize the central ring of the tricyclic scaffold. This route thus provides an alternative method for preparation of compounds of Formula I, where Z¹—Z² is —CH═CH—.

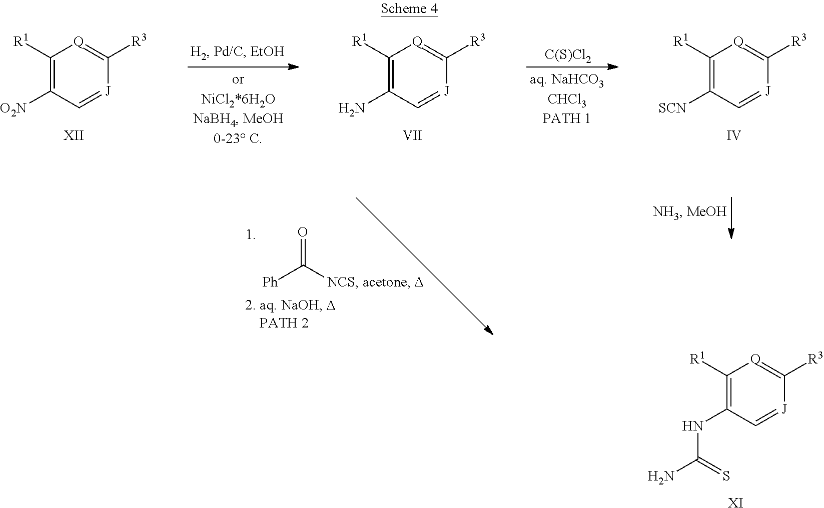

Intermediates VII, IV, and XI (as used in Schemes 1-3) may be commercially available, or may be prepared as detailed in Scheme 4. Nitro aromatic XII is reduced to the corresponding aniline VII, for instance by catalytic reduction under a hydrogen atmosphere or using NiCl₂*6H₂O/NaBH₄. As shown in Path 1, VII is converted to aryl isothiocyanate IV on treatment with thiophosgene and an inorganic base, such as sodium carbonate, in a biphasic mixture of chloroform and water. Reaction of IV with ammonia provides thiourea XI. Alternatively, VII may be converted to thiourea XI by reaction with benzoyl isothiocyanate, typically in refluxing acetone, followed by hydrolysis under basic aqueous conditions (Path 2).

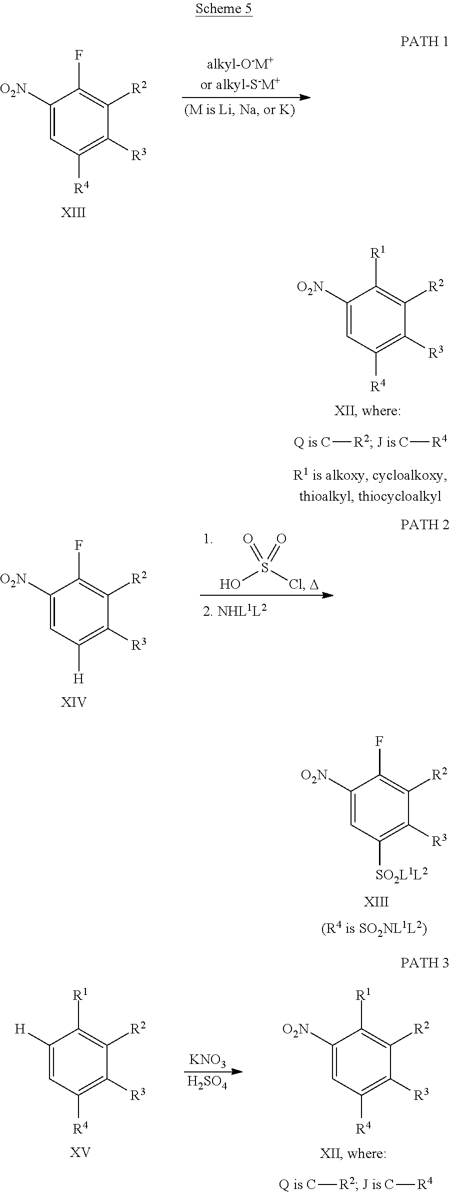

Additional nitro aromatic compounds XII, used as starting materials in Scheme 4, may be prepared by the routes shown in Scheme 5. A 2-nitrofluoro benzene XIII can be reacted with a metal alkoxide or thiolate to yield XII, where R¹ is alkoxy, cycloalkoxy, thialkyl, or thicycloalkyl as defined in Formula I (path 1). As shown in path 2, in the case where R⁴ is SO₂NL¹L², the required starting material XIII may be obtained by heating 2-fluoronitro benzene XIV (unsubstituted para to the fluorine) in neat chlorosulfonic acid, typically at reflux, followed by treatment of the aryl sulfonyl chloride intermediate with an amine NHL¹L². Additional nitro aromatic compounds XII may be obtained by treatment of substituted aryls XV with a nitrating reagent, such as KNO₃/H₂SO₄, HNO₃/H₂SO₄, or HNO₃/Ac₂O (path 3). Those skilled in the art will recognize that path 3 is preferably employed when nitration is desired to occur at a position ortho or para to electron-donating substituents, such as alkoxy or alkyl, and meta to electron-withdrawing substituents, such as CONH₂.

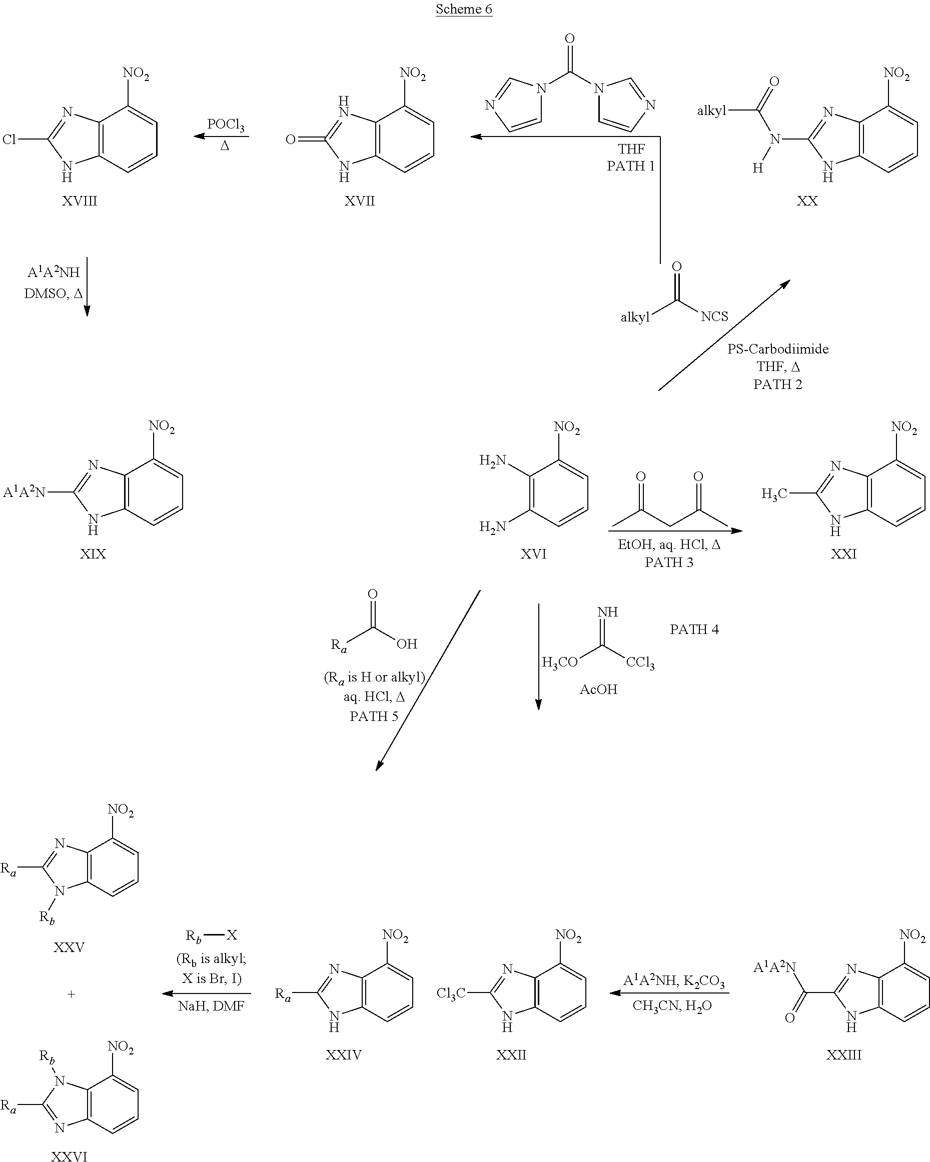

Scheme 6 illustrates routes to nitro heteroaromatics XVII to XXVI starting from 3-nitro-1,2-phenylenediamine XVI. These intermediates can each be elaborated to compounds of Formula I, where Z¹—Z² is —CH═CH—, by the methods described in Schemes 1 and 2. Following path 1, XVI is converted to 1,3-dihydro-benzoimidazol-2-one XVII by reaction with 1,1′-carbonyldiimidazole. Heating XVII in neat POCl₃ results in formation of 2-chlorobenzimidazole XVIII. Reaction of XVIII with amine A¹A²NH affords 2-aminobenzimidazole XIX. Phenylenediamine XVI can be converted into acylated 2-aminobenzimidazole XX by heating with an acyl isothiocyanate in the presence of a resin-bound carbodiimide in THF (Path 2). It will be recognized that such compounds also provide access to compounds of Formula I, where —C¹═C²— and the ring to which they are attached is imidazolyl and Rₐ is NH₂, by hydrolysis of the acyl group, which occurs upon heating in aqueous base, such as 1 N sodium hydroxide, in the presence of an organic co-solvent, such as methanol. According to path 3,2-methylbenzimidazole XXI is accessible by heating XVI to reflux with pentanedione in a mixture of ethanol and aqueous hydrochloric acid. Treatment of XVI with methyl 2,2,2-trichloroacetimidate in acetic acid affords 2-trichlorobenzimidazole XXII, which undergoes reaction with an amine A¹A²NH and an inorganic base, such as potassium carbonate, in a mixture of water and acetonitrile, to form 2-amido substituted benzimidazoles XXIII (path 4). Finally, XVI can be condensed with a carboxylic acid Rₐ—CO₂H, where Rₐ is H or alkyl, by heating in aqueous hydrochloric acid to yield benzimidazole XXIV (path 5). XXIV may be alkylated by reaction with an alkyl bromide or iodide using a base, such as sodium hydride, in a polar aprotic solvent. The regioisomeric alkylation products XXV and XXVI can be separated by chromatography before elaboration to compounds of Formula I.

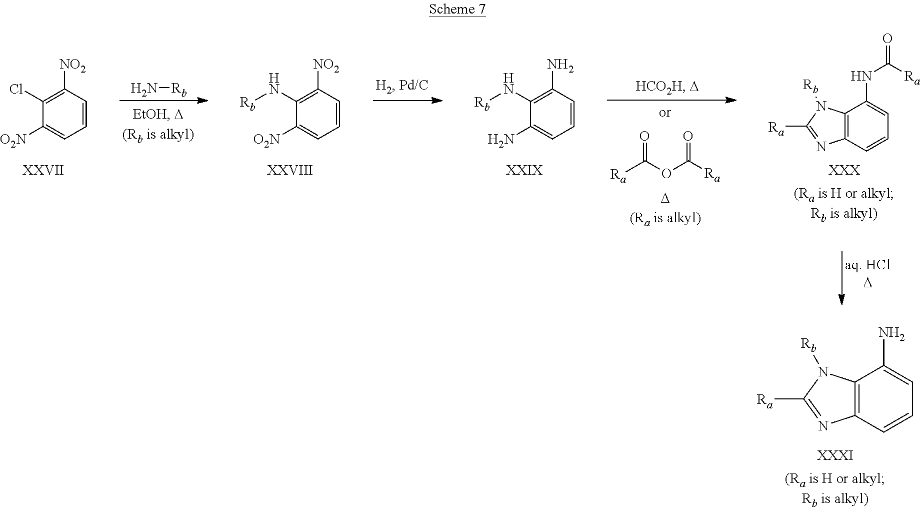

Scheme 7 depicts a route to aminobenzimidazoles XXXI, intermediates which can be converted to compounds of Formula I, where Z¹—Z² is —CH═CH—, by the routes shown in Scheme 1 and 2. 2-Chloro-1,3-dinitrobenzene XXVII is heated with an amine R_{b}—NH₂, where R_{b} is alkyl, to provide 2-amino-1,3-dinitrobenzene XXVIII. The nitro groups are both reduced, for instance by hydrogenation in the presence of a palladium catalyst, to provide XXIX. Condensation with formic acid or a carboxylic acid anhydride then yields N-acyl benzimidazole XXX. Heating with aqueous hydrochloric acid results in hydrolysis of the acyl group, affording aminobenzimidazoles XXXI.

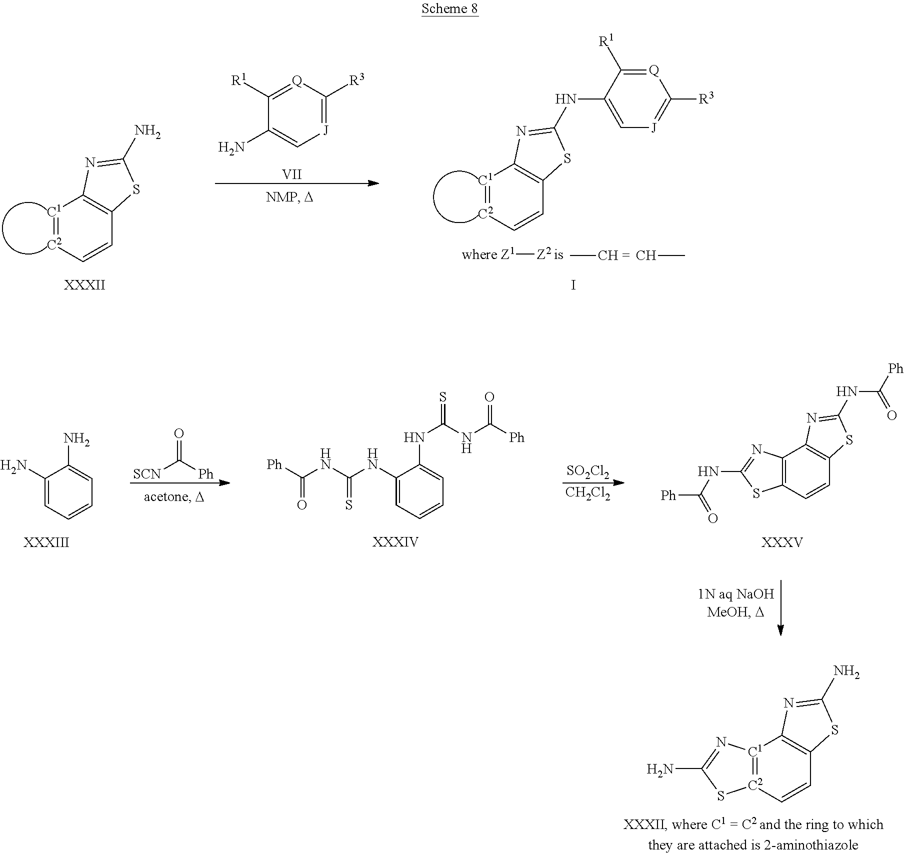

Fused 2-aminothiazole XXXII may be heated to reflux in NMP with aniline VII as an alternative route to compounds of Formula I, where Z¹—Z² is —CH═CH—, as illustrated in Scheme 8. Scheme 8 also shows an example preparation of intermediate XXXII for the case in which —C¹═C²— and the ring to which they are attached is 2-aminothiazole. 1,2-phenylenediamine XXXIII is heated with benzoyl isothiocyanate in acetone to afford XXXIV. This intermediate undergoes cyclization on exposure to sulfuryl chloride in dichloromethane to provide tricyclic bisamide XXXV. Basic aqueous hydrolysis of the amide groups then affords the fused 2-aminothiazole intermediate XXXII.

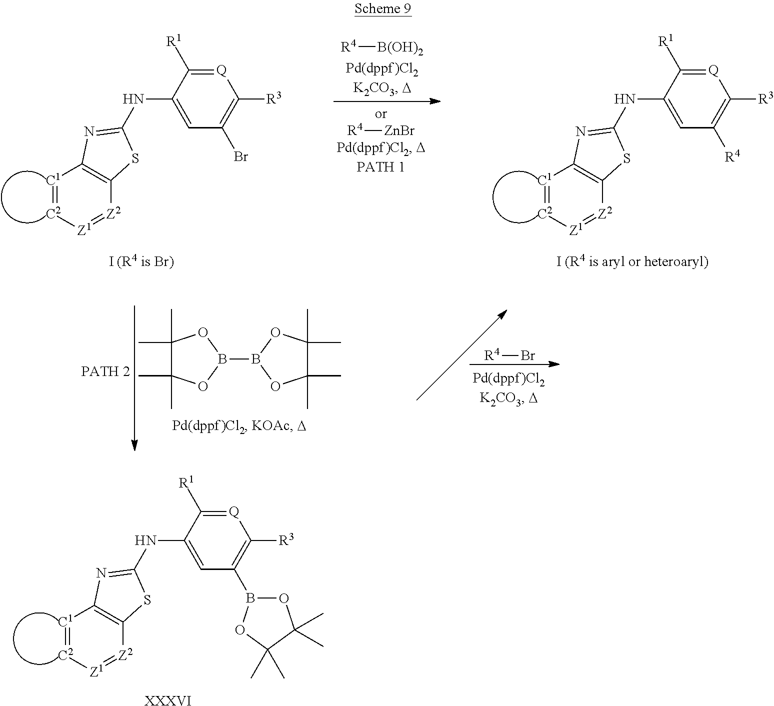

Additional routes to compounds of Formula I, where R⁴ is aryl or heteroaryl, are depicted in Scheme 9. A compound of Formula I, where R⁴ is bromo, prepared as described in Schemes 1-3 or Scheme 8, can react with a boronic acid (or ester), or a zinc reagent, in the presence of a palladium catalyst to yield a compound of Formula I, where R⁴ is aryl or heteroaryl, as shown in path 1. Alternatively, as path 2 illustrates, the compound of Formula I, where R⁴ is bromo, can be converted to the corresponding boronate ester XXXVI by treatment with bis(pinacolato)diboron and a palladium catalyst. The boronate ester XXXVI may be converted to a compound of Formula I, where R⁴ is aryl or heteroaryl, by reaction with an aryl or heteroaryl bromide under palladium catalyzed conditions.

### EXAMPLES

### Intermediate 1: 1-Benzoyl-3-[2-(3-benzoyl-thioureido)-phenyl]thiourea

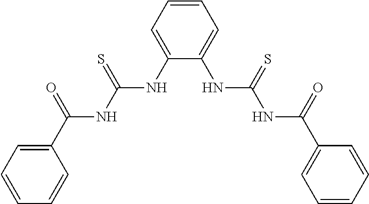

To a clear solution of 1,2-phenylenediamine (3.5 g, 32.4 mmol) in acetone (40 mL) was added benzoyl isothiocyanate (8.9 mL, 66.42 mmol) rapidly dropwise. Reflux temperature was maintained with a heating mantle for thirty minutes. The thick suspension was poured into ice water and the mixture filtered to collect an orange solid, which was washed three times with water, then three times with acetone. The resulting off-white solid was dried under nitrogen/vacuum to yield the title compound.

### Intermediate 2: N-(2,7-Diamino-benzo[2,1-d;3,4d′]bisthiazolyl)-bisbenzamide

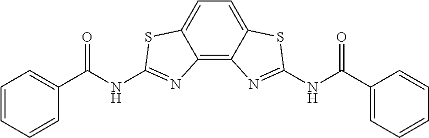

To a clear solution of Intermediate 1 (2.5 g, 5.76 mmol) in CH₂Cl₂ (200 mL) under argon, chilled on a dry ice/acetone bath was added sulfuryl chloride (0.93 mL, 11.52 mmol) and the ice bath removed. The reaction was allowed to warm to 20° C., then heated to reflux for 20 minutes. The reaction was cooled to room temperature and concentrated. The resulting solid was resuspended in dichloromethane and a bright yellow solid collected. This solid was stirred with MeCN and a solid collected. This solid was boiled with MeCN until acidic vapors were no longer given off. The mixture was cooled and the title compound collected as a pale yellow solid.

### Intermediate 3: Benzo[2,1-d;3,4-d′]bisthiazole-2,7-diamine

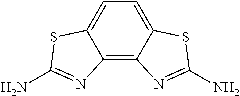

A solution of Intermediate 2 (0.64 g, 1.49 mmol) in MeOH (25 mL) and 1N NaOH (25 mL) was heated to reflux for 24 hours, then cooled to rt. The title compound, a white powder, was collected, rinsed well with water, and dried under nitrogen/vacuum.

### Intermediate 4: 4-Methoxy-2-methyl-benzamide

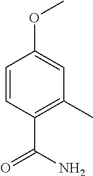

The title compound was prepared as a white powder using 4-methoxy-2-methyl-benzoic acid in place of 3-methoxy-4-nitro-benzoic acid according to the procedure of Intermediate 27.

### Intermediate 5: 4-Isobutyl-benzamide

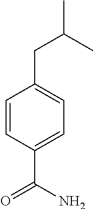

The title compound was prepared as a white powder using 4-isobutyl-benzoic acid in place of 3-methoxy-4-nitro-benzoic acid according to the procedure of intermediate 27.

### Intermediate 6: N-(4-Nitro-1H-benzoimidazol-2-yl)-benzamide

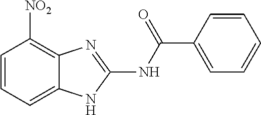

A clear red solution of 3-nitro-1,2-phenylenediamine (0.5 g, 3.27 mmol) and benzoylisothiocyanate (0.44 mL, 3.27 mmol) in THF (50 mL) in a pressure flask under nitrogen was heated to 100° C. for 4 hours. The reaction was filtered and concentrated. The residue was stirred with MeCN and the title compound collected as a pale yellow powder.

### Intermediate 7: N-(4-Nitro-1H-benzoimidazol-2-yl)-acetamide

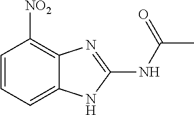

The title compound was prepared by the general method described in Tet. Letters 2006, 47(22), 3747-3750: N-cyclohexylcarbodiimide-N′-Me PS resin was added to a clear red solution of 3-nitro-1,2-phenylenediamine (0.5 g, 3.27 mmol) and acetyl isothiocyanate (0.29 mL, 3.27 mmol) in THF (50 mL) in a pressure flask under nitrogen. The mixture was heated to 100° C. for 2 hours. The reaction was filtered and concentrated. The residue was stirred with MeCN and the title compound collected as a pale yellow powder. The crude product was recrystallized from EtOH.

### Intermediate 8: 2-Methyl-4-nitro-1H-benzoimidazole

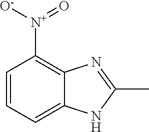

The title compound was prepared according to the method described in Synthesis 1992, 12, 1283-1286.

### Intermediate 9: (4-Nitro-1H-benzoimidazol-2-yl)-methanol

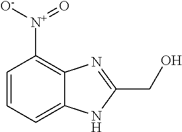

The title compound was prepared according to the method described in Chem. Pharm. Bull. 1995, 43(3), 493-498.

### Intermediate 10: 4-Nitro-1,3-dihydro-benzoimidazol-2-one

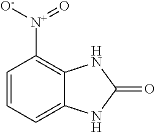

The title compound was prepared by the general method described in J. Med. Chem. 2006, 49(12), 3719-3742: 1,1′-carbonyldiimidazole (0.574 g, 3.75 mmol) was added to a solution of 3-nitro-1,2-phenylenediamine (0.729 g, 4.50 mmol) in THF (10 mL). The reaction mixture was stirred at room temperature for 2 hours, then was concentrated. The residue was stirred in water and the yellow solid precipitate was collected by vacuum filtration, washed with water, and dried to afford the title compound.

### Intermediate 11: 2-Chloro-4-nitro-1H-benzoimidazole

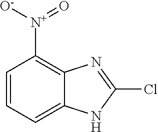

Intermediate 10 (0.224 g, 0.125 mmol) was suspended in phosphorus oxychloride (5 mL) and heated to 95° C. for 2 days. The reaction was concentrated, dried under vacuum, stirred with MeCN, and the title compound collected as a yellow/brown solid.

### Intermediate 12: 4-Nitro-2-trichloromethyl-1H-benzoimidazole

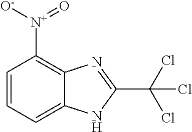

The title compound was prepared according to the procedure described in J. Med. Chem. 2005, 48(26), 8289-8298.

### Intermediate 13: (4-Methyl-piperazin-1-yl)-(4-nitro-1H-benzoimidazol-2-yl)-methanone

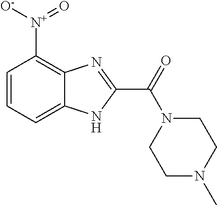

The title compound was prepared from Intermediate 12 according to the procedure described in J. Med. Chem. 2005, 48(26), 8289-8298.

### Intermediate 14: 1,2-Dimethyl-4-nitro-1H-benzoimidazole

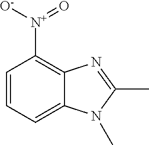

To a suspension of sodium hydride (0.036 g, 1.55 mmol) in dry DMF (5 mL) under Ar in an ice bath was added a clear yellow/brown solution of Intermediate 8 (0.229 g, 1.29 mmol) in dry DMF (5 mL) dropwise and the resulting mixture stirred at 0° C. for 15 minutes, then methyl iodide (0.089 mL, 1.42 mmol) added dropwise and the bath removed. After 1.5 hours, the reaction was poured into brine and extracted four times with EtOAc. The combined organics were washed five times with brine to remove DMF, filtered through cotton, dried over Na₂SO₄, filtered and concentrated to yield the crude product, which was purified by flash column chromatography (Silica gel, 0-5% MeOH/CH₂Cl₂) to yield the title compound.

### Intermediate 15: 4-Nitro-1H-benzoimidazole

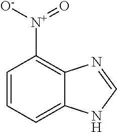

The title compound was prepared from 3-nitro-1,2-phenylenediamine according to the procedure described in J. Org. Chem. 1958, 23, 1944-1946.

### Intermediate 16: 2-(4-Methyl-piperazin-1-yl)-4-nitro-1H-benzoimidazole

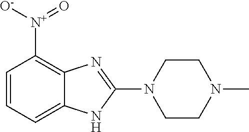

The title compound was prepared by the general method described in J. Med. Chem. 2006, 49(12), 3719-3742: A solution of Intermediate 11 (0.227 g, 1.15 mmol) and N-methylpiperazine (0.255 mL, 2.30 mmol) in DMSO (2 mL) was heated at 80° C. for 17 hours. The reaction mixture was added to ice-water and the resulting precipitate was collected by vacuum filtration, washed with water, and dried to afford the title compound as an orange solid.

### Intermediate 17: 1-Ethyl-7-nitro-1H-benzoimidazole

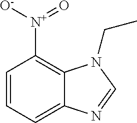

To a suspension of sodium hydride (0.014 g, 0.607 mmol) in dry DMF (5 mL) under Ar in an ice bath was added solid Intermediate 15 (0.090 g, 0.552 mmol) in one portion, and then the ice bath was removed. After 30 minutes at room temperature, the reaction was returned to an ice bath, then ethyl iodide (0.049 ml, 0.607 mmol) added dropwise, and the reaction stirred cold. The bath was removed, and the reaction stirred at room temperature for 12 hours, then heated to 65° C. The reaction was concentrated, taken up in EtOAc and washed five times with brine, filtered through cotton, dried (Na₂SO₄), filtered, and concentrated. The resulting brown oil was purified by flash column chromatography (Silica gel, 0-60% EtOAc/Heptane). The product fractions were concentrated to yield the title compound.

### Intermediate 18: 1-Ethyl-2-methyl-4-nitro-1H-benzoimidazole

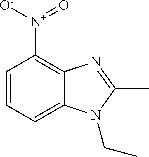

The title compound was prepared from Intermediate 8 by the method of Intermediate 17.

### Intermediate 19: 1-Ethyl-2-methyl-7-nitro-1H-benzoimidazole

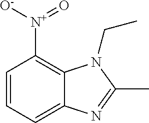

The title compound was isolated from the reaction that produced Intermediate 18 by flash column chromatography.

### Intermediate 20: 2-Methyl-4-nitro-1-propyl-1H-benzoimidazole

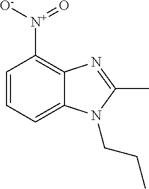

The title compound was prepared from Intermediate 8 and iodopropane by the method of Intermediate 17.

### Intermediate 21: 4-Nitro-1H-benzotriazole

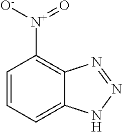

The title compound was prepared according to the procedure described in Bioorg. Med. Chem. Lett. 2007, 17, 4791-4794.

### Intermediate 22: (4-Methoxy-3-nitro-phenyl)-phosphonic acid diethyl ester

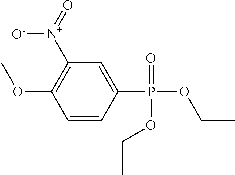

The title compound was prepared according to the procedure described in JACS 1953, 75, 4901-4903.

### Intermediate 23: 4-Methoxy-N-methyl-3-nitro-benzenesulfonamide

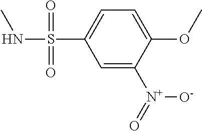

To a clear brown solution of 4-methoxy-3-nitrobenzenesulfonyl chloride (0.227 g, 0.902 mmol) in THF (10 mL) under Ar was added 2M methylamine in THF (1.13 mL, 2.26 mmol) and the mixture heated to reflux. After 45 minutes the reaction was cooled to room temperature and concentrated to a yellow solid which was partitioned between EtOAc/1N HCl. The layers were separated, and the aqueous portion extracted twice with EtOAc. The combined organics were washed once with brine, filtered through cotton, dried over Na₂SO₄, filtered and concentrated to yield a yellow oil which crystallizes.

### Intermediate 24: 4-Fluoro-3-nitro-benzamide

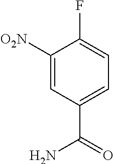

A round bottom flask fitted with a reflux condenser vented through an aqueous sodium hydroxide solution was charged with 4-fluoro-3-nitro-benzoic acid (5.0 g, 27.0 mmol). Thionyl chloride (20 mL) was added and the resulting suspension was heated in an 80° C. oil bath for 3 h. The mixture was concentrated and the residual oil was dissolved in THF (20 mL) and added slowly via pipette to an ice-cold solution of concentrated aqueous ammonium hydroxide (20 mL). The resulting bright yellow mixture was stirred at 0° C. for 35 min. The mixture was partially concentrated to remove THF and the residual solution was extracted with EtOAc. The organic phase was dried (Na₂SO₄), filtered, and concentrated. The residue was purified by flash column chromatography (Silica gel, 1-3% EtOH/CH₂Cl₂) to afford the title compound as a white solid.

### Intermediate 25: 4-Isopropoxy-3-nitro-benzamide

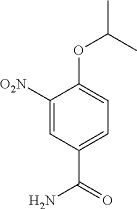

To a solution of iPrOH (0.619 mL, 8.09 mmol) in THF (25 mL) at 0° C., added a 0.5 M solution of KHMDS in toluene (16.2 mL, 8.09 mmol) followed by Intermediate 24 (0.993 g, 5.39 mmol). The resulting brown suspension was stirred at 0° C. for 1 h, then was allowed to warm to 23° C. and was stirred for an additional 4 h. The mixture was partially concentrated to remove THF and was diluted with water and extracted with EtOAc. The organic phase was dried (Na₂SO₄), filtered, and concentrated, affording the crude title compound as an orange solid which was used without further purification.

### Intermediate 26: 3-Fluoro-4-methoxy-5-nitro-benzamide

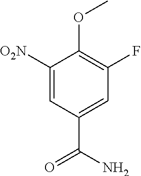

3-Fluoro-4-methoxy-benzamide (3.27 g, 19.3 mmol) was cooled to 0° C. and concentrated sulfuric acid (30 mL) was added followed by potassium nitrate (2.15 g, 21.3 mmol) and the resulting brown solution was stirred at room temperature for 1.5 hours, then was slowly added to ice. The precipitate was collected by vacuum filtration affording the crude title compound as a sticky white solid.

### Intermediate 27: 3-Methoxy-4-nitro-benzamide

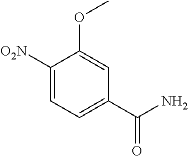

A round bottom flask fitted with a reflux condenser vented through an aqueous sodium hydroxide solution was charged with 3-methoxy-4-nitro-benzoic acid (3.07 g, 15.6 mmol). Thionyl chloride (10 mL) was added and the resulting suspension was heated in an 80° C. oil bath for 30 min. The mixture was concentrated and the residue was dissolved in THF (10 mL) and added slowly via pipette to an ice-cold solution of concentrated aqueous ammonium hydroxide (10 mL). The resulting yellow suspension was stirred at 0° C. for 30 min. The suspension was partially concentrated to remove THF and was filtered to afford the crude title compound as a light yellow solid.

### Intermediate 28: 4-Methoxy-2-methyl-5-nitro-benzamide

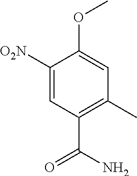

Intermediate 4 (7.75 g, 46.9 mmol) was cooled to 0° C. and concentrated sulfuric acid (40 mL) was added followed by potassium nitrate (4.74 g, 46.9 mmol) and the resulting brown suspension was stirred at room temperature for 40 min, then was slowly added to ice. The cream-colored precipitate was collected by vacuum filtration. The solid was dissolved in a mixture of THF and CH₂Cl₂ and was dried over Na₂SO₄, filtered, and concentrated, affording the crude title compound as a white solid.

### Intermediate 29: 2,3-difluoro-4-methoxy-5-nitro-benzamide

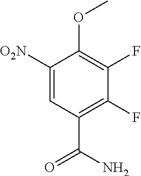

The title compound was prepared from 2,3-difluoro-4-methoxy-benzamide according to the procedure of Intermediate 26.

### Intermediate 30: 4-Methoxy-3-nitro-5-trifluoromethyl-benzamide

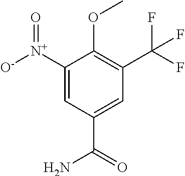

The title compound was prepared from 4-methoxy-3-trifluoromethyl-benzamide according to the procedure of Intermediate 26.

### Intermediate 31: 4-Fluoro-3-nitro-benzenesulfonamide

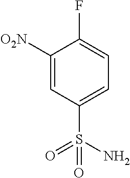

Following the procedure of J. Med. Chem. 2006, 49, 1173, a solution of commercially available 2-fluoronitrobenzene (10.00 g, 70.87 mmol) and chlorosulfonic acid (21 mL) was heated to reflux for 18 hours at 95° C. and then cooled to room temperature. The solution was then added dropwise over a 1 hour period to a solution of iPrOH (225 mL) and concentrated aqueous NH₄OH (54 mL) at −35° C. and stirred for 0.5 hours. The solution was maintained at −35° C. while concentrated aqueous HCl was added until the pH was acidic. The solution was then evaporated to remove some iPrOH, water was added and the solution was evaporated again to remove most of the iPrOH. More water was added, the solution was filtered and the solid was washed with 1 N aqueous HCl and water to give the title compound.

### Intermediate 32: 4-Isopropoxy-3-nitro-benzenesulfonamide

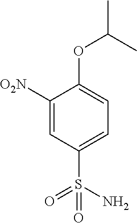

A solution of isopropanol (225 mL) and small chunks of sodium metal (1.92 g, 83.6 mmol) were heated to reflux for 2.5 hours, until the sodium was consumed. The resulting solution was added while still hot to a solution of Intermediate 31 (8.37 g, 38.0 mmol) in THF/iPrOH (1/1, v/v, 150 mL) over a 10 minute period and stirred at room temperature for 3.5 hours. The reaction mixture was partitioned between EtOAc and brine and 1 N aqueous HCl. The organic phase was then washed with brine, dried with Na₂SO₄ and evaporated to give the title compound.

### Intermediate 33: 3-Nitro-4-trifluoromethoxy-benzamide

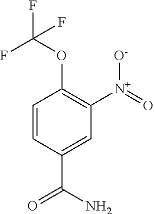

To concentrated aqueous sulfuric acid (3 mL) was slowly added 90% aqueous nitric acid (3 mL) and the resulting solution was cooled in an ice-bath. Solid 4-trifluoromethoxy-benzamide (1.0 g, 4.88 mmol) was slowly added and the reaction mixture was stirred at room temperature for 10 min, then was poured into a stirred ice/water mixture. The white precipitate was collected by vacuum filtration and washed with water, affording the crude title compound, which was used without further purification.

### Intermediate 34: 4-Isobutyl-3-nitro-benzamide

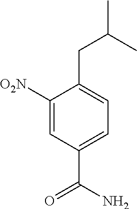

The title compound was prepared using Intermediate 5 in place of Intermediate 4 according to the procedure of Intermediate 28.

### Intermediate 35: 3-Nitro-4-trifluoromethoxy-benzenesulfonamide

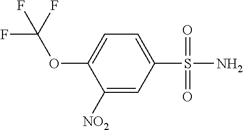

To chlorosulfonic acid (11.3 mL, 170 mmol) was slowly added commercially available 2-trifluoromethoxy-nitrobenzene (8 g, 38.6 mmol). The reaction mixture was heated at 120° C. for 4 h and then cooled down. The above crude mixture was added to a stirred solution of conc. aq. NH₄OH (34.7 mL, 514 mmol, 14.8 M) in iPrOH (100 mL) at −45° C. dropwise over 30 min. The reaction mixture was stirred at −45° C. for 1 h, and 2 N HCl was added to acidify the mixture. Concentration to remove iPrOH was followed by suspension in water, and filtration of the solid. The solid was washed successively with 1 N HCl and water, then air dried to yield the title compound as a white solid.

### Intermediate 36: 1-Ethyl-4-nitro-1H-indazole

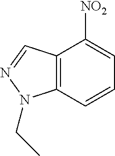

To a suspension of sodium hydride (0.196 g, 4.9 mmol) in dry DMF (5 mL) under Ar in an ice bath was added 4-nitro-1H-indazole (0.5 g, 3.065 mmol) in DMF (2 mL) dropwise. After 30 minutes at room temperature, the reaction was returned to an ice bath, then ethyl iodide (0.272 ml, 3.37 mmol) was added dropwise. The bath was removed, and the reaction stirred at room temperature for 3 hours and poured into ice water. The precipitate was filtered and was purified by flash column chromatography (Silica gel, 0-30% EtOAc/Heptane) to yield the title compound.

### Intermediate 37: 2-Ethyl-4-nitro-2H-indazole

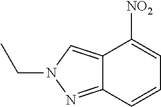

The title compound was isolated from the preparation of Intermediate 36.

### Intermediate 38: 1-Isobutyl-4-nitro-1H-indazole

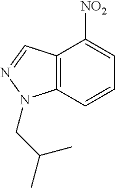

The title compound was prepared from 4-nitro-1H-indazole and 1-iodo-2-methyl-propane by the procedure of Intermediate 36.

### Intermediate 39: 1-(3-Methyl-butyl)-4-nitro-1H-indazole

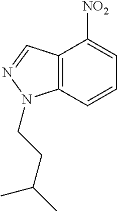

The title compound was prepared from 4-nitro-1H-indazole and 1-iodo-3-methyl-butane by the procedure of Intermediate 36.

### Intermediate 40: 4-Nitro-1-propyl-1H-indazole

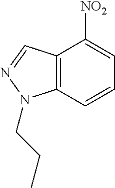

The title compound was prepared from 4-nitro-1H-indazole and 1-iodo-propane by the procedure of Intermediate 36.

### Intermediate 41: (2,6-Dinitro-phenyl)-ethyl-amine

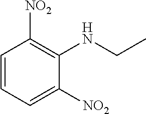

A mixture of 2-chloro-1,3-dinitro-benzene (2.00 g, 9.87 mmol), ethylamine (19.7 mL, 39.5 mmol) and EtOH (15 mL) in a sealed tube was heated at 80° C. for 3 h, and cooled to RT. The solid was filtered, washed with water, and dried to give the title compound as bright yellow crystalline material.

### Intermediate 42: (2,6-Dinitro-phenyl)-methyl-amine

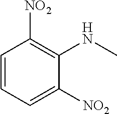

The title compound was prepared according to the procedure of Intermediate 41, using methylamine in place of ethylamine

### Intermediate 43: (2,6-Dinitro-phenyl)-(2,2,2-trifluoro-ethyl)-amine

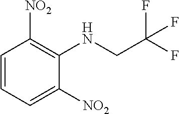

The title compound was prepared according to the procedure of Intermediate 41, using 2,2,2-trifluoroethylamine in place of ethylamine.

### Intermediate 44: 1-Isopropoxy-4-methanesulfonyl-2-nitro-benzene

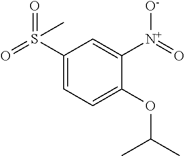

To a solution of 1-fluoro-4-methanesulfonyl-2-nitro-benzene (3.30 g, 15.1 mmol) in THF at 4° C. was added 20% w/w sodium isopropoxide (8.40 g, 20.5 mmol) in THF. The mixture was stirred at 4° C. to room temperature overnight. Brine was added, and the organic layer was separated. The aqueous layer was extracted with CH₂Cl₂. The combined organic phases were dried over Na₂SO₄, filtered, and concentrated to give the title compound as a brown solid.

### Intermediate 45: (2-Methoxy-5-ureido-phenyl)-carbamic acid tert-butyl ester

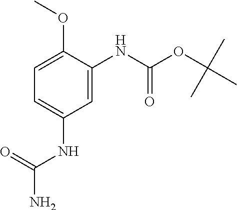

The title compound was prepared from (5-amino-2-methoxyphenyl)-carbamic acid tert-butyl ester according to Org. Syn. 1963, 4, 49-51.

### Intermediate 46: N-(4-Amino-1H-benzoimidazol-2-yl)-benzamide

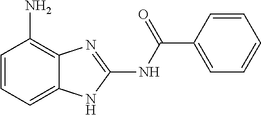

A suspension of Intermediate 6 (0.65 g, 2.30 mmol) and 10% Pd/C (0.06 g) in MeOH (50 mL) was hydrogenated at 30 psi H₂ for 48 hours. The reaction mixture was diluted with 175 mL MeOH, heated to boiling, and filtered hot through a 0.45 μm filter and concentrated to yield the title compound.

### Intermediate 47: N-(4-Amino-1H-benzoimidazol-2-yl)-acetamide

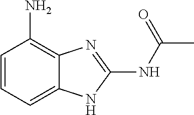

The title compound was prepared according to the method of Intermediate 46, substituting Intermediate 7 for Intermediate 6.

### Intermediate 48: 2-Methyl-1H-benzoimidazol-4-ylamine

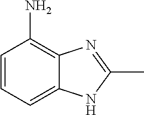

A suspension of Intermediate 8 (0.40 g, 2.29 mmol) and 10% Pd/C (0.04 g) in EtOH (12 mL) and water (3 mL) was hydrogenated at 50 psi H₂ for 12 hours. The reaction was filtered and concentrated, and purified by flash column chromatography (Silica gel, 0-5% MeOH/CH₂Cl₂) to yield the title compound.

### Intermediate 49: (4-Amino-1H-benzoimidazol-2-yl)-methanol

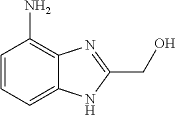

A suspension of Intermediate 9 (0.128 g; 0.663 mmol) and 10% Pd/C (0.012 g) in MeOH (35) was hydrogenated at 35 psi for 2 hours. The catalyst was filtered and the filtrate concentrated to give the title compound.

### Intermediate 50: 4-Amino-1,3-dihydro-benzoimidazol-2-one.HCl

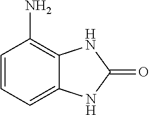

The title compound was prepared from Intermediate 10 by the method of Intermediate 49, with the addition of 1N HCl (2 mL) to the reaction mixture.

### Intermediate 51: 2-Chloro-1H-benzoimidazol-4-ylamine

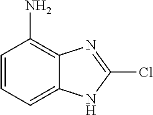

The title compound was prepared from Intermediate 11 by the method of Intermediate 49, purifying the crude product by flash column chromatography (Silica gel, 0-5% MeOH/CH₂Cl₂).

### Intermediate 52: (4-Amino-1H-benzoimidazol-2-yl)-(4-methyl-piperazin-1-yl)-methanone

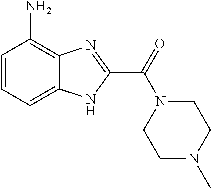

The title compound was prepared from Intermediate 13 by the method of Intermediate 49.

### Intermediate 53: 1,2-Dimethyl-1H-benzoimidazol-4-ylamine

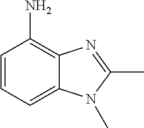

The title compound was prepared from Intermediate 14 by the method of Intermediate 49.

### Intermediate 54: 1H-Benzoimidazol-4-ylamine

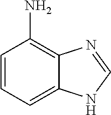

The title compound was prepared from Intermediate 15 by the method of Intermediate 49.

### Intermediate 55: 2-(4-Methyl-piperazin-1-yl)-1H-benzoimidazol-4-ylamine

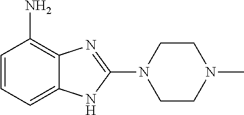

The title compound was prepared from Intermediate 16 by the method of Intermediate 49.

### Intermediate 56: 3-Ethyl-3H-benzoimidazol-4-ylamine.HCl

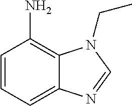

The title compound was prepared from Intermediate 17 by the method of Intermediate 49, with the addition of 1N HCl (2 mL) to the reaction mixture.

### Intermediate 57: 1-Ethyl-2-methyl-1H-benzoimidazol-4-ylamine.HCl

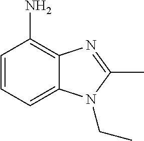

The title compound was prepared from Intermediate 18 by the method of Intermediate 49, with the addition of 1N HCl (2 mL) to the reaction mixture.

### Intermediate 58: 3-Ethyl-2-methyl-3H-benzoimidazol-4-ylamine.HCl

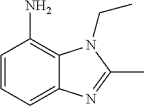

The title compound was prepared from Intermediate 19 by the method of Intermediate 49, with the addition of 1N HCl (2 mL) to the reaction mixture.

### Intermediate 59: 2-Methyl-1-propyl-1H-benzoimidazol-4-ylamine.HCl

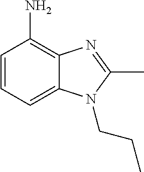

The title compound was prepared from Intermediate 20 by the method of Intermediate 49, with the addition of 1N HCl (2 mL) to the reaction mixture.

### Intermediate 60: 2,3-Dimethyl-1H-indol-7-ylamine

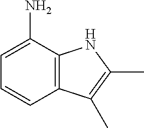

The title compound was prepared from 2,3-dimethyl-7-nitro-1H-indole by the method of Intermediate 49.

### Intermediate 61: 1H-Benzotriazol-4-ylamine.HCl

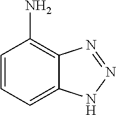

The title compound was prepared from Intermediate 21 by the method of Intermediate 49, with the addition of 1N HCl (2 mL) to the reaction mixture.

### Intermediate 62: Benzooxazol-4-ylamine

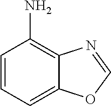

The title compound was prepared according to Heterocycles 1995, 41(2), 345-352.

### Intermediate 63: 5-Fluoro-2-methoxy-phenylamine

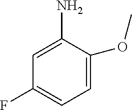

The title compound was prepared from 4-fluoro-2-nitro-anisole by the method of Intermediate 49.

### Intermediate 64: (3-Amino-4-methoxy-phenyl)-phosphonic acid diethyl ester

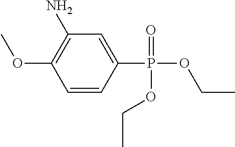

The title compound was prepared from Intermediate 22 by the method of Intermediate 49.

### Intermediate 65: 3-Amino-4-methoxy-benzonitrile

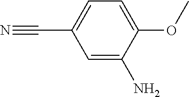

The title compound was prepared according to U.S. Pat. No. 6,403,594, 2002.

### Intermediate 66: 2-Methoxy-5-(pyrrolidine-1-sulfonyl)-phenylamine

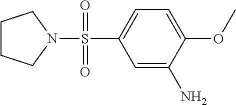

To a solution of 3-amino-4-methoxy-benzenesulfonyl fluoride (0.50 g, 2.44 mmol) in THF (10 mL) under Ar was added DIPEA (0.42 mmol, 2.44 mmol), then pyrrolidine (0.20 mL, 2.44 mmol) dropwise. The reaction was heated at reflux for 48 hours. The reaction was concentrated and partitioned between EtOAc/water. A thick solid was collected, washed well with water then EtOAc and dried under nitrogen/vacuum to yield the title compound.

### Intermediate 67: 4-Methoxy-pyridin-3-ylamine

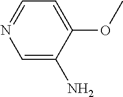

The title compound was prepared from 4-methoxy-3-nitropyridine by the method of Intermediate 49.

### Intermediate 68: 3-Amino-4-methoxy-N-methyl-benzenesulfonamide

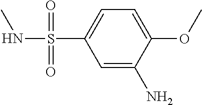

The title compound was prepared from Intermediate 23 by the method of Intermediate 49.

### Intermediate 69: (3-Amino-4-methoxy-phenyl)-urea

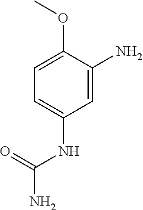

Intermediate 45 (0.126 g, 0.448 mmol) was dissolved in trifluoroacetic acid (2 mL) and the reaction was allowed to stand for 25 minutes, then concentrated. The residue was triturated with Et₂O, and the resulting tan solid dried under vacuum to yield the title compound.

### Intermediate 70: 3-Amino-4-isopropoxy-benzamide

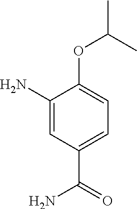

Sodium borohydride (250 mg, 6.60 mmol) was added slowly to a solution of nickel (II) chloride hexahydrate (567 mg, 2.20 mmol) in MeOH (30 mL) at 0° C. and the resulting black suspension was stirred for 30 min at 23° C. The mixture was cooled to 0° C. and to it was added a suspension of crude Intermediate 25 (0.987 g, 4.40 mmol) in MeOH (20 mL), followed by sodium borohydride (583 mg, 15.4 mmol). The mixture was stirred for 1 hour at 23° C. The mixture was partially concentrated to remove most of the MeOH, water was added to quench excess NaBH₄, and the mixture was partitioned between EtOAc and water. The aqueous phase was extracted with EtOAc. The organic phase was dried (Na₂SO₄), filtered, and concentrated. The residue was purified by flash column chromatography (Silica gel, 1-6% MeOH—CH₂Cl₂), yielding the title compound as a white powder.

### Intermediate 71: 3-Amino-5-fluoro-4-methoxy-benzamide

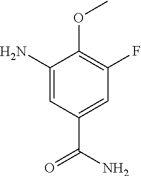

Sodium borohydride (911 mg, 24.1 mmol) was added slowly to a solution of nickel (II) chloride hexahydrate (1.91 g, 8.03 mmol) in MeOH (50 mL) at 0° C. and the resulting black suspension was stirred for 30 min at 23° C. The mixture was cooled to 0° C. and to it was added crude Intermediate 26 (5.73 g) followed by sodium borohydride (2.13 g, 56.2 mmol). The mixture was stirred for 4 h at 23° C. TLC analysis indicated incomplete reduction, so the mixture was cooled to 0° C. and an additional portion of sodium borohydride (600 mg, 15.9 mmol) was added. The reaction was allowed to proceed for another 3 h at room temperature. A small amount of water was added to quench excess NaBH₄ and the mixture was diluted with sat. aq. NaHCO₃ and was filtered through Celite. The filtrate was saturated with NaCl and was extracted with EtOAc. The organic phase was dried (Na₂SO₄), filtered, and concentrated, affording the crude title compound.

### Intermediate 72: 4-Amino-3-methoxy-benzamide

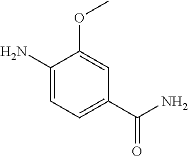

Sodium borohydride (0.764 g, 20.2 mmol) was added slowly to a solution of nickel (II) chloride hexahydrate (1.60 g, 6.73 mmol) in MeOH (25 mL) at 0° C. and the resulting black suspension was stirred for 20 min at 23° C. The mixture was cooled to 0° C. and to it was added crude Intermediate 27 (2.64 g, 13.5 mmol) followed by sodium borohydride (1.78 g, 47.1 mmol). The mixture was stirred for 30 min at 23° C. A small amount of water was added to quench excess NaBH₄ and the mixture was diluted with sat. aq. NaHCO₃ and was filtered through Celite. The filtrate was extracted with EtOAc. The organic phase was dried (Na₂SO₄), filtered, and concentrated, affording the crude title compound as an off-white solid.

### Intermediate 73: 5-Amino-4-methoxy-2-methyl-benzamide

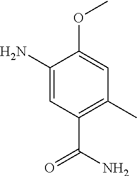

The title compound was prepared using Intermediate 28 in place of Intermediate 27 according to the procedure of Intermediate 72. The crude product was purified by flash column chromatography (Silica gel, 0-7.5% MeOH/CH₂Cl₂), affording the title compound as a cream-colored solid.

### Intermediate 74: 5-Amino-2,3-difluoro-4-methoxy-benzamide

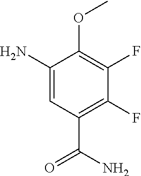

The title compound was prepared from Intermediate 29 according to the procedure of Intermediate 72.

### Intermediate 75: 3-Amino-4-methoxy-5-trifluoromethyl-benzamide

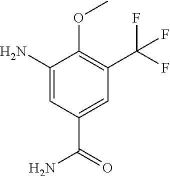

The title compound was prepared from Intermediate 30 according to the procedure of Intermediate 72.

### Intermediate 76: 3-Amino-4-isopropoxy-benzenesulfonamide

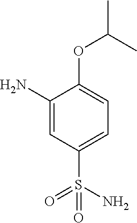

Sodium borohydride (1.88 g, 49.6 mmol) was added slowly to a solution of nickel (II) chloride hexahydrate (3.93 g, 16.5 mmol) in methanol (60 mL) at 0° C. and the resulting black suspension was stirred for 30 min at 23° C. The mixture was cooled to 0° C. and Intermediate 32 (8.6 g, 33.0 mmol) was added followed by sodium borohydride (4.38 g, 115.6 mmol). The resulting black suspension was stirred for 30 min at 23° C. Water was added to the reaction mixture to quench excess NaBH₄, followed by addition of saturated aqueous NaHCO₃. The product was extracted with dichloromethane and the organic phase was washed with brine, dried with Na₂SO₄ and evaporated to give the title compound.

### Intermediate 77: 3-Amino-4-trifluoromethoxy-benzamide

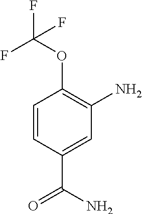

To a solution of nickel(II) chloride hexahydrate (470 mg, 1.98 mmol) in MeOH (10 mL) at 0° C. was slowly added sodium borohydride (225 mg, 5.94 mmol) (caution: gas evolution). The resulting black suspension was stirred at room temperature for 30 min, then was cooled to 0° C. before addition of crude Intermediate 33 (0.99 g, 3.96 mmol) and a second portion of sodium borohydride (524 mg, 13.9 mmol). The resulting black suspension was stirred at room temperature for 0.5 h before addition of a small amount of water to quench remaining borohydride. The mixture was diluted with sat. aq. NaHCO₃ and extracted with CH₂Cl₂. To facilitate extraction of the polar product, the aqueous phase was saturated with NaCl, then was further extracted with CH₂Cl₂. The organic phase was washed with saturated aqueous NaCl and was dried (Na₂SO₄), filtered, and concentrated. The residual white solid was purified by flash column chromatography (Silica gel, 20-100% EtOAc-Hept), affording the title compound as an off-white solid.

### Intermediate 78: 3-Amino-4-isobutyl-benzamide

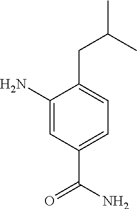

The title compound was prepared using Intermediate 34 in place of Intermediate 27 according to the procedure of intermediate 72.

### Intermediate 79: 3-Amino-4-trifluoromethoxy-benzenesulfonamide

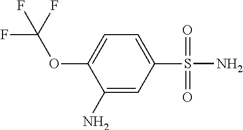

The title compound was prepared from Intermediate 35 according to the procedure of Intermediate 70.

### Intermediate 80:1-Ethyl-1H-indazol-4-ylamine

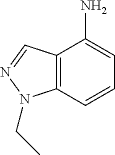

The title compound was prepared from Intermediate 36 according to the procedure of Intermediate 70.

### Intermediate 81: 2-Ethyl-2H-indazol-4-ylamine

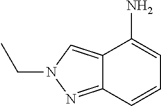

The title compound was prepared from Intermediate 37 according to the procedure of Intermediate 70.

### Intermediate 82: 1-Isobutyl-1H-indazol-4-ylamine

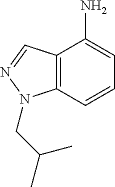

The title compound was prepared from Intermediate 38 according to the procedure of Intermediate 70.

### Intermediate 83: 1-(3-Methyl-butyl)-1H-indazol-4-ylamine

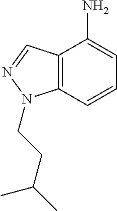

The title compound was prepared from Intermediate 39 according to the procedure of Intermediate 70.

### Intermediate 84: 1-Propyl-1H-indazol-4-ylamine

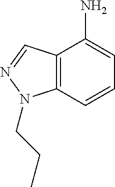

The title compound was prepared from Intermediate 40 according to the procedure of Intermediate 70.

### Intermediate 85: N-Ethyl-benzene-1,2,3-triamine

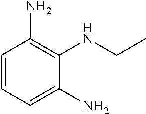

To a Parr bottle containing Intermediate 41 (1.98 g, 9.38 mmol) and EtOH was added 10% Pd/C (0.38 g). The mixture was shaken under 35 psi H₂ for 7 hours and filtered through Celite. The filtrate was concentrated to give the title compound as oil.

### Intermediate 86: N-Methyl-benzene-1,2,3-triamine

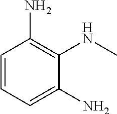

The title compound was prepared from Intermediate 42 by the procedure of Intermediate 85.

### Intermediate 87: N-(2,2,2-Trifluoro-ethyl)-benzene-1,2,3-triamine

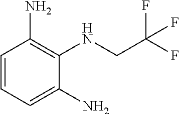

The title compound was prepared from Intermediate 43 by the procedure of Intermediate 85.

### Intermediate 88: N-(3-Ethyl-2-methyl-3H-benzoimidazol-4-yl)-acetamide

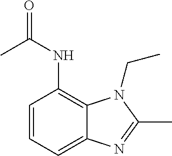

A solution of Intermediate 85 (1.40 g, 9.26 mmol) in acetic anhydride (5.0 mL, 52.9 mmol) was heated at 85° C. for 1.5 hours and concentrated. Column chromatography (Silica gel, EtOAc, then 5%-10% MeOH in CH₂Cl₂) provided the title compound as brown solid.

### Intermediate 89: N-(2,3-Dimethyl-3H-benzoimidazol-4-yl)-acetamide

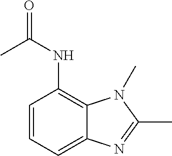

The title compound was prepared from Intermediate 86 according to the procedure of Intermediate 88.

### Intermediate 90: N-[3-Methyl-2-(2,2,2-trifluoro-ethyl)-3H-benzoimidazol-4-yl]-acetamide

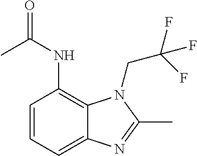

The title compound was prepared from Intermediate 87 according to the procedure of Intermediate 88.

### Intermediate 91: N-(3-Methyl-3H-benzoimidazol-4-yl)-formamide

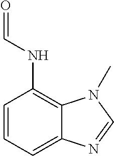

The title compound was prepared from Intermediate 86 by the procedure of Intermediate 88, substituting formic acid for acetic anhydride.

### Intermediate 92: N-[3-(2,2,2-Trifluoro-ethyl)-3H-benzoimidazol-4-yl]-formamide

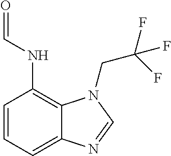

The title compound was prepared from Intermediate 87 by the procedure of Intermediate 88, substituting formic acid for acetic anhydride.

### Intermediate 93: 3-Ethyl-2-methyl-3H-benzoimidazol-4-ylamine

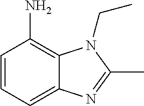

Intermediate 88 (1.12 g, 5.16 mmol) in 6 N HCl (8 mL) was heated at 90° C. for 3 hours. After cooling down to room temperature, the mixture was basified with concentrated NH₄OH and extracted with CH₂Cl₂. The organic phases were dried over Na₂SO₄, filtered, and concentrated to give the title compound as brown oil.

### Intermediate 94: 2,3-Dimethyl-3H-benzoimidazol-4-ylamine

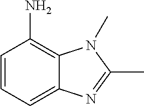

The title compound was prepared from Intermediate 89 according to the procedure of Intermediate 93.

### Intermediate 95: 3-Methyl-3H-benzoimidazol-4-ylamine

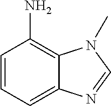

The title compound was prepared from Intermediate 91 according to the procedure of Intermediate 93.

### Intermediate 96: 2-Methyl-3-(2,2,2-trifluoro-ethyl)-3H-benzoimidazol-4-ylamine

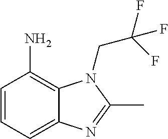

The title compound was prepared from Intermediate 90 according to the procedure of Intermediate 93.

### Intermediate 97: 3-(2,2,2-Trifluoro-ethyl)-3H-benzoimidazol-4-ylamine

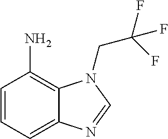

The title compound was prepared from Intermediate 92 according to the procedure of Intermediate 93.

### Intermediate 98: 2-Isopropoxy-5-methanesulfonyl-phenylamine

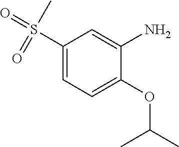

To a Parr bottle containing Intermediate 44 (4.10 g, 15.8 mmol) in EtOAc and EtOH (1:3) was added 10% Pd/C (0.30 g). The mixture was shaken under 37 psi H₂ for 3 days and filtered through Celite. The filtrate was concentrated to give the title compound as oil.

### Intermediate 99: 4-Isopropoxy-3-isothiocyanato-benzamide

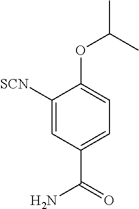

A solution of sodium bicarbonate (0.645 g, 7.68 mmol) in water (15 mL) was added to Intermediate 70 (0.497 g, 2.56 mmol) in a mixture of chloroform (15 mL) and water (15 mL). Thiophosgene (0.206 mL, 2.69 mmol) was then added. The biphasic solution was stirred at room temperature for 2.5 h. TLC analysis indicated slight remaining starting material, so an additional 0.030 mL portion of thiophosgene was added and the mixture was stirred for 40 min. The phases were separated and the aqueous phase was extracted with CH₂Cl₂. The organic phase was dried (Na₂SO₄), filtered, and concentrated, yielding the crude title compound as an off-white solid.

### Intermediate 100: 4-Isopropoxy-3-isothiocyanato-benzenesulfonamide

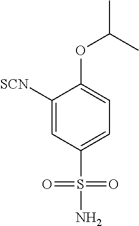

A solution of sodium bicarbonate (16.8 g, 199.5 mmol) in water (400 mL) was added to Intermediate 76 (15.3 g, 66.5 mmol) in a mixture of chloroform (200 mL) and water (200 mL). Thiophosgene (6.37 mL, 83.1 mmol) was then added. The biphasic solution was stirred at room temperature for 1.5 h. The phases were separated and the aqueous phase was extracted with CH₂Cl₂. The organic phase was washed with water, dried (Na₂SO₄), filtered, and concentrated, yielding the crude title compound as a tan solid.

### Intermediate 101: 1-Isopropoxy-2-isothiocyanato-benzene

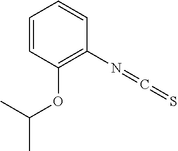

A suspension of 2-isopropoxyaniline hydrochloride (0.524 g, 2.79 mmol) in CHCl₃ (13 mL) was heated until a clear solution formed. A solution of Na₂CO₃ (0.939 g; 11.2 mmol) in water was added and the biphasic mixture stirred vigorously and chilled on an ice bath. Thiophosgene (0.236 mL, 3.07 mmol) was added dropwise, and the ice bath removed. After 35 minutes the layers were separated. The aqueous layer was extracted once with CHCl₃. The combined organics were washed once with brine, filtered through cotton, dried over Na₂SO₄, filtered and concentrated to yield the title compound as an orange oil.

### Intermediate 102: 1-Ethoxy-2-isothiocyanato-benzene

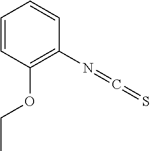

The title compound was prepared from 2-ethoxy aniline by the procedure of Intermediate 99.

### Intermediate 103: 3-Isothiocyanato-4-methoxy-benzamide

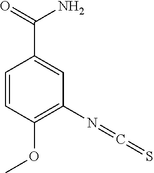

The title compound was prepared from 3-amino-4-methoxybenzamide by the procedure of Intermediate 99.

### Intermediate 104: 1-Isothiocyanato-2-(2,2,2-trifluoro-ethoxy)-benzene

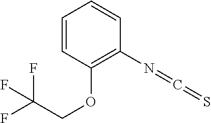

The title compound was prepared from 2-(2,2,2-Trifluoro-ethoxy)-phenylamine by the procedure of Intermediate 99.

### Intermediate 105: 2,3-Difluoro-5-isothiocyanato-4-methoxy-benzamide

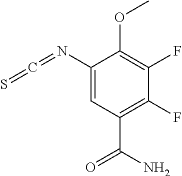

The title compound was prepared from Intermediate 74 according to the procedure of Intermediate 99. The product was purified by flash column chromatography (Silica gel, 0-2% MeOH/CH₂Cl₂) and subsequent trituration of the product with EtOAc/heptanes.

### Intermediate 106: 3-Fluoro-5-isothiocyanato-4-methoxy-benzamide

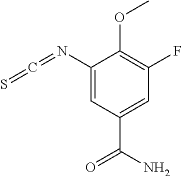

The title compound was prepared using Intermediate 71 in place of Intermediate 70 according to the procedure described for Intermediate 99. The crude product was purified by flash column chromatography (Silica gel, 50% EtOAc/CH₂Cl₂), affording the title compound as an off-white powder.

### Intermediate 107: N-(3-Isothiocyanato-4-methoxy-phenyl)-acetamide

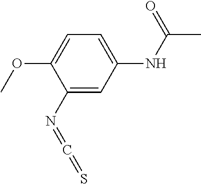

The title compound was prepared from 3-amino-4-methoxy acetanilide by the procedure of Intermediate 99.

### Intermediate 108: 2-Isothiocyanato-1-methoxy-4-trifluoromethyl-benzene

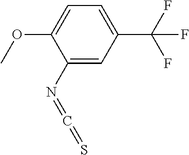

The title compound was prepared from 2-methoxy-5-(trifluoromethyl)aniline by the procedure of Intermediate 99.

### Intermediate 109: 4-Isothiocyanato-3-methoxy-benzamide

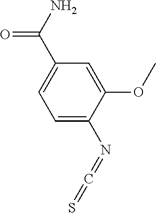

The title compound was prepared using Intermediate 72 in place of Intermediate 70 according to the procedure described for Intermediate 99.

### Intermediate 110: 2-Isothiocyanato-4-methanesulfonyl-1-methoxy-benzene

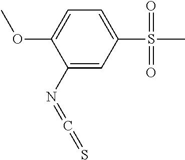

The title compound was prepared from 2-methoxy-5-methylsulfonylaniline by the procedure of Intermediate 99.

### Intermediate 111: 3-Isothiocyanato-4-methoxy-5-trifluoromethyl-benzamide

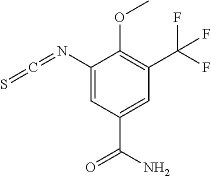

The title compound was prepared from Intermediate 75 by the procedure of Intermediate 99.

### Intermediate 112: 3-Isothiocyanato-4-methoxy-benzenesulfonamide

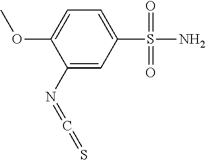

The title compound was prepared from 3-amino-4-methoxy-benzenesulfonamide by the procedure of Intermediate 99.

### Intermediate 113: 3-Isothiocyanato-4-methoxy-N,N-dimethyl-benzenesulfonamide

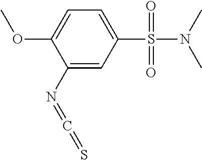

The title compound was prepared from 3-amino-4-methoxy-N,N-dimethylbenzenesulfonamide by the procedure of Intermediate 99.

### Intermediate 114: 5-Isothiocyanato-4-methoxy-2-methyl-benzamide

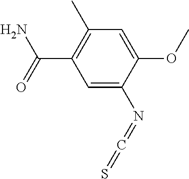

The title compound was prepared using Intermediate 73 in place of Intermediate 70 according to the procedure described for Intermediate 99. During the extraction, precipitated solid made separation of the phases difficult; the solid was collected by vacuum filtration and was combined with the organic extracts. The crude title compound was obtained as a cream colored solid.

### Intermediate 115: 4-Fluoro-2-isothiocyanato-1-methoxy-benzene

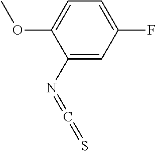

The title compound was prepared from Intermediate 63 by the procedure of Intermediate 99.

### Intermediate 116: 1-Isothiocyanato-2-trifluoromethoxy-benzene

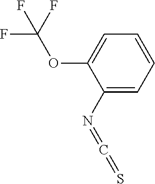

The title compound was prepared from 2-(trifluoromethyl)-aniline by the procedure of Intermediate 99.

### Intermediate 117: 4-Fluoro-1-isopropoxy-2-isothiocyanato-benzene

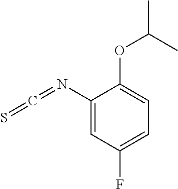

The title compound was prepared using 5-fluoro-2-isopropoxy-phenylamine in place of Intermediate 70 according to the procedure described for Intermediate 99.

### Intermediate 118: 4-Ethanesulfonyl-2-isothiocyanato-1-methoxy-benzene

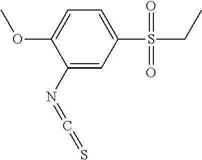

The title compound was prepared from 2-methoxy-5-ethylsulfonylaniline by the procedure of Intermediate 99.

### Intermediate 119: 3-Isothiocyanato-4-methoxy-benzoic acid methyl ester

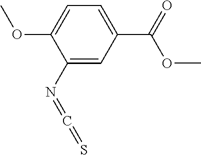

The title compound was prepared from methyl 3-amino-4-methoxybenzoate by the procedure of Intermediate 101.

### Intermediate 120: 1,5-Difluoro-3-isothiocyanato-2-methoxy-benzene

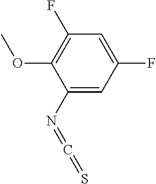

The title compound was prepared from methyl 3,5-difluoro-2-methoxyaniline by the procedure of Intermediate 99.

### Intermediate 121: 3-Isothiocyanato-4-trifluoromethoxy-benzenesulfonamide

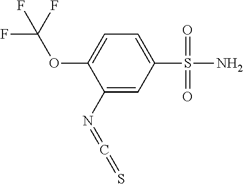

A solution of sodium bicarbonate (3.8 g, 45.2 mmol) in water (50 mL) was added to Intermediate 79 (3.84 g, 15.0 mmol) in chloroform (100 mL). Thiophosgene (1.44 mL, 18.7 mmol) was then added. The biphasic solution was stirred at room temperature for 2 h. TLC analysis indicated slight remaining starting material, so an additional 0.5 mL portion of thiophosgene was added and the mixture was stirred for 40 min. The reaction mixture was partially concentrated to get rid of most chloroform. The precipitated solid was filtered, washed with water, and air dried, yielding the crude title compound as an off-white solid.

### Intermediate 122: 3-Isothiocyanato-4-trifluoromethoxy-benzamide

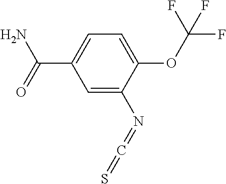

The title compound was prepared from Intermediate 77 by the procedure of Intermediate 99.

### Intermediate 123: (3-Isothiocyanato-4-methoxy-phenyl)-phosphonic acid diethyl ester

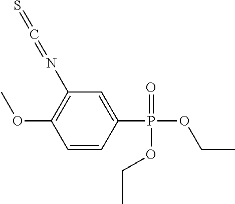

The title compound was prepared from Intermediate 64 by the procedure of Intermediate 99.

### Intermediate 124: 3-Isothiocyanato-4-methoxy-benzonitrile

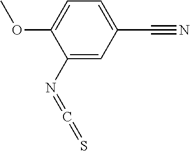

Thiophosgene (0.112 mL, 1.46 mmol) was added to a solution of Intermediate 65 (0.216 g, 1.46 mmol) and sodium bicarbonate (0.368 g, 4.38 mmol) in chloroform (1 mL) and water (10 mL) and stirred at room temperature overnight. An additional 0.1 eq of thiophosgene was added to the reaction mixture and stirred for several hours. Excess ethyl acetate was added and the product was extracted, dried with sodium sulfate and purified via column chromatography with heptanes: ethyl acetate to give the title compound.

### Intermediate 125: 4-Bromo-2-isothiocyanato-1-methoxy-benzene

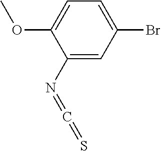

A solution of sodium bicarbonate (3.74 g, 44.5 mmol) in water (75 mL) was added to commercially available 5-bromo-2-methoxy-phenylamine (3 g, 14.8 mmol) in chloroform (75 mL). Thiophosgene (1.42 mL, 18.6 mmol) was then added. The biphasic solution was stirred at room temperature for 1 h. The phases were separated and the aqueous phase was extracted with CH₂Cl₂. The organic phase was dried (Na₂SO₄), filtered, and concentrated, yielding the crude title compound as an off-white solid.

### Intermediate 126: N,N-Diethyl-3-isothiocyanato-4-methoxy-benzenesulfonamide

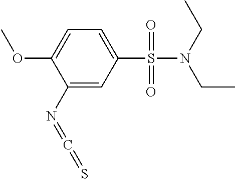

The title compound was prepared from 3-amino-N,N-diethyl-4-methoxy-benzenesulfonamide by the procedure of Intermediate 99.

### Intermediate 127: 1-(3-Isothiocyanato-4-methoxy-benzenesulfonyl)-pyrrolidine

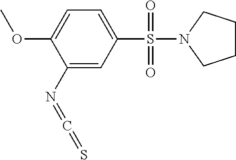

The title compound was prepared from Intermediate 66 by the procedure of Intermediate 99.

### Intermediate 128: 4-Isothiocyanato-1-methyl-1H-benzoimidazole

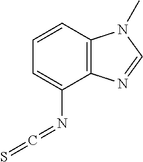

To 4-amino-1-methylbenzimidazole (0.210 g, 1.43 mmol) and thiocarbonyldiimidazole (0.254 g, 1.43 mmol) under nitrogen was added CH₂Cl₂ (10 mL) and the mixture stirred at room temperature for 2 hours. The reaction was concentrated to yield the title compound which was used without further purification.

### Intermediate 129: 1-(3-Isothiocyanato-4-methoxy-benzenesulfonyl)-piperidine

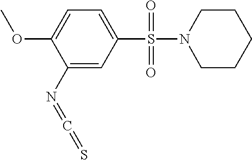

The title compound was prepared from 2-methoxy-5-(piperidine-1-sulfonyl)phenylamine by the procedure of Intermediate 101.

### Intermediate 130: 4-(3-Isothiocyanato-4-methoxy-benzenesulfonyl)-morpholine

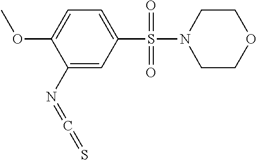

The title compound was prepared from 2-methoxy-5-(morpholine-1-sulfonyl)phenylamine by the procedure of Intermediate 101.

### Intermediate 131: 3-Isothiocyanato-4-methoxy-N-methyl-benzenesulfonamide

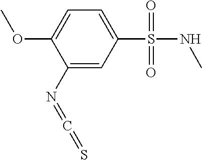

The title compound was prepared from Intermediate 68 by the procedure of Intermediate 101.

### Intermediate 132: 4-Isobutyl-3-isothiocyanato-benzamide

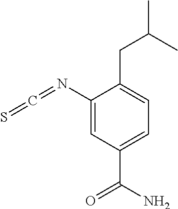

The title compound was prepared using Intermediate 78 in place of Intermediate 70 according to the procedure described for intermediate 99. (The reaction was monitored by TLC and several additional portions of thiophosgene were added until the reaction approached complete conversion).

### Intermediate 133: 1-Isopropoxy-2-isothiocyanato-4-methanesulfonyl-benzene

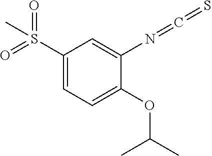

To a stirred mixture of Intermediate 98 (2.89 g, 12.6 mmol) and NaHCO₃ (3.18 g, 37.8 mmol) in CHCl₃ and water (1:1) at 4° C. was added thiophosgene (1.16 mL, 15.1 mmol) dropwise. After completion of the addition, the ice bath was removed. The mixture was stirred for 4 hours, the organic layer was separated, and the aqueous layer was extracted with CH₂Cl₂. The combined organic phases were washed with water, dried over Na₂SO₄, filtered, and concentrated to give the title compound as brown solid.

### Intermediate 134: 3-Isothiocyanato-4-methoxy-pyridine

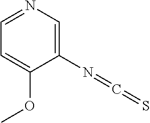

The title compound was prepared from 4-methoxy-pyridin-3-ylamine in place of Intermediate 98 according to the procedure of Intermediate 133.

### Intermediate 135: 4-Isopropoxy-3-thioureido-benzamide

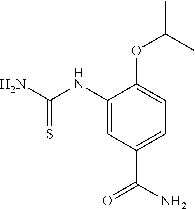

Crude Intermediate 99 (0.608 g) was suspended in MeOH (2 mL). A 2 M solution of ammonia in MeOH (2 mL) was added and the resulting yellow solution was stirred at room temperature for 16 h. The reaction mixture was concentrated and the residue was purified by flash column chromatography (3-8% MeOH/CH₂Cl₂), affording the title compound as a white powder.

### Intermediate 136: (2-Isopropoxy-phenyl)-thiourea

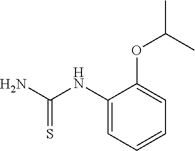

Crude Intermediate 101 (1.03 g, 5.33 mmol) was treated with 2 M ammonia in MeOH (20 mL) and the resulting solution was stirred at 23° C. for 3 h. The mixture was concentrated and the residue was purified by flash column chromatography (Silica gel, 30-50% EtOAc/hept), affording the title compound.

### Intermediate 137: 4-Methoxy-3-thioureido-benzamide

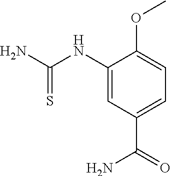

To a solution of 3-amino-4-methoxybenzamide (2.49 g, 15.0 mmol) in acetone (30 mL) at reflux was added benzoyl isothiocyanate (2.22 mL, 16.5 mmol) and the mixture was stirred at reflux for 30 minutes, then was poured into water. The precipitate was collected by vacuum filtration and was treated with 10% aq. NaOH (15 mL). The mixture was refluxed for 40 min, was cooled to room temperature, and was poured into a mixture of ice and 6 N aq. HCl. The mixture was basified to pH 10 with conc. aq. NH₄OH and the resulting white solid precipitate was collected by vacuum filtration, affording the title compound, which was used without further purification.

### Intermediate 138: N-{4-[3-(2-Methoxy-phenyl)-thioureido]-1H-benzoimidazol-2-yl}-benzamide

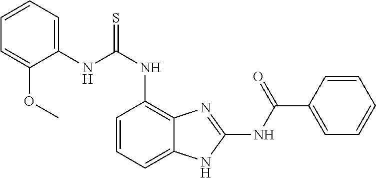

To a suspension of Intermediate 46 (0.58 g, 2.3 mmol) in THF (30 mL) was added 2-methoxyphenyl isothiocyanate (0.32 mL, 2.3 mmol) and the mixture was stirred at room temperature for 7 days. The reaction mixture was filtered of a small amount of a bright yellow solid, and the filtrate concentrated and purified by flash column chromatography (Silica gel, 0-2.5% MeOH/CH₂Cl₂) to yield the title compound.

### Intermediate 139: 3-[3-(2-Benzoylamino-1H-benzoimidazol-4-yl)-thioureido]benzoic acid methyl ester

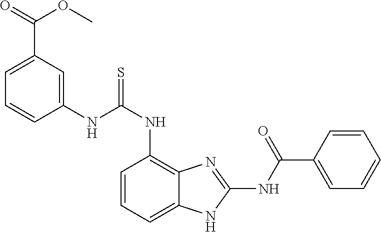

The title compound was prepared according to the procedure of Intermediate 138, substituting 3-isothiocyanato-benzoic acid methyl ester for 2-methoxyphenyl isothiocyanate.

### Intermediate 140: N-{4-[3-(2-Methoxy-phenyl)-thioureido]1H-benzoimidazol-2-yl}-acetamide

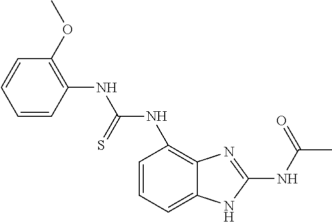

The title compound was prepared from Intermediate 47 according to the method of Intermediate 138, with the following changes. The reaction was performed in DMSO at 60° C., and the product was precipitated from the reaction mixture with water.

### Intermediate 141: 3-[3-(2-Acetylamino-1H-benzoimidazol-4-yl)-thioureido]-4-isopropoxy-benzamide

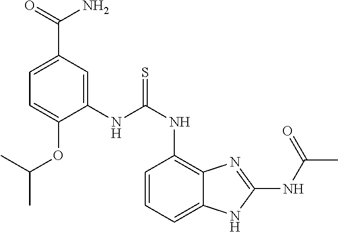

The title compound was prepared according to the procedure of Intermediate 138, substituting Intermediate 99 for 2-methoxyphenyl isothiocyanate, and Intermediate 47 for Intermediate 46.

### Intermediate 142: 4-Isopropoxy-3-[3-(2-methyl-1H-benzoimidazol-4-yl)-thioureido]-benzamide

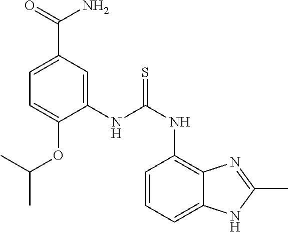

The title compound was prepared according to the procedure of Intermediate 138, substituting Intermediate 99 for 2-methoxyphenyl isothiocyanate, and Intermediate 48 for Intermediate 46.

### Intermediate 143: 3-[3-(2-Hydroxymethyl-1H-benzoimidazol-4-yl)-thioureido]-4-isopropoxy-benzamide

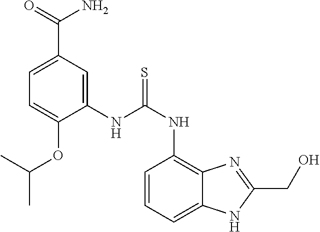

The title compound was prepared according to the procedure of Intermediate 138, substituting Intermediate 99 for 2-methoxyphenyl isothiocyanate, and Intermediate 49 for Intermediate 46.

### Intermediate 144: 4-Isopropoxy-3-[3-(2-oxo-2,3-dihydro-1H-benzoimidazol-4-yl)-thioureido]-benzamide

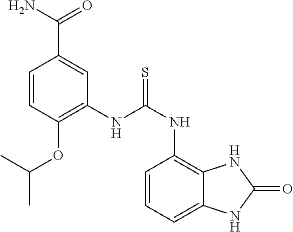

A solution of Intermediate 50 (0.066 g, 0.274 mmol) and Intermediate 99 (0.065 g, 0.275 mmol) and DIPEA (0.048 mL, 0.274 mmol) in DMSO (2 mL) was heated at 65° C. for 2 hours, then cooled to rt. The title compound was precipitated by addition of ice water.

### Intermediate 145: 3-[3-(2-Chloro-1H-benzoimidazol-4-yl)-thioureido]-4-isopropoxy-benzamide

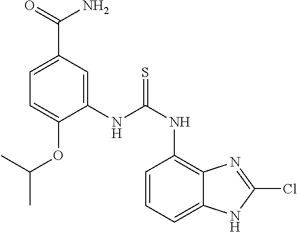

To Intermediate 51 (0.042 g, 0.251 mmol) under N₂ was added Intermediate 99 (0.059 g, 0.251 mmol) and the mixture dissolved in DMSO (1 mL). The reaction was heated in 65° for 5 hours. The title compound was precipitated with ice water, collected, and dried under nitrogen/vacuum.

### Intermediate 146: 4-Isopropoxy-3-{3-[2-(4-methyl-piperazine-1-carbonyl)-1H-benzoimidazol-4-yl]-thioureido}-benzamide

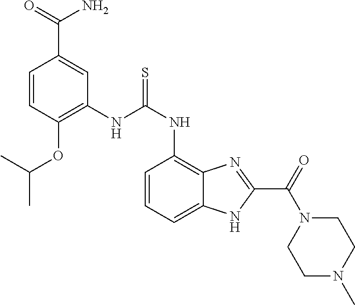

The title compound was prepared from Intermediate 52 and Intermediate 99 according to the method of Intermediate 145.

### Intermediate 147: 3-[3-(1,2-Dimethyl-1H-benzoimidazol-4-yl)-thioureido]-4-isopropoxy-benzamide

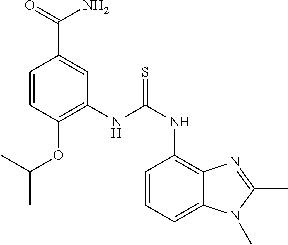

The title compound was prepared from Intermediate 53 and Intermediate 99 according to the method of Intermediate 145.

### Intermediate 148: 4-Isopropoxy-3-[3-(1-methyl-1H-benzoimidazol-4-yl)-thioureido]-benzenesulfonamide.TFA

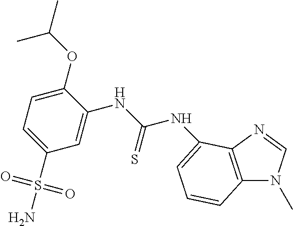

The title compound was prepared from 4-amino-1-methylbenzimidazole and Intermediate 100 according to the method of Intermediate 145. The product was purified by HPLC, eluting with water/acetonitrile/0.2% trifluoroacetic acid.

### Intermediate 149: 3-[3-(1H-Benzoimidazol-4-yl)-thioureido]-4-isopropoxy-benzamide.TFA

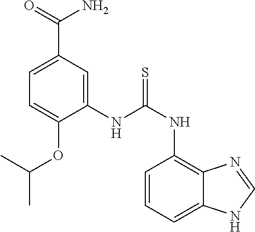

The title compound was prepared from Intermediate 54 and Intermediate 99 according to the method of Intermediate 145. The product was purified by HPLC, eluting with water/acetonitrile/0.2% trifluoroacetic acid.

### Intermediate 150: 4-Isopropoxy-3-[3-(1-methyl-1H-indazol-4-yl)-thioureido]-benzamide

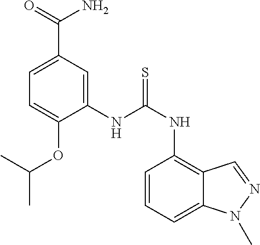

The title compound was prepared from 1-methyl-1H-indazol-4-ylamine and Intermediate 99 according to the method of Intermediate 145.

### Intermediate 151: 4-Isopropoxy-3-{3-[2-(4-methyl-piperazin-1-yl)-1H-benzoimidazol-4-yl]-thioureido}-benzamide

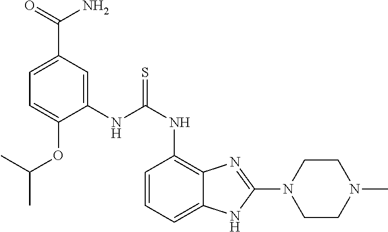

The title compound was prepared from Intermediate 55 and Intermediate 99 according to the method of Intermediate 145.

### Intermediate 152: 3-[3-(2,3-Dimethyl-1H-indol-7-yl)-thioureido]-4-isopropoxy-benzamide.TFA

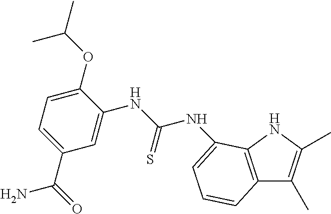

A solution of Intermediate 60 (0.103 g, 0.40 mmol) and Intermediate 99 (0.10 g, 0.40 mmol) in DMSO (1 mL) was heated at 65° C. for 12 hours. The reaction was purified by HPLC, eluting with water/acetonitrile/0.2% trifluoroacetic acid. The product fractions were concentrated to dryness, EtOH was added, the mixture was heated, allowed to cool, and the title compound collected as tan crystals.

### Intermediate 153: 1-(2-Methoxy-phenyl)-3-(1-methyl-1H-benzoimidazol-4-yl)-thiourea

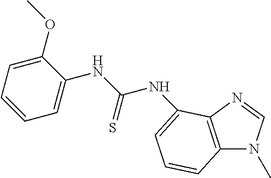

A mixture of 2-methoxyphenylisothiocyanate (0.20 mL, 1.45 mmol) and 4-amino-1-methylbenzimidazole (0.213 g, 1.45 mmol) in DMF (1 mL) was stirred at room temperature for 60 hours. The reaction was concentrated and the residue stirred with water. The product was collected and dried in a 50° C. vacuum oven overnight to yield the title compound.

### Intermediate 154: 1-(5-Methanesulfonyl-2-methoxy-phenyl)-3-(1-methyl-1H-benzoimidazol-4-yl)-thiourea

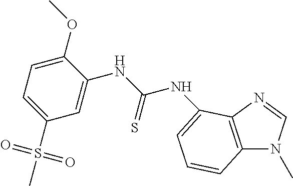

The title compound was prepared from Intermediate 110 by the procedure of Intermediate 153.

### Intermediate 155: 1-(5-Fluoro-2-methoxy-phenyl)-3-(1-methyl-1H-benzoimidazol-4-yl)-thiourea

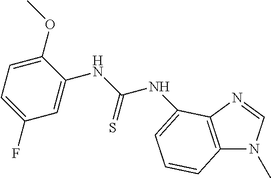

The title compound was prepared from Intermediate 115 by the procedure of Intermediate 153.

### Intermediate 156: 3-[3-(1-Methyl-1H-benzoimidazol-4-yl)-thioureido]-4-trifluoromethoxy-benzenesulfonamide

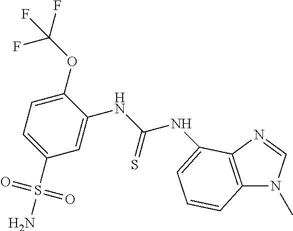

The title compound was prepared from Intermediate 121 by the procedure of Intermediate 153.

### Intermediate 157: 3-[3-(1-Methyl-1H-benzoimidazol-4-yl)-thioureido]-4-trifluoromethoxy-benzamide

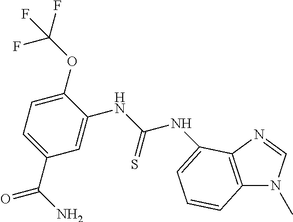

The title compound was prepared from Intermediate 122 by the procedure of Intermediate 153.

### Intermediate 158: {4-Methoxy-3-[3-(1-methyl-1H-benzoimidazol-4-yl)-thioureido-]phenyl}-phosphonic acid diethyl ester.TFA

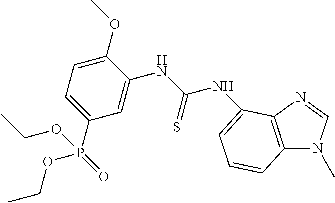

The title compound was prepared from Intermediate 123 and 4-amino-1-methylbenzimidazole by the procedure of Intermediate 152.

### Intermediate 159: 1-(5-Cyano-2-methoxy-phenyl)-3-(1-methyl-1H-benzoimidazol-4-yl)-thiourea.TFA

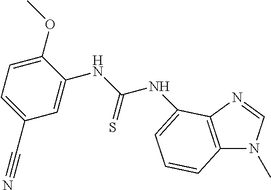

The title compound was prepared from Intermediate 124 and 4-amino-1-methylbenzimidazole by the procedure of Intermediate 152.

### Intermediate 160:1-(5-Bromo-2-methoxy-phenyl)-3-(1-methyl-1H-benzoimidazol-4-yl)-thiourea.TFA

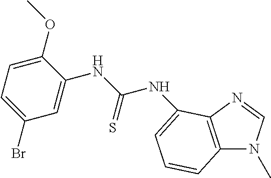

The title compound was prepared from Intermediate 125 and 4-amino-1-methylbenzimidazole by the procedure of Intermediate 152.

### Intermediate 161: N,N-Diethyl-4-methoxy-3-[3-(1-methyl-1H-benzoimidazol-4-yl)-thioureido]-benzenesulfonamide

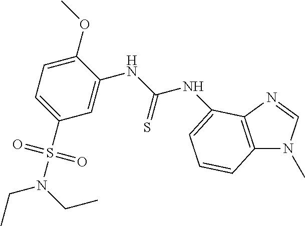

The title compound was prepared from Intermediate 126 by the procedure of Intermediate 153.

### Intermediate 162: 1-[2-Methoxy-5-(pyrrolidine-1-sulfonyl)-phenyl]-3-(1-methyl-1H-benzoimidazol-4-yl)-thiourea

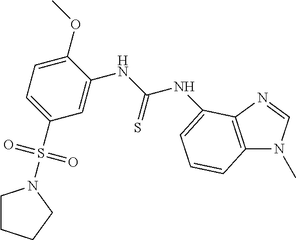

The title compound was prepared from Intermediate 127 by the procedure of Intermediate 153.

### Intermediate 163: 1-(4-Methoxy-pyridin-3-yl)-3-(1-methyl-1H-benzoimidazol-4-yl)-thiourea

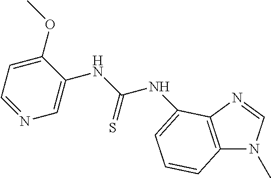

A mixture of Intermediate 128 (0.125 g, 0.661 mmol) and Intermediate 67 (0.082 g, 0.661 mmol) in DMF (3 mL) was stirred at room temperature for 15 hours. The reaction was concentrated and the residue treated with MeCN, reconcentrating and adding more MeCN until a solid resulted. The product was collected and dried under nitrogen/vacuum to yield the title compound.

### Intermediate 164: 1,3-Bis-(1-methyl-1H-benzoimidazol-4-yl)-thiourea

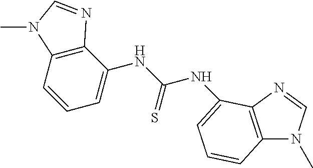

The title compound was isolated from the reaction that produced Intermediate 163.

### Intermediate 165: 1-(2-Methoxy-pyridin-3-yl)-3-(1-methyl-1H-benzoimidazol-4-yl)-thiourea

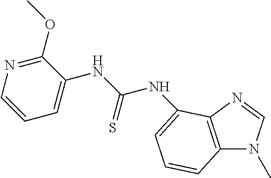

The title compound was prepared from 2-methoxy-pyridin-3-ylamine and Intermediate 128 by the procedure of Intermediate 163.

### Intermediate 166: 1-(1-Methyl-1H-benzoimidazol-4-yl)-3-pyridin-3-yl-thiourea

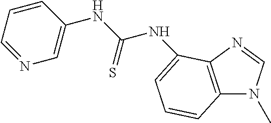

The title compound was prepared from 3-aminopyridine and Intermediate 128 by the procedure of Intermediate 163.

### Intermediate 167: 1-(2-Methoxy-5-nitro-phenyl)-3-(1-methyl-1H-benzoimidazol-4-yl)-thiourea

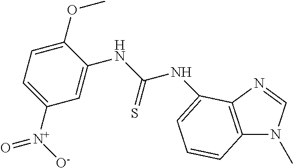

The title compound was prepared from 2-methoxy-5-nitrophenyl isothiocyanate and 4-amino-1-methylbenzimidazole by the procedure of Intermediate 163.

### Intermediate 168: 1-(1-Methyl-1H-benzoimidazol-4-yl)-3-(2-nitro-phenyl)-thiourea

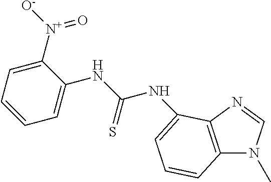

The title compound was prepared from 2-nitrophenyl isothiocyanate and 4-amino-1-methylbenzimidazole by the procedure of Intermediate 163.

### Intermediate 169: 1-(2,4-Dimethoxy-phenyl)-3-(1-methyl-1H-benzoimidazol-4-yl)-thiourea

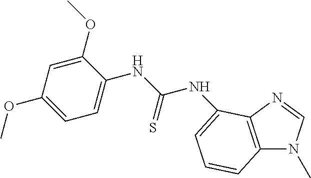

The title compound was prepared from 2,4-dimethoxyphenyl isothiocyanate and 4-amino-1-methylbenzimidazole by the procedure of Intermediate 163.

### Intermediate 170:1-[2-Methoxy-5-(piperidine-1-sulfonyl)-phenyl]-3-(1-methyl-1H-benzoimidazol-4-yl)-thiourea

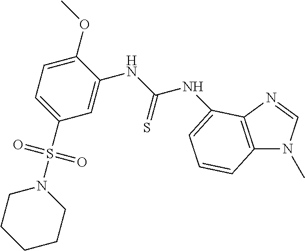

The title compound was prepared from Intermediate 129 and 4-amino-1-methylbenzimidazole by the procedure of Intermediate 163.

### Intermediate 171: 1-[2-Methoxy-5-(morpholine-4-sulfonyl)-phenyl]-3-(1-methyl-1H-benzoimidazol-4-yl)-thiourea

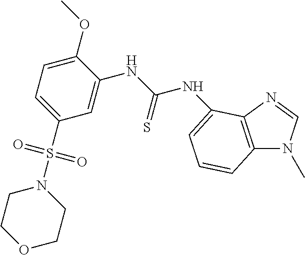

The title compound was prepared from Intermediate 130 and 4-amino-1-methylbenzimidazole by the procedure of Intermediate 163.

### Intermediate 172: 4-Methoxy-N-methyl-3-[3-(1-methyl-1H-benzoimidazol-4-yl)-thioureido]-benzenesulfonamide

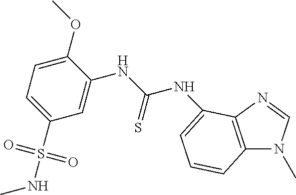

The title compound was prepared from Intermediate 131 and 4-amino-1-methylbenzimidazole by the procedure of Intermediate 163.

### Intermediate 173: 1-(2-Methoxy-4-nitro-phenyl)-3-(1-methyl-1H-benzoimidazol-4-yl)-thiourea

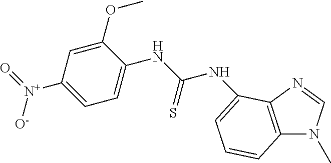

The title compound was prepared from 2-methoxy-4-nitrophenyl isothiocyanate and 4-amino-1-methylbenzimidazole by the procedure of Intermediate 163.

### Intermediate 174: 1-Benzooxazol-4-yl-3-(5-cyano-2-methoxy-phenyl)-thiourea

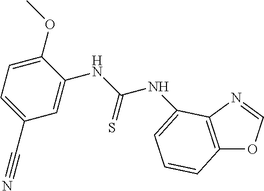

The title compound was prepared from Intermediate 124 and Intermediate 62 by the procedure of Intermediate 163.

### Intermediate 175: 1-(5-Cyano-2-methoxy-phenyl)-3-(1-methyl-1H-indol-4-yl)-thiourea

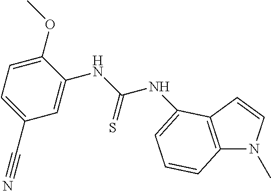

The title compound was prepared from Intermediate 124 and 1-methyl-1H-indol-4-ylamine by the procedure of Intermediate 163.

### Intermediate 176: 1-(4-Cyano-2-trifluoromethoxy-phenyl)-3-(1-methyl-1H-benzoimidazol-4-yl)-thiourea

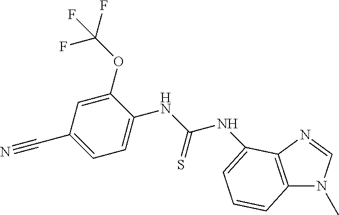

To NaH (0.0125 g, 0.495 mmol) under Ar was added dry DMF (2 mL) and the resulting suspension chilled in an ice bath. 4-amino-3-trifluoromethoxybenzonitrile (0.10 g, 0.495 mmol) was added in one portion, and the ice bath removed and the reaction allowed to warmed to room temperature. After 25 minutes, Intermediate 128 (0.094 g, 0.495 mmol) was added portion-wise. After 15 minutes, the reaction was concentrated under high vacuum, and the resulting gummy yellow solid was used without further purification.

### Intermediate 177: {4-Methoxy-3-[3-(1-methyl-1H-benzoimidazol-4-yl)-thioureido]-phenyl}-urea

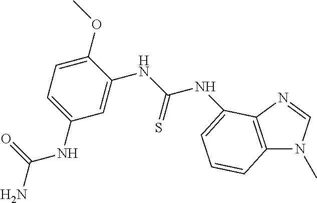

The title compound was prepared from Intermediate 69 and Intermediate 128 by the procedure of Intermediate 163.

### Intermediate 178: 4-Isobutyl-3-[3-(1-methyl-1H-benzoimidazol-4-yl)-thioureido]-benzamide

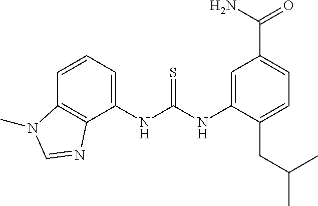

The title compound was prepared from Intermediate 132 and 4-amino-1-methylbenzimidazole according to the procedure described for Intermediate 163. The crude product was purified by flash column chromatography (Silica gel, 80-100% EtOAc/hept then 0-4% MeOH/EtOAc), affording the title compound as a white solid.

### Intermediate 179: 1-(5-Methanesulfonyl-2-methoxy-phenyl)-3-(1-methyl-1H-indazol-4-yl)-thiourea

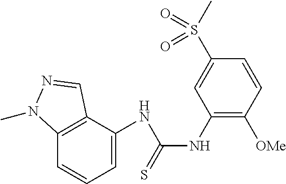

To 1-methyl-1H-indazol-4-ylamine (0.2 g, 1.36 mmol) under N₂ was added Intermediate 110 (0.33 g, 1.36 mmol) and the mixture was dissolved in DMSO (1 mL). The reaction was stirred at room temperature for 20 hours. The title compound was precipitated with ice water, collected, and dried under nitrogen/vacuum.

### Intermediate 180:1-(5-Fluoro-2-methoxy-phenyl)-3-(1-methyl-1H-indazol-4-yl)-thiourea

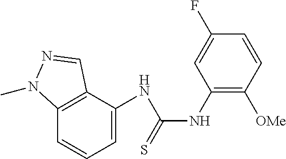

The title compound was prepared from 1-methyl-1H-indazol-4-ylamine and Intermediate 115 by the procedure of Intermediate 179.

### Intermediate 181: 1-(2-Methoxy-phenyl)-3-(1-methyl-1H-indazol-4-yl)-thiourea

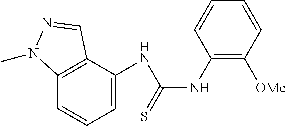

The title compound was prepared from 1-methyl-1H-indazol-4-ylamine and 2-isothiocyanato-1-methoxy-benzene by the procedure of Intermediate 179.

### Intermediate 182: 4-Methoxy-3-[3-(1-methyl-1H-indazol-4-yl)-thioureido]-benzamide

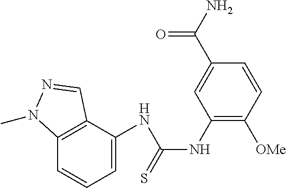

The title compound was prepared from 1-methyl-1H-indazol-4-ylamine and Intermediate 103 by the procedure of Intermediate 179.

### Intermediate 183: 3-(3-Isoquinolin-5-yl-thioureido)-4-methoxy-benzamide

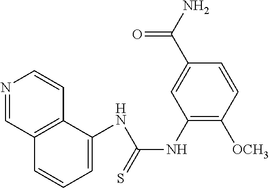

The title compound was prepared from isoquinolin-5-ylamine and Intermediate 103 by the procedure of Intermediate 179.

### Intermediate 184: 1-Isoquinolin-5-yl-3-(5-methanesulfonyl-2-methoxy-phenyl)-thiourea

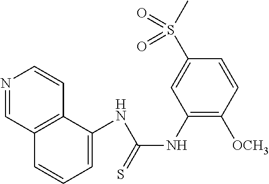

The title compound was prepared from isoquinolin-5-ylamine and Intermediate 110 by the procedure of Intermediate 179.

### Intermediate 185: 3-(3-Isoquinolin-5-yl-thioureido)-4-trifluoromethoxy-benzenesulfonamide

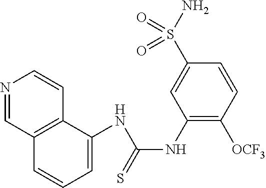

The title compound was prepared from isoquinolin-5-ylamine and Intermediate 121 by the procedure of Intermediate 179.

### Intermediate 186: 1-(5-Fluoro-2-methoxy-phenyl)-3-isoquinolin-5-yl-thiourea

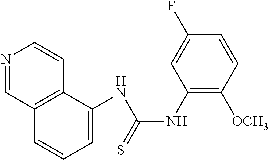

The title compound was prepared from isoquinolin-5-ylamine and Intermediate 115 by the procedure of Intermediate 179.

### Intermediate 187: 1-Isoquinolin-5-yl-3-(2-methoxy-phenyl)-thiourea

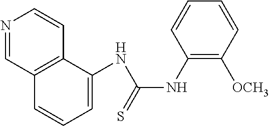

The title compound was prepared from isoquinolin-5-ylamine and 2-isothiocyanato-1-methoxy-benzene by the procedure of Intermediate 179.

### Intermediate 188: 3-[3-(1-Methyl-1H-indazol-4-yl)-thioureido]-4-trifluoromethoxy-benzenesulfonamide

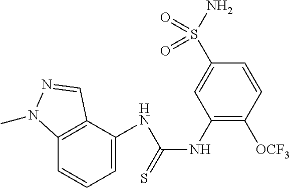

The title compound was prepared from 1-methyl-1H-indazol-4-ylamine and Intermediate 121 by the procedure of Intermediate 179.

### Intermediate 189: 4-Isopropoxy-3-[3-(1-methyl-1H-indazol-4-yl)-thioureido]-benzenesulfonamide

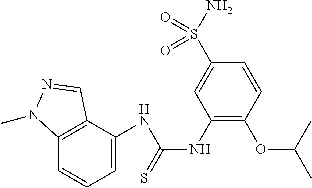

The title compound was prepared from 1-methyl-1H-indazol-4-ylamine and Intermediate 100 by the procedure of Intermediate 179.

### Intermediate 190: 4-Methoxy-3-[3-(1-methyl-1H-indazol-4-yl)-thioureido]-benzenesulfonamide

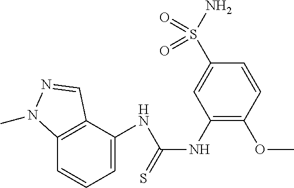

The title compound was prepared from 1-methyl-1H-indazol-4-ylamine and Intermediate 112 by the procedure of Intermediate 179.

### Intermediate 191: 3-(3-Isoquinolin-5-yl-thioureido)-4-methoxy-benzenesulfonamide

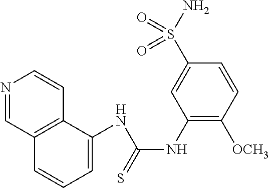

The title compound was prepared from isoquinolin-5-ylamine and Intermediate 112 by the procedure of Intermediate 179.

### Intermediate 192: 4-Isopropoxy-3-(3-isoquinolin-5-yl-thioureido)-benzenesulfonamide

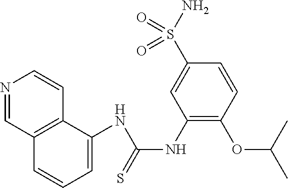

The title compound was prepared from isoquinolin-5-ylamine and Intermediate 100 by the procedure of Intermediate 179.

### Intermediate 193: 1-(1-Ethyl-1H-indazol-4-yl)-3-(5-methanesulfonyl-2-methoxy-phenyl)-thiourea

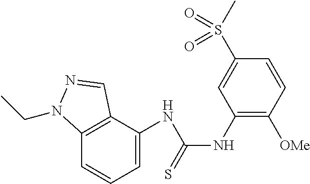

The title compound was prepared from 80 and Intermediate 110 by the procedure of Intermediate 179.

### Intermediate 194: 1-(2-Ethyl-2H-indazol-4-yl)-3-(5-methanesulfonyl-2-methoxy-phenyl)-thiourea

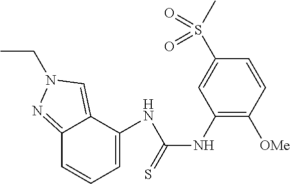

The title compound was prepared from 81 and Intermediate 110 by the procedure of Intermediate 179.

### Intermediate 195: 1-(1-Isobutyl-1H-indazol-4-yl)-3-(5-methanesulfonyl-2-methoxy-phenyl)-thiourea

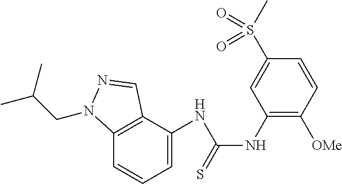

The title compound was prepared from 82 and Intermediate 110 by the procedure of Intermediate 179.

### Intermediate 196: 1-(5-Methanesulfonyl-2-methoxy-phenyl)-3-[1-(3-methyl-butyl)-1H-indazol-4-yl]-thiourea

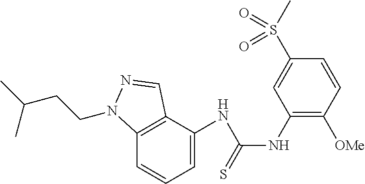

The title compound was prepared from Intermediate 83 and Intermediate 110 by the procedure of Intermediate 179.

### Intermediate 197: 1-(1H-Indazol-4-yl)-3-(5-methanesulfonyl-2-methoxy-phenyl)-thiourea

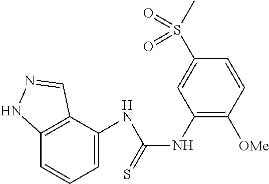

The title compound was prepared from 1H-indazol-4-ylamine and Intermediate 110 by the procedure of Intermediate 179.

### Intermediate 198: 1-(5-Methanesulfonyl-2-methoxy-phenyl)-3-(1-propyl-1H-indazol-4-yl)-thiourea

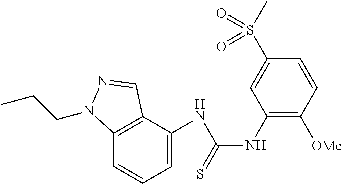

The title compound was prepared from Intermediate 84 and Intermediate 110 by the procedure of Intermediate 179.

### Intermediate 199: 1-(5-Cyano-2-methoxy-phenyl)-3-(1-methyl-1H-indazol-4-yl)-thiourea

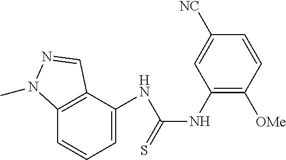

The title compound was prepared from 1H-indazol-4-ylamine and Intermediate 124 by the procedure of Intermediate 179.

### Intermediate 200: 3-[3-(1-Methyl-1H-indazol-4-yl)-thioureido]-4-trifluoromethoxy-benzamide

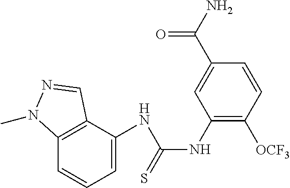

The title compound was prepared from 1-methyl-1H-indazol-4-ylamine and Intermediate 122 by the procedure of Intermediate 179.

### Intermediate 201: 3-(3-Isoquinolin-5-yl-thioureido)-4-trifluoromethoxy-benzamide

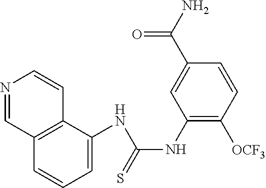

The title compound was prepared from isoquinolin-5-ylamine and Intermediate 122 by the procedure of Intermediate 179.

### Intermediate 202: 3-[3-(3-Ethyl-2-methyl-3H-benzoimidazol-4-yl)-thioureido]-4-isopropoxy-benzenesulfonamide

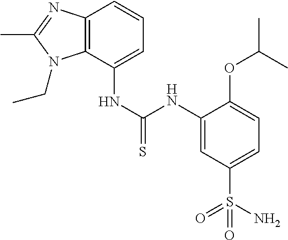

A mixture of Intermediate 93 (50 mg, 0.285 mmol) and Intermediate 100 (78 mg, 0.285 mmol) in DMF was stirred overnight and concentrated. The residue was purified by column chromatography (Silica gel, 3%-10% MeOH in CH₂Cl₂) to provide the title compound as a brown solid.

### Intermediate 203: 3-[3-(2,3-Dimethyl-3H-benzoimidazol-4-yl)-thioureido]-4-isopropoxy-benzenesulfonamide

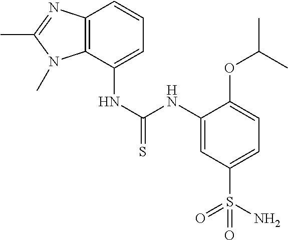

A mixture of Intermediate 94 (50 mg, 0.31 mmol) and Intermediate 100 (84 mg, 0.31 mmol) in DMF was stirred overnight and concentrated. To the residue was added water, and the precipitated solid was filtered, washed with water, and dried to obtain the title compound as light brown solid.

### Intermediate 204: 1-(3-Ethyl-2-methyl-3H-benzoimidazol-4-yl)-3-(2-methoxy-phenyl)-thiourea

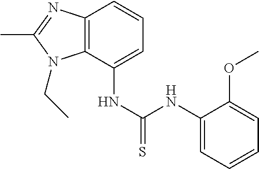

The title compound was prepared from Intermediate 93 and 2-isothiocyanato-1-methoxy-benzene by the method of Intermediate 203.

### Intermediate 205: 1-(3-Ethyl-2-methyl-3H-benzoimidazol-4-yl)-3-(5-fluoro-2-methoxy-phenyl)-thiourea

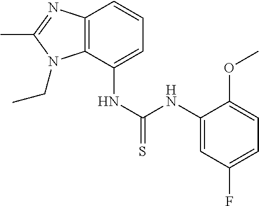

The title compound was prepared from Intermediate 93 and Intermediate 115 by the method of intermediate 203.

### Intermediate 206: 3-[3-(3-Ethyl-2-methyl-3H-benzoimidazol-4-yl)-thioureido]-4-trifluoromethoxy-benzenesulfonamide

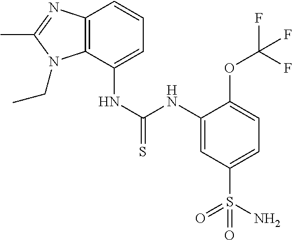

The title compound was prepared from Intermediate 93 and Intermediate 121 by the method of Intermediate 203.

### Intermediate 207: 3-[3-(2,3-Dimethyl-3H-benzoimidazol-4-yl)-thioureido]-4-methoxy-benzenesulfonamide.TFA

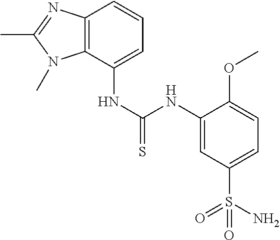

A mixture of Intermediate 94 (0.053 g, 0.33 mmol) and intermediate 112 (0.080 g, 0.33 mmol) in DMF was stirred overnight and purified by HPLC eluting with water/acetonitrile/0.2% trifluoroacetic acid to provide the title compound as an oil.

### Intermediate 208: 1-(2,3-Dimethyl-3H-benzoimidazol-4-yl)-3-(2-methoxy-phenyl)-thiourea

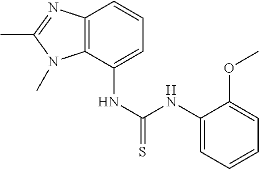

The title compound was prepared using 2-isothiocyanato-1-methoxy-benzene in place of Intermediate 100 by the method of intermediate 203.

### Intermediate 209: 1-(2,3-Dimethyl-3H-benzoimidazol-4-yl)-3-(5-fluoro-2-methoxy-phenyl)-thiourea

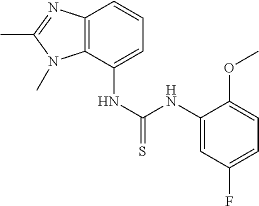

The title compound was prepared using Intermediate 115 in place of Intermediate 100 by the method of Intermediate 203.

### Intermediate 210: 3-[3-(2,3-Dimethyl-3H-benzoimidazol-4-yl)-thioureido]-4-trifluoromethoxy-benzenesulfonamide

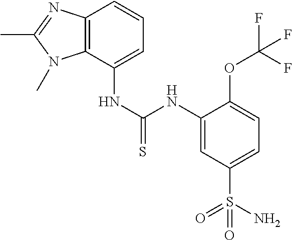

The title compound was prepared using Intermediate 121 in place of Intermediate 100 by the method of Intermediate 203.

### Intermediate 211: 1-(5-Fluoro-2-methoxy-phenyl)-3-(3-methyl-3H-benzoimidazol-4-yl)-thiourea

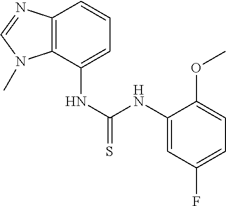

The title compound was prepared from Intermediate 95 and Intermediate 115 by the method of Intermediate 203.

### Intermediate 212: 3-[3-(3-Methyl-3H-benzoimidazol-4-yl)-thioureido]-4-trifluoromethoxy-benzenesulfonamide

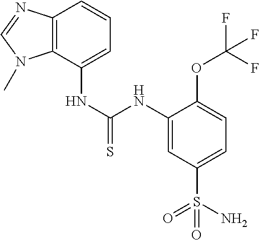

The title compound was prepared from Intermediate 95 and Intermediate 121 by the method of Intermediate 203.

### Intermediate 213: 3-[3-(3-Ethyl-2-methyl-3H-benzoimidazol-4-yl)-thioureido]-4-methoxy-benzenesulfonamide

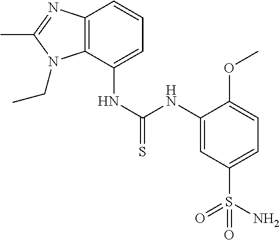

The title compound was prepared from Intermediate 93 and Intermediate 112 by the method of Intermediate 203.

### Intermediate 214: 4-Methoxy-3-[3-(3-methyl-3H-benzoimidazol-4-yl)-thioureido]-benzenesulfonamide

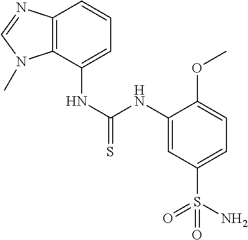

The title compound was prepared from Intermediate 95 and Intermediate 112 by the method of Intermediate 203.

### Intermediate 215: 4-Isopropoxy-3-[3-(3-methyl-3H-benzoimidazol-4-yl)-thioureido]-benzenesulfonamide

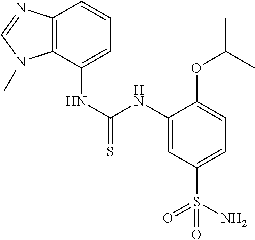

The title compound was prepared using Intermediate 95 in place of Intermediate 94 by the method of Intermediate 203.

### Intermediate 216: 1-(2-Methoxy-phenyl)-3-(3-methyl-3H-benzoimidazol-4-yl)-thiourea

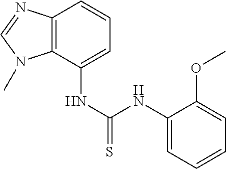

The title compound was prepared from Intermediate 95 and 2-isothiocyanato-1-methoxy-benzene by the method of Intermediate 203.

### Intermediate 217: 1-(3-Ethyl-2-methyl-3H-benzoimidazol-4-yl)-3-(5-methanesulfonyl-2-methoxy-phenyl)-thiourea

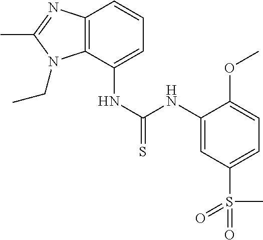

The title compound was prepared from Intermediate 93 and Intermediate 110 by the method of Intermediate 203.

### Intermediate 218: 1-(2,3-Dimethyl-3H-benzoimidazol-4-yl)-3-(5-methanesulfonyl-2-methoxy-phenyl)-thiourea

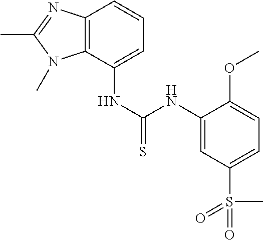

The title compound was prepared using Intermediate 110 in place of Intermediate 100 by the method of Intermediate 203.

### Intermediate 219: 1-(5-Methanesulfonyl-2-methoxy-phenyl)-3-(3-methyl-3H-benzoimidazol-4-yl)-thiourea

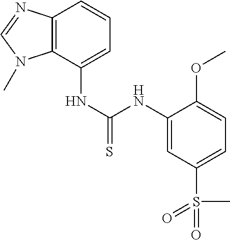

The title compound was prepared from Intermediate 95 and Intermediate 110 by the method of Intermediate 203.

### Intermediate 220: 4-Isopropoxy-3-{3-[2-methyl-3-(2,2,2-trifluoro-ethyl)-3H-benzoimidazol-4-yl]-thioureido}-benzenesulfonamide

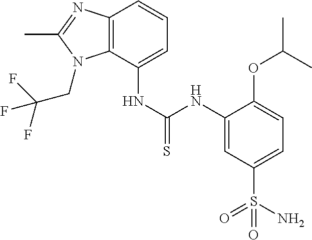

The title compound was prepared using Intermediate 96 in place of Intermediate 94 by the method of Intermediate 203.

### Intermediate 221: 4-Isopropoxy-3-{3-[3-(2,2,2-trifluoro-ethyl)-3H-benzoimidazol-4-yf]-thioureido}-benzenesulfonamide

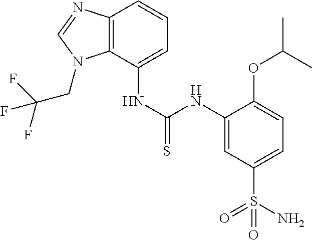

The title compound was prepared using Intermediate 97 in place of Intermediate 94 by the method of Intermediate 203.

### Intermediate 222: 1-(5-Methanesulfonyl-2-methoxy-phenyl)-3-[2-methyl-3-(2,2,2-trifluoro-ethyl)-3H-benzoimidazol-4-yf]-thiourea

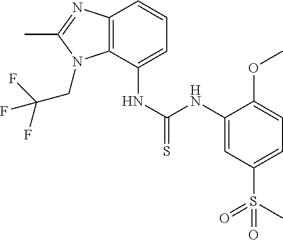

A mixture of intermediate 96 (0.019 g, 0.083 mmol) and Intermediate 110 (0.020 g, 0.082 mmol) in DMF was stirred overnight and concentrated to obtain the title compound as a light brown solid.

### Intermediate 223: 1-(3-Ethyl-2-methyl-3H-benzoimidazol-4-yl)-3-(5-fluoro-2-isopropoxy-phenyl)-thiourea

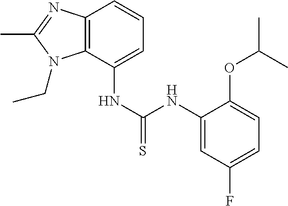

A mixture of Intermediate 93 (0.030 g, 0.17 mmol) and Intermediate 117 (0.036 g, 0.17 mmol) in DMF was stirred at room temperature overnight and then heated at 65° C. for 4 hours. After removal of DMF in vacuo, the residue was purified by flash column chromatography (Silica gel; 50%-100% EtOAc/heptanes) to give the title compound as a yellow oil.

### Intermediate 224: 1-(2,3-Dimethyl-3H-benzoimidazol-4-yl)-3-(5-fluoro-2-isopropoxy-phenyl)-thiourea

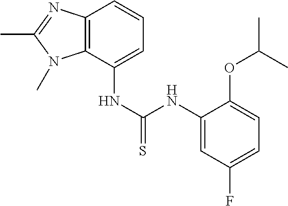

The title compound was prepared using Intermediate 94 in place of Intermediate 93 by the method of Intermediate 223.

### Intermediate 225: 1-(5-Fluoro-2-isopropoxy-phenyl)-3-[2-methyl-3-(2,2,2-trifluoro-ethyl)-3H-benzoimidazol-4-yl]-thiourea

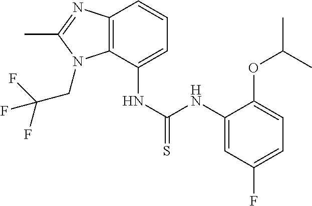

The title compound was prepared using Intermediate 96 in place of Intermediate 93 by the method of Intermediate 223, with purification by flash column chromatography (Silica gel; 2-10% MeOH/CH₂Cl₂).

### Intermediate 226: 1-(5-Fluoro-2-isopropoxy-phenyl)-3-[3-(2,2,2-trifluoro-ethyl)-3H-benzoimidazol-4-yl]-thiourea

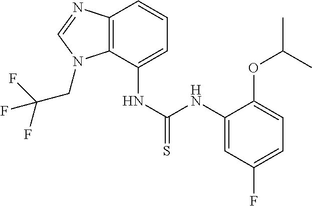

The title compound was prepared using Intermediate 97 in place of Intermediate 96 by the method of Intermediate 225.

### Intermediate 227: 1-(3-Ethyl-2-methyl-3H-benzoimidazol-4-yl)-3-(2-isopropoxy-5-methanesulfonyl-phenyl)-thiourea

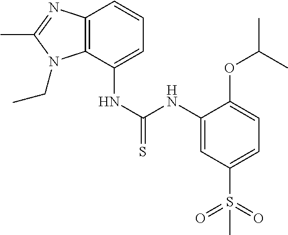

The title compound was prepared from Intermediate 93 and Intermediate 133 by the method of Intermediate 203.

### Intermediate 228: 1-(2,3-Dimethyl-3H-benzoimidazol-4-yl)-3-(2-isopropoxy-5-methanesulfonyl-phenyl)-thiourea

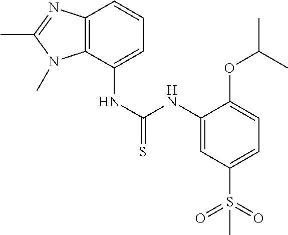

The title compound was prepared using Intermediate 133 in place of Intermediate 100 by the method of Intermediate 203.

### Intermediate 229: 1-(2-Isopropoxy-5-methanesulfonyl-phenyl)-3-[2-methyl-3-(2,2,2-trifluoro-ethyl)-3H-benzoimidazol-4-yl]-thiourea

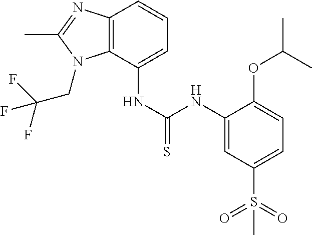

The title compound was prepared from Intermediate 96 and Intermediate 133 by the method of Intermediate 203.

### Intermediate 230:1-(2-Isopropoxy-5-methanesulfonyl-phenyl)-3-[3-(2,2,2-trifluoro-ethyl)-3H-benzoimidazol-4-yl]-thiourea

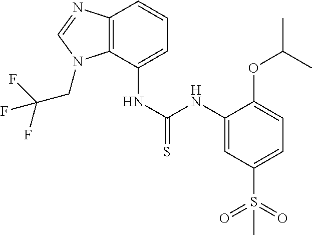

The title compound was prepared from Intermediate 97 and Intermediate 133 by the method of Intermediate 203.

### Intermediate 231: 1-(2-Isopropoxy-5-methanesulfonyl-phenyl)-3-(1-methyl-1H-benzoimidazol-4-yl)-thiourea

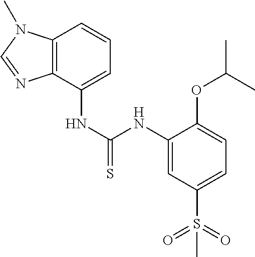

The title compound was prepared from 4-amino-1-methylbenzimidazole and Intermediate 133 by the method of Intermediate 203.

### Intermediate 232: 1-(4-Methoxy-pyridin-3-yl)-3-[2-methyl-3-(2,2,2-trifluoro-ethyl)-3H-benzoimidazol-4-yl]-thiourea

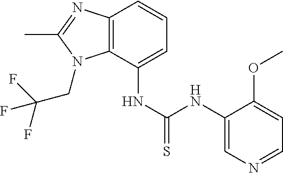

A mixture of Intermediate 96 (0.104 g, 0.454 mmol) and Intermediate 134 (0.075 g, 0.45 mmol) in DMF was stirred at room temperature for 64 hours. After removal of DMF in vacuo, the residue was purified by flash column chromatography (Silica gel; 1-10% MeOH/CH₂Cl₂) to give the title compound as a light brown solid.

### Intermediate 233: 1-(4-Methoxy-pyridin-3-yl)-3-(1-methyl-1H-benzoimidazol-4-yl)-thiourea

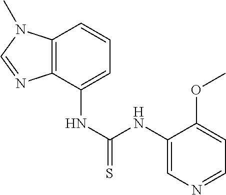

A mixture of 4-amino-1-methylbenzimidazole (0.100 g, 0.679 mmol) and Intermediate 134 (0.113 mg, 0.680 mmol) in DMF was stirred at room temperature for 64 hours. After removal of DMF in vacuo, the residue was treated with water. The precipitated solid was filtered, washed with water, and dried to give a portion of the title compound. The filtrate was concentrated and the oily brown residue was dried under vacuum to provide a second portion of the title compound.

### Intermediate 234: N-[2-(2-Methoxy-phenylamino)-6H-imidazo[4′,5′:3,4]benzo[2,1-d]thiazol-7-yl]-benzamide

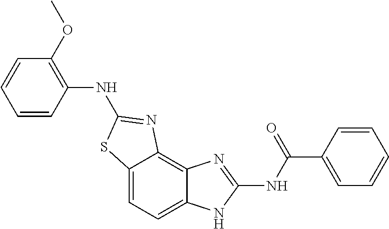

A solution of Intermediate 138 (0.036 g, 0.086 mmol) in CHCl₃ (5 mL) was chilled on a dry ice/acetone bath to yield an opaque mixture. Sulfuryl chloride (0.007 mL, 0.086 mmol) was added and the bath removed. When the reaction warmed to room temperature, it was concentrated and the residue stirred with MeCN. A solid was collected and purified by flash column chromatography (Silica gel, 0-2.5% MeOH/CH₂Cl₂) to yield the title compound.

### Intermediate 235: 3-(7-Benzoylamino-6H-imidazo[4′,5′:3,4]benzo[2,1-d]thiazol-2-ylamino)-benzoic acid methyl ester

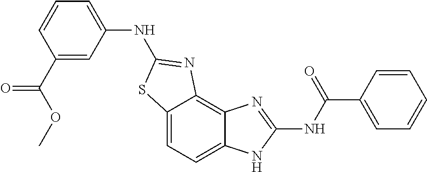

The title compound was prepared from Intermediate 139 according to the method of Intermediate 234, with the following changes. Dioxane was used in place of CHCl₃. The crude product was partially purified by trituration with MeCN.

### Intermediate 236: N-[2-(2-Isopropoxy-5-carboxamido-phenylamino)-6H-imidazo[4′,5′:3,4]-benzo[2,1-d]thiazol-7-yl]-benzamide

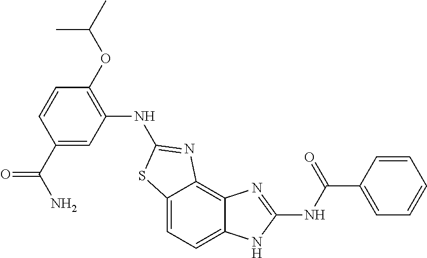

A solution of Intermediate 46 (0.153 g, 0.610 mmol) and Intermediate 99 (0.143 g, 0.61 mmol) in DMSO (4 mL) was heated to 60° C. for 15 hours. To 3 mL of the crude thiourea solution was added DDQ (0.10 g, 0.44 mmol) in DMSO (3.5 mL) via syringe pump over 1 hour. The reaction was poured into water (60 mL) and the crude product was collected, dissolved in DMSO and purified by HPLC, eluting with water/acetonitrile/0.2% trifluoroacetic acid. The product fractions were concentrated to remove MeCN, and the title compound was collected and used without further purification.

### Intermediate 237: 2-Amino-6,6-dibromo-5,6-dihydro-4H-benzothiazol-7-one

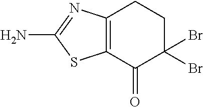

A solution of 2-amino-5,6-dihydro-4H-benzothiazol-7-one (0.29 g, 1.71 mmol) in 48% HBr (7 mL) was heated to 60° C. in an oil bath. A solution of bromine (0.18 mL, 3.43 mmol) in dioxane (7 mL) was added dropwise and the resulting red/orange solution stirred at 60° C. for 2 hours, then at rt overnight. The reaction mixture was carefully poured into excess saturated aqueous NaHCO₃ solution, and a brown solid was collected, washed with water, and dried under nitrogen/vacuum. The solid was stirred with MeCN and the title compound was collected as a brown solid.

### Intermediate 238: 2-Amino-6-bromo-5,6-dihydro-4H-benzothiazol-7-one.HBr

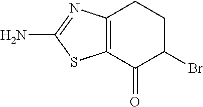

To a solution of 2-amino-5,6-dihydro-4H-benzothiazol-7-one (0.46 g, 2.74 mmol) in 48% HBr (10 mL) at 60° C. was added a solution of bromine (0.15 mL, 3.0 mmol) in dioxane (10 mL) by addition funnel. After the addition was complete the reaction was stirred at 60° C. for 2 hours. The reaction was concentrated, taken up in EtOH and reconcentrated. The residue was stirred with MeCN, collected, rinsed with MeCN and dried under nitrogen/vacuum to yield the title compound as an off-white solid.

### Intermediate 239: 2-Bromo-cyclohexane-1,3-dione

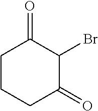

The title compound was prepared according to J. Chem. Soc. Perkin Trans. I 1987, 2153-2155.

### Intermediate 240: 2-Bromo-cycloheptane-1,3-dione

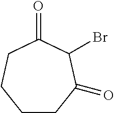

The title compound was made by the method of Intermediate 239, using cycloheptanedione in place of cyclohexanedione.

### Intermediate 241: 2-Methyl-5,6-dihydro-4H-benzothiazol-7-one

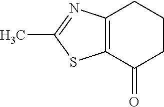

Intermediate 239 (2.85 g, 14.9 mmol) and thioacetamide (1.12 g, 14.9 mmol) were dissolved in dry pyridine (40 mL) and stirred for 12 hours. The reaction was concentrated and the residue partitioned between ethyl acetate and enough 1N NaOH to make the aqueous layer basic. The layers were separated, and the organics were washed twice with water, once with brine, filtered through cotton, dried over Na₂SO₄, filtered, and concentrated. The residue was purified by flash column chromatography (Silica gel, 0-50% EtOAc/heptane) to yield the title compound.

### Intermediate 242: 2-Methyl-4,5,6,7-tetrahydro-cycloheptathiazol-8-one

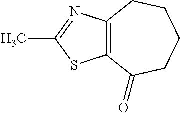

The title compound was prepared by the method of Intermediate 241, substituting Intermediate 240 for Intermediate 239.

### Intermediate 243: 6-Bromo-2-methyl-5,6-dihydro-4H-benzothiazol-7-one.HBr

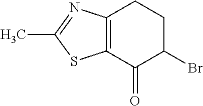

To a solution of Intermediate 241 (0.59 g, 3.53 mmol) in dioxane (10 mL) under Ar at 60° C. was added a solution of bromine (0.182 mL) in dioxane (10 mL) dropwise. The reaction was heated at 60° C. for 2 hours, then at room temperature for 12 hours, then concentrated to a brown solid. This solid was triturated with CH₂Cl₂ to yield the title compound as a tan solid.

### Intermediate 244: 7-Bromo-2-methyl-4,5,6,7-tetrahydro-cycloheptathiazol-8-one.HBr

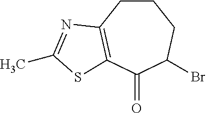

The title compound was prepared by the method of Intermediate 243, substituting Intermediate 242 for Intermediate 241.

### Intermediate 245: (2-Isopropoxy-phenyl)-(2-methyl-4,5-dihydro-benzo[1,2-d;3,4-d′]-bisthiazol-7-yl)-amine

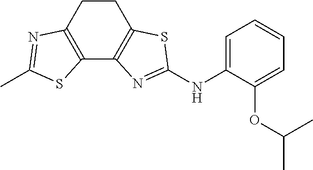

The title compound was prepared according to the method of Example 12, replacing Intermediate 135 with Intermediate 136.

### Intermediate 246: 5-(tert-Butyl-dimethyl-silanyloxy)-7,8-dihydro-quinoline

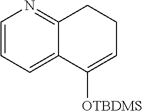

tert-Butyldimethylsilyl trifluoromethanesulfonate (0.683 g, 2.58 mmol) was added to a solution of 7,8-dihydro-6H-quinolin-5-one (0.200 g, 1.36 mmol), triethylamine (0.379 mL, 0.272 mmol) and dichloromethane (20 mL) at room temperature. The reaction mixture was then quenched with saturated aqueous NaHCO₃, extracted with dichloromethane, dried with sodium sulfate and purified via flash column chromatography (Silica gel, heptanes/EtOAc) to give the title compound.

### Intermediate 247: 6-Bromo-7,8-dihydro-6H-quinolin-5-one

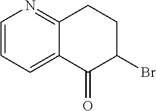

A solution of Intermediate 246 (0.295 g, 1.13 mmol) and pyridinium tribromide (0.399 g, 1.06 mmol) in acetic acid (5 mL) was stirred at room temperature overnight. The reaction mixture was evaporated in vacuo, saturated aqueous NaHCO₃ was added and the crude product was extracted with ethyl acetate. The product was purified via flash column chromatography (Silica gel, heptanes/EtOAc) to give the title compound.

### Intermediate 248: 6-Bromo-7,8-dihydro-6H-isoquinolin-5-one.HBr

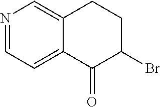

0.778 M bromine in 1,4-dioxane (1 mL, 0.778 mmol) was added to a stirred solution of 7,8-dihydro-6H-isoquinolin-5-one (0.121 g, 0.819 mmol) in 1,4-dioxane (1 mL). The mixture was stirred at 50° C. for 24 h and the resulting cream-colored suspension was allowed to cool to room temperature and was filtered, washed with 2:1 heptane:EtOAc (v/v) and air dried to give the title compound.

### Intermediate 249: 7-Bromo-6,7-dihydro-5H-isoquinolin-8-one.HBr

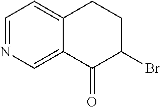

The title compound was prepared using 6,7-dihydro-5H-isoquinolin-8-one in place of 7,8-dihydro-6H-isoquinolin-5-one according to the procedure described in Intermediate 248.

### Intermediate 250:1-(5-Methanesulfonyl-2-methoxy-phenyl)-3-(1-methyl-1H-indazol-7-yl)-thiourea

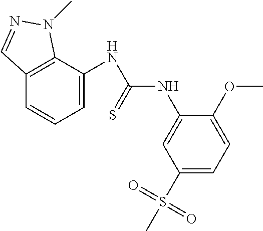

The title compound was prepared from 1-methyl-1H-indazol-7-ylamine and Intermediate 110 by the procedure of Intermediate 179.

### Intermediate 251: 1-(5-Cyano-2-methoxy-phenyl)-3-isoquinolin-5-yl-thiourea

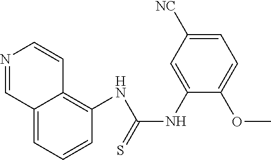

The title compound was prepared from isoquinolin-5-ylamine and Intermediate 124 by the procedure of Intermediate 179.

### Intermediate 252: N-{4-[3-(5-carboxamido-2-isopropopoxy-phenyl)-thioureido]-1H-benzoimidazol-2-yl}-benzamide

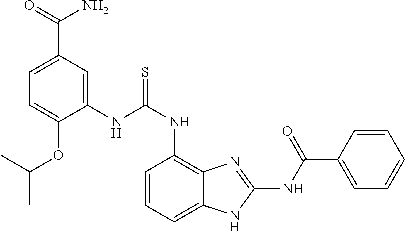

The title compound was prepared according to the procedure of Intermediate 138, substituting Intermediate 99 for 2-methoxyphenyl isothiocyanate.

### Intermediate 253: 4-Isopropoxy-3-nitro-benzonitrile

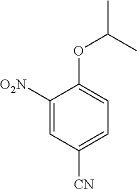

Potassium hexamethyldisilazane [0.5 M in toluene] (12.04 mL, 24.08 mmol) was added to a solution of isopropanol (0.921 mL, 12.04 mmol) in tetrahydrofuran (20 mL) at 0° C. and stirred for 10 minutes. The resulting solution was added dropwise to a solution of commercially available 4-fluoro-3-nitrobenzonitrile at room temperature and stirred for several hours. Water was added and the product was extracted with ethyl acetate, dried with sodium sulfate and evaporated in vacuo to give the title compound.

### Intermediate 254: 3-amino-4-isopropoxybenzonitrile

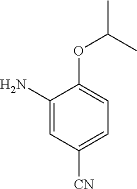

Sodium borohydride (0.297 g, 7.86 mmol) was added slowly to a solution of nickel (II) chloride hexahydrate (0.675 g, 2.62 mmol) in methanol (30 mL) at room temperature and stirred for 0.5 h. To the resulting solution was added 4-isopropoxy-3-nitro-benzonitrile (1.08 g, 5.24 mmol), followed by sodium borohydride (0.694 g, 18.34 mmol) and stirred for 0.5 h. The solution was then filtered through celite, water was added and the product was extracted with ethyl acetate, dried with sodium sulfate and evaporated in vacuo to give the title compound.

### Intermediate 255: 4-Ethoxy-3-isothiocyanatopyridine

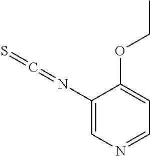

To a stirred mixture of 4-ethoxypyridin-3-amine (1.04 g, 7.53 mmol) and sodium bicarbonate (1.90 g, 22.8 mmol) in chloroform (15 mL) and water (10 mL) at 4° C. was added thiophosgene (0.70 mL, 9.13 mmol) dropwise. The cooling bath was removed and the biphasic mixture was stirred at room temperature for 4 h. TLC analysis indicated some of the starting material still present. More thiophosgene (0.2 mL, 2.6 mmol) was added and stirring was continued for another 1 h and filtered. The organic layer was separated, and the aqueous layer was extracted with CH₂Cl₂. The combined organic layers were dried (Na₂SO₄) and concentrated to give the crude title compound as a brown solid.

### Intermediate 256, step a: 4-Isopropoxy-3-nitropyridine

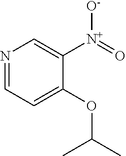

To a mixture of 3-nitropyridin-4-ol (10.3 g, 73.2 mmol) and K₂CO₃ (16.3 g, 118 mmol) in DMF (100 mL) at RT was added 2-iodopropane (11.2 mL, 112 mmol), and the mixture was stirred overnight. TLC analysis showed some of starting material still present. More 2-iodopropane (2.20 mL, 22.0 mmol) and K₂CO₃ (3.00 g, 21.7 mmol) were added and heated at 80° C. for ˜4 h. The solid was filtered off, and the filtrate was concentrated in vacuo. The residue was purified by flash chromatography (200 g silica gel column, 100% EtOAc, 10% MeOH—CH₂Cl₂) to afford the title compound as a yellow oil.

### Intermediate 256, step b: 4-Isopropoxypyridin-3-amine

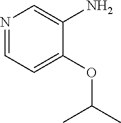

To a Parr-bottle containing 4-isopropoxy-3-nitropyridine (4.32 g, 23.7 mmol, Intermediate 256, step a) and EtOH (36 mL) was added 10% Pd/C (102 mg) and was shaken under 40 psi H₂ for 21 h. The mixture was filtered through Celite and washed with EtOH. The filtrate was concentrated to give the title compound as brown oil.

### Intermediate 256, step c: 4-Isopropoxy-3-isothiocyanatopyridine

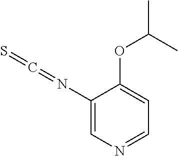

The title compound was prepared from 4-isopropoxypyridin-3-amine (Intermediate 256, step b) according to the procedure of Intermediate 255. The product was purified by flash chromatography (40 g silica gel column, 50-100% EtOAc-heptane) to obtain the title compound as a brown oil.

### Intermediate 257: 1-(5-cyano-2-isopropoxyphenyl)-3-(1-methyl-1H-benzo[d]imidazol-4-yl)thiourea

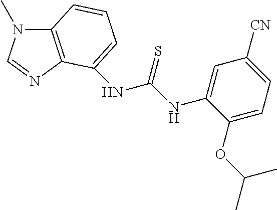

The title compound was prepared from 3-amino-4-isopropoxybenzonitrile (Intermediate 254) and Intermediate 128 by the procedure of Intermediate 163, except that after stirring at room temperature overnight, the reaction mixture was additionally heated to 60° C. for 4 h.

### Intermediate 258: 4-isopropoxy-3-(3-(1-methyl-1H-indol-4-yl)thioureido)benzenesulfonamide

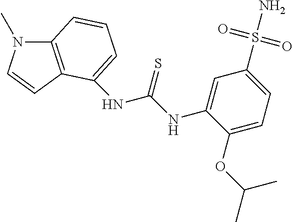

The title compound was prepared from Intermediate 100 and 1-methyl-1H-indol-4-ylamine by the procedure of Intermediate 163.

### Intermediate 259: 3-(3-(1-methyl-1H-indol-4-yl)thioureido)-4-(trifluoromethoxy)benzenesulfonamide

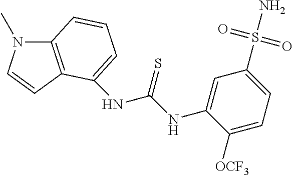

The title compound was prepared from Intermediate 121 and 1-methyl-1H-indol-4-ylamine by the procedure of Intermediate 163.

### Intermediate 260:1-(2-methoxy-5-(methylsulfonyl)phenyl)-3-(1-methyl-1H-indol-4-yl)thiourea

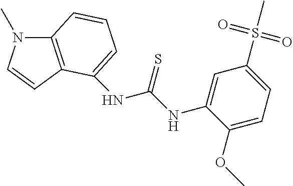

The title compound was prepared from Intermediate 110 and 1-methyl-1H-indol-4-ylamine by the procedure of Intermediate 163.

### Intermediate 261: 3-(3-(1-methyl-1H-benzo[d]imidazol-4-yl)thioureido)-4-(trifluoromethoxy)benzamide

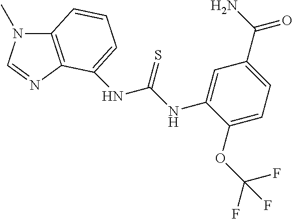

The title compound was prepared from Intermediate 122 and 4-amino-1-methylbenzimidazole by the procedure of Intermediate 163.

### Intermediate 262: 1-(1-Ethyl-2-methyl-1H-benzo[d]imidazol-7-yl)-3-(4-methoxypyridin-3-yl)thiourea

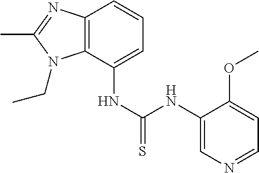

The title compound was prepared from Intermediate 93 and Intermediate 134 by the procedure of Intermediate 202.

### Intermediate 263: 1-(4-Methoxypyridin-3-yl)-3-(1-(2,2,2-trifluoroethyl)-1H-benzo[d]imidazol-7-yl)thiourea

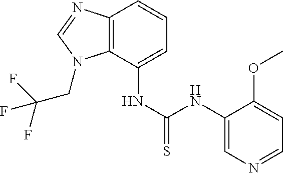

The title compound was prepared from Intermediate 97 and Intermediate 134 by the procedure of Intermediate 202.

### Intermediate 264: 1-(1,2-Dimethyl-1H-benzo[d]imidazol-7-yl)-3-(4-methoxypyridin-3-yl)thiourea

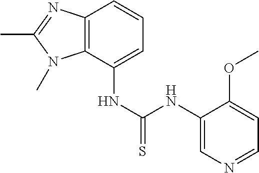

The title compound was prepared from Intermediate 94 and Intermediate 134 by the procedure of Intermediate 202.

### Intermediate 265: 1-(4-Ethoxypyridin-3-yl)-3-(1-ethyl-2-methyl-1H-benzo[d]imidazol-7-yl)thiourea

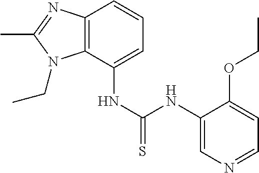

The title compound was prepared from Intermediate 58 and Intermediate 255 by the procedure of Intermediate 222.

### Intermediate 266: 1-(4-Ethoxypyridin-3-yl)-3-(1-methyl-1H-benzo[d]imidazol-4-yl)thiourea

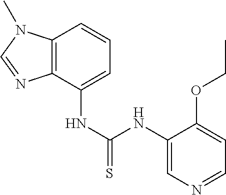

The title compound was prepared from 1-methyl-1H-benzo[d]imidazol-4-amine and Intermediate 255 by the procedure of Intermediate 222.

### Intermediate 267: 1-(4-Isopropoxypyridin-3-yl)-3-(1-methyl-1H-benzo[d]imidazol-4-yl)thiourea

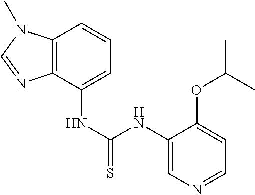

The title compound was prepared from 1-methyl-1H-benzo[d]imidazol-4-amine and Intermediate 256, step c by the procedure of Intermediate 222.

### Intermediate 268: 1-(1,2-Dimethyl-1H-benzo[d]imidazol-4-yl)-3-(4-isopropoxypyridin-3-yl)thiourea

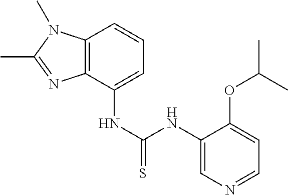

The title compound was prepared from Intermediate 53 and Intermediate 256, step c by the procedure of Intermediate 222.

### Intermediate 269: 1-(1-Ethyl-2-methyl-1H-benzo[d]imidazol-7-yl)-3-(4-isopropoxypyridin-3-yl)thiourea

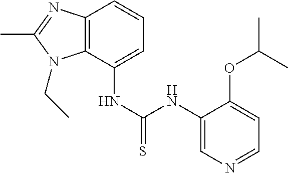

The title compound was prepared from Intermediate 58 and Intermediate 256, step c by the procedure of Intermediate 222.

### EXAMPLES

### Example 1

### N⁷-(2-Methoxy-phenyl)-benzo[2,1-d;3,4-d′]-bisthiazole-2,7-<diamine.TFA

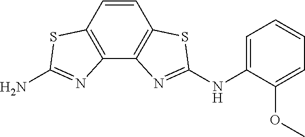

A slurry of Intermediate 3 (0.05 g, 0.225 mmol) and 2-methoxyaniline (0.25 mL, 2.22 mmol) in NMP (1.5 mL) was heated to 200° C. in an oil bath for 5 days. The reaction mixture was directly purified by HPLC, eluting with water/acetonitrile/0.2% trifluoroacetic acid to yield the title compound. ¹H NMR (DMSO-d₆, 400 MHz): δ=8.67 (d, J=7.6 Hz, 1 H), 7.47-7.61 (m, 2 H), 6.93-7.13 (m, 3 H), 3.89 ppm (s, 3 H); MS m/e 329 (M+H).

### Example 2

### N⁷-(2-Methoxy-phenyl)-benzo[1,2-d;3,4-d′]-bisthiazole-2,7-diamine.TFA

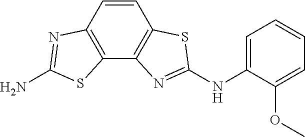

A solution of Intermediate 237 (0.14 g, 0.42 mmol) and 2-methoxyphenyl thiourea (0.076 g, 0.42 mmol) in EtOH (5 mL) was heated in a sealed tube at 120° C. for 3 days. The reaction mixture was directly purified by HPLC, eluting with water/acetonitrile/0.2% trifluoroacetic acid to yield the title compound. ¹H NMR (DMSO-d₆, 300 MHz): δ=8.33 (d, J=7.5 Hz, 1 H), 7.74 (d, J=8.3 Hz, 1 H), 7.24 (d, J=8.3 Hz, 1 H), 7.07-7.17 (m, 2 H), 6.98-7.07 (m, 1 H), 3.88 ppm (s, 3 H); MS m/e 329 (M+H).

### Example 3

### N⁷-(2-Ethoxy-phenyl)-benzo[2,1-d;3,4-d′]bisthiazole-2,7-diamine.TFA

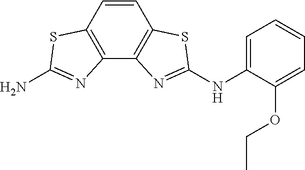

A mixture of Intermediate 3 (0.050 g, 0.225 mmol) and 2-ethoxyaniline (0.40 mL, 3.09 mmol) in NMP (1.5 mL) was refluxed for 3 days. The reaction mixture was directly purified by HPLC, eluting with water/acetonitrile/0.2% trifluoroacetic acid to yield the title compound. ¹H NMR (300 MHz, DMSO-d₆): δ 9.73 (br. s., 1 H), 8.54-8.71 (m, 1 H), 8.18 (br. s., 1 H), 7.43-7.65 (m, 2 H), 6.86-7.13 (m, 3 H), 4.15 (q, J=7.2 Hz, 2 H), 1.40 ppm (t, J=7.0 Hz, 3 H); MS m/e 343 (M+H).

### Example 4

### N,N′-Bis-(2-ethoxy-phenyl)-benzo[2,1-d;3,4-d′]bisthiazole-2,7-diamine.TFA

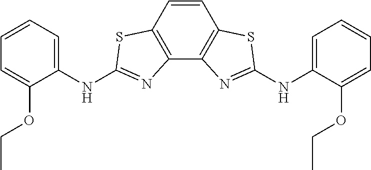

The title compound was isolated from the reaction described in Example 3. ¹H NMR (300 MHz, DMSO-d₆) δ 9.77 (br. s., 2H), 8.45 (d, J=7.54 Hz, 2H), 7.55 (s, 2H), 6.89-7.16 (m, 6H), 4.15 (q, J=6.78 Hz, 4H), 1.39 (t, J=6.97 Hz, 6H); MS m/e 463 (M+H).

### Example 5

### 3-(2-Amino-benzo[1,2-d;3,4-d′]bisthiazol-7-ylamino)-4-isopropoxy-benzamide.TFA

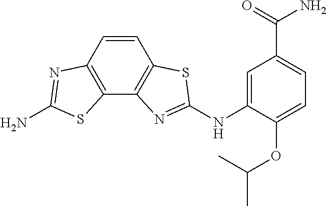

A suspension of Intermediate 237 (0.064 g, 0.198 mmol) and Intermediate 135 (0.050 g, 0.198 mmol) in EtOH (4 mL) was heated to 120° C. in a sealed tube for 12 hours. The reaction mixture was directly purified by HPLC, eluting with water/acetonitrile/0.2% trifluoroacetic acid to yield the title compound. ¹H NMR (400 MHz, DMSO-d₆) δ 8.69 (d, J=1.96 Hz, 1H), 7.63 (d, J=8.56 Hz, 1H), 7.56 (dd, J=2.08, 8.44 Hz, 1H), 7.15 (d, J=8.56 Hz, 1H), 7.08 (d, J=8.56 Hz, 1H), 4.71 (spt, J=6.11 Hz, 1H), 1.26 (d, J=5.87 Hz, 6H); MS m/e 400 (M+H).

### Example 6

### N²-(2-Methoxy-phenyl)-6H-imidazo[4′,5′:3,4]benzo[2,1-d]thiazole-2,7-diamine

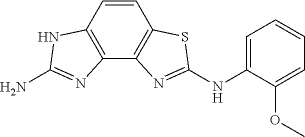

A solution of Intermediate 234 (0.077 g, 0.185 mmol) in MeOH (8 mL) and 1N NaOH (8 mL) was heated to reflux for 12 hours. The reaction was cooled, the MeOH removed in vacuo, and the resulting precipitate collected, washed with water and dried under nitrogen/vacuum to yield the title compound. ¹H NMR (300 MHz, DMSO-d₆) δ 8.70 (m, 1H), 7.20 (d, J=7.91 Hz, 1H), 6.89-7.10 (m, 4H), 3.87 (s, 3H); MS m/e 312 (M+H).

### Example 7

### N⁷-(5-Chloro-2-methoxy-phenyl)-benzo[1,2-d;3,4-d′]-bisthiazole-2,7-diamine.TFA

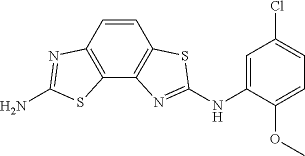

A mixture of Intermediate 238 (0.097 g, 0.296 mmol) and 2-methoxy-5-chloro-phenyl thiourea (0.064 g, 0.296 mmol) in dioxane (4 mL) and water (1 mL) was heated to 100° C. for 12 hours. The reaction mixture was partitioned between saturated aqueous NaHCO₃ solution and CHCl₃. The layers were separated, and the organics were washed once with brine, filtered through cotton, dried over Na₅SO₄, and filtered. The crude product solution was treated with excess DDQ until aromatization of the central ring was complete. The mixture was concentrated, dissolved in a small volume of DMSO, and purified by HPLC, eluting with water/acetonitrile/0.2% trifluoroacetic acid. The product was recrystallized from MeCN to yield the title compound. ¹H NMR (300 MHz, DMSO-d₆) δ 10.27 (s, 1H), 8.75 (s, 1H), 8.13 (br. s., 2H), 7.72 (d, J=8.29 Hz, 1H), 7.25 (d, J=8.29 Hz, 1H), 7.09 (s, 2H), 3.90 (s, 3H); MS m/e 363 (M+H).

### Example 8

### N⁷-(2,5-Dimethoxy-phenyl)-benzo[1,2-d;3,4-d′]-bisthiazole-2,7-diamine.TFA

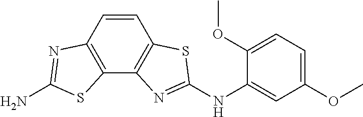

The title compound was prepared by the method of Example 7, substituting 2,5-dimethoxyphenyl thiourea for 2-methoxy-5-chloro-phenyl thiourea. ¹H NMR (300 MHz, DMSO-d₆) δ 10.13 (s, 1H), 8.16-8.54 (m, 3H), 7.72 (d, J=8.67 Hz, 1H), 7.24 (d, J=8.29 Hz, 1H), 7.00 (d, J=9.04 Hz, 1H), 6.61 (dd, J=3.01, 9.04 Hz, 1H), 3.83 (s, 3H), 3.78 (s, 3H); MS m/e 359 (M+H).

### Example 9

### N⁷-(2-Isopropoxy-phenyl)-benzo[1,2-d;3,4-d′]-bisthiazole-2,7-diamine.TFA

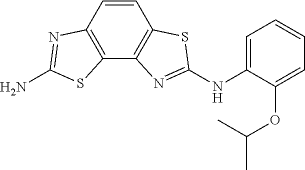

The title compound was prepared by the method of Example 7, substituting Intermediate 136 for 2-methoxy-5-chloro-phenyl thiourea. ¹H NMR (300 MHz, DMSO-d₆) δ 9.76 (s, 1H), 8.24 (d, J=7.53 Hz, 1H), 8.12 (br. s., 1H), 7.67 (d, J=8.29 Hz, 1H), 7.20 (d, J=8.29 Hz, 1H), 7.03-7.15 (m, 2H), 6.92-7.03 (m, 1H), 4.67 (spt, J=6.03 Hz, 1H), 1.31 (d, J=6.03 Hz, 6H); MS m/e 357 (M+H).

### Example 10

### 3-(7-Amino-6H-imidazo[4′,5′:3,4]benzo[2,1-d]thiazol-2-ylamino)-4-isopropoxy-benzoic acid.HCl

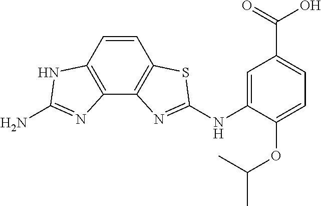

The title compound was prepared from Intermediate 236 by the procedure of Example 6, with the exception that the crude product was purified by HPLC, eluting with water/acetonitrile/0.2% trifluoroacetic acid. The HCl salt was made by repeated exposure of the TFA salt to MeOH/1N HCl. ¹H NMR (400 MHz, DMSO-d₆) δ 8.60 (d, J=2.20 Hz, 1H), 7.73 (dd, J=2.20, 8.56 Hz, 1H), 7.67 (d, J=8.56 Hz, 1H), 7.21 (dd, J=4.77, 8.68 Hz, 2H), 4.86 (spt, J=6.11 Hz, 1H), 1.33 (d, J=6.11 Hz, 6H); MS m/e 384 (M+H).

### Example 11

### 3-(7-Amino-6H-imidazo[4′,5′:3,4]benzo[2,1-d]thiazol-2-ylamino)-4-isopropoxy-benzamide.HCl

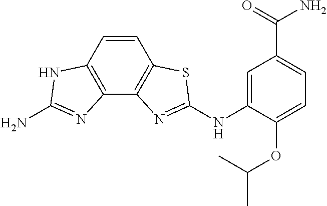

The title compound was prepared by the method of Example 10, with a shorter reaction time (3.5 hours) to prevent hydrolysis to the acid. ¹H NMR (400 MHz, DMSO-d₆) δ 13.13 (br. s., 1H), 12.56 (br. s., 1H), 9.76 (s, 1H), 8.49 (d, J=1.96 Hz, 1H), 8.17 (s, 2H), 7.82 (br. s., 1H), 7.68 (dd, J=2.20, 8.56 Hz, 1H), 7.64 (d, J=8.56 Hz, 1H), 7.27 (br. s., 1H), 7.18 (d, J=8.56 Hz, 2H), 4.77 (spt, J=6.11 Hz, 1H), 1.31 (d, J=5.87 Hz, 6H); MS m/e 383 (M+H).

### Example 12

### 4-Isopropoxy-3-(2-methyl-4,5-dihydro-benzo[1,2-d;3,4-d′]-bisthiazol-7-ylamino)-benzamide.TFA

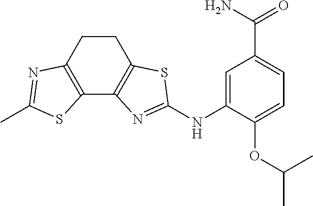

Intermediate 243 (0.25 g, 0.68 mmol) and Intermediate 135 (0.173 g, 0.68 mmol) were dissolved in dioxane (16 mL) and water (4 mL) and refluxed for 7 hours. The reaction mixture was concentrated and the residue dissolved in DMSO and purified by HPLC, eluting with water/acetonitrile/0.2% trifluoroacetic acid to yield the title compound. ¹H NMR (300 MHz, DMSO-d₆) δ 9.29 (s, 1H), 8.62 (d, J=1.88 Hz, 1H), 7.75 (br. s., 1H), 7.51 (dd, J=2.07, 8.48 Hz, 1H), 7.09-7.14 (br. s., 1H), 7.07 (d, J=8.67 Hz, 1H), 4.75 (spt, J=6.03 Hz, 1H), 3.02 (t, J=4.90 Hz, 5H), 2.64 (s, 3H), 1.32 (d, J=6.03 Hz, 6H); MS m/e 401 (M+H).

### Example 13

### 4-Isopropoxy-3-(2-methyl-benzo[1,2-d;3,4-d′]bisthiazol-7-ylamino)-benzamide.TFA

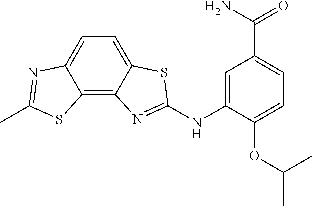

Example 12 (0.087 g, 0.217 mmol) was dissolved in DMSO (3 mL) and DDQ (0.049 g, 0.217 mmol) was added. After 1 hour, the reaction mixture was directly purified by HPLC, eluting with water/acetonitrile/0.2% trifluoroacetic acid to yield the title compound. ¹H NMR (300 MHz, DMSO-d₆) δ 9.89 (s, 1H), 8.74 (d, J=1.88 Hz, 1H), 7.88 (d, J=8.29 Hz, 1H), 7.83 (br. s., 1H), 7.69 (d, J=8.29 Hz, 1H), 7.64 (dd, J=2.07, 8.48 Hz, 1H), 7.15 (d, J=9.04 Hz, 2H), 4.78 (spt, J=6.03 Hz, 1H), 2.83 (s, 4H), 1.33 (d, J=6.03 Hz, 6H); MS m/e 399 (M+H).

### Example 14

### N⁸-(2-Methoxy-phenyl)-5,6-dihydro-4H-1,7-dithia-3,9-diaza-cyclopenta[e]azulene-2,8-diamine.TFA

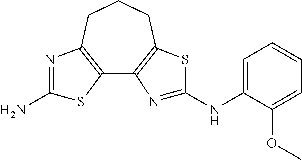

Intermediate 244 (0.10 g, 0.29 mmol) and 2-methoxyphenyl thiourea (0.053 g, 0.29 mmol) were dissolved in dioxane (4 mL) and water (1 mL) and heated to 100° C. for 1 hour. The reaction mixture was cooled and directly purified by HPLC, eluting with water/acetonitrile/0.2% trifluoroacetic acid to yield the title compound. ¹H NMR (300 MHz, DMSO-d₆) δ 9.54 (s, 1H), 8.79 (br. s., 2H), 8.27 (m, J=7.91 Hz, 1H), 6.77-7.09 (m, 3H), 3.85 (s, 3H), 2.89 (d, J=4.90 Hz, 4H), 2.00 (br. s., 2H); MS m/e 345 (M+H).

### Example 15

### 3-(7-Amino-6H-imidazo[4′,5′:3,4]benzo[2,1-d]thiazol-2-ylamino)-benzoic acid.TFA

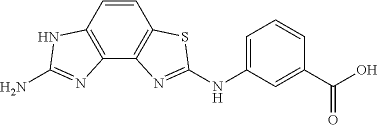

The title compound was prepared from Intermediate 235 according to the procedure of Example 6, with the exception that the crude product was purified by HPLC, eluting with water/acetonitrile/0.2% trifluoroacetic acid. ¹H NMR (400 MHz, DMSO-d₆) δ 13.30 (br. s., 1H), 13.04 (br. s., 1H), 12.57 (br. s., 1H), 10.84 (s, 1H), 8.59 (d, J=8.07 Hz, 1H), 8.19 (br. s., 2H), 7.98 (s, 1H), 7.68 (d, J=8.56 Hz, 1H), 7.63 (d, J=7.58 Hz, 1H), 7.54 (t, J=7.95 Hz, 1H), 7.22 (d, J=8.31 Hz, 1H); MS m/e 326 (M+H).

### Example 16

### N-[2-(2-Methoxy-phenylamino)-6H-imidazo[4′,5′:3,4]benzo[2,1-d]thiazol-7-yl]-acetamide.TFA

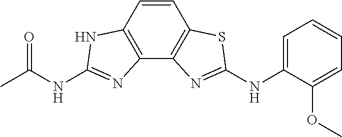

The title compound was prepared from Intermediate 140 according to the method of Intermediate 234, using a mixture of dioxane and CHCl₃ and purifying the product by HPLC, eluting with water/acetonitrile/0.2% trifluoroacetic acid. ¹H NMR (300 MHz, DMSO-d₆) δ 12.08 (br. s., 1H), 9.93 (br. s., 1H), 8.61-8.82 (m, 1H), 7.58 (d, J=8.29 Hz, 1H), 7.38 (d, J=8.67 Hz, 1H), 6.89-7.17 (m, 3H), 3.89 (s, 3H), 2.22 (s, 3H); MS m/e 354 (M+H).

### Example 17

### (2-Isopropoxy-phenyl)-(2-methyl-benzo[1,2-d;3,4-d′]-bisthiazol-7-yl)-amine.HCl

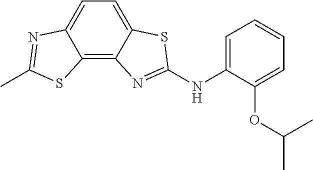

The title compound was prepared by the method of Example 13, replacing Example 12 with Intermediate 245. The HCl salt was made by repeated exposure of the TFA salt to EtOH/1N HCl. ¹H NMR (300 MHz, DMSO-d₆) δ 9.86 (br. s., 1H), 8.29 (d, J=7.91 Hz, 1H), 7.88 (d, J=8.29 Hz, 1H), 7.69 (d, J=8.67 Hz, 1H), 7.05-7.15 (m, 2H), 6.94-7.05 (m, 1H), 4.68 (spt, J=6.03 Hz, 1H), 2.83 (s, 3H), 1.32 (d, J=6.03 Hz, 6H); MS m/e 356 (M+H).

### Example 18

### 3-(7-Acetylamino-6H-imidazo[4′,5′:3,4]-benzo[2,1-d]thiazol-2-ylamino)-4-isopropoxy-benzamide.HCl

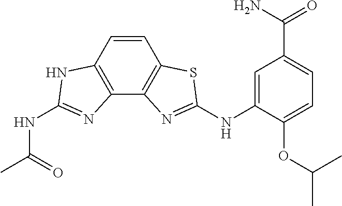

The title compound was prepared from Intermediate 141 according to the method of Intermediate 234, using dioxane as solvent, and purifying the product by HPLC, eluting with water/acetonitrile/0.2% trifluoroacetic acid. The HCl salt was made by repeated exposure of the TFA salt to EtOH/1N HCl. ¹H NMR (400 MHz, DMSO-d₆) δ 12.30 (br. s., 1H), 9.78 (br. s., 1H), 8.67 (s, 1H), 7.84 (br. s., 1H), 7.69 (d, J=8.31 Hz, 1H), 7.63 (dd, J=1.96, 8.56 Hz, 1H), 7.43 (d, J=8.31 Hz, 1H), 7.26 (br. s., 1H), 7.16 (d, J=8.80 Hz, 1H), 4.78 (spt, J=6.11 Hz, 1H), 1.33 (d, J=6.11 Hz, 6H); MS m/e 425 (M+H).

### Example 19

### 4-Isopropoxy-3-(7-methyl-6H-imidazo[4′,5′:3,4]benzo[2,1-d]thiazol-2-ylamino)-benzamide.HCl

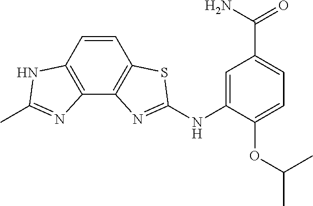

To a hazy ice-bath chilled solution of Intermediate 142 (0.138 g; 0.263 mmol) in dioxane (10 mL) was added sulfuryl chloride (0.040 mL; 0.493 mmol) dropwise, and the bath removed. The reaction mixture was heated to 65° C. on an oil bath for 2 hours, and an additional portion of sulfuryl chloride (0.012 mL, 0.148 mmol) was added. Heating at 65° C. was continued for an hour, and the mixture was concentrated. The residue was dissolved in MeOH and purified by HPLC, eluting with water/acetonitrile/0.2% trifluoroacetic acid. The HCl salt was made by repeated exposure of the TFA salt to EtOH/1N HCl to yield the title compound. ¹H NMR (300 MHz, DMSO-d₆) δ 8.50 (d, J=1.88 Hz, 1H), 7.94 (d, J=8.67 Hz, 1H), 7.70 (dd, J=2.07, 8.48 Hz, 1H), 7.50 (d, J=8.67 Hz, 1H), 7.20 (d, J=8.67 Hz, 1H), 4.79 (m, J=6.03 Hz, 1H), 2.79 (s, 4H), 1.30 (d, J=6.03 Hz, 6H); MS m/e 382 (M+H).

### Example 20

### 4-Isopropoxy-3-(7-methyl-6H-imidazo[4′,5′:3,4]-benzo[2,1-d]thiazol-2-ylamino)-benzoic acid.HCl

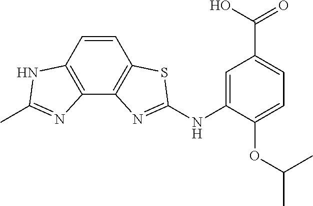

A clear, pale yellow solution of Example 19 (0.034 g, 0.081 mmol) in MeOH (8 mL) and 1N NaOH (8 mL) was heated at reflux for 12 hours. The reaction was cooled and acidified with 1N HCl until acidic by pH paper. The precipitated solid was collected, suspended in hot MeCN, cooled, and the title compound collected as a pale greenish solid. ¹H NMR (300 MHz, DMSO-d₆) δ 9.95 (s, 1H), 8.53 (d, J=1.51 Hz, 1H), 7.93 (d, J=8.67 Hz, 1H), 7.76 (dd, J=1.88, 8.67 Hz, 1H), 7.50 (d, J=8.67 Hz, 1H), 7.23 (d, J=8.67 Hz, 1H), 4.70-4.89 (m, 1H), 2.77 (s, 3H), 1.24-1.41 (d, 6H); MS m/e 383 (M+H).

### Example 21

### 3-(7-Hydroxymethyl-6H-imidazo[4′,5′:3,4]-benzo[2,1-d]thiazol-2-ylamino)-4-isopropoxy-benzamide.HCl

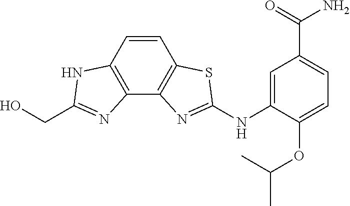

The title compound was prepared from Intermediate 143 according to the method of Intermediate 234, using dioxane as solvent, and purifying the product by HPLC, eluting with water/acetonitrile/0.2% trifluoroacetic acid. The HCl salt was made by repeated exposure of the TFA salt to EtOH/1N HCl. ¹H NMR (300 MHz, DMSO-d₆) δ 8.54 (d, J=1.88 Hz, 1H), 7.97 (d, J=8.67 Hz, 1H), 7.69 (dd, J=2.07, 8.48 Hz, 1H), 7.50 (d, J=8.67 Hz, 1H), 7.20 (d, J=8.67 Hz, 1H), 4.99 (s, 2H), 4.71-4.87 (m, 1H), 1.31 (d, J=6.03 Hz, 6H); MS m/e 398 (M+H).

### Example 22

### 4-Isopropoxy-3-(6-methyl-6H-imidazo[4′,5′:3,4]benzo[2,1-d]thiazol-2-ylamino)-benzamide.HCl

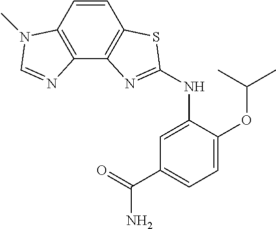

To a solution of 4-amino-1-methylbenzimidazole.2HCl (0.259 g, 1.18 mmol) in DMSO (3 mL) was added Intermediate 99 (0.301 g, 1.18 mmol) and the solution heated to 60° C. for 60 hours. The reaction was cooled and diluted with a small volume of MeOH until a clear solution resulted. This was purified by HPLC, eluting with water/acetonitrile/0.2% trifluoroacetic acid. The product fractions were concentrated, dissolved in hot EtOH/1N HCl and concentrated (twice), then with EtOH once. The resulting solid was recrystallized from boiling EtOH, cooled, and the title compound collected as a pale yellow shiny crystalline solid. ¹H NMR (400 MHz, DMSO-d₆) δ 9.98 (s, 1H), 9.47 (br. s., 1H), 8.48 (d, J=2.20 Hz, 1H), 8.03 (d, J=8.80 Hz, 1H), 7.83 (br. s., 1H), 7.59-7.76 (m, 2H), 7.28 (br. s., 1H), 7.20 (d, J=8.80 Hz, 1H), 4.68-4.85 (m, 1H), 4.07 (s, 3H), 1.30 (d, J=5.87 Hz, 6H); MS m/e 382 (M+H).

### Example 23

### 4-Isopropoxy-3-(7-oxo-7,8-dihydro-6H-imidazo[4′,5′:3,4]benzo[2,1-d]thiazol-2-ylamino)-benzamide.TFA

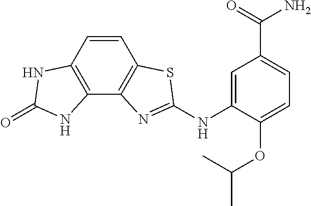

To a purple milky suspension of Intermediate 144 (0.11 g, 0.20 mmol) in dioxane (15 mL) under N₂, briefly chilled on an ice bath, was added sulfuryl chloride (0.033 mL, 0.40 mmol) and the bath removed. The reaction was allowed to warm to room temperature, then heated to 65° C. for 45 minutes. The reaction was cooled to room temperature and sulfuryl chloride (0.033 mL, 0.40 mmol) was added. After 30 minutes the reaction was concentrated, dissolved in DMSO and purified by HPLC, eluting with water/acetonitrile/0.2% trifluoroacetic acid. The product fractions were concentrated until most MeCN was removed, and a purplish solid was collected and dried. The solid was heated with MeCN and sonicated until evenly dispersed, and the title compound collected as a purple-grey solid. ¹H NMR (400 MHz, DMSO-d₆) δ 11.17 (s, 1H), 10.64 (s, 1H), 9.52 (s, 1H), 8.65 (d, J=1.96 Hz, 1H), 7.70 (br. s., 1H), 7.61 (dd, J=2.08, 8.44 Hz, 1H), 7.24-7.41 (m, 2H), 7.15 (d, J=8.56 Hz, 1H), 6.80 (d, J=8.31 Hz, 1H), 4.64-4.87 (m, 1H), 1.33 (d, J=6.11 Hz, 6H); MS m/e 384 (M+H).

### Example 24

### 4-Isopropoxy-3-(2-methyl-benzo[1,2-d;3,4-d′]bisthiazol-7-ylamino)-benzoic acid.HCl

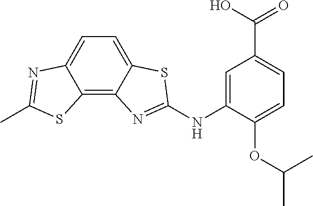

The title compound was prepared from Example 13 by the method of Example 20. ¹H NMR (300 MHz, DMSO-d₆) δ 12.62 (br. s., 1H), 9.95 (s, 1H), 9.07 (d, J=1.88 Hz, 1H), 7.91 (d, J=8.29 Hz, 1H), 7.59-7.82 (m, 2H), 7.19 (d, J=8.67 Hz, 1H), 4.79-4.87 (m, 1H), 2.84 (s, 3H), 1.22-1.44 (d, 6H); MS m/e 400 (M+H).

### Example 25

### 3-(7-Chloro-6H-imidazo[4′,5′:3,4]benzo[2,1-d]thiazol-2-ylamino)-4-isopropoxy-benzamide.HCl

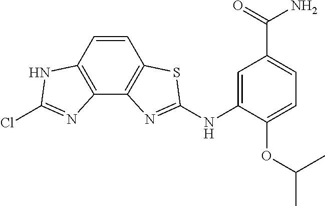

The title compound was prepared from Intermediate 145 by the method of Intermediate 234, using dioxane as solvent, and purifying the product by HPLC, eluting with water/acetonitrile/0.2% trifluoroacetic acid. The HCl salt was made by repeated exposure of the TFA salt to EtOH/1N HCl. ¹H NMR (400 MHz, DMSO-d₆) δ 9.63 (s, 1H), 8.62 (br. s., 1H), 7.79 (br. s., 1H), 7.56-7.67 (m, 2H), 7.30 (d, J=8.56 Hz, 1H), 7.25 (br. s., 1H), 7.15 (d, J=8.80 Hz, 1H), 4.76 (m, J=5.87 Hz, 1H), 1.32 (d, J=6.11 Hz, 6H); MS m/e 402 (M+H).

### Example 26

### 4-Isopropoxy-3-[7-(4-methyl-piperazine-1-carbonyl)-8H imidazo[4′,5′:3,4]benzo[2,1-d]thiazol-2-ylamino]-benzamide.HCl

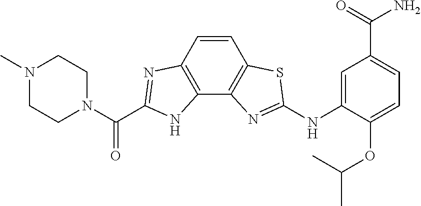

The title compound was prepared from Intermediate 146 by the method of Example 23. ¹H NMR (300 MHz, DMSO-d₆) δ 8.84 (d, J=1.88 Hz, 1H), 7.76 (d, J=8.67 Hz, 1H), 7.64 (dd, J=2.07, 8.48 Hz, 1H), 7.40 (d, J=8.67 Hz, 1H), 7.16 (d, J=8.67 Hz, 1H), 6.01 (d, J=15.45 Hz, 1H), 4.74-4.88 (m, 1H), 4.68 (d, J=14.69 Hz, 1H), 2.86 (s, 4H), 1.34 (d, J=5.65 Hz, 6H); MS m/e 494 (M+H).

### Example 27

### 3-(6,7-Dimethyl-6H-imidazo[4′,5′:3,4]benzo[2,1-d]thiazol-2-ylamino)-4-isopropoxy-benzamide.HCl

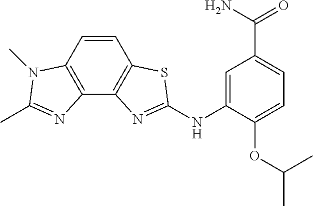

The title compound was prepared from 147 by the method of Intermediate 234, using dioxane as solvent, and purifying the product by HPLC, eluting with water/acetonitrile/0.2% trifluoroacetic acid. The HCl salt was made by repeated exposure of the TFA salt to EtOH/1N HCl. ¹H NMR (300 MHz, DMSO-d₆) δ 9.96 (s, 1H), 8.31-8.45 (d, 1H), 7.99 (d, J=8.67 Hz, 1H), 7.85 (br. s., 1H), 7.71 (dd, J=1.88, 8.67 Hz, 1H), 7.65 (d, J=8.67 Hz, 1H), 7.28 (br. s., 1H), 7.20 (d, J=8.67 Hz, 1H), 4.73-4.81 (m, 1H), 1.29 (d, J=6.03 Hz, 6H); MS m/e 396 (M+H).

### Example 28

### 4-Isopropoxy-3-(6-methyl-6H-imidazo[4′,5′:3,4]benzo[2,1-d]thiazol-2-ylamino)-benzenesulfonamide.HCl

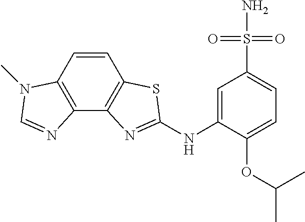

To Intermediate 148 (0.61 g, 1.45 mmol) under Ar was added glacial acetic acid (3 mL) and the mixture was sonicated to yield a slurry. To this stirred mixture was added 1.49 mL of a 5% solution of Br₂/HOAc (1 equivalent of Br₂) dropwise. The mixture was stirred for 5 minutes at room temperature, then placed in oil bath which was then heated to 60° C. After 100 minutes at 60° C., the reaction was cooled to room temperature and carefully neutralized with a saturated aqueous solution of NaHCO₃, added slowly to the vigorously stirred reaction mixture, simultaneously adding EtOAc to prevent foaming. The biphasic mixture containing solids was vigorously stirred until an evenly divided suspension results. The solid was collected, rinsed well with water, then with EtOAc, and dried under nitrogen/vacuum. The solid was treated with 1N HCl and EtOH and concentrated, then several times with EtOH to remove residual water. The title compound was collected from EtOH. ¹H NMR (400 MHz, DMSO-d₆) δ 10.18 (s, 1H), 9.59 (s, 1H), 8.77 (d, J=2.20 Hz, 1H), 8.10 (d, J=8.80 Hz, 1H), 7.73 (d, J=8.56 Hz, 1H), 7.57 (dd, J=2.32, 8.68 Hz, 1H), 7.40 (s, 2H), 7.29 (d, J=8.80 Hz, 1H), 4.82 (spt, J=6.11, 1H), 1.33 (d, J=6.11 Hz, 6H); MS m/e 418 (M+H).

### Example 29

### 3-(6H-Imidazo[4′,5′:3,4]-benzo[2,1-d]thiazol-2-ylamino)-4-isopropoxy-benzamide.HCl

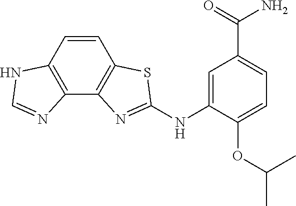

The title compound was prepared from Intermediate 149 by the method of Intermediate 234, using dioxane as solvent, and purifying the product by HPLC, eluting with water/acetonitrile/0.2% trifluoroacetic acid. The HCl salt was made by repeated exposure of the TFA salt to EtOH/1N HCl. ¹H NMR (400 MHz, DMSO-d₆) δ 9.99 (s, 1H), 9.60 (s, 1H), 8.47 (d, J=2.20 Hz, 1H), 8.01 (d, J=8.56 Hz, 1H), 7.83 (br. s., 1H), 7.71 (dd, J=2.20, 8.56 Hz, 1H), 7.59 (d, J=8.80 Hz, 1H), 7.31 (br. s., 1H), 7.20 (d, J=8.56 Hz, 1H), 4.75-4.81 (m, 1H), 1.30 (d, J=6.11 Hz, 6H); MS m/e 368 (M+H).

### Example 30

### 4-Isopropoxy-3-(6-methyl-6H-3-thia-1,6,7-triaza-as-indacen-2-ylamino)-benzamide.HCl

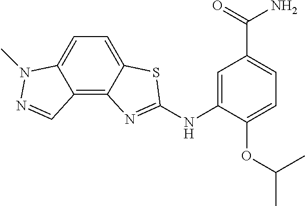

The title compound was prepared from Intermediate 150 by the method of Intermediate 234, using dioxane as solvent, and purifying the product by HPLC, eluting with water/acetonitrile/0.2% trifluoroacetic acid. The HCl salt was made by repeated exposure of the TFA salt to EtOH/1N HCl. ¹H NMR (400 MHz, DMSO-d₆) δ 9.90 (br. s., 1H), 8.75 (s, 1H), 8.12 (s, 1H), 7.88 (br. s., 1H), 7.81 (d, J=8.80 Hz, 1H), 7.66 (dd, J=1.71, 8.56 Hz, 1H), 7.45 (d, J=8.80 Hz, 1H), 7.24 (br. s., 1H), 7.16 (d, J=8.56 Hz, 1H), 4.76-4.82 (m, 1H), 1.27-1.37 (m, 6H); MS m/e 382 (M+H).

### Example 31

### 4-Isopropoxy-3-[7-(4-methyl-piperazin-1-yl)-6H-imidazo[4′,5′:3,4]benzo[2,1-d]thiazol-2-ylamino]-benzamide.HCl

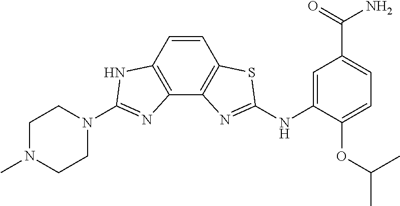

The title compound was prepared from Intermediate 151 by the method of Intermediate 234, using dioxane as solvent, and purifying the product by HPLC, eluting with water/acetonitrile/0.2% trifluoroacetic acid. The HCl salt was made by repeated exposure of the TFA salt to EtOH/1N HCl. ¹H NMR (400 MHz, DMSO-d₆) δ 8.47 (d, J=2.20 Hz, 1H), 7.74 (d, J=8.31 Hz, 1H), 7.69 (dd, J=1.96, 8.56 Hz, 1H), 7.27 (d, J=8.56 Hz, 1H), 7.19 (d, J=8.80 Hz, 1H), 4.78 (dt, J=5.90, 11.92 Hz, 1H), 4.38 (d, J=13.94 Hz, 2H), 3.69-3.81 (m, 2H), 3.62 (d, J=12.72 Hz, 2H), 3.30 (t, J=11.25 Hz, 3H), 2.83 (s, 3H), 1.30 (d, J=5.87 Hz, 6H); MS m/e 466 (M+H).

### Example 32

### (2-Isopropoxy-phenyl)-(6-methyl-6H-imidazo[4′,5′:3,4]benzo[2,1-d]thiazol-2-yl)-amine.HCl

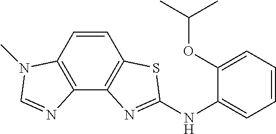

The title compound was prepared by the method of Example 22, substituting Intermediate 101 for Intermediate 99. The reaction was heated for 12 hours. ¹H NMR (400 MHz, DMSO-d₆) δ 9.99 (s, 1H), 9.59 (s, 1H), 8.40-8.59 (m, 1H), 8.06 (d, J=8.80 Hz, 1H), 7.69 (d, J=8.56 Hz, 1H), 7.07-7.16 (m, 2H), 7.01 (td, J=2.08, 7.27 Hz, 1H), 4.69 (spt, J=5.99 Hz, 1H), 1.32 (d, J=6.11 Hz, 6H); MS m/e 339 (M+H).

### Example 33

### 3-(8-Ethyl-8H-imidazo[4′,5′:3,4]-benzo[2,1-d]thiazol-2-ylamino)-4-isopropoxy-benzamide.HCl

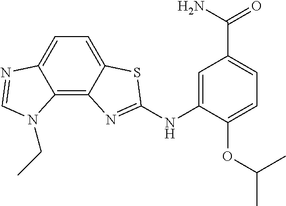

The title compound was prepared from Intermediate 56 by the method of Example 22. The reaction was heated for 15 hours. ¹H NMR (400 MHz, DMSO-d₆) δ 10.15 (s, 1H), 9.60 (s, 1H), 9.30 (d, J=1.96 Hz, 1H), 8.07 (d, J=8.80 Hz, 1H), 7.87 (br. s., 1H), 7.52-7.71 (m, 2H), 7.23 (br. s., 1H), 7.16 (d, J=8.80 Hz, 1H), 4.93 (q, J=7.09 Hz, 2H), 4.83 (spt, J=6.07 Hz, 1H), 1.64 (t, J=7.21 Hz, 3H), 1.38 (d, J=5.87 Hz, 6H); MS m/e 396 (M+H).

### Example 34

### 3-(6-Ethyl-7-methyl-6H-imidazo[4′,5′:3,4]benzo[2,1-d]thiazol-2-ylamino)-4-isopropoxy-benzamide.HCl

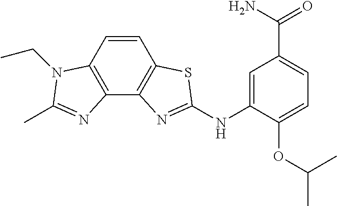

The title compound was prepared from Intermediate 57 by the method of Example 22. The reaction was heated for 24 hours. ¹H NMR (400 MHz, DMSO-d₆) δ 9.98 (s, 1H), 8.40 (d, J=2.20 Hz, 1H), 7.99 (d, J=8.80 Hz, 1H), 7.86 (br. s., 1H), 7.63-7.77 (m, 2H), 7.27 (br. s., 1H), 7.20 (d, J=8.80 Hz, 1H), 4.78 (spt, J=6.03 Hz, 1H), 4.47 (q, J=7.09 Hz, 2H), 2.84 (s, 3H), 1.41 (t, J=7.21 Hz, 3H), 1.29 (d, J=6.11 Hz, 6H); MS m/e 410 (M+H).

### Example 35

### 3-(8-Ethyl-7-methyl-8H-imidazo[4′,5′:3,4]benzo[2,1-d]thiazol-2-ylamino)-4-isopropoxy-benzamide.HCl

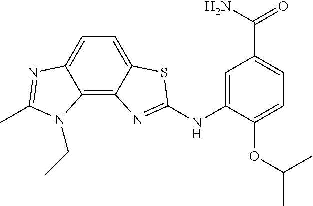

The title compound was prepared from Intermediate 58 by the method of Example 22. The reaction was heated for 18 hours. ¹H NMR (400 MHz, DMSO-d₆) δ 10.12 (s, 1H), 9.24 (d, J=2.20 Hz, 1H), 8.01 (d, J=8.56 Hz, 1H), 7.87 (br. s., 1H), 7.62 (dd, J=1.96, 8.56 Hz, 1H), 7.55 (d, J=8.56 Hz, 1H), 7.14-7.19 (m, 2H), 4.77-4.92 (m, 3H), 2.88 (s, 3H), 1.53 (t, J=7.09 Hz, 3H), 1.38 (d, J=6.11 Hz, 6H); MS m/e 410 (M+H).

### Example 36

### 4-Isopropoxy-3-(7-methyl-6-propyl-6H-imidazo[4′,5′:3,4]benzo[2,1-d]thiazol-2-ylamino)-benzamide.HCl

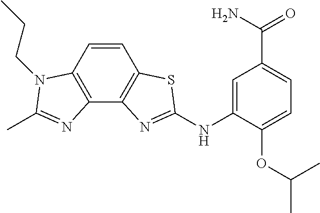

The title compound was prepared from Intermediate 59 by the method of Example 22. ¹H NMR (400 MHz, DMSO-d₆) δ 9.97 (s, 1H), 8.40 (d, J=1.96 Hz, 1H), 7.98 (d, J=8.56 Hz, 1H), 7.85 (br. s., 1H), 7.71-7.74 (m, 1H), 7.68-7.71 (m, 1H), 7.27 (br. s., 1H), 7.20 (d, J=8.80 Hz, 1H), 4.77 (spt, J=6.03 Hz, 1H), 4.40 (t, J=7.09 Hz, 2H), 2.84 (s, 3H), 1.30 (d, J=5.87 Hz, 6H), 0.95 (t, J=7.34 Hz, 3H); MS m/e 424 (M+H).

### Example 37

### (2-Ethoxy-phenyl)-(6-methyl-6H-imidazo[4′,5′:3,4]benzo[2,1-d]thiazol-2-yl)-amine.HCl

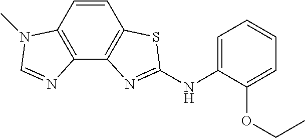

The title compound was prepared from Intermediate 102 and 4-amino-1-methylbenzimidazole.2HCl according to the procedure of Example 22. ¹H NMR (400 MHz, DMSO-d₆) δ 10.14 (s, 1H), 9.68 (s, 1H), 8.56 (d, J=7.83 Hz, 1H), 8.08 (d, J=8.80 Hz, 1H), 7.70 (d, J=8.80 Hz, 1H), 7.11 (d, J=3.91 Hz, 2H), 6.98-7.06 (m, 1H), 4.17 (q, J=6.85 Hz, 2H), 4.10 (s, 3H), 1.39 (t, J=6.97 Hz, 3H); MS m/e 325 (M+H).

### Example 38

### 4-Isopropoxy-3-(7-methyl-8-oxa-3-thia-1-aza-as-indacen-2-ylamino)-benzamide.HCl

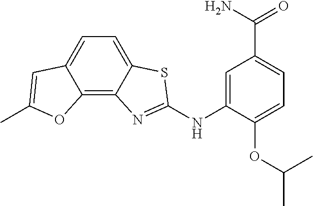

The title compound was prepared from 2-methyl-1-benzofuran-yl-amine.HCl by the procedure of Example 22. ¹H NMR (400 MHz, DMSO-d₆) δ 9.69 (br. s., 1H), 8.71 (d, J=1.96 Hz, 1H), 7.85 (br. s., 1H), 7.62 (dd, J=2.20, 8.56 Hz, 1H), 7.58 (d, J=8.07 Hz, 1H), 7.30 (d, J=8.07 Hz, 1H), 7.21 (br. s., 1H), 7.14 (d, J=8.80 Hz, 1H), 6.63 (s, 1H), 4.75-4.81 (m, 1H), 1.33 (d, J=5.87 Hz, 6H); MS m/e 382 (M+H).

### Example 39

### 4-Methoxy-3-(6-methyl-6H-imidazo[4′,5′:3,4]benzo[2,1-d]thiazol-2-ylamino)-benzamide.HCl

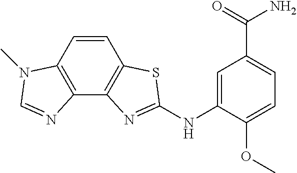

The title compound was prepared from Intermediate 103 and 4-amino-1-methylbenzimidazole.2HCl according to the procedure of Example 22. ¹H NMR (400 MHz, DMSO-d₆) δ 10.25 (s, 1H), 9.59 (s, 1H), 8.56 (d, J=2.20 Hz, 1H), 8.05 (d, J=8.80 Hz, 1H), 7.85 (br. s., 1H), 7.74 (dd, J=2.08, 8.68 Hz, 1H), 7.69 (d, J=8.80 Hz, 1H), 7.31 (br. s., 1H), 7.20 (d, J=8.80 Hz, 1H), 4.08 (s, 3H), 3.93 (s, 3H); MS m/e 344 (M+H).

### Example 40

### (5-Chloro-2-methoxy-phenyl)-(6-methyl-6H-imidazo[4′,5′:3,4]benzo[2,1-d]thiazol-2-yl)-amine.HCl

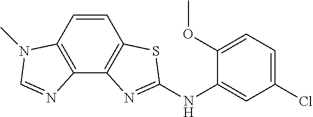

The title compound was prepared from 5-chloro-2-methoxyphenyl isothiocyanate and 4-amino-1-methylbenzimidazole.2HCl according to the procedure of Example 22. The reaction was heated for 18 hours. ¹H NMR (400 MHz, DMSO-d₆) δ 10.43 (s, 1H), 9.59 (br. s., 1H), 8.81 (s, 1H), 8.08 (d, J=8.80 Hz, 1H), 7.73 (d, J=8.56 Hz, 1H), 7.12 (s, 2H), 4.10 (s, 3H), 3.92 (s, 3H); MS m/e 345 (M+H).

### Example 41

### (2-Methoxy-5-methyl-phenyl)-(6-methyl-6H-imidazo[4′,5′:3,4]benzo[2,1-d]thiazol-2-yl)-amine.HCl

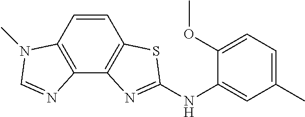

The title compound was prepared from 2-methoxy-5-methylphenyl isothiocyanate and 4-amino-1-methylbenzimidazole.2HCl according to the procedure of Example 22. The reaction was heated for 18 hours. ¹H NMR (400 MHz, DMSO-d₆) δ 10.21 (s, 1H), 9.71 (s, 1H), 8.38 (d, J=1.47 Hz, 1H), 8.07 (d, J=8.80 Hz, 1H), 7.70 (d, J=8.80 Hz, 1H), 7.00 (d, J=8.31 Hz, 1H), 6.93 (dd, J=1.71, 8.31 Hz, 1H), 4.11 (s, 3H), 3.85 (s, 3H), 2.38 (s, 3H); MS m/e 325 (M+H).

### Example 42

### (2,5-Dimethoxy-phenyl)-(6-methyl-6H-imidazo[4′,5′:3,4]-benzo[2,1-d]thiazol-2-yl)-amine.HCl

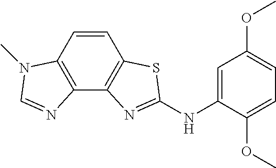

The title compound was prepared from 2,5-dimethoxyphenyl isothiocyanate and 4-amino-1-methylbenzimidazole.2HCl according to the procedure of Example 22. The reaction was heated for 18 hours. ¹H NMR (400 MHz, DMSO-d₆) δ 10.28 (s, 1H), 9.69 (s, 1H), 8.24 (d, J=2.93 Hz, 1H), 8.09 (d, J=8.80 Hz, 1H), 7.72 (d, J=8.80 Hz, 1H), 7.03 (d, J=9.05 Hz, 1H), 6.67 (dd, J=3.06, 8.93 Hz, 1H), 4.11 (s, 3H), 3.85 (s, 3H), 3.80 (s, 3H); MS m/e 341 (M+H).

### Example 43

### (6-Methyl-6H-imidazo[4′,5′:3,4]-benzo[2,1-d]thiazol-2-yl)-[2-(2,2,2-trifluoro-ethoxy)-phenyl]-amine.HCl

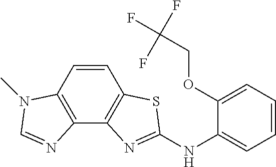

The title compound was prepared from Intermediate 104 and 4-amino-1-methylbenzimidazole.2HCl according to the procedure of Example 22. The reaction was heated for 18 hours. ¹H NMR (400 MHz, DMSO-d₆) δ 10.27 (s, 1H), 9.67 (s, 1H), 8.38 (dd, J=1.71, 7.83 Hz, 1H), 8.08 (d, J=8.80 Hz, 1H), 7.71 (d, J=8.80 Hz, 1H), 7.26-7.31 (m, 1H), 7.10-7.24 (m, 2H), 4.87 (q, J=8.80 Hz, 2H), 4.10 (s, 3H); MS m/e 379 (M+H).

### Example 44

### 2,3-Difluoro-4-methoxy-5-(6-methyl-6H-imidazo[4′,5′:3,4]-benzo[2,1-d]thiazol-2-ylamino)-benzamide.HCl

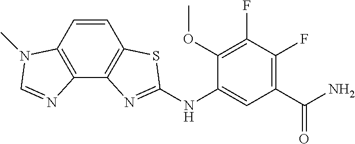

The title compound was prepared from Intermediate 105 and 4-amino-1-methylbenzimidazole.2HCl according to the procedure of Example 22. The reaction was heated for 18 hours. ¹H NMR (400 MHz, DMSO-d₆) δ 10.43 (s, 1H), 8.41 (d, J=5.62 Hz, 1H), 8.05 (d, J=8.80 Hz, 1H), 7.83 (br. s., 1H), 7.76 (s, 1H), 7.70 (d, J=8.80 Hz, 1H), 4.06 (s, 3H), 4.01 (d, J=1.71 Hz, 3H); MS m/e 390 (M+H).

### Example 45

### 3-(6,7-Dimethyl-8H-3-thia-1,8-diaza-as-indacen-2-ylamino)-4-isopropoxy-benzamide.HCl

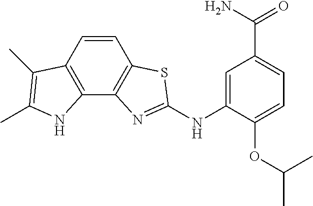

The title compound was prepared from Intermediate 152 by the method of Example 19, performing the reaction at room temperature. ¹H NMR (400 MHz, DMSO-d₆) δ 10.95 (s, 1H), 9.50 (br. s., 1H), 8.53 (d, J=1.96 Hz, 1H), 7.80 (br. s., 1H), 7.63 (dd, J=1.96, 8.56 Hz, 1H), 7.32 (d, J=8.31 Hz, 1H), 7.16 (d, J=8.56 Hz, 1H), 7.21 (d, J=8.31 Hz, 1H), 4.72-4.81 (m, 1H), 1.31 (d, J=6.11 Hz, 6H); MS m/e 395 (M+H).

### Example 46

### (2-Methoxy-phenyl)-(6-methyl-6H-imidazo[4′,5′:3,4]benzo[2,1-d]thiazol-2-yl)-amine.HCl

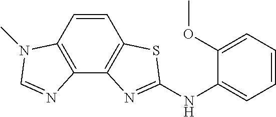

The title compound was prepared from Intermediate 153 by the method of Example 28. The product did not precipitate in the workup, so the layers were separated, and the organic layer concentrated and purified by HPLC eluting with water/acetonitrile/0.2% trifluoroacetic acid to provide the title compound. The HCl salt was made by repeated exposure to 1N HCl in EtOH. ¹H NMR (400 MHz, DMSO-d₆) δ 10.31 (s, 1H), 9.71 (s, 1H), 8.65 (d, J=7.58 Hz, 1H), 8.07 (d, J=8.80 Hz, 1H), 7.71 (d, J=8.80 Hz, 1H), 7.09-7.17 (m, 2H), 6.99-7.08 (m, 1H), 4.10 (s, 3H), 3.90 (s, 3H); MS m/e 311 (M+H).

### Example 47

### 4-Isopropoxy-3-(3H-thiazolo[4′,5′:3,4]benzo[1,2-d][1,2,3]triazol-7-ylamino)-benzamide.HCl

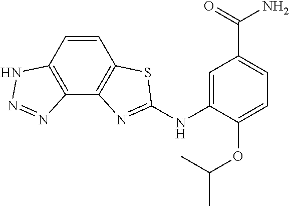

The title compound was prepared from Intermediate 61 and Intermediate 99 by the method of Example 22. ¹H NMR (400 MHz, DMSO-d₆) δ 9.82 (br. s., 1H), 8.60 (br. s., 1H), 7.80 (s, 1H), 7.84 (s, 1H), 7.66 (dd, J=1.96, 8.56 Hz, 2H), 7.17 (d, J=8.80 Hz, 1H), 4.65-4.89 (m, 1H), 1.32 (d, J=6.11 Hz, 6H); MS m/e 369 (M+H).

### Example 48

### 3-Fluoro-4-methoxy-5-(6-methyl-6H-imidazo[4′,5′:3,4]benzo[2,1-d]thiazol-2-ylamino)-benzamide.HCl

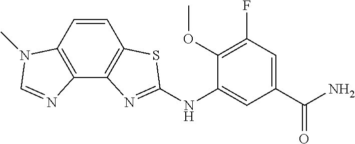

The title compound was prepared from Intermediate 106 by the procedure of Example 22. The reaction was heated for 5 hours. ¹H NMR (400 MHz, DMSO-d₆) δ 10.49 (s, 1H), 9.48 (br. s., 1H), 8.58 (s, 1H), 8.08 (d, J=8.80 Hz, 1H), 7.93 (br. s., 1H), 7.72 (d, J=8.80 Hz, 1H), 7.47-7.59 (m, 2H), 4.08 (s, 3H), 3.96 (d, J=1.71 Hz, 3H); MS m/e 372 (M+H).

### Example 49

### N-[4-Methoxy-3-(6-methyl-6H-imidazo[4′,5′:3,4]benzo[2,1-d]thiazol-2-ylamino)-phenyl]-acetamide.HCl

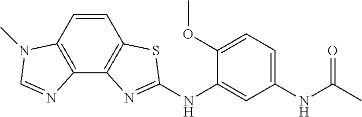

The title compound was prepared from Intermediate 107 by the method of Example 22. ¹H NMR (400 MHz, DMSO-d₆) δ 10.28 (s, 1H), 10.20 (s, 1H), 9.59 (s, 1H), 8.86 (d, J=2.45 Hz, 1H), 8.05 (d, J=8.80 Hz, 1H), 7.61-7.74 (m, 2H), 7.01 (d, J=8.80 Hz, 1H), 4.08 (s, 3H), 3.86 (s, 3H), 2.11 (s, 3H); MS m/e 368 (M+H).

### Example 50

### (2-Methoxy-5-trifluoromethyl-phenyl)-(6-methyl-6H-imidazo[4′,5′: 3,4]benzo[2,1-d]thiazol-2-yl)-amine.HCl

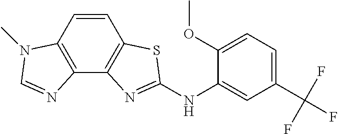

The title compound was prepared from Intermediate 108 by the method of Example 22. ¹H NMR (400 MHz, DMSO-d₆) δ 10.49 (s, 1H), 9.53 (br. s., 1H), 8.76-9.00 (m, 1H), 8.08 (d, J=8.80 Hz, 1H), 7.73 (d, J=8.80 Hz, 1H), 7.47 (dd, J=1.59, 8.68 Hz, 1H), 7.30 (d, J=8.56 Hz, 1H), 4.09 (s, 3H), 3.99 (s, 3H); MS m/e 379 (M+H).

### Example 51

### 3-Methoxy-4-(6-methyl-6H-imidazo[4′,5′:3,4]benzo[2,1-d]thiazol-2-ylamino)-benzamide.HCl

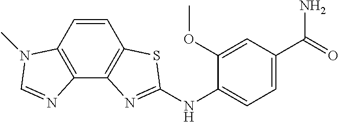

The title compound was prepared from Intermediate 109 by the method of Example 22. The reaction was heated for 18 hours. ¹H NMR (400 MHz, DMSO-d₆) δ 10.49 (s, 1H), 9.60 (s, 1H), 8.89 (d, J=9.05 Hz, 1H), 8.09 (d, J=8.56 Hz, 1H), 8.02 (br. s., 1H), 7.73 (d, J=8.80 Hz, 1H), 7.56-7.64 (m, 2H), 7.34 (br. s., 1H), 4.10 (s, 3H), 3.97 (s, 3H); MS m/e 354 (M+H).

### Example 52

### (5-Methanesulfonyl-2-methoxy-phenyl)-(6-methyl-6H-imidazo[4′,5′:3,4]benzo[2,1-d]thiazol-2-yl)-amine.HCl

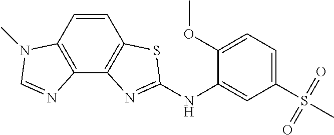

The title compound was prepared from Intermediate 154 by the method of Example 28. ¹H NMR (400 MHz, DMSO-d₆) δ 10.48 (s, 1H), 9.44 (br. s., 1H), 8.88 (s, 1H), 8.07 (d, J=8.80 Hz, 1H), 7.64-7.76 (m, 2H), 7.35 (d, J=8.80 Hz, 1H), 4.08 (s, 3H), 4.01 (s, 3H); MS m/e 389 (M+H).

### Example 53

### 4-Methoxy-3-(6-methyl-6H-imidazo[4′,5′:3,4]benzo[2,1-d]thiazol-2-ylamino)-5-trifluoromethyl-benzamide.HCl

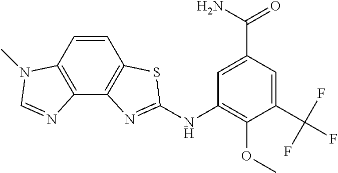

The title compound was prepared from Intermediate 111 by the method of Example 22. The reaction was heated for 18 hours. ¹H NMR (400 MHz, DMSO-d₆) δ 10.66 (br. s., 1H), 9.51 (br. s., 1H), 8.91 (s, 1H), 8.04-8.18 (m, 2H), 7.90-7.98 (m, 1H), 7.74 (d, J=8.56 Hz, 1H), 7.63 (s, 1H), 4.08 (s, 3H), 3.86 (s, 3H); MS m/e 422 (M+H).

### Example 54

### 4-Methoxy-3-(6-methyl-6H-imidazo[4′,5′:3,4]benzo[2,1-d]thiazol-2-ylamino)-benzenesulfonamide.HCl

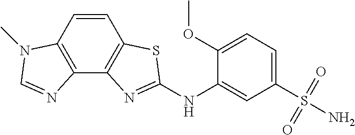

The title compound was prepared from Intermediate 112 by the method of Example 22. The reaction was heated for 18 hours. ¹H NMR (400 MHz, DMSO-d₆) δ 10.45 (s, 1H), 9.59 (s, 1H), 8.89 (d, J=2.20 Hz, 1H), 8.09 (d, J=8.56 Hz, 1H), 7.73 (d, J=8.80 Hz, 1H), 7.59 (dd, J=2.45, 8.56 Hz, 1H), 7.41 (s, 2H), 7.27 (d, J=8.80 Hz, 1H), 4.10 (s, 3H), 3.97 (s, 3H); MS m/e 390 (M+H).

### Example 55

### 4-Methoxy-N,N-dimethyl-3-(6-methyl-6H-imidazo[4′,5′:3,4]benzo[2,1-d]thiazol-2-ylamino)-benzenesulfonamide.HCl

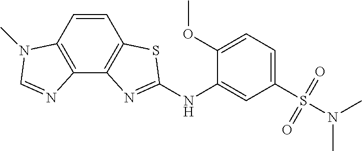

The title compound was prepared from Intermediate 113 by the method of Example 22. The reaction was heated for 1.25 hours. ¹H NMR (400 MHz, DMSO-d₆) δ 10.44 (s, 1H), 9.50 (br. s., 1H), 8.63 (s, 1H), 8.08 (d, J=8.80 Hz, 1H), 7.73 (d, J=8.80 Hz, 1H), 7.45-7.63 (m, 1H), 7.36 (d, J=8.56 Hz, 1H), 4.08 (s, 3H), 4.00 (s, 3H), 2.66 (s, 6H); MS m/e 418 (M+H).

### Example 56

### 4-Methoxy-2-methyl-5-(6-methyl-6H-imidazo[4′,5′:3,4]benzo[2,1-d]thiazol-2-ylamino)-benzamide.HCl

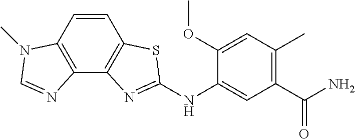

The title compound was prepared from Intermediate 114 by the method of Example 22. The reaction was heated for 24 hours. ¹H NMR (400 MHz, DMSO-d₆) δ 10.17 (s, 1H), 9.57 (s, 1H), 8.19 (s, 1H), 8.03 (d, J=8.80 Hz, 1H), 7.67 (d, J=8.56 Hz, 1H), 7.58 (br. s., 1H), 7.28 (br. s., 1H), 7.00 (s, 1H), 4.08 (s, 3H), 3.89 (s, 3H), 2.42 (s, 3H); MS m/e 368 (M+H).

### Example 57

### (5-Fluoro-2-methoxy-phenyl)-(6-methyl-6H-imidazo[4′,5′:3,4]benzo[2,1-d]thiazol-2-yl)-amine.HCl

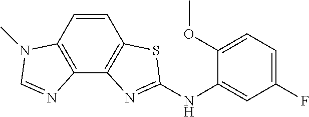

The title compound was prepared from Intermediate 155 by the method of Example 28. The product did not precipitate in the workup, so the layers were separated, and the organic layer concentrated and purified by HPLC eluting with water/acetonitrile/0.2% trifluoroacetic acid to provide the title compound. The HCl salt was made by repeated exposure to 1N HCl in EtOH. ¹H NMR (400 MHz, DMSO-d₆) δ 10.49 (s, 1H), 9.59 (br. s., 1H), 8.79 (dd, J=2.93, 11.25 Hz, 1H), 8.09 (d, J=8.80 Hz, 1H), 7.73 (d, J=8.80 Hz, 1H), 7.09 (dd, J=5.26, 8.93 Hz, 1H), 6.89 (td, J=2.93, 8.56 Hz, 1H), 4.10 (s, 3H), 3.91 (s, 3H); MS m/e 329 (M+H).

### Example 58

### (6-Methyl-6H-imidazo[4′,5′:3,4]-benzo[2,1-d]thiazol-2-yl)-(2-trifluoromethoxy-phenyl)-amine.HCl

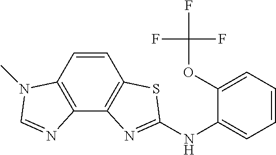

The title compound was prepared from Intermediate 116 by the method of Example 22. The reaction was run at 90° C. for 1.25 hours. ¹H NMR (400 MHz, DMSO-d₆) δ 10.72 (br. s., 1H), 9.72 (s, 1H), 8.85 (dd, J=1.47, 8.56 Hz, 1H), 8.13 (d, J=8.56 Hz, 1H), 7.77 (d, J=8.80 Hz, 1H), 7.44-7.53 (m, 2H), 7.20-7.29 (m, 1H), 4.12 (s, 3H); MS m/e 351 (M+H).

### Example 59

### (5-Fluoro-2-isopropoxy-phenyl)-(6-methyl-6H-imidazo[4′,5′:3,4]-benzo[2,1-d]thiazol-2-yl)-amine.HCl

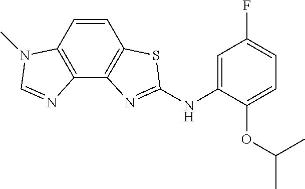

The title compound was prepared from Intermediate 117 by the method of Example 22. The reaction was heated at 90° C. for 18 hours. ¹H NMR (400 MHz, DMSO-d₆) δ 9.98 (s, 1H), 9.58 (s, 1H), 8.44 (dd, J=6.60, 8.80 Hz, 1H), 8.05 (d, J=8.80 Hz, 1H), 7.68 (d, J=8.80 Hz, 1H), 7.09 (dd, J=2.69, 11.00 Hz, 1H), 6.70-6.90 (m, 1H), 4.73 (spt, J=6.07 Hz, 1H), 4.08 (s, 3H), 1.31 (d, J=6.11 Hz, 6H); MS m/e 357 (M+H).

### Example 60

### (5-Ethanesulfonyl-2-methoxy-phenyl)-(6-methyl-6H-imidazo[4′,5′:3,4]-benzo[2,1-d]thiazol-2-yl)-amine.HCl

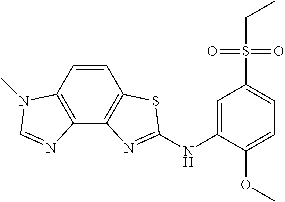

The title compound was prepared from Intermediate 118 by the method of Example 22. The reaction was heated at 90° C. for 18 hours. ¹H NMR (400 MHz, DMSO-d₆) δ 10.55 (br. s., 1H), 9.73 (s, 1H), 8.73 (d, J=1.71 Hz, 1H), 8.12 (d, J=8.80 Hz, 1H), 7.76 (d, J=8.80 Hz, 1H), 7.66 (dd, J=1.71, 8.56 Hz, 1H), 7.37 (d, J=8.56 Hz, 1H), 4.12 (s, 3H), 4.01 (s, 3H), 3.35-3.50 (m, 2H), 1.17 (t, J=7.21 Hz, 3H); MS m/e 403 (M+H).

### Example 61

### 4-Methoxy-3-(6-methyl-6H-imidazo[4′,5′:3,4]benzo[2,1-d]thiazol-2-ylamino)-benzoic acid methyl ester.HCl

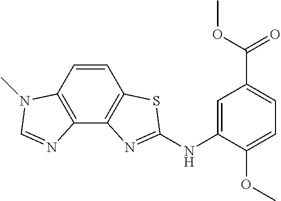

The title compound was prepared from Intermediate 119 by the method of Example 22. The reaction was heated at 90° C. for 18 hours. ¹H NMR (400 MHz, DMSO-d₆) δ 10.37 (s, 1H), 9.63 (s, 1H), 8.81 (d, J=1.47 Hz, 1H), 8.09 (d, J=8.80 Hz, 1H), 7.80 (dd, J=1.71, 8.56 Hz, 1H), 7.73 (d, J=8.80 Hz, 1H), 7.26 (d, J=8.80 Hz, 1H), 4.10 (s, 3H), 3.97 (s, 3H), 3.87 (s, 3H); MS m/e 369 (M+H).

### Example 62

### (2-Ethyl-phenyl)-(6-methyl-6H-imidazo[4′,5′:3,4]benzo[2,1-d]thiazol-2-yl)-amine.HCl

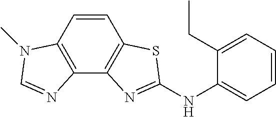

The title compound was prepared from 2-ethyl-phenylisothiocyanate by the method of Example 22. The reaction was heated at 90° C. for 4 hours. ¹H NMR (400 MHz, DMSO-d₆) δ 10.27 (br. s., 1H), 9.64 (s, 1H), 8.03 (d, J=8.80 Hz, 1H), 7.73-7.81 (m, 1H), 7.66 (d, J=8.56 Hz, 1H), 7.20-7.41 (m, 3H), 4.08 (s, 3H), 2.72 (q, J=7.42 Hz, 2H), 1.15 (t, J=7.46 Hz, 3H); MS m/e 309 (M+H).

### Example 63

### (3,5-Difluoro-2-methoxy-phenyl)-(6-methyl-6H-imidazo[4′,5′:3,4]benzo[2,1-d]thiazol-2-yl)-amine.HCl

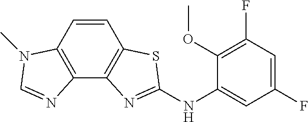

The title compound was prepared from Intermediate 120 by the method of Example 22. The reaction was heated at 90° C. for 1 hour. ¹H NMR (400 MHz, DMSO-d₆) δ 10.73 (s, 1H), 9.50 (br. s., 1H), 8.66-8.80 (m, 1H), 8.10 (d, J=8.80 Hz, 1H), 7.75 (d, J=8.80 Hz, 1H), 7.04 (ddd, J=3.06, 8.74, 11.43 Hz, 1H), 4.09 (s, 3H), 3.89 (s, 3H); MS m/e 347 (M+H).

### Example 64

### 3-(6-Methyl-6H-imidazo[4′,5′:3,4]benzo[2,1-d]thiazol-2-ylamino)-4-trifluoromethoxy-benzenesulfonamide.HCl

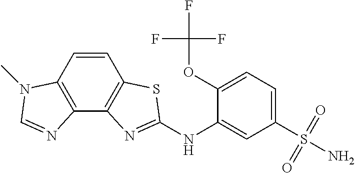

The title compound was prepared from Intermediate 156 by the method of Example 28. ¹H NMR (400 MHz, DMSO-d₆) δ 10.91 (br. s., 1H), 9.58 (br. s., 1H), 9.04 (s, 1H), 8.14 (d, J=8.80 Hz, 1H), 7.79 (d, J=8.80 Hz, 1H), 7.62-7.75 (m, 4H), 4.10 (s, 3H); MS m/e 444 (M+H).

### Example 65

### 3-(6-Methyl-6H-imidazo[4′,5′:3,4]-benzo[2,1-d]thiazol-2-ylamino)-4-trifluoromethoxy-benzamide.HCl

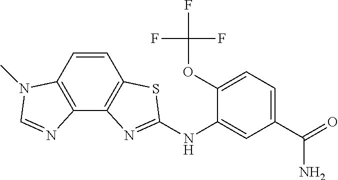

The title compound was prepared from Intermediate 157 by the method of Example 28. The reaction was heated at 60° C. for 18 hours, then an additional portion of Br₂/HOAc was added until an orange color remained. The product did not precipitate in the workup, so the layers were separated, and the organic layer concentrated and purified by HPLC eluting with water/acetonitrile/0.2% trifluoroacetic acid to provide the title compound. The HCl salt was made by repeated exposure to 1N HCl in EtOH. 1H NMR (400 MHz, DMSO-d₆) δ 10.65 (br. s., 1H), 9.44 (br. s., 1H), 8.74 (s, 1H), 7.98-8.15 (m, 2H), 7.73 (dd, J=2.69, 8.07 Hz, 2H), 7.48-7.65 (m, 2H), 4.07 (s, 3H); MS m/e 408 (M+H).

### Example 66

### [4-Methoxy-3-(6-methyl-6H-imidazo[4′,5′:3,4]benzo[2,1-d]thiazol-2-ylamino)-phenyl]-phosphonic acid diethyl ester.HCl

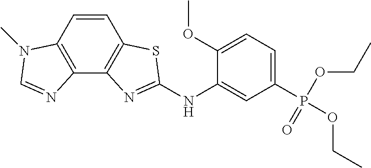

The title compound was prepared from Intermediate 158 by the method of Example 28. The reaction was concentrated and purified by HPLC eluting with water/acetonitrile/0.2% trifluoroacetic acid to provide the title compound. The HCl salt was made by repeated exposure to 1N HCl in EtOH. ¹H NMR (400 MHz, DMSO-d₆) δ 10.30 (s, 1H), 9.53 (br. s., 1H), 8.32 (d, J=13.45 Hz, 1H), 8.06 (d, J=8.56 Hz, 1H), 7.71 (d, J=8.80 Hz, 1H), 7.54 (ddd, J=1.83, 8.38, 12.78 Hz, 1H), 7.30 (dd, J=4.03, 8.44 Hz, 1H), 3.99-4.12 (m, 7H), 3.94 (s, 3H), 1.25 (t, J=6.97 Hz, 6H); MS m/e 431 (M+H).

### Example 67

### 4-Methoxy-3-(6-methyl-6H-imidazo[4′,5′:3,4]benzo[2,1-d]thiazol-2-ylamino)-benzonitrile.HCl

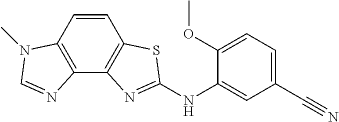

The title compound was prepared from Intermediate 159 by the method of Example 28. The precipitated product was purified by HPLC eluting with water/acetonitrile/0.2% trifluoroacetic acid to provide the title compound. The HCl salt was made by repeated exposure to 1N HCl in EtOH. ¹H NMR (400 MHz, DMSO-d₆) δ 10.63 (s, 1H), 9.74 (s, 1H), 9.19 (d, J=1.71 Hz, 1H), 8.13 (d, J=8.56 Hz, 1H), 7.78 (d, J=8.80 Hz, 1H), 7.61 (dd, J=1.71, 8.31 Hz, 1H), 7.29 (d, J=8.56 Hz, 1H), 4.13 (s, 3H), 4.01 (s, 3H); MS m/e 336 (M+H).

### Example 68

### [4-Methoxy-3-(6-methyl-6H-imidazo[4′,5′:3,4]benzo[2,1-d]thiazol-2-ylamino)-phenyl]-phosphonic acid.HCl

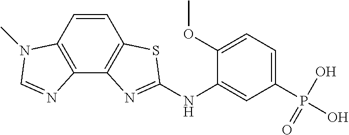

Example 66 (0.049 g, 0.11 mmol) under Ar was dissolved in dry pyridine (1 mL). Trimethylsilylbromide (0.116 mL, 0.878 mmol) was added in 3 equal portions 15 minutes apart via syringe. After 30 minutes an additional portion of trimethylsilylbromide (0.15 mL, 1.14 mmol) was added and stirred 30 minutes. The reaction was concentrated, and the residue stirred with 1N HCl for 12 hours. The mixture was concentrated and purified by HPLC, eluting with water/acetonitrile/0.2% trifluoroacetic acid. The product fractions were concentrated, and the HCl salt made by repeated exposure to 1N HCl in EtOH. ¹H NMR (400 MHz, DMSO-d₆) δ 10.01 (br. s., 1H), 8.80 (br. s., 1H), 8.49 (d, J=13.45 Hz, 1H), 7.85 (d, J=8.56 Hz, 1H), 7.41-7.60 (m, 2H), 7.19 (dd, J=3.55, 8.44 Hz, 1H), 3.97 (s, 3H), 3.91 (s, 3H); MS m/e 391 (M+H).

### Example 69

### (5-Bromo-2-methoxy-phenyl)-(6-methyl-6H-imidazo[4′,5%3,4]benzo[2,1-d]thiazol-2-yl)-amine.HCl

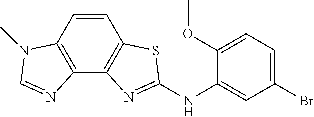

The title compound was prepared from Intermediate 160 by the method of Example 28. The reaction mixture was concentrated and purified by HPLC eluting with water/acetonitrile/0.2% trifluoroacetic acid to provide the title compound. The HCl salt was made by repeated exposure to 1N HCl in EtOH. ¹H NMR (400 MHz, DMSO-d₆) δ 10.43 (s, 1H), 9.63 (s, 1H), 8.84 (d, J=2.20 Hz, 1H), 8.10 (d, J=8.80 Hz, 1H), 7.75 (d, J=8.80 Hz, 1H), 7.27 (dd, J=2.45, 8.56 Hz, 1H), 7.08 (d, J=8.56 Hz, 1H), 4.11 (s, 3H), 3.91 (s, 3H); MS m/e 390 (M+H).

### Example 70

### [2-Methoxy-5-(1H-pyrazol-4-yl)-phenyl]-(6-methyl-6H-imidazo[4′,5%3,4]benzo[2,1-d]thiazol-2-yl)-amine.HCl

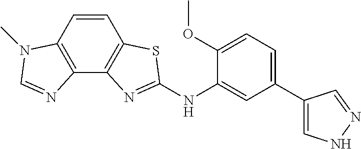

To Example 69 (0.035 g, 0.076 mmol), Pd(dppf)Cl₂ (0.0055 g, 0.076 mmol), and 4-(4,4,5,5-tetramethyl-1,3,2-dioxaborolan-2-yl)-1H-pyrazole (0.022 g, 0.114 mmol) in dioxane (2 mL) under Ar was added a solution of K₂CO₃ (0.031 g, 0.227 mmol) in water (2 mL) and the mixture was heated to 85° C. After 16 hours at 85° C., the reaction was cooled to room temperature, and an additional portion of 4-(4,4,5,5-tetramethyl-1,3,2-dioxaborolan-2-yl)-1H-pyrazole (0.022 g, 0.114 mmol), K₂CO₃ (0.031 g, 0.227 mmol) in water (1 mL), and Pd(dppf)Cl₂ (0.0055 g, 0.076 mmol) was added, and the reaction heated to 85° C. After 16 hours, the reaction was treated with 1N HCl and concentrated. The residue was dissolved in DMSO, filtered and purified by HPLC, eluting with water/acetonitrile/0.2% trifluoroacetic acid. The product fractions were concentrated, and the HCl salt made by repeated exposure to 1N HCl in EtOH. ¹H NMR (400 MHz, DMSO-d₆) δ 10.20 (s, 1H), 9.56 (br. s., 1H), 8.48 (s, 1H), 7.99-8.15 (m, 3H), 7.70 (d, J=8.80 Hz, 1H), 7.39 (dd, J=2.08, 8.44 Hz, 1H), 7.12 (d, J=8.56 Hz, 1H), 4.09 (s, 3H), 3.89 (s, 3H); MS m/e 377 (M+H).

### Example 71

### N,N-Diethyl-4-methoxy-3-(6-methyl-6H-imidazo[4′,5′:3,4]-benzo[2,1-d]thiazol-2-ylamino)-benzenesulfonamide.HCl

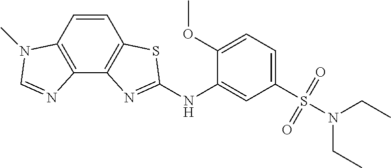

The title compound was prepared from Intermediate 161 by the method of Example 28. The product did not precipitate in the workup, so the layers were separated, and the organic layer concentrated and purified by HPLC eluting with water/acetonitrile/0.2% trifluoroacetic acid to provide the title compound. The HCl salt was made by repeated exposure to 1N HCl in EtOH. ¹H NMR (400 MHz, DMSO-d₆) δ 10.44 (br. s., 1H), 9.61 (s, 1H), 8.51-8.61 (m, 1H), 8.10 (d, J=8.80 Hz, 1H), 7.74 (d, J=8.80 Hz, 1H), 7.59 (dd, J=2.20, 8.80 Hz, 1H), 7.31 (d, J=8.80 Hz, 1H), 4.10 (s, 3H), 3.97 (s, 3H), 3.22 (q, J=7.01 Hz, 4H), 1.05 (t, J=7.09 Hz, 6H); MS m/e 446 (M+H).

### Example 72

### [2-Methoxy-5-(pyrrolidine-1-sulfonyl)-phenyl]-(6-methyl-6H-imidazo[4′,5′:3,4]benzo[2,1-d]thiazol-2-yl)-amine.HCl

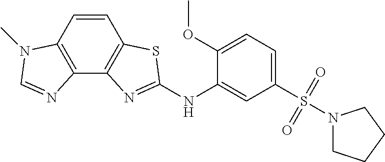

The title compound was prepared from Intermediate 162 by the method of Example 28. ¹H NMR (400 MHz, DMSO-d₆) δ 10.42 (s, 1H), 9.44 (br. s., 1H), 8.66 (s, 1H), 8.05 (d, J=8.80 Hz, 1H), 7.71 (d, J=8.56 Hz, 1H), 7.52-7.64 (m, 1H), 7.34 (d, J=8.56 Hz, 1H), 4.07 (s, 3H), 3.99 (s, 3H), 3.21 (t, J=6.36 Hz, 4H), 1.67 (t, J=6.48 Hz, 4H); MS m/e 444 (M+H).

### Example 73

### (1-Methyl-1H-benzoimidazol-4-yl)-(6-methyl-6H-imidazo[4′,5′:3,4]-benzo[2,1-d]thiazol-2-yl)-amine.HCl

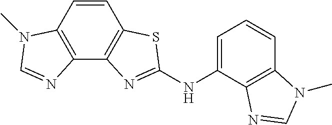

The title compound was prepared from Intermediate 164 by the method of Example 28. ¹H NMR (400 MHz, DMSO-d₆) δ 12.04 (br. s., 1H), 9.72 (s, 1H), 9.55 (br. s., 1H), 8.65-8.79 (m, 1H), 8.16 (d, J=8.56 Hz, 1H), 7.79 (d, J=8.56 Hz, 1H), 7.57-7.70 (m, 2H), 4.12 (s, 3H), 4.08 (s, 3H); MS m/e 335 (M+H).

### Example 74

### (2-Methoxy-pyridin-3-yl)-(6-methyl-6H-imidazo[4′,5′:3,4]-benzo[2,1-d]thiazol-2-yl)-amine.HCl

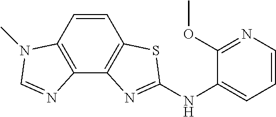

The title compound was prepared from Intermediate 165 by the method of Example 28. The reaction mixture was diluted with MeOH and purified by HPLC eluting with water/acetonitrile/0.2% trifluoroacetic acid to provide the title compound. The HCl salt was made by repeated exposure to 1N HCl in EtOH. ¹H NMR (400 MHz, DMSO-d₆) δ 10.56 (s, 1H), 9.66 (s, 1H), 9.08-9.20 (m, 1H), 8.11 (d, J=8.80 Hz, 1H), 7.90 (dd, J=1.59, 5.01 Hz, 1H), 7.75 (d, J=8.56 Hz, 1H), 7.09 (dd, J=4.89, 7.83 Hz, 1H), 4.11 (s, 3H), 4.01 (s, 3H); MS m/e 314 (M+H).

### Example 75

### (6-Methyl-6H-imidazo[4′,5′:3,4]-benzo[2,1-d]thiazol-2-yl)-pyridin-3-yl-amine.HCl

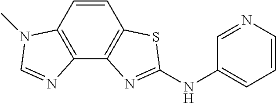

The title compound was prepared from Intermediate 166 by the method of Example 28. The product did not precipitate in the workup, so the layers were separated, and the organic layer concentrated and purified by HPLC eluting with water/acetonitrile/0.2% trifluoroacetic acid to provide the title compound. The HCl salt was made by repeated exposure to 1N HCl in EtOH. ¹H NMR (400 MHz, DMSO-d₆) δ 12.42 (br. s., 1H), 9.67 (s, 1H), 9.61 (br. s., 1H), 8.86 (d, J=8.80 Hz, 1H), 8.51 (d, J=4.65 Hz, 1H), 8.15 (d, J=8.80 Hz, 1H), 7.92 (dd, J=5.38, 8.56 Hz, 1H), 7.81 (d, J=8.80 Hz, 1H), 4.12 (s, 3H); MS m/e 284 (M+H).

### Example 76

### (2-Methoxy-5-nitro-phenyl)-(6-methyl-6H-imidazo[4′,5′:3,4]benzo[2,1-d]thiazol-2-yl)-amine.HCl

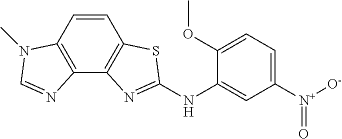

The title compound was prepared from Intermediate 167 by the method of Example 28. ¹H NMR (400 MHz, DMSO-d₆) δ 10.65 (s, 1H), 9.58 (s, 1H), 9.45 (d, J=2.69 Hz, 1H), 8.11 (d, J=8.80 Hz, 1H), 8.05 (dd, J=2.93, 9.05 Hz, 1H), 7.76 (d, J=8.80 Hz, 1H), 7.34 (d, J=9.05 Hz, 1H), 4.10 (s, 3H), 4.06 (s, 3H); MS m/e 356 (M+H).

### Example 77

### (6-Methyl-6H-imidazo[4′,5′:3,4]-benzo[2,1-d]thiazol-2-yl)-(2-nitro-phenyl)-amine.HCl

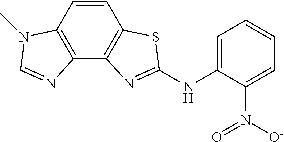

The title compound was prepared from Intermediate 168 by the method of Example 28. ¹H NMR (400 MHz, DMSO-d₆) δ 10.98 (br. s., 1H), 9.47 (br. s., 1H), 8.37 (d, J=8.07 Hz, 1H), 8.02-8.17 (m, 2H), 7.78-7.84 (m, 1H), 7.76 (d, J=8.80 Hz, 1H), 7.36-7.42 (m, 1H), 4.07 (s, 3H); MS m/e 326 (M+H).

### Example 78

### (2,4-Dimethoxy-phenyl)-(6-methyl-6H-imidazo[4′,5′:3,4]-benzo[2,1-d]thiazol-2-yl)-amine.HCl

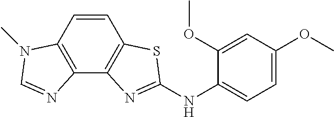

The title compound was prepared from Intermediate 169 by the method of Example 28. The product did not precipitate in the workup, so the layers were separated, and the organic layer concentrated and purified by HPLC eluting with water/acetonitrile/0.2% trifluoroacetic acid to provide the title compound. The HCl salt was made by repeated exposure to 1N HCl in EtOH. ¹H NMR (400 MHz, DMSO-d₆) δ 10.04 (s, 1H), 9.62 (s, 1H), 8.11 (d, J=8.56 Hz, 1H), 8.01 (d, J=8.80 Hz, 1H), 7.65 (d, J=8.80 Hz, 1H), 6.73 (d, J=2.45 Hz, 1H), 6.59 (dd, J=2.45, 8.80 Hz, 1H), 4.08 (s, 3H), 3.86 (s, 3H), 3.81 (s, 3H); MS m/e 341 (M+H).

### Example 79

### 4-Methoxy-N³-(6-methyl-6H-imidazo[4′,5′:3,4]-benzo[2,1-d]thiazol-2-yl)-benzene-1,3-diamine.HCl

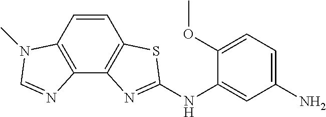

The title compound was prepared from Example 76 by the method of Intermediate 49, with the addition of 1N HCl (3 mL) to the reaction mixture. ¹H NMR (400 MHz, DMSO-d₆) δ 10.59 (br. s., 1H), 10.21 (br. s., 2H), 9.53 (br. s., 2H), 8.06 (br. s., 1H), 7.71 (d, J=8.56 Hz, 1H), 7.18 (d, J=8.80 Hz, 1H), 7.02 (dd, J=2.08, 8.68 Hz, 1H), 4.08 (s, 3H), 3.94 (s, 3H); MS m/e 326 (M+H).

### Example 80

### [2-Methoxy-5-(piperidine-1-sulfonyl)-phenyl]-(6-methyl-6H-imidazo[4′,5′:3,4]-benzo[2,1-d]thiazol-2-yl)-amine.HCl

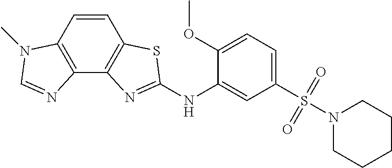

The title compound was prepared from Intermediate 170 by the method of Example 28. ¹H NMR (400 MHz, DMSO-d₆) δ 10.47 (br. s., 1H), 9.59 (br. s., 1H), 8.62 (d, J=1.71 Hz, 1H), 8.10 (d, J=8.80 Hz, 1H), 7.75 (d, J=8.80 Hz, 1H), 7.51 (dd, J=2.20, 8.56 Hz, 1H), 7.35 (d, J=8.56 Hz, 1H), 4.10 (s, 3H), 3.99 (s, 3H), 2.95 (t, J=4.52 Hz, 4H), 1.47-1.62 (m, 4H), 1.30-1.40 (m, 2H); MS m/e 458 (M+H).

### Example 81

### [2-Methoxy-5-(morpholine-4-sulfonyl)-phenyl]-(6-methyl-6H-imidazo[4′,5′:3,4]benzo[2,1-d]thiazol-2-yl)-amine.HCl

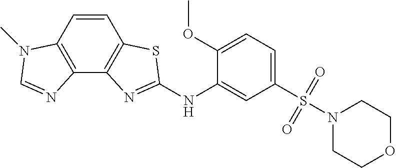

The title compound was prepared from Intermediate 171 by the method of Example 28. ¹H NMR (400 MHz, DMSO-d₆) δ 10.48 (s, 1H), 9.39 (br. s., 1H), 8.74 (br. s., 1H), 8.05 (d, J=8.80 Hz, 1H), 7.71 (d, J=8.56 Hz, 1H), 7.46-7.55 (m, 1H), 7.36 (d, J=8.80 Hz, 1H), 4.07 (s, 3H), 4.01 (s, 3H), 3.58-3.74 (m, 4H), 2.95-3.01 (m, 4H); MS m/e 460 (M+H).

### Example 82

### 4-Methoxy-N-methyl-3-(6-methyl-6H-imidazo[4′,5′:3,4]-benzo[2,1-d]thiazol-2-ylamino)-benzenesulfonamide.HCl

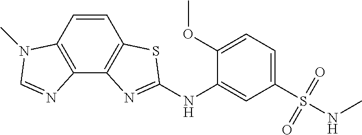

The title compound was prepared from Intermediate 172 by the method of Example 28. ¹H NMR (400 MHz, DMSO-d₆) δ 10.47 (s, 1H), 9.59 (s, 1H), 8.81 (d, J=1.96 Hz, 1H), 8.09 (d, J=8.80 Hz, 1H), 7.73 (d, J=8.56 Hz, 1H), 7.65 (d, J=5.13 Hz, 1H), 7.56 (dd, J=2.20, 8.56 Hz, 1H), 7.30 (d, J=8.56 Hz, 1H), 4.09 (s, 3H), 3.99 (s, 3H), 2.48-2.53 (m, 4H), 2.46 (d, J=5.14 Hz, 3H); MS m/e 404 (M+H).

### Example 83

### (2-Methoxy-4-nitro-phenyl)-(6-methyl-6H-imidazo[4′,5′:3,4]benzo[2,1-d]thiazol-2-yl)-amine.HCl

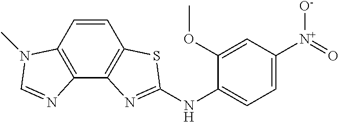

The title compound was prepared from Intermediate 173 by the method of Example 28. ¹H NMR (400 MHz, DMSO-d₆) δ 10.62 (s, 1H), 9.16 (d, J=9.05 Hz, 1H), 8.28 (s, 1H), 8.06 (d, J=8.80 Hz, 1H), 7.85 (s, 1H), 7.78 (d, J=8.56 Hz, 1H), 7.51 (d, J=8.56 Hz, 1H), 4.06 (s, 3H), 3.91 (s, 3H); MS m/e 356 (M+H).

### Example 84

### 4-Methoxy-3-(thiazolo[4′,5′:3,4]-benzo[2,1-d]oxazol-7-ylamino)-benzonitrile

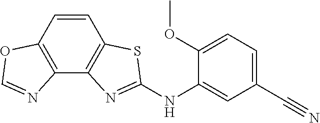

The title compound was prepared from Intermediate 174 by the method of Example 28, without transformation to the HCl salt. ¹H NMR (400 MHz, DMSO-d₆) δ 10.46 (br. s., 1H), 9.17 (d, J=1.96 Hz, 1H), 8.79 (s, 1H), 7.91 (d, J=8.56 Hz, 1H), 7.64 (d, J=8.56 Hz, 1H), 7.58 (dd, J=1.96, 8.56 Hz, 1H), 7.28 (d, J=8.56 Hz, 1H), 4.01 (s, 3H); MS m/e 323 (M+H).

### Example 85

### 4-Methoxy-3-(6-methyl-6H-thiazolo[4,5-e]indol-2-ylamino)-benzonitrile.HCl

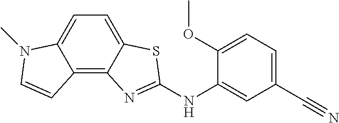

The title compound was prepared from Intermediate 175 by the method of Example 28. The product was purified by HPLC eluting with water/acetonitrile/0.2% trifluoroacetic acid to provide the title compound. The HCl salt was made by repeated exposure to 1N HCl in EtOH. ¹H NMR (400 MHz, DMSO-d₆) δ 9.18 (br. s., 1H), 7.45-7.62 (m, 2H), 7.28-7.43 (m, 2H), 7.25 (d, J=8.31 Hz, 1H), 6.66 (d, J=2.45 Hz, 1H), 3.99 (s, 3H), 3.85 (s, 3H); MS m/e 335 (M+H).

### Example 86

### 4-(6-Methyl-6H-imidazo[4′,5′:3,4]benzo[2,1-d]thiazol-2-ylamino)-3-trifluoromethoxy-benzonitrile.HCl

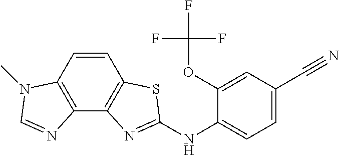

The title compound was prepared from Intermediate 176 by the method of Example 28. ¹H NMR (400 MHz, DMSO-d₆) δ 11.15 (s, 1H), 9.54 (br. s., 1H), 9.34 (d, J=8.80 Hz, 1H), 8.15 (d, J=8.80 Hz, 1H), 8.08 (s, 1H), 7.95 (dd, J=1.71, 8.80 Hz, 1H), 7.81 (d, J=8.80 Hz, 1H), 4.10 (s, 3H); MS m/e 390 (M+H).

### Example 87

### [4-Methoxy-3-(6-methyl-6H-imidazo[4′,5′:3,4]benzo[2,1-d]thiazol-2-ylamino)-phenyl]-urea.HCl

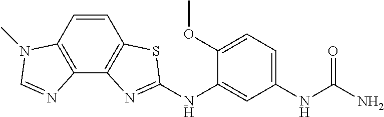

The title compound was prepared from Intermediate 177 by the method of Example 28. The solid product and the organic layer were combined, concentrated, and purified by HPLC eluting with water/acetonitrile/0.2% trifluoroacetic acid to provide the title compound. The HCl salt was made by repeated exposure to 1N HCl in EtOH. ¹H NMR (400 MHz, DMSO-d₆) δ 10.24 (s, 1H), 9.57 (br. s., 1H), 8.89 (s, 1H), 8.54 (d, J=2.45 Hz, 1H), 8.04 (d, J=8.80 Hz, 1H), 7.67 (d, J=8.56 Hz, 1H), 7.42 (dd, J=2.69, 8.80 Hz, 1H), 6.97 (d, J=9.05 Hz, 1H), 5.83 (br. s., 1H), 4.07 (s, 3H), 3.83 (s, 3H); MS m/e 369 (M+H).

### Example 88

### 4-Isobutyl-3-(6-methyl-6H-imidazo[4′,5′:3,4]-benzo[2,1-d]thiazol-2-ylamino)-benzamide

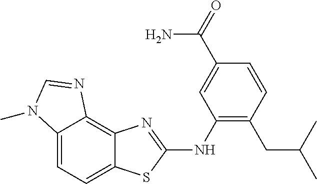

To a solution of Intermediate 178 (0.180 g, 0.472 mmol) in acetic acid (4 mL) was slowly added a solution of bromine (0.0218 mL, 0.425 mmol) in acetic acid (0.44 mL). The reaction mixture was heated in a 60° C. oil bath for 30 min. The mixture was cooled to room temperature and was carefully neutralized by addition of sat. aq. NaHCO₃ and EtOAc. The phases were separated and the aq. phase was extracted with EtOAc. The organic phase was dried (Na₂SO₄), filtered, and concentrated and the crude product was purified by flash column chromatography (Silica gel, 0-10% MeOH/EtOAc), affording the title compound as a white powder. ¹H NMR (400 MHz, DMSO-d₆) δ 9.82 (br. s., 1H), 8.18 (s, 1H), 8.12 (s, 1H), 7.95 (br. s., 1H), 7.69 (dd, J=1.71, 7.83 Hz, 1H), 7.60 (d, J=8.56 Hz, 1H), 7.30-7.38 (m, 3H), 3.86 (s, 3H), 2.62 (d, J=7.09 Hz, 2H), 1.82-1.95 (m, 1H), 0.83 (d, J=6.60 Hz, 6H). MS m/e 380.2 (M+H).

### Example 89

### 4-Isopropoxy-3-(thiazolo[5,4-h]isoquinolin-2-ylamino)-benzamide.TFA

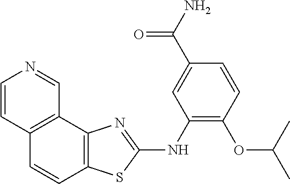

A slurry of isoquinolin-8-ylamine.HCl (0.053 g, 0.245 mmol) and Intermediate 99 (0.05 g, 0.192 mmol) in DMSO (1.5 mL) was heated to 90° C. in an oil bath for 4 hours. The reaction mixture was directly purified by HPLC, eluting with water/acetonitrile/0.2% trifluoroacetic acid to yield the title compound. ¹H NMR (400 MHz, MeOD) δ 10.09 (s, 1H), 9.24 (d, J=2.20 Hz, 1H), 8.45-8.56 (m, 2H), 8.35-8.45 (m, 1H), 7.92 (d, J=8.56 Hz, 1H), 7.66 (dd, J=2.20, 8.56 Hz, 1H), 7.14 (d, J=8.80 Hz, 1H), 4.73-4.90 (m, 1H), 1.39-1.48 (m, 6H); MS m/e 379.1 (M+H).

### Example 90

### 4-Isopropoxy-3-(thiazolo[4,5-f]isoquinolin-2-ylamino)-benzamide.TFA

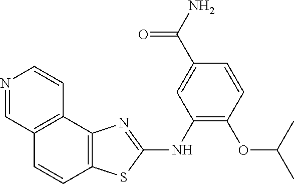

The title compound was prepared from isoquinolin-5-ylamine.HCl by the procedure of Example 89. ¹H NMR (400 MHz, MeOD) δ 9.54 (s, 1H), 9.17 (d, J=2.20 Hz, 1H), 8.82 (d, J=6.60 Hz, 1H), 8.50 (d, J=6.60 Hz, 1H), 8.24 (d, J=8.80 Hz, 1H), 8.01 (d, J=8.56 Hz, 1H), 7.60 (dd, J=2.20, 8.56 Hz, 1H), 7.09 (d, J=8.80 Hz, 1H), 4.84 (spt, J=6.11 Hz, 1H), 1.46 (d, J=6.11 Hz, 6H); MS m/e 379.1 (M+H).

### Example 91

### 4-Isopropoxy-3-(thiazolo[4,5-f]quinolin-2-ylamino)-benzamide.TFA

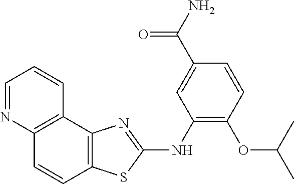

The title compound was prepared from quinolin-5-ylamine.HCl by the procedure of Example 89. ¹H NMR (400 MHz, MeOD) δ 9.56 (d, J=8.56 Hz, 1H), 9.13 (s, 1H), 8.97 (d, J=5.38 Hz, 1H), 8.30 (dd, J=1.96, 8.80 Hz, 1H), 7.94 (dd, J=5.38, 8.31 Hz, 1H), 7.73 (d, J=9.05 Hz, 1H), 7.53 (dd, J=2.20, 8.56 Hz, 1H), 7.02 (d, J=8.56 Hz, 1H), 4.69-4.94 (m, 1H), 1.17-1.44 (m, 6H); MS m/e 379.1 (M+H).

### Example 92

### (5-Methanesulfonyl-2-methoxy-phenyl)-(6-methyl-6H-3-thia-1,6,7-triaza-as-indacen-2-yl)-amine.HCl

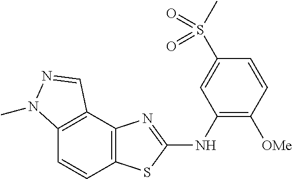

To Intermediate 179 (0.44 g, 1.13 mmol) under Ar was added glacial acetic acid (8 mL) and the mixture was sonicated to yield a slurry. To this stirred mixture was added 1.69 mL of a 0.6 M solution of Br₂/HOAc (1.01 mmol, 0.9 equivalent of Br₂) dropwise. The mixture was stirred for 30 minutes at room temperature, then placed in an oil bath which was then heated to 60° C. After 100 minutes at 60° C., the reaction was cooled to room temperature and carefully neutralized with a 3N NaOH. The solid was filtered, dissolved in DMSO and purified by HPLC, eluting with water/acetonitrile/0.2% trifluoroacetic acid to yield the product as a TFA salt. The solid was treated with 1N HCl and MeOH and concentrated, then several times with MeOH to remove residual water. ¹H NMR (400 MHz, MeOD) δ 8.31 (s, 1H), 8.18-8.28 (m, 1H), 7.93-8.11 (m, 1H), 7.76 (d, J=8.80 Hz, 1H), 7.56 (d, J=8.80 Hz, 1H), 7.49 (d, J=8.80 Hz, 1H), 4.06 (s, 3H), 4.11 (s, 3H); MS m/e 389.0 (M+H).

### Example 93

### (5-Fluoro-2-methoxy-phenyl)-(6-methyl-6H-3-thia-1,6,7-triaza-as-indacen-2-yl)-amine.HCl

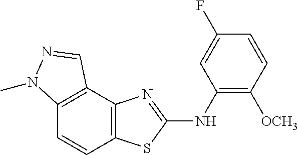

The title compound was prepared from Intermediate 180 by the procedure of Example 92. ¹H NMR (400 MHz, DMSO-d₆) δ 10.22 (s, 1H), 8.67 (dd, J=2.93, 11.49 Hz, 1H), 8.24 (s, 1H), 7.83 (d, J=8.80 Hz, 1H), 7.48 (d, J=8.80 Hz, 1H), 7.07 (dd, J=5.26, 8.93 Hz, 1H), 6.84 (td, J=3.18, 8.56 Hz, 1H), 4.10 (s, 3H); MS m/e 329.0 (M+H).

### Example 94

### (2-Methoxy-phenyl)-(6-methyl-6H-3-thia-1,6,7-triaza-as-indacen-2-yl)-amine.TFA

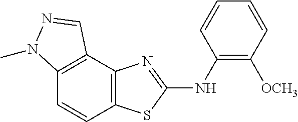

The title compound was prepared from Intermediate 181 by the procedure of Example 92 without conversion to the HCl salt. ¹H NMR (400 MHz, CHLOROFORM-d) δ 8.38 (s, 1H), 7.43-7.57 (m, 2H), 7.28-7.37 (m, 2H), 6.99-7.10 (m, 2H), 4.13 (s, 3H), 3.90 (s, 3H); MS m/e 311.1 (M+H).

### Example 95

### 4-Methoxy-3-(6-methyl-6H-3-thia-1,6,7-triaza-as-indacen-2-ylamino)-benzamide.HCl

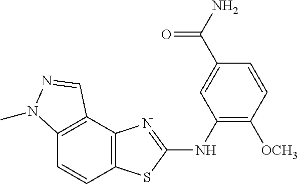

The title compound was prepared from Intermediate 182 by the procedure of Example 92. ¹H NMR (400 MHz, MeOH) δ 8.28 (s, 1H), 8.06-8.16 (m, 2H), 7.86 (d, J=9.05 Hz, 1H), 7.71 (dd, J=0.98, 9.05 Hz, 1H), 7.40 (d, J=8.80 Hz, 1H), 4.18 (s, 3H), 4.02 (s, 3H); MS m/e 354.1 (M+H).

### Example 96

### 4-Methoxy-3-(thiazolo[4,5-f]isoquinolin-2-ylamino)-benzamide.TFA

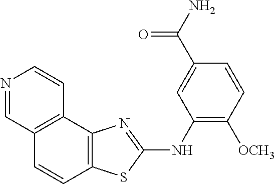

The title compound was prepared from Intermediate 183 by the procedure of Example 92 without conversion to the HCl salt. ¹H NMR (400 MHz, MeOD) δ 9.64 (s, 1H), 9.31 (d, J=2.20 Hz, 1H), 8.95 (d, J=6.60 Hz, 1H), 8.58 (d, J=6.60 Hz, 1H), 8.32 (d, J=8.56 Hz, 1H), 8.10 (d, J=8.56 Hz, 1H), 7.65 (dd, J=2.08, 8.44 Hz, 1H), 7.12 (d, J=8.56 Hz, 1H), 4.04 (s, 3H); MS m/e 351.1 (M+H).

### Example 97

### (5-Methanesulfonyl-2-methoxy-phenyl)-thiazolo[4,5-f]isoquinolin-2-yl-amine.TFA

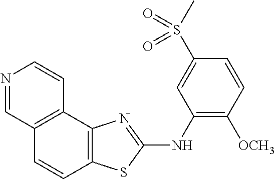

The title compound was prepared from Intermediate 184 by the procedure of Example 92 without conversion to the HCl salt. ¹H NMR (400 MHz, MeOD) δ 9.46 (s, 1H), 9.24 (d, J=2.45 Hz, 1H), 8.58 (d, J=6.60 Hz, 1H), 8.47 (d, J=6.60 Hz, 1H), 8.12 (d, J=8.56 Hz, 1H), 7.89 (d, J=8.80 Hz, 1H), 7.53 (dd, J=2.20, 8.56 Hz, 1H), 7.12 (d, J=8.56 Hz, 1H), 3.97 (s, 3H), 3.05 (s, 3H); MS m/e 386.0 (M+H).

### Example 98

### 3-(Thiazolo[4,5-f]isoquinolin-2-ylamino)-4-trifluoromethoxy-benzenesulfonamide.TFA

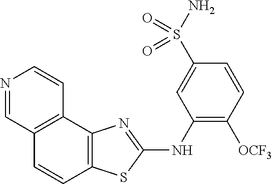

The title compound was prepared from Intermediate 185 by the procedure of Example 92 without conversion to the HCl salt. ¹H NMR (400 MHz, MeOD) δ 9.61 (s, 2H), 8.72 (d, J=6.36 Hz, 1H), 8.60 (d, J=6.36 Hz, 1H), 8.28 (d, J=8.56 Hz, 1H), 8.06 (d, J=8.56 Hz, 1H), 7.67-7.72 (m, 1H), 7.49-7.62 (m, 1H); MS m/e 441.0 (M+H).

### Example 99

### (5-Fluoro-2-methoxy-phenyl)-thiazolo[4,5-f]isoquinolin-2-yl-amine.TFA

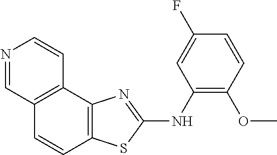

The title compound was prepared from Intermediate 186 by the procedure of Example 92 without conversion to the HCl salt. ¹H NMR (400 MHz, MeOD) δ 9.64 (s, 1H), 8.82 (d, J=6.60 Hz, 1H), 8.61 (d, J=6.60 Hz, 1H), 8.52 (dd, J=3.06, 11.13 Hz, 1H), 8.33 (d, J=8.80 Hz, 1H), 8.11 (d, J=8.56 Hz, 1H), 7.01 (dd, J=5.14, 9.05 Hz, 1H), 6.79 (td, J=3.18, 8.56 Hz, 1H), 3.96 (s, 3H); MS m/e 326.0 (M+H).

### Example 100

### (2-Methoxy-phenyl)-thiazolo[4,5-f]isoquinolin-2-yl-amine.TFA

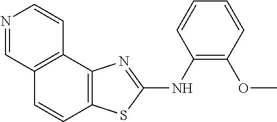

The title compound was prepared from Intermediate 187 by the procedure of Example 92 without conversion to the HCl salt. ¹H NMR (400 MHz, MeOD) δ 9.51 (s, 1H), 8.74 (d, J=6.36 Hz, 1H), 8.37-8.47 (m, 2H), 8.21 (d, J=8.80 Hz, 1H), 7.99 (d, J=8.56 Hz, 1H), 6.89-7.05 (m, 3H), 3.86 (s, 3H); MS m/e 308.0 (M+H).

### Example 101

### 3-(6-Methyl-6H-3-thia-1,6,7-triaza-as-indacen-2-ylamino)-4-trifluoromethoxy-benzenesulfonamide.TFA

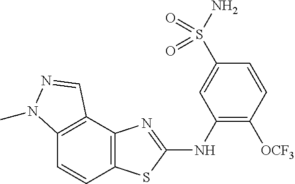

The title compound was prepared from Intermediate 188 by the procedure of Example 92 without conversion to HCl salt. ¹H NMR (400 MHz, MeOD) δ 9.47 (d, J=2.20 Hz, 1H), 8.21 (s, 1H), 7.61-7.71 (m, 1H), 7.56 (dd, J=2.32, 8.68 Hz, 1H), 7.45 (dd, J=1.47, 8.56 Hz, 1H), 7.32 (d, J=8.80 Hz, 1H), 4.00 (s, 3H); MS m/e 444.0 (M+H).

### Example 102

### 4-Isopropoxy-3-(6-methyl-6H-3-thia-1,6,7-triaza-as-indacen-2-ylamino)-benzenesulfonamide.TFA

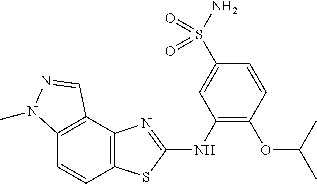

The title compound was prepared from Intermediate 189 by the procedure of Example 92 without conversion to the HCl salt. ¹H NMR (400 MHz, MeOD) δ 9.05 (d, J=2.20 Hz, 1H), 8.32 (s, 1H), 7.78 (d, J=8.80 Hz, 1H), 7.69 (dd, J=2.32, 8.68 Hz, 1H), 7.46 (d, J=8.80 Hz, 1H), 7.24 (d, J=8.80 Hz, 1H), 4.82-4.90 (m, 1H), 4.14 (s, 3H), 1.40-1.49 (m, 6H); MS m/e 418.1 (M+H).

### Example 103

### 4-Methoxy-3-(6-methyl-6H-3-thia-1,6,7-triaza-as-indacen-2-ylamino)-benzenesulfonamide.TFA

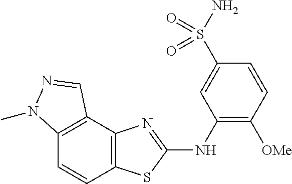

The title compound was prepared from Intermediate 190 by the procedure of Example 92 without conversion to the HCl salt. ¹H NMR (400 MHz, MeOD) δ 9.18 (d, J=2.45 Hz, 1H), 8.24 (s, 1H), 7.66 (d, J=8.80 Hz, 1H), 7.53 (d, J=2.20 Hz, 1H), 7.33 (s, 1H), 7.09 (d, J=8.56 Hz, 1H), 4.85-4.88 (m, 3H), 4.02 (s, 3H), 3.93 (s, 3H); MS m/e 390.1 (M+H).

### Example 104

### 4-Methoxy-3-(thiazolo[4,5-f]isoquinolin-2-ylamino)-benzenesulfonamide.TFA

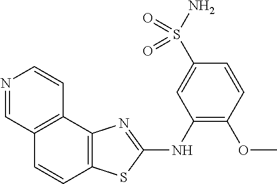

The title compound was prepared from Intermediate 191 by the procedure of Example 92 without conversion to the HCl salt. ¹H NMR (400 MHz, MeOD) δ 9.70 (s, 1H), 9.65 (d, J=2.20 Hz, 1H), 8.97 (d, J=6.36 Hz, 1H), 8.66 (d, J=6.60 Hz, 1H), 8.40 (d, J=8.56 Hz, 1H), 8.19 (d, J=8.56 Hz, 1H), 7.70 (dd, J=2.20, 8.56 Hz, 1H), 7.25 (d, J=8.56 Hz, 1H), 4.08 (s, 3H); MS m/e 387.0 (M+H).

### Example 105

### 4-Isopropoxy-3-(thiazolo[4,5-f]isoquinolin-2-ylamino)-benzenesulfonamide.TFA

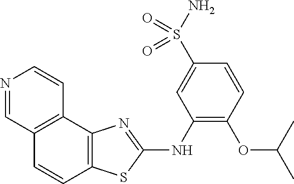

The title compound was prepared from Intermediate 192 by the procedure of Example 92 without conversion to HCl salt. ¹H NMR (400 MHz, MeOD) δ 9.57 (s, 1H), 9.40 (d, J=2.20 Hz, 1H), 8.78 (d, J=6.60 Hz, 1H), 8.57 (d, J=6.36 Hz, 1H), 8.29 (d, J=8.56 Hz, 1H), 8.04 (d, J=8.80 Hz, 1H), 7.64 (dd, J=2.45, 8.56 Hz, 1H), 7.22 (d, J=8.80 Hz, 1H), 4.82-4.97 (m, 1H), 1.51 (d, J=6.11 Hz, 6H); MS m/e 415.1 (M+H).

### Example 106

### (6-Ethyl-6H-3-thia-1,6,7-triaza-as-indacen-2-yl)-(5-methanesulfonyl-2-methoxy-phenyl)-amine.TFA

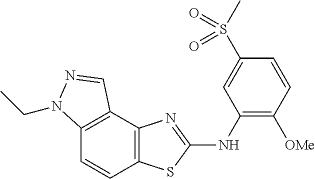

The title compound was prepared from Intermediate 193 by the procedure of Example 92 without conversion to the HCl salt. ¹H NMR (400 MHz, CHLOROFORM-d) δ 11.94 (br. s., 3H), 8.40 (d, J=0.98 Hz, 1H), 8.18 (d, J=2.20 Hz, 1H), 7.90 (dd, J=2.20, 8.80 Hz, 1H), 7.55 (d, J=9.05 Hz, 1H), 7.41 (d, J=8.07 Hz, 1H), 7.18 (d, J=8.80 Hz, 1H), 4.52 (q, J=7.17 Hz, 2H), 4.03 (s, 3H), 3.11 (s, 3H), 1.55 (t, J=7.34 Hz, 3H); MS m/e 403.2 (M+H).

### Example 107

### (7-Ethyl-7H-3-thia-1,6,7-triaza-as-indacen-2-yl)-(5-methanesulfonyl-2-methoxy-phenyl)-amine.TFA

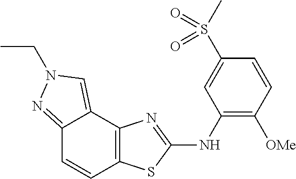

The title compound was prepared from Intermediate 194 by the procedure of Example 92 without conversion to the HCl salt. ¹H NMR (400 MHz, CHLOROFORM-d) δ 9.04 (d, J=2.20 Hz, 1H), 8.30 (s, 1H), 7.53-7.67 (m, 2H), 7.38-7.53 (m, 1H), 7.00 (d, J=8.80 Hz, 1H), 4.52 (q, J=7.34 Hz, 2H), 3.98 (s, 3H), 3.13 (s, 3H), 1.66 (t, J=7.34 Hz, 3H); MS m/e 403.2 (M+H).

### Example 108

### (6-Isobutyl-6H-3-thia-1,6,7-triaza-as-indacen-2-yl)-(5-methanesulfonyl-2-methoxy-phenyl)-amine.TFA

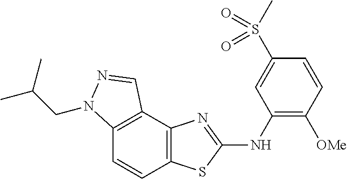

The title compound was prepared from Intermediate 195 by the procedure of Example 92 without conversion to the HCl salt. ¹H NMR (400 MHz, CHLOROFORM-d) δ 8.97 (d, J=2.20 Hz, 1H), 8.35 (s, 1H), 7.58-7.71 (m, 2H), 7.25-7.32 (m, 1H), 7.05 (d, J=8.56 Hz, 1H), 4.24 (d, J=7.34 Hz, 2H), 4.04 (s, 3H), 3.15 (s, 3H), 2.25-2.51 (m, 1H), 0.94 (d, J=6.60 Hz, 6H); MS m/e 431.3 (M+H).

### Example 109

### (5-Methanesulfonyl-2-methoxy-phenyl)-[6-(3-methyl-butyl)-6H-3-thia-1,6,7-triaza-as-indacen-2-yl]-amine.TFA

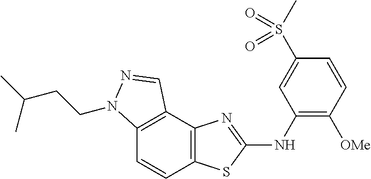

The title compound was prepared from Intermediate 196 by the procedure of Example 92 without conversion to the HCl salt. ¹H NMR (400 MHz, CHLOROFORM-d) δ 11.21 (br. s., 2H), 8.43 (s, 1H), 8.17 (d, J=2.20 Hz, 1H), 7.91 (dd, J=2.20, 8.56 Hz, 1H), 7.55 (d, J=9.05 Hz, 1H), 7.41 (d, J=9.05 Hz, 1H), 7.18 (d, J=8.80 Hz, 1H), 4.48 (t, J=7.46 Hz, 2H), 4.02 (s, 3H), 3.11 (s, 3H), 1.71-1.90 (m, 2H), 1.51-1.65 (m, 1H), 0.97 (d, J=6.60 Hz, 6H); MS m/e 445.2 (M+H).

### Example 110

### (5-Methanesulfonyl-2-methoxy-phenyl)-(6H-3-thia-1,6,7-triaza-as-indacen-2-yl)-amine.TFA

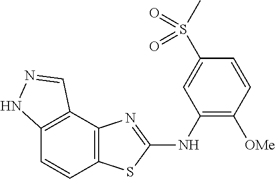

The title compound was prepared from Intermediate 197 by the procedure of Example 92 without conversion to the HCl salt. ¹H NMR (400 MHz, CHLOROFORM-d) δ 8.56 (d, J=1.96 Hz, 1H), 7.82 (dd, J=2.20, 8.56 Hz, 1H), 7.63 (d, J=9.05 Hz, 1H), 7.49 (d, J=8.80 Hz, 1H), 7.27 (s, 1H), 7.13 (d, J=8.56 Hz, 1H), 4.08-4.15 (m, 1H), 4.04 (s, 3H), 3.13 (s, 3H); MS m/e 375.1 (M+H).

### Example 111

### (5-Methanesulfonyl-2-methoxy-phenyl)-(6-propyl-6H-3-thia-1,6,7-triaza-as-indacen-2-yl)-amine.TFA

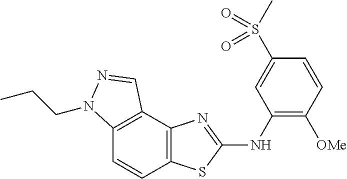

The title compound was prepared from Intermediate 198 by the procedure of Example 92 without conversion to the HCl salt. ¹H NMR (400 MHz, CHLOROFORM-d) δ 8.39-8.47 (m, 2H), 7.82 (dd, J=2.20, 8.80 Hz, 1H), 7.55 (d, J=8.80 Hz, 1H), 7.34-7.39 (m, 1H), 7.13 (d, J=8.56 Hz, 1H), 4.42 (t, J=7.09 Hz, 2H), 4.03 (s, 3H), 3.12 (s, 3H), 1.93-2.05 (m, 2H), 0.93 (t, J=7.34 Hz, 3H); MS m/e 417.1 (M+H).

### Example 112

### 4-Methoxy-3-(6-methyl-6H-3-thia-1,6,7-triaza-as-indacen-2-ylamino)-benzonitrile.TFA

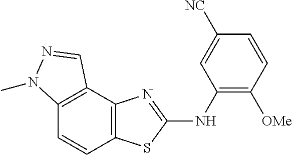

The title compound was prepared from Intermediate 199 by the procedure of Example 92 without conversion to the HCl salt. ¹H NMR (400 MHz, CHLOROFORM-d) δ 8.42 (s, 1H), 8.11 (s, 1H), 7.58 (d, J=8.80 Hz, 2H), 7.37 (d, J=8.80 Hz, 1H), 7.07 (d, J=8.56 Hz, 1H), 4.17 (s, 3H), 4.01 (s, 3H); MS m/e 336.1 (M+H).

### Example 113

### 3-(6-Methyl-6H-3-thia-1,6,7-triaza-as-indacen-2-ylamino)-4-trifluoromethoxy-benzamide.TFA

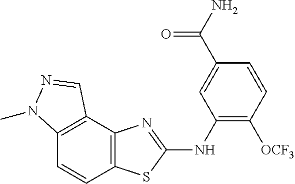

The title compound was prepared from Intermediate 200 by the procedure of Example 92 without conversion to the HCl salt. ¹H NMR (400 MHz, CHLOROFORM-d) δ 8.32-8.45 (m, 1H), 8.19 (s, 1H), 7.82 (dd, J=2.08, 8.68 Hz, 1H), 7.49-7.60 (m, 1H), 7.31-7.47 (m, 1H), 7.16 (d, J=8.07 Hz, 1H), 4.14 (s, 3H); MS m/e 408.1 (M+H).

### Example 114

### 3-(Thiazolo[4,5-f]isoquinolin-2-ylamino)-4-trifluoromethoxy-benzamide.TFA

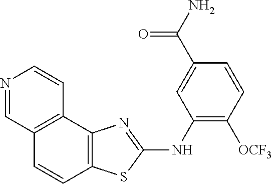

The title compound was prepared from Intermediate 201 by the procedure of Example 92 without conversion to the HCl salt. MS m/e 405.1 (M+H).

### Example 115

### 3-(4,5-Dihydro-thiazolo[4,5-f]isoquinolin-2-ylamino)-4-isopropoxy-benzamide.TFA

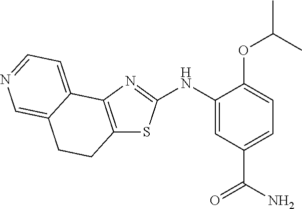

A mixture of Intermediate 248 (0.0319 g, 0.104 mmol) and Intermediate 135 (0.021 g, 0.083 mmol) in EtOH (1 mL) was stirred at room temperature for 24 hours. The mixture was purified by HPLC eluting with water/acetonitrile/0.2% trifluoroacetic acid to give the title compound. ¹H NMR (400 MHz, CHLOROFORM-d) δ 8.76 (d, J=1.96 Hz, 1H), 8.53 (d, J=4.89 Hz, 1H), 8.44 (s, 1H), 7.65-7.74 (m, 2H), 7.50-7.54 (m, 1H), 6.95 (d, J=8.56 Hz, 1H), 4.72-4.79 (m, 1H), 2.96-3.15 (m, 4H), 1.12-1.35 (m, 6H); MS m/e 381.1 (M+H).

### Example 116

### 3-(4,5-Dihydro-thiazolo[5,4-h]isoquinolin-2-ylamino)-4-isopropoxy-benzamide.TFA

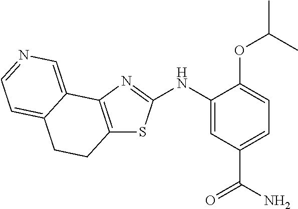

A mixture of Intermediate 249 (0.0303 g, 0.099 mmol) and Intermediate 135 (0.020 g, 0.079 mmol) in EtOH (1 mL) was stirred at room temperature for 24 hours. The mixture was purified by HPLC eluting with water/acetonitrile/0.2% trifluoroacetic acid to give the title compound. ¹H NMR (400 MHz, CHLOROFORM-d) δ 8.98 (s, 1H), 8.76 (d, J=1.96 Hz, 1H), 8.41 (d, J=4.89 Hz, 1H), 7.70 (s, 1H), 7.52-7.58 (m, 1H), 7.14 (d, J=4.89 Hz, 1H), 6.95 (d, J=8.56 Hz, 1H), 4.72-4.79 (m, 1H), 2.93-3.30 (m, 4H), 1.18-1.33 (m, 6H); MS m/e 381.1 (M+H).

### Example 117

### 3-(4,5-Dihydro-thiazolo[4,5-f]quinolin-2-ylamino)-4-methoxy-benzamide

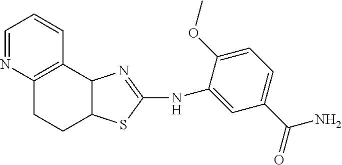

A solution of Intermediate 247 (0.050 g, 0.140 mmol) and Intermediate 137 (0.031 g, 0.140 mmol) in ethanol (2 mL) was stirred at room temperature overnight. The reaction mixture was evaporated and purified via reverse phase HPLC with water/acetonitrile/0.1% TFA. Saturated aqueous NaHCO₃ was added and the crude product was extracted with ethyl acetate, dried with sodium sulfate and purified via flash column chromatography (Silica gel, 10% MeOH/CH₂Cl₂) to give the title compound. ¹H NMR (300 MHz, MeOD) δ 8.88 (d, J=1.88 Hz, 1H), 8.25 (dd, J=1.70, 5.09 Hz, 1H), 8.14 (dd, J=1.51, 7.91 Hz, 1H), 7.57 (s, 2H), 7.54 (dd, J=2.26, 8.29 Hz, 1H), 7.30 (dd, J=5.09, 7.72 Hz, 1H), 7.01 (d, J=8.29 Hz, 1H), 4.01 (s, 3H), 3.20-3.30 (m, 2H), 3.03-3.14 (m, 2H); MS m/e 353.1 (M+H).

### Example 118

### 3-(8-Ethyl-7-methyl-8H-imidazo[4′,5′:3,4]benzo[2,1-d]thiazol-2-ylamino)-4-isopropoxy-benzenesulfonamide.TFA

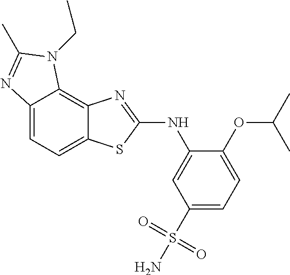

Intermediate 202 (113 mg, 0.252 mmol) in acetic acid was treated with 0.50 M Br₂ (0.505 mL, 0.252 mmol) in acetic acid for 30 min, then heated at 60° C. for 30 min. After evaporation of HOAc in vacuo, the residue was basified with 1 N NaOH. The precipitated solid was filtered, washed with water, dissolved in a small amount of DMF and purified by HPLC eluting with water/acetonitrile/0.2% trifluoroacetic acid to provide the title compound. ¹H NMR (400 MHz, MeOH-d₄) δ=9.23 (br. s., 1 H), 7.78-7.89 (m, 1 H), 7.51-7.61 (m, 1 H), 7.35-7.46 (m, 1 H), 7.10-7.20 (m, 1 H), 4.77-5.07 (m, 3 H), 2.87 (s, 3 H), 1.53-1.67 (m, 3 H), 1.48 (d, J=6.1 Hz, 6 H); MS m/e 446 (M+H).

### Example 119

### 3-(7,8-Dimethyl-8H-imidazo[4′,5′:3,4]-benzo[2,1-d]thiazol-2-ylamino)-4-isopropoxy-benzenesulfonamide.TFA

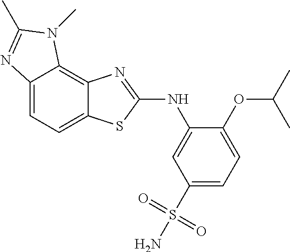

Intermediate 203 (100 mg, 0.231 mmol) in acetic acid was treated with 0.50 M Br₂ (0.46 mL, 0.23 mmol) in acetic acid for 30 min, then heated at 60° C. for 30 min. After evaporation of HOAc in vacuo, the residue was basified with 1 N NaOH. The precipitated solid was filtered, washed with water, and dried to obtain a grey solid. To a suspension of the solid in CH₂Cl₂ and MeOH was added 5 μL of CF₃CO₂H. The mixture became clear and solvents were evaporated. The residue was dried to yield the title compound as a brown solid. Some sticky material remained in the reaction container. This material was dissolved in a small amount of DMF and purified by HPLC eluting with water/acetonitrile/0.2% trifluoroacetic acid to provide a second portion of the title compound. ¹H NMR (400 MHz, MeOH-d₄) δ=9.41-9.54 (m, 1 H), 7.84 (m, 1 H), 7.46-7.58 (m, 1 H), 7.33-7.46 (m, 1 H), 7.08-7.19 (m, 1 H), 4.76-5.06 (m, 1 H), 4.47 (s, 3 H), 2.84 (s, 3 H), 1.48 (m, 6 H); MS m/e 432 (M+H).

### Example 120

### (8-Ethyl-7-methyl-8H-imidazo[4′,5′:3,4]benzo[2,1-d]thiazol-2-yl)-(2-methoxy-phenyl)-amine.TFA

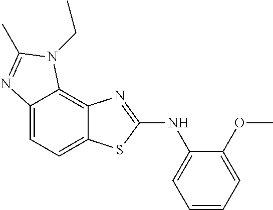

Intermediate 204 (81 mg, 0.24 mmol) in acetic acid was treated with 0.50 M Br₂ (0.43 mL, 0.22 mmol) in acetic acid for 30 min, then heated at 60° C. for 30 min. After evaporation of HOAc in vacuo, CF₃CO₂H was added and then removed in vacuo. The residue was dissolved in a small amount of DMF and purified by HPLC eluting with water/acetonitrile/0.2% trifluoroacetic acid to provide the title compound. ¹H NMR (400 MHz, CDCl₃) δ=8.29 (d, J=7.6 Hz, 1 H), 8.28 (br. s, 1 H), 7.61-7.79 (m, 1 H), 7.59 (d, J=8.6 Hz, 1 H), 7.08-7.21 (m, 1 H), 6.84-7.08 (m, 2 H), 4.64-4.87 (m, 2 H), 3.98 (s, 3 H), 2.88 (s., 3 H), 1.65 (t, J=6.6 Hz, 3 H); MS m/e 339 (M+H).

### Example 121

### (8-Ethyl-7-methyl-8H-imidazo[4′,5′:3,4]benzo[2,1-d]thiazol-2-yl)-(5-fluoro-2-methoxy-phenyl)-amine.TFA

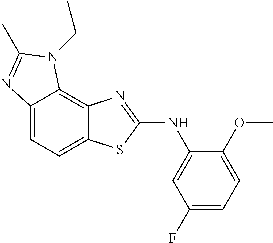

The title compound was prepared using Intermediate 205 in place of Intermediate 204 by the method of Example 120. ¹H NMR (400 MHz, CDCl₃) δ=8.38 (dd, J=2.9, 10.8 Hz, 1 H), 8.18 (s, 1 H), 7.61-7.78 (m, 2 H), 6.89 (dd, J=4.9, 9.0 Hz, 1 H), 6.78 (td, J=3.1, 8.4 Hz, 1 H), 4.81 (q, J=7.2 Hz, 2 H), 3.97 (s, 3 H), 2.91 (s, 3 H), 1.70 (t, J=7.2 Hz, 3 H); MS m/e 357 (M+H).

### Example 122

### 3-(8-Ethyl-7-methyl-8H-imidazo[4′,5′:3,4]benzo[2,1-d]thiazol-2-ylamino)-4-trifluoromethoxy-benzenesulfonamide.TFA

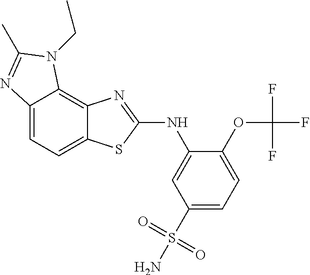

The title compound was prepared using Intermediate 206 in place of Intermediate 204 by the method of Example 120. ¹H NMR (400 MHz, MeOH-d₄) δ=9.40-9.56 (m, 1 H), 7.88-7.98 (m, 1 H), 7.62-7.72 (m, 1 H), 7.47-7.61 (m, 2 H), 4.78-5.08 (m, 2 H), 2.92 (s, 3 H), 1.56-1.70 (m, 3 H); MS m/e 472 (M+H).

### Example 123

### 3-(7,8-Dimethyl-8H-imidazo[4′,5′:3,4]-benzo[2,1-d]thiazol-2-ylamino)-4-methoxy-benzenesulfonamide.TFA

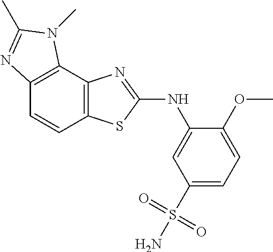

The title compound was prepared using Intermediate 207 in place of Intermediate 202 by the method of Example 118. ¹H NMR (400 MHz, MeOH-d₄) δ=9.47 (m, 1 H), 7.81-7.88 (m, 1 H), 7.50-7.58 (m, 1 H), 7.38-7.45 (m, 1 H), 7.09-7.16 (m, 1 H), 4.48 (s, 3 H), 4.03 (s, 3 H), 2.85 (s, 3 H); MS m/e 404 (M+H).

### Example 124

### (7,8-Dimethyl-8H-imidazo[4′,5′:3,4]benzo[2,1-d]thiazol-2-yl)-(2-methoxy-phenyl)-amine.TFA

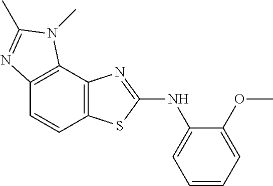

Intermediate 208 (0.090 g, 0.28 mmol) in acetic acid was treated with 0.50 M Br₂ (0.50 mL, 0.25 mmol) in acetic acid for 30 min, then heated at 60° C. for 30 min. After evaporation of HOAc in vacuo, CF₃CO₂H was added and then removed in vacuo. The residue was dissolved in a small amount of DMSO and purified by HPLC eluting with water/acetonitrile/0.2% trifluoroacetic acid to provide the title compound as solid. ¹H NMR (400 MHz, MeOH-d₄) 6=8.44 (dd, J=1.5, 8.1 Hz, 1 H), 7.86 (d, J=8.8 Hz, 1 H), 7.44 (d, J=8.8 Hz, 1 H), 7.00-7.16 (m, 3 H), 4.44 (s, 3 H), 3.95 (s, 3 H), 2.86 (s, 3 H); MS m/e 325 (M+H).

### Example 125

### (7,8-Dimethyl-8H-imidazo[4′,5′:3,4]benzo[2,1-d]thiazol-2-yl)-(5-fluoro-2-methoxy-phenyl)-amine.TFA

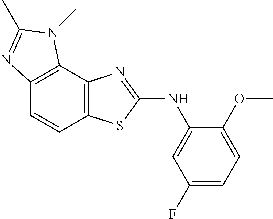

The title compound was prepared using Intermediate 209 in place of Intermediate 208 by the method of Example 124. ¹H NMR (400 MHz, MeOH-d₄) δ=8.40 (dd, J=2.9, 11.2 Hz, 1 H), 7.83 (d, J=8.6 Hz, 1 H), 7.42 (d, J=8.8 Hz, 1 H), 6.97 (dd, J=5.0, 8.9 Hz, 1 H), 6.74 (td, J=2.9, 8.4 Hz, 1 H), 4.39 (s, 3 H), 3.92 (s, 3 H), 2.84 (s, 3 H); MS m/e 343 (M+H).

### Example 126

### 3-(7,8-Dimethyl-8H-imidazo[4′,5′:3,4]-benzo[2,1-d]thiazol-2-ylamino)-4-trifluoromethoxy-benzenesulfonamide.TFA

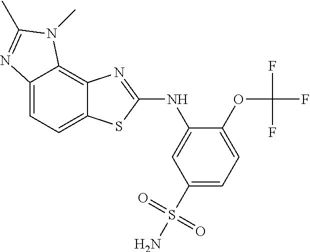

Intermediate 210 (0.113 g, 0.246 mmol) in acetic acid was treated with 0.50 M Br₂ (0.44 mL, 0.22 mmol) in acetic acid for 30 min, then heated at 60° C. for 30 min. After evaporation of HOAc in vacuo, CF₃CO₂H was added and then removed in vacuo. To the residue was added approximately 1 mL of DMSO and water. The precipitated solid was filtered, washed with water, and dried to obtain the title compound as white solid. ¹H NMR (400 MHz, MeOH-d₄) δ=9.71 (d, J=2.2 Hz, 1 H), 7.88 (d, J=8.8 Hz, 1 H), 7.62 (dd, J=2.2, 8.6 Hz, 1 H), 7.52-7.56 (m, 1 H), 7.48 (d, J=8.6 Hz, 1 H), 4.47 (s, 3 H), 2.86 (s, 3 H); MS m/e 458 (M+H).

### Example 127

### (5-Fluoro-2-methoxy-phenyl)-(8-methyl-8H-imidazo[4′,5′:3,4]benzo[2,1-d]thiazol 2-yl)-amine.TFA

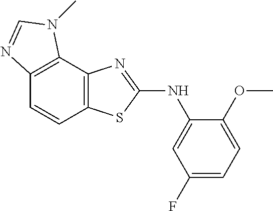

The title compound was prepared using Intermediate 211 in place Intermediate 210 by the method of Example 126. ¹H NMR (400 MHz, MeOH-d₄) δ=9.26 (s, 1 H), 8.55 (d, J=3.1, 11.1 Hz, 1 H), 7.94 (d, J=8.8 Hz, 1 H), 7.56 (d, J=8.8 Hz, 1 H), 7.01 (dd, J=5.0, 8.9 Hz, 1 H), 6.77 (td, J=3.2, 8.4 Hz, 1 H), 4.54 (s, 3 H), 3.94 (s, 3 H); MS m/e 329 (M+H).

### Example 128

### 3-(8-Methyl-8H-imidazo[4′,5′:3,4]benzo[2,1-d]thiazol-2-ylamino)-4-trifluoromethoxy-benzenesulfonamide.TFA

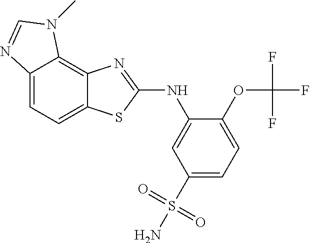

The title compound was prepared using Intermediate 212 in place of Intermediate 208 by the method of Example 124. ¹H NMR (400 MHz, MeOH-d₄) δ 9.67 (m, 1 H), 9.20-9.32 (m, 1 H), 7.89-8.02 (m, 1 H), 7.47-7.75 (m, 3 H), 4.54 (s, 3 H); MS m/e 444 (M+H).

### Example 129

### 3-(8-Ethyl-7-methyl-8H-imidazo[4′,5′:3,4]benzo[2,1-d]thiazol-2-ylamino)-4-methoxy-benzenesulfonamide.TFA

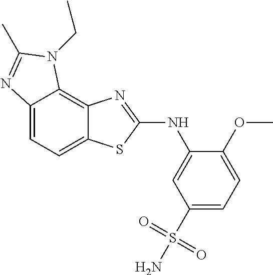

The title compound was prepared using Intermediate 213 in place of Intermediate 208 by the method of Example 124. ¹H NMR (400 MHz, MeOH-d₄) δ 9.00 (m, 1 H), 7.60-7.81 (m, 1 H), 7.51 (s, 1 H), 7.15-7.38 (m, 1 H), 6.86-7.15 (m, 1 H), 4.78 (m, 2 H), 4.00 (s, 3 H), 2.82 (s, 3 H), 1.40-1.65 (m, 3 H); MS m/e 418 (M+H).

### Example 130

### 4-Methoxy-3-(8-methyl-8H-imidazo[4′,5′:3,4]benzo[2,1-d]thiazol-2-ylamino)-benzenesulfonamide.TFA

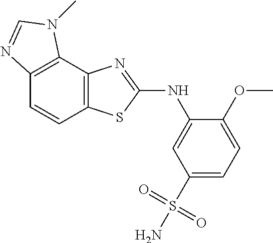

The title compound was prepared using Intermediate 214 in place of Intermediate 208 by the method of Example 124. ¹H NMR (400 MHz, MeOH-d₄) δ 9.56 (d, J=2.2 Hz, 1 H), 9.22 (s, 1 H), 7.92 (d, J=8.6 Hz, 1 H), 7.58 (dd, J=2.2, 8.6 Hz, 1 H), 7.53 (d, J=8.6 Hz, 1 H), 7.15 (d, J=8.8 Hz, 1 H), 4.58 (s, 3 H), 4.04 (s, 3 H); MS m/e 390 (M+H).

### Example 131

### 4-Isopropoxy-3-(8-methyl-8H-imidazo[4′,5′:3,4]benzo[2,1-d]thiazol-2-ylamino)-benzenesulfonamide.TFA

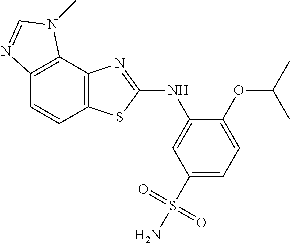

The title compound was prepared using Intermediate 215 in place of Intermediate 208 by the method of Example 124. ¹H NMR (400 MHz, MeOH) δ 9.57 (d, J=2.45 Hz, 1H), 9.21 (s, 1H), 7.92 (d, J=8.56 Hz, 1H), 7.56 (dd, J=2.45, 8.56 Hz, 1H), 7.53 (d, J=8.80 Hz, 1H), 7.17 (d, J=8.80 Hz, 1H), 4.82-4.90 (m, 1H), 4.58 (s, 3H), 1.47 (d, J=6.11 Hz, 6H); MS m/e 418 (M+H).

### Example 132

### (2-Methoxy-phenyl)-(8-methyl-8H-imidazo[4′,5′:3,4]benzo[2,1-d]thiazol-2-yl)-amine.TFA

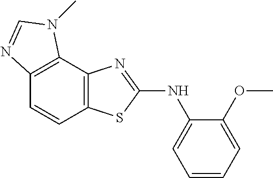

The title compound was prepared using Intermediate 216 in place of Intermediate 208 by the method of Example 124. ¹H NMR (400 MHz, MeOH-d₄) δ 9.28 (m 1 H), 8.47 (d, J=7.3 Hz, 1 H), 7.91-7.93 (m, 1 H), 7.52-7.54 (m, 1 H), 7.01-7.13 (m, 3 H), 4.52 (s, 3H), 3.94 (s, 3 H); MS m/e 311 (M+H).

### Example 133

### (8-Ethyl-7-methyl-8H-imidazo[4′,5′:3,4]benzo[2,1-d]thiazol-2-yl)-(5-methanesulfonyl-2-methoxy-phenyl)-amine.TFA

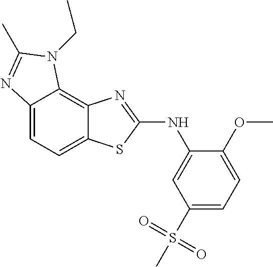

Intermediate 217 (0.085 g, 0.20 mmol) in acetic acid was treated with 0.50 M Br₂ (0.37 mL, 0.19 mmol) in acetic acid overnight. After evaporation of HOAc in vacuo, CF₃CO₂H was added and then removed in vacuo. The residue was dissolved in a small amount of DMSO and purified by HPLC eluting with water/acetonitrile/0.2% trifluoroacetic acid to provide the title compound as a light brown solid. ¹H NMR (400 MHz, MeOH-d₄) δ 9.33 (d, J=2.2 Hz, 1 H), 7.87 (d, J=8.8 Hz, 1 H), 7.60 (dd, J=2.2, 8.6 Hz, 1 H), 7.44 (d, J=8.6 Hz, 1 H), 7.22 (d, J=8.6 Hz, 1 H), 4.95-5.01 (m, 2 H), 4.07 (s, 3 H), 3.09 (s, 3 H), 2.90 (s, 3 H), 1.64 (t, J=7.2 Hz, 3 H); MS m/e 417 (M+H).

### Example 134

### (7,8-Dimethyl-8H-imidazo[4′,5′:3,4]benzo[2,1-d]thiazol-2-yl)-(5-methanesulfonyl-2-methoxy-phenyl)-amine.TFA

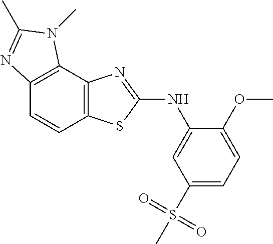

Intermediate 218 (0.076 g, 0.19 mmol) in acetic acid was treated with 0.50 M Br₂ (0.34 mL, 0.17 mmol) in acetic acid overnight. After evaporation of HOAc in vacuo, CF₃CO₂H was added and then removed in vacuo. To the residue was added a small amount of DMSO, and some white solid precipitated. Water was added. The white solid was filtered, washed with water, and dried to provide the title compound. ¹H NMR (400 MHz, DMSO-d₆) δ 10.72 (s, 1 H), 9.47 (d, J=2.2 Hz, 1 H), 7.99 (d, J=8.6 Hz, 1 H), 7.65 (dd, J=2.5, 8.6 Hz, 1 H), 7.56 (d, J=8.6 Hz, 1 H), 7.35 (d, J=8.8 Hz, 1 H), 4.37 (s., 3 H), 4.03 (s, 3 H), 3.18 (s, 3 H), 2.83 (s, 3 H); MS m/e 403 (M+H).

### Example 135

### (5-Methanesulfonyl-2-methoxy-phenyl)-(8-methyl-8H-imidazo[4′,5′:3,4]benzo[2,1-d]thiazol-2-yl)-amine.TFA

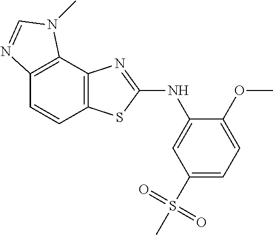

The title compound was prepared using Intermediate 219 in place of Intermediate 218 by the method of Example 134. ¹H NMR (400 MHz, MeOH-d₄) δ 9.61 (d, J=2.2 Hz, 1H), 9.24 (s, 1 H), 7.93 (d, J=8.8 Hz, 1 H), 7.60 (dd, J=2.3, 8.4 Hz, 1 H), 7.54 (d, J=8.8 Hz, 1 H), 7.23 (d, J=8.6 Hz, 1 H), 4.59 (s, 3 H), 4.07 (s, 3 H), 3.12 (s, 3 H); MS m/e 389 (M+H).

### Example 136

### 4-Isopropoxy-3-[7-methyl-8-(2,2,2-trifluoro-ethyl)-8H-imidazo[4′,5′:3,4]benzo[2,1-d]thiazol-2-ylamino]-benzenesulfonamide.TFA

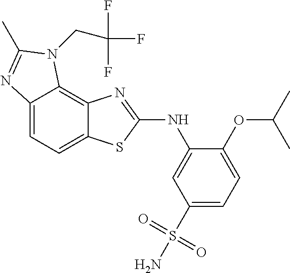

The title compound was prepared using Intermediate 220 in place of Intermediate 217 by the method of Example 133. ¹H NMR (400 MHz, MeOH-d₄) δ 9.19 (s, 1 H), 7.95 (d, J=8.3 Hz, 1 H), 7.58-7.61 (m, 1 H), 7.53 (d, J=8.3 Hz, 1 H), 7.19-7.21 (m, 1 H), 5.83-5.96 (m, 2 H), 4.84-4.87 (m, 1 H), 2.98 (s, 3 H), 1.46 (d, J=6.1 Hz, 6 H); MS m/e 500 (M+H).

### Example 137

### 4-Isopropoxy-3-[8-(2,2,2-trifluoro-ethyl)-8H-imidazo[4′,5′:3,4]-benzo[2,1-d]thiazol-2-ylamino]-benzenesulfonamide.TFA

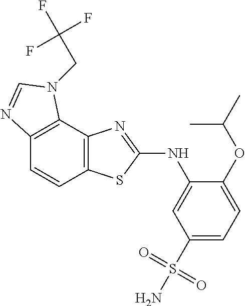

The title compound was prepared using Intermediate 221 in place of Intermediate 217 by the method of Example 133. ¹H NMR (400 MHz, MeOH-d₄) δ 9.26 (s, 1 H), 9.20 (s, 1 H), 7.89 (d, J=8.8 Hz, 1 H), 7.59 (m, 2 H), 7.19 (d, J=8.8 Hz, 1 H), 5.80 (q, J=8.3 Hz, 2 H), 4.82-4.88 (m, 1 H), 1.46 (d, J=6.1 Hz, 6 H); MS m/e 486 (M+H).

### Example 138

### (5-Methanesulfonyl-2-methoxy-phenyl)-[7-methyl-8-(2,2,2-trifluoro-ethyl)-8H-imidazo[4′,5′:3,4]benzo[2,1-d]thiazol-2-yl]-amine.TFA

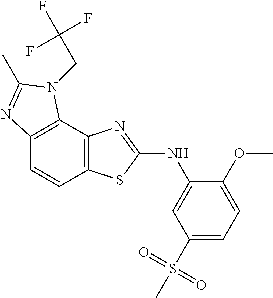

The title compound was prepared using Intermediate 222 in place of Intermediate 217 by the method of Example 133. ¹H NMR (400 MHz, MeOH-d₄) δ 9.17 (d, J=2.2 Hz, 1 H), 7.88 (d, J=8.6 Hz, 1 H), 7.48 (d, J=8.8 Hz, 1 H), 7.38 (dd, J=2.3, 8.4 Hz, 1 H), 7.08 (d, J=8.6 Hz, 1 H), 5.87 (q, J=8.1 Hz, 2 H), 4.05 (s, 3 H), 3.01 (s, 3 H), 2.98 (s, 3 H); MS m/e 471 (M+H).

### Example 139

### (8-Ethyl-7-methyl-8H-imidazo[4′,5′:3,4]benzo[2,1-d]thiazol-2-yl)-(5-fluoro-2-isopropoxy-phenyl)-amine.TFA

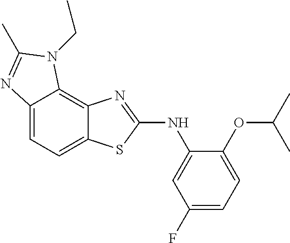

The title compound was prepared using Intermediate 223 in place of Intermediate 217 by the method of Example 133. ¹H NMR (400 MHz, MeOH-d₄) δ 8.45 (dd, J=3.2, 11.5 Hz, 1 H), 7.79 (d, J=8.6 Hz, 1 H), 7.42 (d, J=8.6 Hz, 1 H), 6.99 (dd, J=5.1, 9.1 Hz, 1 H), 6.72 (td, J=3.2, 8.4 Hz, 1 H), 4.79 (q, J=7.1 Hz, 2 H), 4.65 (m, 1 H), 2.85 (s, 3 H), 1.63 (t, J=7.2 Hz, 3 H), 1.40 (d, J=6.1 Hz, 6 H); MS m/e 385 (M+H).

### Example 140

### (7,8-Dimethyl-8H-imidazo[4′,5′:3,4]benzo[2,1-d]thiazol-2-yl)-(5-fluoro-2-isopropoxy-phenyl)-amine.TFA

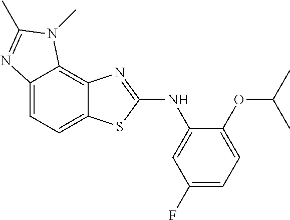

The title compound was prepared using Intermediate 224 in place of Intermediate 217 by the method of Example 133. ¹H NMR (400 MHz, MeOH-d₄) δ 8.24-8.32 (m., 1 H), 7.69-7.84 (m, 1 H), 7.31-7.48 (m, 1 H), 6.90-7.03 (m, 1 H), 6.65-6.77 (m, 1 H), 4.50-4.75 (m, 1 H), 4.21-4.49 (m, 3 H), 2.80 (s, 3 H), 1.39 (d, J=5.9 Hz, 6 H); MS m/e 371 (M+H).

### Example 141

### (5-Fluoro-2-isopropoxy-phenyl)-[7-methyl-8-(2,2,2-trifluoro-ethyl)-8H-imidazo[4′,5′:3,4]benzo[2,1-d]thiazol-2-yl]-amine.TFA

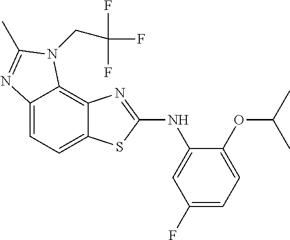

The title compound was prepared using Intermediate 225 in place of Intermediate 217 by the method of Example 133. ¹H NMR (400 MHz, MeOH-d₄) δ=8.36 (dd, J=3.1, 10.9 Hz, 1 H), 7.90 (d, J=8.8 Hz, 1 H), 7.52 (d, J=8.6 Hz, 1 H), 7.04 (dd, J=5.1, 9.0 Hz, 1 H), 6.75-6.80 (m, 1 H), 5.82 (q, J=8.3 Hz, 2 H), 4.62-4.68 (m, 1 H), 2.89 (s, 3 H), 1.40 (d, 6 H); MS m/e 439 (M+H).

### Example 142

### (5-Fluoro-2-isopropoxy-phenyl)-[8-(2,2,2-trifluoro-ethyl)-8H-imidazo[4′,5′:3,4]-benzo[2,1-d]thiazol-2-yl]-amine.TFA

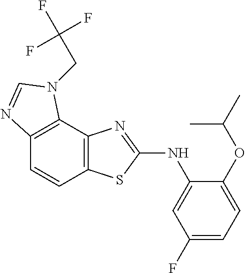

The title compound was prepared using Intermediate 226 in place of Intermediate 217 by the method of Example 133. ¹H NMR (400 MHz, MeOH-d₄) δ=9.06 (br. s., 1 H), 8.34-8.41 (m, 1 H), 7.86 (d, J=8.8 Hz, 1 H), 7.58 (d, J=8.6 Hz, 1 H), 6.97-7.05 (m, 1 H), 6.75 (t, J=8.3 Hz, 1 H), 5.74 (q, J=8.3 Hz, 2 H), 4.60-4.69 (m, 1 H), 1.39 (d, J=5.9 Hz, 6 H); MS m/e 425 (M+H).

### Example 143

### (8-Ethyl-7-methyl-8H-imidazo[4′,5′:3,4]benzo[2,1-d]thiazol-2-yl)-(2-isopropoxy-5-methanesulfonyl-phenyl)-amine.TFA

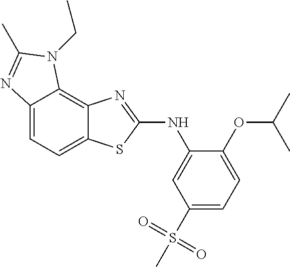

The title compound was prepared using Intermediate 227 in place of Intermediate 217 by the method of Example 133. ¹H NMR (400 MHz, MeOH-d₄) δ=9.26 (s, 1 H), 7.82 (d, J=8.1 Hz, 1 H), 7.52 (d, J=6.8 Hz, 1 H), 7.39 (d, J=7.1 Hz, 1 H), 7.19 (d, J=7.8 Hz, 1 H), 4.91-4.98 (m, 3 H), 3.06 (s, 3 H), 2.90 (s, 3 H), 1.61 (t, J=6.2 Hz, 3 H), 1.50 (d, J=6.1 Hz, 6 H); MS m/e 445 (M+H).

### Example 144

### (7,8-Dimethyl-8H-imidazo[4′,5′:3,4]benzo[2,1-d]thiazol-2-yl)-(2-isopropoxy-5-methanesulfonyl-phenyl)-amine.TFA

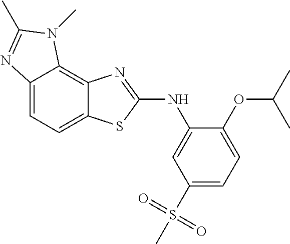

The title compound was prepared using Intermediate 228 in place of Intermediate 217 by the method of Example 133. ¹H NMR (400 MHz, MeOH-d₄) δ=9.30 (s, 1 H), 7.68 (d, J=8.3 Hz, 1 H), 7.38 (d, J=8.6 Hz, 1 H), 7.25 (d, J=8.6 Hz, 1 H), 7.07 (d, J=8.6 Hz, 1 H), 4.82-4.88 (m, 1 H), 4.36 (s, 3 H), 3.00 (s, 3 H), 2.78 (s, 3 H), 1.50 (d, J=5.9 Hz, 6 H); MS m/e 431 (M+H).

### Example 145

### (2-Isopropoxy-5-methanesulfonyl-phenyl)-[7-methyl-8-(2,2,2-trifluoro-ethyl)-8H-imidazo[4′,5′:3,4]benzo[2,1-d]thiazol-2-yl]-amine.TFA

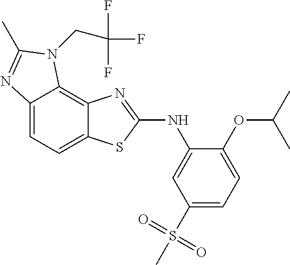

The title compound was prepared using Intermediate 229 in place of Intermediate 217 by the method of Example 133. ¹H NMR (400 MHz, MeOH-d₄) δ=9.20 (d, J=2.4 Hz, 1 H), 7.74 (d, J=8.6 Hz, 1 H), 7.42 (d, J=8.6 Hz, 1 H), 7.38 (dd, J=2.2, 8.6 Hz, 1 H), 7.08 (d, J=8.8 Hz, 1 H), 5.76 (q, J=8.2 Hz, 2 H), 4.84 (spt, J=6.1 Hz, 1 H), 3.02 (s, 3 H), 2.88 (s, 3 H), 1.49 (d, J=5.9 Hz, 6 H); MS m/e 499 (M+H).

### Example 146

### (2-Isopropoxy-5-methanesulfonyl-phenyl)-[8-(2,2,2-trifluoro-ethyl)-8H-imidazo[4′,5′:3,4]-benzo[2,1-d]thiazol-2-yl]-amine.TFA

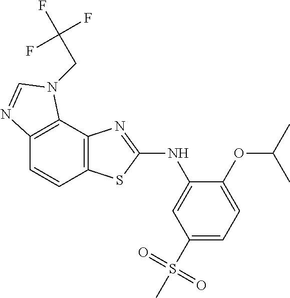

The title compound was prepared using Intermediate 230 in place of Intermediate 217 by the method of Example 133. ¹H NMR (400 MHz, MeOH-d₄) δ=9.37-9.44 (m, 1 H), 9.08 (s, 1 H), 7.87 (d, J=8.6 Hz, 1 H), 7.58 (d, J=8.1 Hz, 2 H), 7.21-7.29 (m, 1 H), 5.80 (q, J=8.0 Hz, 2 H), 4.91 (spt, J=6.1 Hz, 1 H), 3.08 (s, 3 H), 1.49 (d, J=6.1 Hz, 6 H); MS m/e 485 (M+H).

### Example 147

### (2-Isopropoxy-5-methanesulfonyl-phenyl)-(6-methyl-6H-imidazo[4′,5′:3,4]benzo[2,1-d]thiazol-2-yl)-amine.TFA

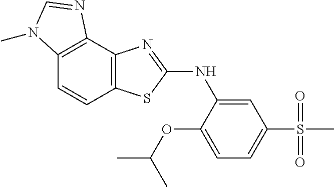

The title compound was prepared using Intermediate 231 in place of Intermediate 217 by the method of Example 133. ¹H NMR (400 MHz, MeOH-d₄) δ=9.43 (s, 1 H), 9.05-9.17 (m, 1 H), 7.83-7.97 (m, 1 H), 7.64 (d, J=7.3 Hz, 1 H), 7.57 (br. s., 1 H), 7.27 (d, J=8.6 Hz, 1 H), 4.91 (spt, J=6.1 Hz, 1 H), 4.14 (s, 3 H), 3.20 (s, 3 H), 1.48 (d, J=5.9 Hz, 6 H); MS m/e 417 (M+H).

### Example 148

### (4-Methoxy-pyridin-3-yl)-[7-methyl-8-(2,2,2-trifluoro-ethyl)-8H-imidazo[4′,5′:3,4]benzo[2,1-d]thiazol-2-yl]-amine.TFA

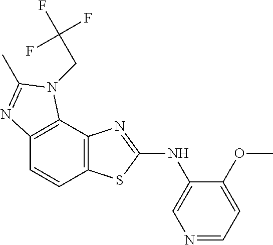

The title compound was prepared using Intermediate 232 in place of Intermediate 217 by the method of Example 133. ¹H NMR (400 MHz, MeOH-d₄) δ=9.87 (d, J=1.0 Hz, 1 H), 8.54 (dd, J=1.2, 6.6 Hz, 1 H), 8.03 (d, J=8.8 Hz, 1 H), 7.73 (d, J=6.6 Hz, 1 H), 7.67 (d, J=8.8 Hz, 1 H), 5.86 (q, J=8.3 Hz, 2 H), 4.32 (s, 3 H), 2.99 (s, 3 H); MS m/e 394 (M+H).

### Example 149

### (4-Methoxy-pyridin-3-yl)-(6-methyl-6H-imidazo[4′,5′:3,4]benzo[2,1-d]thiazol-2-yl)-amine.TFA

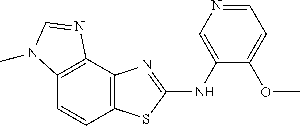

The title compound was prepared using Intermediate 233 in place of Intermediate 217 by the method of Example 133. ¹H NMR (400 MHz, MeOH-d₄) δ=10.47 (d, J=1.0 Hz, 1 H), 9.41 (s, 1 H), 8.50 (dd, J=1.2, 6.6 Hz, 1 H), 7.99 (d, J=8.8 Hz, 1 H), 7.71 (d, J=8.8 Hz, 1 H), 7.66 (d, J=6.6 Hz, 1 H), 4.30 (s, 3 H), 4.19 (s, 3 H); MS m/e 312 (M+H).

### Example 150

### (5-Methanesulfonyl-2-methoxy-phenyl)-(1-methyl-1H-6-thia-1,2,8-triaza-as-indacen-7-yl)-amine.TFA

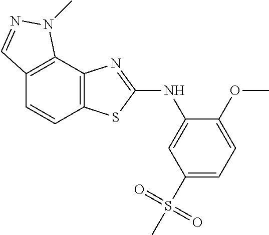

The title compound was prepared from Intermediate 250 by the procedure of Example 92 without conversion to the HCl salt, with the exception that the reaction was conducted at room temperature for 1 hour. ¹H NMR (400 MHz, CHLOROFORM-d) δ 9.51 (d, J=2.45 Hz, 1H), 8.03 (s, 1H), 7.89 (br. s., 1H), 7.64 (dd, J=2.20, 8.56 Hz, 1H), 7.56 (d, J=8.56 Hz, 1H), 7.40 (d, J=8.56 Hz, 1H), 7.06 (d, J=8.56 Hz, 1H), 4.64 (s, 3H), 4.07 (s, 3H), 3.08 (s, 3H); MS m/e 389.0 (M+H).

### Example 151

### 4-Methoxy-3-(thiazolo[4,5-f]isoquinolin-2-ylamino)-benzonitrile.TFA

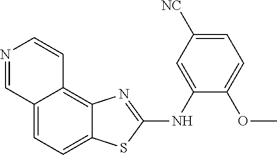

The title compound was prepared from Intermediate 251 by the procedure of Example 92 without conversion to the HCl salt, with the exception that the reaction was conducted at room temperature for 1 hour. ¹H NMR (400 MHz, CHLOROFORM-d) δ 9.80 (s, 1H), 8.99 (d, J=1.96 Hz, 1H), 8.86 (d, J=6.60 Hz, 1H), 8.64 (d, J=6.36 Hz, 1H), 8.20 (d, J=8.80 Hz, 1H), 8.08 (d, J=8.56 Hz, 1H), 7.49 (dd, J=1.96, 8.56 Hz, 1H), 7.05 (d, J=8.31 Hz, 1H), 4.08 (s, 3H); MS m/e 333.0 (M+H).

### Example 152

### 8-Ethyl-N-(4-methoxypyridin-3-yl)-7-methyl-8H-imidazo[4′,5′:5,6]benzo[1,2-d]thiazol-2-amine

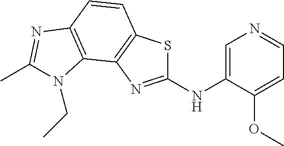

The title compound was prepared using Intermediate 262 in place of Intermediate 217 by the method of Example 133. ¹H NMR (400 MHz, MeOH-d4) δ 10.03 (d, J=1.22 Hz, 1H), 8.57 (dd, J=1.22, 6.60 Hz, 1H), 8.02 (d, J=8.80 Hz, 1H), 7.72 (d, J=6.85 Hz, 1H), 7.64 (d, J=8.80 Hz, 1H), 4.59 (q, J=7.09 Hz, 2H), 4.33 (s, 3H), 2.95 (s, 3H), 1.72 (t, 3H); MS m/e 340 (M+H).

### Example 153

### N-(4-Methoxypyridin-3-yl)-8-(2,2,2-trifluoroethyl)-8H-imidazo[4′,5′:5,6]-benzo[1,2-d]thiazol-2-amine

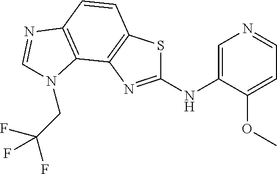

The title compound was prepared using Intermediate 263 in place of Intermediate 217 by the method of Example 133. ¹H NMR (400 MHz, MeOH-d4) δ 9.93 (s, 1H), 9.23 (s, 1H), 8.53 (dd, J=0.98, 6.60 Hz, 1H), 7.99 (d, J=8.80 Hz, 1H), 7.69-7.74 (m, 2H), 5.81 (q, J=8.31 Hz, 2H), 4.32 (s, 3H); MS m/e 380 (M+H).

### Example 154

### N-(4-Methoxypyridin-3-yl)-7,8-dimethyl-8H-imidazo[4′,5′:5,6]benzo[1,2-d]thiazol-2-amine.TFA

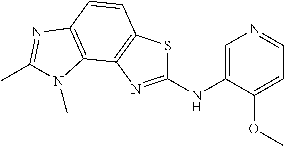

The title compound was prepared using Intermediate 264 in place of Intermediate 217 by the method of Example 133. ¹H NMR (400 MHz, MeOH-d4) δ 10.12 (d, J=0.98 Hz, 1H), 8.55 (dd, J=1.10, 6.72 Hz, 1H), 8.01 (d, J=8.80 Hz, 1H), 7.71 (d, J=6.85 Hz, 1H), 7.62 (d, J=8.80 Hz, 1H), 4.48 (s, 3H), 4.32 (s, 3H), 2.92 (s, 3H); MS m/e 326 (M+H).

### Example 155

### N-(4-Ethoxypyridin-3-yl)-8-ethyl-7-methyl-8H-imidazo[4′,5′:5,6]benzo[1,2-d]thiazol-2-amine.TFA

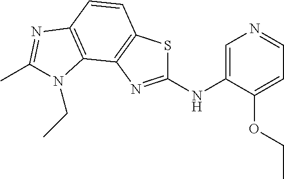

The title compound was prepared using Intermediate 265 in place of Intermediate 217 by the method of Example 133. ¹H NMR (400 MHz, MeOH-d4) δ 10.03 (d, J=1.22 Hz, 1H), 8.53 (dd, J=1.10, 6.72 Hz, 1H), 8.03 (d, J=8.80 Hz, 1H), 7.69 (d, J=6.85 Hz, 1H), 7.63 (d, J=8.80 Hz, 1H), 4.94 (q, J=7.17 Hz, 2H), 4.61 (q, J=7.01 Hz, 2H), 2.94 (s, 3H), 1.72 (t, J=7.21 Hz, 3H), 1.65 (t, J=7.09 Hz, 3H); MS m/e 354 (M+H).

### Example 156

### N-(4-Ethoxypyridin-3-yl)-6-methyl-6H-imidazo[4′,5′:5,6]benzo[1,2-d]thiazol-2-amine.HCl

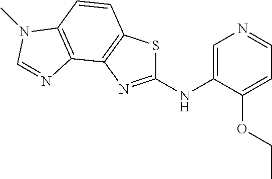

To a suspension of Intermediate 266 (316 mg, 0.965 mmol) in HOAc (4 mL) was added a solution of 0.5 M Br₂ in HOAc (1.9 mL, 0.95 mmol) dropwise. After stirring at RT overnight, HOAc was removed in vacuo. The residue was dissolved in DMSO, and purified by RP-HPLC (90-10% H₂O—CH₃CN, 0.1% TFA). The purified material was dissolve in MeOH, 0.5 mL of 36% aq. HCl was added, and concentrated in vacuo. This process was repeated twice and the residue was dried under vacuum to obtain the title compound as a white HCl salt. ¹H NMR (400 MHz, MeOH-d4) δ 10.31 (d, J=1.22 Hz, 1H), 9.47 (s, 1H), 8.50 (dd, J=1.22, 6.60 Hz, 1H), 8.12 (d, J=8.80 Hz, 1H), 7.81 (d, J=8.80 Hz, 1H), 7.70 (d, J=6.85 Hz, 1H), 4.62 (q, J=7.01 Hz, 2H), 4.22 (s, 3H), 1.65 (t, J=6.97 Hz, 3H); MS m/e 326 (M+H).

### Example 157

### N-(4-Isopropoxypyridin-3-yl)-6-methyl-6H-imidazo[4′,5′:5,6]benzo[1,2-d]thiazol-2-amine.HCl

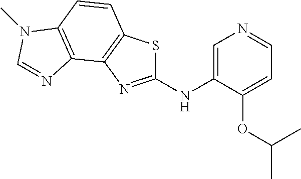

The title compound was prepared using Intermediate 267 in place of Intermediate 266 by the method of Example 156. ¹H NMR (400 MHz, MeOH-d4) δ 10.30 (d, J=0.98 Hz, 1H), 9.51 (s, 1H), 8.49 (dd, J=0.98, 6.60 Hz, 1H), 8.05 (d, J=8.80 Hz, 1H), 7.76 (d, J=8.80 Hz, 1H), 7.72 (d, J=6.60 Hz, 1H), 5.20-5.28 (m, 1H), 4.22 (s, 3H), 1.60 (d, J=6.11, 6H); MS m/e 340 (M+H).

### Example 158

### N-(4-Isopropoxypyridin-3-yl)-6,7-dimethyl-6H-imidazo[4′,5′:5,6]benzo[1,2-d]thiazol-2-amine.HCl

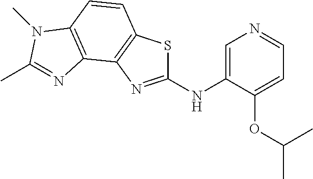

The title compound was prepared using Intermediate 268 in place of Intermediate 266 by the method of Example 156. ¹H NMR (400 MHz, MeOH-d4) δ 10.25 (s, 1H), 8.47 (d, J=6.60 Hz, 1H), 7.90 (d, J=8.80 Hz, 1H), 7.71 (d, J=6.85 Hz, 1H), 7.64 (d, J=8.80 Hz, 1H), 5.30-5.32 (m, 1H), 4.06 (s, 3H), 2.99 (s, 3H), 1.60 (d, J=6.11 Hz, 6H); MS m/e 354 (M+H).

### Example 159

### 8-Ethyl-N-(4-isopropoxypyridin-3-yl)-7-methyl-8H-imidazo[4′,5′:5,6]benzo[1,2-d]thiazol-2-amine.HCl

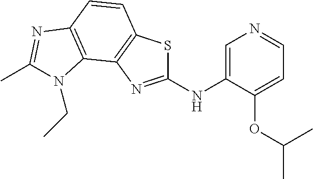

The title compound was prepared using Intermediate 269 in place of Intermediate 266 by the method of Example 156. ¹H NMR (400 MHz, MeOH-d4) δ 10.04 (s, 1H), 8.53 (d, J=6.85 Hz, 1H), 8.03 (d, J=8.80 Hz, 1H), 7.75 (d, J=6.85 Hz, 1H), 7.64 (d, J=8.80 Hz, 1H), 5.22-5.29 (m, 1H), 4.95 (q, J=7.22 Hz, 2H), 2.98 (s, 3H), 1.74 (t, J=7.21 Hz, 3H), 1.60 (d, J=5.87 Hz, 6H); MS m/e 368 (M+H).

### Example 160

### 4-isopropoxy-3-((6-methyl-6H-imidazo[4′,5′:5,6]benzo[1,2-d]thiazol-2-yl)amino)benzonitrile.HCl

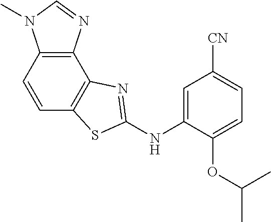

The title compound was prepared from Intermediate 257 by the method of Example 28. ¹H NMR (400 MHz, DMSO-d₆) δ 10.28 (s, 1H), 9.64 (s, 1H), 9.15 (d, J=1.96 Hz, 1H), 8.12 (d, J=8.80 Hz, 1H), 7.76 (d, J=8.56 Hz, 1H), 7.56 (dd, J=1.96, 8.56 Hz, 1H), 7.31 (d, J=8.80 Hz, 1H), 4.85-4.94 (m, 1H), 4.11 (s, 3H), 1.39 (d, J=5.87 Hz, 6H). MS m/e 364 (M+H).

### Example 161

### 4-isopropoxy-3-((6-methyl-6H-thiazolo[4,5-e]indol-2-yl)amino)benzenesulfonamide.HCl

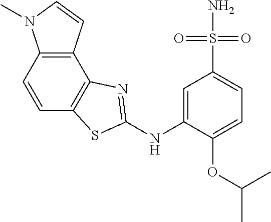

The title compound was prepared from Intermediate 258 by the method of Example 28. The product did not precipitate in the workup, so the layers were separated, the aq. phase was extracted with EtOAc, and the combined organic extracts were concentrated and were triturated with acetonitrile. The solid was treated with 1N HCl and EtOH and concentrated, then re-concentrated several times with EtOH to remove residual water. The solid was then triturated from acetonitrile to afford the title compound. ¹H NMR (400 MHz, DMSO-d₆+D₂O) δ 9.10 (br. s., 1H), 7.59 (d, J=8.56 Hz, 1H), 7.54 (dd, J=2.08, 8.68 Hz, 1H), 7.33-7.42 (m, 2H), 7.28 (d, J=8.80 Hz, 1H), 6.73 (d, J=2.93 Hz, 1H), 4.82 (sept, J=5.99, 1H), 3.85 (s, 3H), 1.34 (d, J=5.87 Hz, 6H). MS m/e 417 (M+H).

### Example 162

### 3-((6-methyl-6H-thiazolo[4,5-e]indol-2-yl)amino)-4-(trifluoromethoxy)benzenesulfonamide.HCl

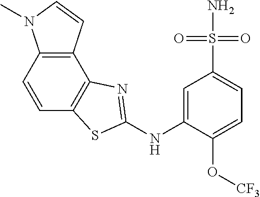

The title compound was prepared from Intermediate 259 by the method of Example 28. The product did not precipitate in the workup, so the layers were separated, the aq. phase was extracted with EtOAc, and the combined organic extracts were washed with sat. aq. NaCl, filtered, dried over Na₂SO₄, filtered, and concentrated. The residue was triturated with acetonitrile. The solid was treated with 1N HCl and EtOH and concentrated, then re-concentrated several times from EtOH to remove residual water. ¹H NMR (400 MHz, DMSO-d₆) δ 9.56 (br. s., 1H), 7.46-7.71 (m, 5H), 7.32-7.43 (m, 2H), 6.72 (d, J=2.93 Hz, 1H), 3.85 (s, 3H). MS m/e 443 (M+H).

### Example 163

### N-(2-methoxy-5-(methylsulfonyl)phenyl)-6-methyl-6H-thiazolo[4,5-e]indol-2-amine.HCl

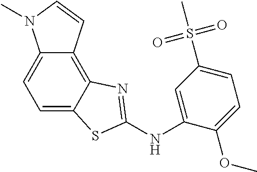

The title compound was prepared from Intermediate 260 by the method of Example 28. The product did not precipitate in the workup, so the layers were separated, and the organic layer concentrated and purified by HPLC eluting with water/acetonitrile/0.2% trifluoroacetic acid. The HCl salt was made by repeated exposure to 1N HCl in EtOH, and the product was re-crystallized from EtOH. ¹H NMR (400 MHz, DMSO-d₆) δ 10.19 (br. s., 1H), 9.53 (s, 1H), 7.53-7.60 (m, 2H), 7.38 (d, J=2.93 Hz, 1H), 7.34 (d, J=8.56 Hz, 1H), 7.29 (d, J=8.56 Hz, 1H), 6.63 (d, J=2.69 Hz, 1H), 4.01 (s, 3H), 3.85 (s, 3H). MS m/e 388 (M+H).

### Example 164

### 3-6-methyl-6H-imidazo[4′,5′:5,6]benzo[1,2-d]thiazol-2-yl)amino)-4-(trifluoromethoxy)benzonitrile.HCl

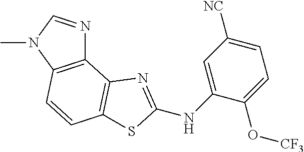

A mixture of Intermediate 261 (0.38 g, 0.72 mmol) and thionyl chloride (2 mL) was heated at 75° C. for 1.5 h. The mixture was concentrated and the residue was concentrated twice from heptanes to remove residual thionyl chloride. The residue was purified by HPLC, eluting with water/acetonitrile/0.2% trifluoroacetic acid. The HCl salt was made by repeated exposure to 1N HCl in EtOH, and the product was re-crystallized from EtOH. ¹H NMR (400 MHz, DMSO-d₆) δ 10.96 (s, 1H), 9.49 (br. s., 1H), 9.40 (s, 1H), 8.12 (d, J=8.56 Hz, 1H), 7.79 (d, J=8.80 Hz, 1H), 7.69-7.72 (m, 2H), 4.10 (s, 3H). MS m/e 390 (M+H).

### Compound α

### 3-(2′,4′-Dimethyl-[4,5′]bithiazolyl-2-ylamino)-4-isopropoxy-benzenesulfonamide.HBr

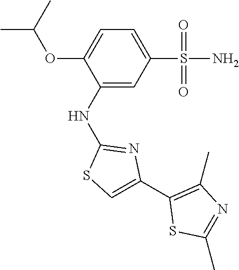

Compound α was tested in cell based and in-vitro assays (vide infra). The cell based and in-vivo activity of Compound α is provided as representative of the activity of the compounds of the present invention, but is not to be construed as limiting the invention in any way.

### Cloning, Expression and Purification

Cloning of Human proMMP9

Amino acid numbering for all human proMMP9 constructs was based on UniProtKB/Swiss-Prot P14780, full-length human matrix metalloproteinase-9 precursor, proMMP9(1-707) (SEQ ID NO:1). One construct, proMMP9(20-445) (SEQ ID NO:2), was based on the previously published crystal structure (Acta Crystallogr D Biol Crystallogr 58(Pt 7): 1182-92). The construct lacked the signal peptide at the N-terminus and also lacked the four hemopexin-like domains at the C-terminus. An N-terminal truncated construct was also designed with an N-terminus truncation after the first observable electron density in the previously published proMMP9 structure and a single amino acid was removed from the C-terminus to produce proMMP9(29-444) (SEQ ID NO:3). Other truncated constructs were also synthesized without the three fibronectin type-II domains (ΔFnII), amino acids 216-390. The ΔFnII constructs were proMMP9(29-444;ΔFnII) (SEQ ID NO:4), proMMP9(67-444;ΔFnII) (SEQ ID NO:5) and proMMP9(20-445;ΔFnII) (SEQ ID NO:6). Binding studies with the proMMP9 proteins without the FnII domains showed that compounds bound with similar affinity compared to the wild-type protein (data not shown).

In order to make the constructs with the FnII domains deleted, proMMP9(29-444;ΔFnII) (SEQ ID NO:4), proMMP9(67-444;ΔFnII) (SEQ ID NO:5) and proMMP9(20-445;ΔFnII) (SEQ ID NO:6), plasmids encoding the different proMMP9 truncations were used as templates for PCR to create two fragments of DNA corresponding to amino acid pairs including: 29-215/391-444, 67-215/391-444, and 20-215/391-445, respectively. Overlapping PCR was used to join the fragments. The 5′ primers had an Nde1 site and a start methionine and the 3′ primers had a stop codon and a Bg12 site. The final PCR products were cloned into the TOPO TA cloning vector (Invitrogen) and the sequences were confirmed. Subsequently the vectors were digested with Nde1 and Bg12 and the sequences were subcloned into Nde1 and BamH1 sites of the T7 expression vector pET11a (Novagen).

Expression of Truncated Forms of Human proMMP9

For expression in E. coli, all of the truncated proMMP9 constructs were transformed into BL21(DE3) RIL cells (Stratagene). Cells were initiated for an overnight culture from glycerol stocks in LB+Ampicillin (100 μg/ml) @ 37° C. shaking at 220 rpms. The overnight culture was subcultured 1:100 in LB+Ampicillin (100 ug/ml) and maintained at 37° C. shaking at 220 rpms. Samples were taken and A600 readings were monitored until an OD of 0.6 was achieved. The culture was induced with 1 mM IPTG and maintained under present growth conditions. Cultures were harvested 3 hours post induction at 6000×g for 10 min. Pellets were washed in 1×PBS with protease inhibitors and stored at −80° C.

Purification of Truncated Forms of Human proMMP9

To purify the truncated proMMP9 proteins from E. coli, cell pellets were suspended in 25 mM Na₂HPO₄ pH 7, 150 mM NaCl, 10 mL/gram cell pellet. The cells were homogenized in a Dounce homogenizer, and then processed twice through a microfluidizer (Microfluidics International Corporation, model M-110Y). The lysate was centrifuged at 32,000×g for 45 minutes at 4° C. The supernatant was discarded. The pellet was suspended in 25 mM Na₂HPO₄ pH 7, 150 mM NaCl, 10 mM DTT, 1 mM EDTA, 10 mL/gram cell pellet. The pellet was homogenized in a Dounce homogenizer, and then centrifuged at 32,000×g for 45 minutes at 4° C. The supernatant was discarded. The pellet was suspended in 7 M urea, 25 mM Tris pH 7.5, 10 mM DTT, 1 mM EDTA, 6.5 mL/gram cell pellet, and then solubilized in a Dounce homogenizer and stirred for approximately 16 hours at ambient temperature. The solubilized protein solution was adjusted to pH 7.5, centrifuged at 45,000×g, 45 minutes at 4° C., and the supernatant, containing the denatured proMMP9, was filtered to 0.8 micron. A 5 mL HiTrap Q Sepharose HP column (GE Healthcare) was prepared according to manufacturer's instructions using Buffer A: 7 M urea, 25 mM Tris pH 7.5 and Buffer B: 7 M urea, 25 mM Tris pH 7.5, 1.0 M NaCl. The protein solution was applied to the HiTrap at 2.5 mL/minute. The column was washed to baseline absorbance with approximately 3.5 CV Buffer A. The proMMP9 was eluted in a 12CV linear gradient from 0% Buffer B to 12% Buffer B. Fractions were collected, analyzed on SDS-PAGE (Novex) and pooled based on purity. The pooled protein was re-natured by drop-wise addition to a solution, stirring and at ambient temperature, of 20 mM Tris pH 7.5, 200 mM NaCl, 5 mM CaCl₂, 1 mM ZnCl₂, 0.7 M L-arginine, 10 mM reduced and 1 mM oxidized glutathione, and was stirred for approximately 16 hours at 4° C. The refolded protein was concentrated to approximately 2.5 mg/mL in Jumbo Sep centrifugal concentrators (Pall) with 10,000 MWCO membranes. The concentrated protein solution was dialyzed at 4° C. for approximately 16 hours against 20 mM Tris pH 7.5, 150 mM NaCl. The dialyzed protein solution was clarified by filtration to 0.8 micron, concentrated to 2 mg/mL as before, centrifuged at 45,000×g for 15 minutes at 4° C. and filtered to 0.2 micron. It was purified on a HiLoad 26/60 Superdex 200 column (GE Healthcare) equilibrated in 20 mM Tris pH 7.5, 200 mM NaCl. Fractions were analyzed by SDS-PAGE and pooled based on purity. The pooled protein was concentrated in a Jumbo Sep concentrator as before and centrifuged at 16,000×g for 10 minutes at 4° C. The protein concentration was determined using Bio-Rad Protein Assay (Bio-Rad Laboratories, Inc.) with bovine serum albumin as a standard. The supernatant was aliquoted, frozen in liquid nitrogen and stored at −80° C.

Full-Length Human proMMP 9

Full-length proMMP9(1-707) (SEQ ID NO:1) was expressed in HEK293 cells or in COS-1 cells as a secreted protein using a pcDNA3.1 expression vector. When expressed as a secreted protein in HEK293 cells or COS-1 cells, there is cotranslational removal of the signal peptide, amino acids 1-19 of full-length proMMP9(1-707) (SEQ ID NO:1). The final purified proMMP9(1-707) (SEQ ID NO:1) protein lacks the signal peptide.

Prior to transfection with the proMMP9(1-707) (SEQ ID NO:1) construct, the HEK293 cells were suspension adapted (shake flasks) in a serum free media (Freestyle 293) supplemented with pluronic acid (F-68) at a final concentration of 0.1%. Once cells reached a density of 1.2×10⁶/mL they were transiently transfected using standard methods. Transient transfection of COS-1 cells was done in flasks with adherent cell cultures and serum free media. For both HEK293 and COS-1 cells, the conditioned media was collected for purification of the proMMP9(1-707) (SEQ ID NO:1) protein. 1.0 M HEPES pH 7.5 was added to 9 L of conditioned media for a final concentration of 50 mM. The media was concentrated to 600 mL in a Kvicklab concentrator fitted with a hollow fiber cartridge of 10,000 MWCO (GE Healthcare). This was clarified by centrifugation at 6,000×g, 15 minutes, at 4° C. and then further concentrated to 400 mL in Jumbo Sep centrifugal concentrators (Pall) with 10,000 MWCO membranes. The concentrated protein was dialyzed against 50 mM HEPES pH 7.5, 10 mM CaCl₂, 0.05% Brij 35, overnight at 4° C. and then dialysis was continued for several hours at 4° C. in fresh dialysis buffer. The dialyzed protein was centrifuged at 6,000×g, 15 minutes, at 4° C., and filtered to 0.45 micron. 12 mL of Gelatin Sepharose 4B resin (GE Healthcare) was equilibrated in 50 mM HEPES pH 7.5, 10 mM CaCl₂, 0.05% Brij 35 in a 2.5 cm diameter Econo-Column (Bio-Rad Laboratories). The filtered protein solution was loaded onto the Gelatin Sepharose resin using gravity flow at approximately 3 mL/minute. The resin was washed with 10CV 50 mM HEPES pH 7.5, 10 mM CaCl₂, 0.05% Brij 35 and eluted with 30 mL 50 mM HEPES pH 7.5, 10 mM CaCl₂, 0.05% Brij 35, 10% DMSO, collected in 5 mL fractions. Fractions containing protein, confirmed by A280 absorbance, were dialyzed, in 500 times the volume of the fractions, against 50 mM HEPES pH 7.5, 10 mM CaCl₂, 0.05% Brij 35, overnight at 4° C. Dialysis was continued for an additional 24 hours in two fresh buffer changes. The dialyzed fractions were analyzed on SDS-PAGE and pooled based on purity. The pooled protein was concentrated to 1.2 mg/mL in Jumbo Sep centrifugal concentrators with 10,000 MWCO membranes. Protein concentration was determined with DC™ protein assay (Bio-Rad Laboratories, Inc.). The protein was aliquoted, frozen in liquid nitrogen and stored at −80° C.

Full-Length Rat proMMP 9

Amino acid numbering for full-length rat proMMP9 was based on UniProtKB/Swiss-Prot P50282, full-length rat matrix metalloproteinase-9 precursor, proMMP9(1-708) (SEQ ID NO:11). The full-length rat proMMP9 was produced with the same methods as described for full-length human proMMP9. In brief, full-length rat proMMP9(1-708) (SEQ ID NO:11) was expressed in HEK293 cells as a secreted protein using a pcDNA3.1 expression vector. When expressed in HEK293 cells and secreted into the media, there is cotranslational removal of the signal peptide, so the final purified full-length rat proMMP9(1-708) (SEQ ID NO:11) protein lacks the signal peptide.

Human proMMP13

The sequence for proMMP13 was amino acids 1-268 from UniProtKB/Swiss-Prot P45452, proMMP13(1-268) (SEQ ID NO:7). The expression construct included a C-terminal Tev cleavage sequence flanking recombination sequences for use in the Invitrogen Gateway system. The construct was recombined into an entry vector using the Invitrogen Gateway recombination reagents. The resulting construct was transferred into a HEK293 expression vector containing a C-terminal 6×-histidine tag. Protein was expressed via transient transfection utilizing HEK293 cells and secreted into the media. When expressed in HEK293 cells and secreted into the media, there is cotranslational removal of the signal peptide, amino acids 1-19 of proMMP13(1-268) (SEQ ID NO:7). The final purified proMMP13(1-268) (SEQ ID NO:7) protein lacks the signal peptide. HEK293 media were harvested and centrifuged. Media were loaded on GE Healthcare HisTrap FF columns, washed with buffer A (20 mM Tris pH 7.5, 200 mM NaCl, 2 mM CaCl₂, 10 mM imidazole), and eluted with buffer B (20 mM Tris pH 7.5, 200 mM NaCl, 2 mM CaCl₂ 200 mM imidazole). The eluted protein was loaded on a Superdex 200 column equilibrated with buffer C (20 mM HEPES pH 7.4, 100 mM NaCl, 0.5 mM CaCl₂). Fractions containing proMMP13(1-268) (SEQ ID NO:7) were pooled and concentrated to >2 mg/mL.

### Human Catalytic MMP3

Catalytic MMP3 was amino acids 100-265 of human MMP3 from UniProtKB/Swiss-Prot P08254, MMP3(100-265) (SEQ ID NO:8). The corresponding nucleotide sequence was subcloned into a pET28b vector to add a C-terminal 6×-Histidine tag and the construct was used for expression in E. coli. The protein was purified to >95% purity from 4.5 M urea solubilized inclusion bodies by standard techniques. Aliquots of purified protein were stored at −70° C. Purified recombinant human catalytic MMP3 is also available from commercial sources (e.g., Calbiochem®, 444217).

### Biological Assays

### ThermoFluor® Assays

### Generalized ThermoFluor® Methods

The ThermoFluor® (TF) assay is a 384-well plate-based binding assay that measures thermal stability of proteins (Biomol Screen 2001, 6, 429-40; Biochemistry 2005, 44, 5258-66). The experiments were carried out using instruments available from Johnson & Johnson Pharmaceutical Research & Development, LLC. TF dye used in all experiments was 1,8-anilinonaphthalene-8-sulfonic acid (1,8-ANS) (Invitrogen: A-47).

Compounds were arranged in a pre-dispensed plate (Greiner Bio-one: 781280), wherein compounds were serially diluted in 100% DMSO across 11 columns within a series. Columns 12 and 24 were used as DMSO reference and contained no compound. For multiple compound concentration-response experiments, the compound aliquots (50 nL) were robotically predispensed directly into black 384-well polypropylene PCR microplates (Abgene: TF-0384/k) using a Cartesian Hummingbird liquid handler (DigiLab, Holliston, Mass.). Following compound dispense, protein and dye solutions were added to achieve the final assay volume of 3 μL. The assay solutions were overlayed with 1 μL of silicone oil (Fluka, type DC 200: 85411) to prevent evaporation.

Assay plates were robotically loaded onto a thermostatically controlled PCR-type thermal block and then heated from 40 to 90° C. at a ramp-rate of 1° C./min for all experiments. Fluorescence was measured by continuous illumination with UV light (Hamamatsu LC6) supplied via fiber optics and filtered through a band-pass filter (380-400 nm; >60D cutoff). Fluorescence emission of the entire 384-well plate was detected by measuring light intensity using a CCD camera (Sensys, Roper Scientific) filtered to detect 500±25 nm, resulting in simultaneous and independent readings of all 384 wells. A single image with 20-sec exposure time was collected at each temperature, and the sum of the pixel intensity in a given area of the assay plate was recorded vs temperature and fit to standard equations to yield the Tₘ (J Biomol Screen 2001, 6, 429-40).

Thermodynamic parameters necessary for fitting compound binding for each proMMP were estimated by differential scanning calorimetry (DSC) and from ThermoFluor® data. The heat capacity of unfolding for each protein was estimated from the molecular weight and from ThermoFluor® dosing data. Unfolding curves were fit singly, then in groups of 12 ligand concentrations the data were fit to a single K_{D} for each compound.

ThermoFluor® with proMMP9(67-444;ΔFnM (SEQ ID NO:5)

The protein sample preparations had to include a desalting buffer exchange step via a PD-10 gravity column (GE Healthcare). The desalting buffer exchange was performed prior to diluting the protein to the final assay concentration of 3.5 μM proMMP9(67-444;ΔFnII) (SEQ ID NO:5). The concentration of proMMP9(67-444;ΔFnII) (SEQ ID NO:5) was determined spectrophotometrically based on a calculated extinction coefficient of ε₂₈₀=33900 M⁻¹ cm⁻¹, a calculated molecular weight of 22.6 kDa, and calculated pI of 5.20. ThermoFluor® reference conditions were defined as follows: 80 μg/mL (3.5 μM) proMMP9(67-444;ΔFnII) (SEQ ID NO:5), 50 μM 1,8-ANS, pH 7.0 Buffer (50 mM HEPES pH 7.0, 100 mM NaCl, 0.001% Tween-20, 2.5 mM MgCl₂, 300 μM CaCl₂). The thermodynamic parameters for proMMP9(67-444;ΔFnII) (SEQ ID NO:5) are as follows: Tₘ (° C.)=63 (+/−0.1), Δ_{U}H_{(Tm)}(cal mol⁻¹)=105000(+/−5000), Δ_{U}S_{(Tm)} (cal mol⁻¹ K⁻¹)=450, Δ_{U}Cₚ (cal mol⁻¹ K⁻¹)=2000.

ThermoFluor® with proMMP9(20-445; ΔFnII (SEQ ID NO:6)

The protein sample preparations included a desalting buffer exchange step via a PD-10 gravity column (GE Healthcare). The desalting buffer exchange was performed prior to diluting the protein to the final assay concentration of 2.8 μM proMMP9(20-445;ΔFnII) (SEQ ID NO:6). The concentration of proMMP9(20-445;ΔFnII) (SEQ ID NO:6) was determined spectrophotometrically based on a calculated extinction coefficient of ε₂₈₀=39880 M⁻¹ cm⁻¹, a calculated molecular weight of 28.2 kDa, and calculated pI of 5.5. ThermoFluor® reference conditions were define as follows: 80 μg/mL (2.8 μM) proMMP9(20-445;ΔFnII) (SEQ ID NO:6), 50 μM 1,8-ANS, pH 7.0 Buffer (50 mM HEPES pH 7.0, 100 mM NaCl, 0.001% Tween-20, 2.5 mM MgCl₂, 300 μM CaCl₂). The thermodynamic parameters for proMMP9(20-445;ΔFnII) (SEQ ID NO:6) are as follows: Tₘ (° C.)=72 (+/−0.1), Δ_{U}H_{(Tm)} (cal mol⁻¹)=160000(+/−5000), Δ_{U}S_{Tm} (cal mol⁻¹ K⁻¹)=434, Δ_{U}Cₚ (cal mol⁻¹ K⁻¹)=2400.

ThermoFluor® with proMMP13(1-268) (SEQ ID NO: 7)

The proMMP13(1-268) (SEQ ID NO:7) protein sample preparations included a desalting buffer exchange step via a PD-10 gravity column (GE Healthcare). The desalting buffer exchange was performed prior to diluting the protein to the final assay concentration of 3.5 μM. The concentration of proMMP13(1-268) (SEQ ID NO:7) was estimated spectrophotometrically based on a calculated extinction coefficient of ε₂₈₀=37000 M⁻¹ cm⁻¹, a calculated molecular weight of 30.8 kDa, and calculated pI of 5.33. ThermoFluor® reference conditions were defined as follows: 100 μg/mL proMMP13(1-268) (SEQ ID NO:7), 25 μM 1,8-ANS, pH 7.0 Buffer (50 mM HEPES pH 7.0, 100 mM NaCl, 0.001% Tween-20, 2.5 mM MgCl₂, 300 μM CaCl₂). The thermodynamic parameters for proMMP13(1-268) (SEQ ID NO:7) are as follows: Tₘ (° C.)=67 (+/−0.1), Δ_{U}H_{(Tm)}) (cal mol⁻¹)=107000(+/−5000), Δ_{U}S_{Tm} (cal mol⁻¹ K⁻¹)=318, Δ_{U}Cₚ (cal mol⁻¹ K⁻¹)=2600.

Thermofluor data for representative compounds of Formula I is shown in Table 1.

TABLE 1

| | proMMP9 | proMMP9 | |
|---|---|---|---|
| | (20-445; ΔFnII) | (67-444; ΔFnII) | proMMP13 (1-268) |
| | (SEQ ID NO: 6) | (SEQ ID NO: 5) | (SEQ ID NO: 7) |
| Example | binding, Kd (μM) | binding, Kd (μM) | binding, Kd (μM) |
| 1 | 3.6 | 0.81 | 7.0 |
| 2 | 2.2 | 0.41 | 8.7 |
| 3 | 3.2 | 1.6 | ND |
| 4 | 67 | 30 | ND |
| 5 | 0.32 | 0.41 | 0.73 |
| 6 | 2.4 | 0.29 | 11 |
| 7 | 2.3 | 0.20 | ND |
| 8 | 3.1 | 0.68 | 1.8 |
| 9 | 27 | 4.4 | 18 |
| 10 | 0.94 | 0.091 | 0.71 |
| 11 | 1.3 | 0.72 | 0.47 |
| 12 | 1.7 | 3.8 | 6.5 |
| 13 | 0.23 | 0.39 | 2.2 |
| 14 | 2.4 | 0.55 | 20 |
| 15 | >95 | 9.9 | 33 |
| 16 | 5.0 | 0.99 | 19 |
| 17 | >95 | >95 | ND |
| 18 | 0.88 | 0.55 | 3.4 |
| 19 | 0.46 | 0.38 | 2.2 |
| 20 | 1.4 | 0.19 | 1.9 |
| 21 | 0.41 | 0.33 | 3.5 |
| 22 | 0.17 | 0.10 | 0.81 |
| 23 | 0.59 | 0.97 | 3.0 |
| 24 | 1.2 | 0.52 | 8.4 |
| 25 | 0.43 | 0.20 | 2.7 |
| 26 | 0.85 | 0.70 | 6.1 |
| 27 | 0.13 | 0.039 | 0.53 |
| 28 | 0.88 | 2.2 | 3.5 |
| 29 | 0.80 | 0.63 | 1.4 |
| 30 | 0.12 | 0.31 | 0.93 |
| 31 | 0.93 | 0.82 | 2.3 |
| 32 | 3.7 | 0.54 | 9.2 |
| 33 | 0.086 | 0.49 | 0.58 |
| 34 | 0.12 | 0.14 | 4.0 |
| 35 | 0.055 | 0.15 | 0.43 |
| 36 | 0.17 | 0.71 | 2.0 |
| 37 | 2.3 | 0.36 | 5.9 |
| 38 | 1.0 | 0.95 | 1.4 |
| 39 | 0.44 | 0.052 | 1.5 |
| 40 | 0.83 | 0.10 | 1.6 |
| 41 | 4.2 | 0.90 | 7.7 |
| 42 | 1.9 | 0.37 | 3.4 |
| 43 | 17 | 2.8 | >95 |
| 44 | 25 | 6.2 | 9.9 |
| 45 | 0.78 | 3.3 | ND |
| 46 | 0.64 | 0.24 | ND |
| 47 | 1.0 | 1.6 | ND |
| 48 | 1.9 | 0.81 | ND |
| 49 | 4.6 | 1.6 | ND |
| 50 | 3.0 | 1.5 | ND |
| 51 | 3.1 | 0.40 | 8.3 |
| 52 | 1.4 | 0.60 | 2.2 |
| 53 | 4.0 | 6.2 | ND |
| 54 | 1.6 | 1.1 | 11 |
| 55 | 6.7 | 2.2 | 2.1 |
| 56 | 1.0 | 3.3 | 9.8 |
| 57 | 0.23 | 0.035 | 2.8 |
| 58 | 2.6 | 0.93 | 21 |
| 59 | 2.0 | 0.68 | 3.2 |
| 60 | 2.8 | 0.97 | 1.5 |
| 61 | 3.2 | 0.86 | 2.1 |
| 62 | 3.9 | 1.6 | 51 |
| 63 | 1.2 | 0.45 | 2.3 |
| 64 | 0.97 | 0.90 | ND |
| 65 | 0.077 | 0.25 | ND |
| 66 | 5.9 | 1.7 | ND |
| 67 | 0.087 | 0.077 | ND |
| 68 | 7.5 | 12 | ND |
| 69 | 0.43 | 0.22 | ND |
| 70 | 0.56 | 0.12 | ND |
| 71 | 2.0 | 1.6 | ND |
| 72 | 2.1 | 1.6 | ND |
| 73 | 13 | 7.5 | ND |
| 74 | 6.0 | 3.7 | ND |
| 75 | >95 | 15 | ND |
| 76 | 0.53 | 0.38 | ND |
| 77 | 3.2 | 2.6 | ND |
| 78 | 10 | 3.4 | ND |
| 79 | 3.2 | 0.93 | ND |
| 80 | 2.3 | 0.96 | ND |
| 81 | 5.2 | 1.2 | ND |
| 82 | 1.5 | 1.1 | ND |
| 83 | ND | ND | ND |
| 84 | ND | ND | ND |
| 85 | 0.58 | 0.15 | ND |
| 86 | >95 | 0.68 | ND |
| 87 | 6.0 | 1.6 | ND |
| 88 | ND | ND | ND |
| 89 | 3.1 | 0.78 | 6.8 |
| 90 | 0.28 | 3.0 | 2.7 |
| 91 | 0.95 | 2.9 | 9.0 |
| 92 | 1.1 | 0.41 | ND |
| 93 | 19 | 8.1 | ND |
| 94 | 15 | 9.2 | ND |
| 95 | 0.19 | 0.36 | ND |
| 96 | 0.73 | 0.44 | ND |
| 97 | 4.4 | 1.0 | ND |
| 98 | 7.6 | 3.0 | ND |
| 99 | 20 | 3.2 | ND |
| 100 | 76 | ND | ND |
| 101 | 0.79 | 0.43 | ND |
| 102 | 0.25 | 0.68 | ND |
| 103 | 0.75 | 0.37 | ND |
| 104 | 7.7 | 6.1 | ND |
| 105 | 65 | 0.80 | ND |
| 106 | 0.60 | 0.53 | ND |
| 107 | 6.8 | 1.5 | ND |
| 108 | >95 | 1.4 | ND |
| 109 | >95 | 2.3 | ND |
| 110 | >95 | 0.34 | ND |
| 111 | 3.1 | 1.7 | ND |
| 112 | >95 | 7.0 | ND |
| 113 | 0.14 | 1.5 | ND |
| 114 | 1.2 | 4.6 | ND |
| 115 | 3.0 | 25 | 14 |
| 116 | 1.8 | 7.3 | 8.7 |
| 117 | 8.8 | 3.5 | 32 |
| 118 | 0.29 | 0.86 | ND |
| 119 | 0.91 | 1.2 | ND |
| 120 | 3.4 | 2.6 | ND |
| 121 | 4.0 | 0.86 | ND |
| 122 | 1.2 | 0.34 | ND |
| 123 | 5.3 | 4.7 | ND |
| 124 | 5.7 | 2.8 | ND |
| 125 | 3.1 | 0.60 | ND |
| 126 | >95 | 2.2 | ND |
| 127 | 4.3 | 0.98 | ND |
| 128 | 4.9 | 1.5 | ND |
| 129 | 1.2 | 0.52 | ND |
| 130 | >95 | 1.2 | ND |
| 131 | >95 | 2.0 | ND |
| 132 | >95 | 26 | ND |
| 133 | 1.3 | 0.31 | ND |
| 134 | 1.6 | 0.90 | ND |
| 135 | >95 | 0.64 | ND |
| 136 | 0.77 | 0.59 | ND |
| 137 | 0.44 | 0.63 | ND |
| 138 | 0.69 | 0.48 | ND |
| 139 | >95 | 0.96 | ND |
| 140 | >95 | 0.81 | ND |
| 141 | 33 | 7.5 | ND |
| 142 | >95 | 2.4 | ND |
| 143 | 1.2 | 0.26 | ND |
| 144 | 0.88 | 0.66 | ND |
| 145 | 2.5 | 0.56 | ND |
| 146 | 3.1 | 0.64 | ND |
| 147 | 1.4 | 0.40 | ND |
| 148 | 0.86 | 0.29 | ND |
| 149 | 1.0 | 0.039 | ND |
| 150 | 8.8 | 1.2 | ND |
| 151 | >95 | ND | ND |
| 152 | ND | ND | ND |
| 153 | 1.4 | 0.6 | ND |
| 154 | 6.6 | 1.1 | ND |
| 155 | 0.72 | 0.14 | ND |
| 156 | 0.65 | 0.040 | ND |
| 157 | 0.35 | 0.018 | ND |
| 158 | 0.26 | 0.014 | ND |
| 159 | 0.38 | 0.043 | ND |
| 160 | 0.29 | 0.075 | ND |
| 161 | 0.48 | ND | ND |
| 162 | 0.43 | ND | ND |
| 163 | 0.42 | ND | ND |
| 164 | 0.36 | 0.11 | ND |

### Enzyme Assays

proMMP9/MMP3 P126 Activation Assay

Compounds were assessed for inhibition of proMMP9 activation by catalytic MMP3, MMP3(100-265) (SEQ ID NO:8) using full-length proMMP9(1-707) (SEQ ID NO:1) purified from HEK293 cells and a peptide (Mca-PLGL-Dpa-AR-NH₂, BioMol P-126) that fluoresces upon cleavage by catalytic MMP9. The assay buffer employed was 50 mM Hepes, pH 7.5, 10 mM CaCl₂, 0.05% Brij-35. DMSO was included at a final concentration of 2%, arising from the test compound addition. On the day of assay, proMMP9(1-707) (SEQ ID NO:1) purified from HEK293 cells and MMP3(100-265) (SEQ ID NO:8) were diluted to 400 nM in assay buffer. The reaction volume was 50 μL. In 96-well black plates (Costar 3915), 44 μL of assay buffer was mixed with 1.0 μL of test compound, 2.5 μL of 400 nM proMMP9(1-707) (SEQ ID NO:1) purified from HEK293 cells and the reaction was initiated with 2.5 μL of 400 nM MMP3(100-265) (SEQ ID NO:8).The plate was sealed and incubated for 80 min at 37° C. Final concentrations were 20 nM proMMP9(1-707) (SEQ ID NO:1) purified from HEK293 cells and 20 nM MMP3(100-265) (SEQ ID NO:8), and concentrations of test compounds were varied to fully bracket the IC₅₀. Immediately following the 80 min incubation, 50 μL of 40 μM P-126 substrate was added (freshly diluted in assay buffer), and the resulting activity associated with catalytic MMP9 was kinetically monitored at 328 nm excitation, 393 nm emission for 10-15 min at 37° C., using a Spectramax Gemini XPS reader (Molecular Devices). Reactivity of residual MMP3 towards P-126 substrate was minimal under these conditions. Initial velocities were plotted by use of a four-parameter logistics equation (GraphPad Prism® software) for determination of IC₅₀.

### ProMMP13/Plasmin P126 Activation Assay

Compounds were assessed for inhibition of proMMP13 activation by plasmin using a peptide (Mca-PLGL-Dpa-AR-NH₂, BioMol P-126) that fluoresces upon cleavage by catalytic MMP13. The assay buffer employed was 50 mM Hepes, pH 7.5, 10 mM CaCl₂, 0.05% Brij-35. DMSO was included at a final concentration of 2%, arising from the test compound addition. On the day of assay, proMMP13(1-268) (SEQ ID NO:7) purified from HEK293 cells and plasmin were diluted to 160 nM and 320 nM, respectively, in assay buffer. The reaction volume was 50 μL. In 96-well black plates (Costar 3915), 44 μL of assay buffer was mixed with 1.0 μL of test compound, 2.5 μL of 160 nM proMMP13(1-268) (SEQ ID NO:7), and the reaction was initiated with 2.5 μL of 320 nM plasmin. The plate was sealed and incubated for 40 min at 37° C. Final concentrations were 8 nM proMMP13(1-268) (SEQ ID NO:7) and 16 nM plasmin, and concentrations of test compounds were varied to fully bracket the IC₅₀. Immediately following the 40 min incubation, 50 μL of 40 μM P-126 substrate was added (freshly diluted in assay buffer), and the resulting activity associated with catalytic MMP13 was kinetically monitored at 328 nm excitation, 393 nm emission for 10-15 min at 37° C., using a Spectramax Gemini XPS reader (Molecular Devices). Plasmin was not reactive towards P-126 substrate under these conditions. Initial velocities were plotted by use of a four-parameter logistics equation (GraphPad Prism® software) for determination of IC₅₀.

### ProMMP9/MMP3 DQ Gelatin Activation Assay

Compounds were assessed for inhibition of proMMP9 activation by catalytic MMP3 using a quenched fluorescein gelatin substrate (DQ gelatin, Invitrogen D12054) that fluoresces upon cleavage by activated MMP9. The assay buffer employed was 50 mM Hepes, pH 7.5, 10 mM CaCl₂, 0.05% Brij-35. DMSO was included at a final concentration of 0.2%, arising from the test compound addition. On the day of assay, full-length proMMP9(1-707) (SEQ ID NO:1) from COS-1 cells and catalytic MMP3(100-265) (SEQ ID NO:8) were diluted to 60 nM and 30 nM, respectively, in assay buffer. Test compounds in DMSO were diluted 250-fold in assay buffer at 4× the final concentration. The reaction volume was 12 μL, and all reactions were conducted in triplicate. In 384-well half-volume plates (Perkin Elmer ProxiPlate 384 F Plus, 6008260), 4 μL of test compound in assay buffer was mixed with 4 μL of 60 nM full-length proMMP9(1-707) (SEQ ID NO:1) from COS-1 cells. The plate was sealed and incubated for 30 min at 37° C. Final concentrations were 20 nM full-length proMMP9(1-707) (SEQ ID NO:1) from COS-1 cells and 10 nM MMP3(100-265) (SEQ ID NO:8), and concentrations of test compounds were varied to fully bracket the IC₅₀. Immediately following the 30 min incubation, 4 μL of 40 μg/ml DQ gelatin substrate was added (freshly diluted in assay buffer), and incubated for 10 min at room temperature. The reaction was stopped by the addition of 4 μL of 50 mM EDTA, and the resulting activity associated with catalytic MMP9 was determined at 485 nm excitation, 535 nm emission using an Envision fluorescent reader (Perkin Elmer). Reactivity of residual MMP3 towards DQ gelatin was minimal under these conditions. Percent inhibition of test compounds were determined from suitable positive (DMSO only in assay buffer) and negative (EDTA added prior to reaction initiation) controls. Plots of % inhibition vs. test compound concentration were fit to a four-parameter logistics equation (GraphPad Prism® software) for determination of IC₅₀.

Enzyme assay data for representative compounds of Formula I is shown in Table 2.

TABLE 2

| | proMMP9/MMP3 P126 | ProMMP13/Plasmin P126 |
|---|---|---|
| | Activation Assay, | Activation Assay, |
| Example | IC₅₀ (μM) | IC₅₀ (μM) |
| 1 | 2.8 | ND |
| 2 | 1.3 | ND |
| 3 | 1.4 | ND |
| 4 | 4.7 | ND |
| 5 | 0.92 | ND |
| 6 | 0.66 | ND |
| 7 | 1.7 | ND |
| 8 | 1.2 | ND |
| 9 | 10 | ND |
| 10 | 0.13 | ND |
| 11 | 0.25 | ND |
| 12 | 1.1 | ND |
| 13 | 0.42 | ND |
| 14 | 1.1 | ND |
| 15 | 3.5 | ND |
| 16 | 0.89 | ND |
| 17 | 5.8 | ND |
| 18 | 0.63 | ND |
| 19 | 0.35 | ND |
| 20 | 0.24 | ND |
| 21 | 0.50 | ND |
| 22 | 0.092 | 1.9 |
| 23 | 0.84 | ND |
| 24 | 0.38 | ND |
| 25 | 0.49 | ND |
| 26 | 0.71 | ND |
| 27 | 0.058 | ND |
| 28 | 0.18 | ND |
| 29 | 0.55 | ND |
| 30 | 0.19 | 4.5 |
| 31 | 0.82 | ND |
| 32 | 0.35 | ND |
| 33 | 0.10 | 1.1 |
| 34 | 0.11 | ND |
| 35 | 0.053 | ND |
| 36 | 0.30 | ND |
| 37 | 0.31 | ND |
| 38 | 1.5 | ND |
| 39 | 0.094 | ND |
| 40 | 0.17 | ND |
| 41 | 0.72 | ND |
| 42 | 0.35 | ND |
| 43 | 2.5 | ND |
| 44 | 3.8 | ND |
| 45 | 3.9 | ND |
| 46 | 0.15 | ND |
| 47 | 0.60 | ND |
| 48 | 0.36 | ND |
| 49 | 1.3 | ND |
| 50 | 1.1 | ND |
| 51 | 0.20 | >20 |
| 52 | 0.16 | ND |
| 53 | 4.2 | ND |
| 54 | 0.24 | ND |
| 55 | 0.53 | ND |
| 56 | 1.1 | ND |
| 57 | 0.070 | ~6 |
| 58 | 0.38 | ND |
| 59 | 0.45 | ND |
| 60 | 0.34 | ND |
| 61 | 0.65 | ND |
| 62 | 1.1 | ND |
| 63 | 0.69 | ND |
| 64 | 0.15 | ND |
| 65 | 0.15 | ND |
| 66 | 2.3 | ND |
| 67 | 0.10 | ND |
| 68 | 7.5 | ND |
| 69 | 0.30 | ND |
| 70 | 0.31 | ND |
| 71 | 1.5 | ND |
| 72 | 0.46 | ND |
| 73 | 5.8 | ND |
| 74 | 4.7 | ND |
| 75 | ~18 | ND |
| 76 | 0.10 | ND |
| 77 | 1.1 | ND |
| 78 | 4.0 | ND |
| 79 | 0.39 | ND |
| 80 | 0.74 | ND |
| 81 | 0.75 | ND |
| 82 | 0.33 | ND |
| 83 | ~0.1 | ND |
| 84 | 0.75 | ND |
| 85 | 0.15 | ND |
| 86 | 0.65 | ND |
| 87 | 0.65 | ND |
| 88 | 3.9 | ND |
| 89 | 0.33 | 2.8 |
| 90 | 0.20 | 2.5 |
| 91 | 1.3 | ND |
| 92 | 0.13 | ~6 |
| 93 | 0.18 | ND |
| 94 | 0.51 | ND |
| 95 | 0.17 | 1.8 |
| 96 | 0.73 | ND |
| 97 | 0.18 | ND |
| 98 | >20 | ND |
| 99 | 0.87 | ND |
| 100 | 1.7 | ND |
| 101 | 0.76 | ND |
| 102 | 0.39 | ND |
| 103 | 0.38 | ND |
| 104 | ND | ND |
| 105 | 0.85 | ND |
| 106 | 0.19 | ND |
| 107 | 0.58 | ND |
| 108 | 0.65 | ND |
| 109 | ~2 | ND |
| 110 | 0.26 | ND |
| 111 | 0.37 | ND |
| 112 | 0.16 | ND |
| 113 | 0.25 | ND |
| 114 | ~2.2 | ND |
| 115 | 1.6 | ND |
| 116 | 0.98 | ND |
| 117 | 6.0 | ND |
| 118 | 0.51 | ND |
| 119 | 0.50 | ND |
| 120 | 0.48 | ND |
| 121 | 0.31 | ND |
| 122 | 0.65 | ND |
| 123 | 0.81 | ND |
| 124 | 0.47 | ND |
| 125 | 0.26 | ND |
| 126 | 0.46 | ND |
| 127 | 0.27 | ND |
| 128 | 0.68 | ND |
| 129 | 0.42 | ND |
| 130 | 0.83 | ND |
| 131 | 0.59 | ND |
| 132 | 1.4 | ND |
| 133 | 0.28 | ND |
| 134 | 0.26 | ND |
| 135 | 0.27 | ND |
| 136 | 1.1 | ND |
| 137 | 1.1 | ND |
| 138 | 0.56 | ND |
| 139 | 0.33 | ND |
| 140 | 0.41 | ND |
| 141 | 1.3 | ND |
| 142 | 1.8 | ND |
| 143 | 0.16 | ND |
| 144 | 0.26 | ND |
| 145 | 0.39 | ND |
| 146 | 0.80 | ND |
| 147 | 0.22 | ND |
| 148 | 0.22 | ND |
| 149 | 0.11 | ND |
| 150 | 0.34 | ND |
| 151 | >20 | ND |
| 152 | 0.55 | ND |
| 153 | 0.65 | ND |
| 154 | 0.63 | ND |
| 155 | 0.15 | ND |
| 156 | 0.10 | ND |
| 157 | 0.042 | ND |
| 158 | 0.064 | ND |
| 159 | 0.066 | ND |
| 160 | 0.16 | ND |
| 161 | 0.34 | ND |
| 162 | 0.64 | ND |
| 163 | 0.11 | ND |
| 164 | 0.18 | ND |

### Cell-Based Assays

Activation of proMMP9 in Rat Synoviocyte Cultures

A primary synoviocytes line was derived from the periarticular tissue of arthritic rats. Arthritis was induced in female Lewis rats following an i.p. administration of streptococcal cell wall peptidoglycan polysaccharides (J Exp Med 1977; 146:1585-1602). Rats with established arthritis were sacrificed, and hind-limbs were severed, immersed briefly in 70% ethanol, and placed in a sterile hood. The skin was removed and the inflamed tissue surrounding the tibia-tarsal joint was harvested using a scalpel. Tissue from six rats was pooled, minced to approximately 8 mm³ pieces, and cultured in Dulbecco's Modified Eagle's Medium (DMEM) containing 15% fetal calf serum (FCS). In the following weeks, cells migrated out of the tissue piece, proliferated, and formed a monolayer of adherent cells. The synoviocytes were lifted from culture plates with 0.05% trypsin and passaged weekly at 1:4 ratios in DMEM containing 10% FCS. Synoviocytes were used at passage 9 to investigate the ability of Compound-α to inhibit the maturation of MMP9 to active form.

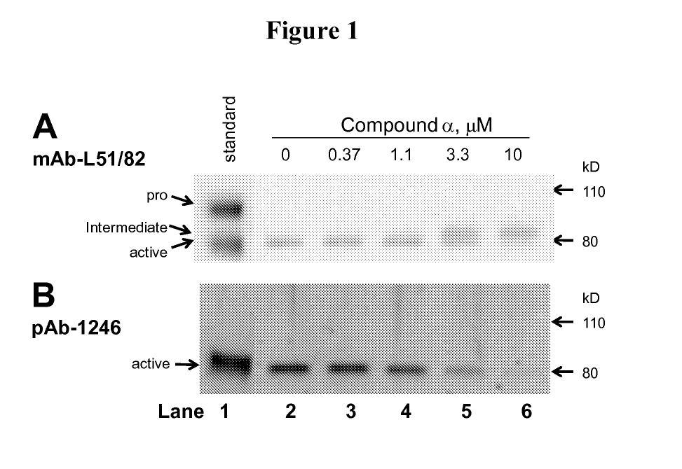

Rat synoviocytes spontaneously expressed and activated MMP9 when cultured in collagen gels and stimulated with tumor necrosis factor-alpha (TNFα) (FIG. 1 and Table 3). Eight volumes of an ice-cold solution of 3.8 mg/mL rat tail collagen (Sigma Cat #C3867-1VL) were mixed with 1 volume of 1 M sodium bicarbonate and 1 volume of 10× Roswell Park Memorial Institute medium. The pH of the mixture was adjusted to pH 7 with 1 N sodium hydroxide and equal volumes of the pH-adjusted collagen solution were mixed with DMEM containing 0.8 million synoviocytes per mL. One half mL volumes were dispensed into Costar 24-well culture dishes and placed for one hr at 37° C. and 5% CO₂, during which time the collagen solution formed a gel. Individual gels were dislodged into wells of 12-well Costar plates containing 1 mL/well of DMEM adjusted to contain 0.05% BSA and 100 ng/mL mouse TNFα (R&D Systems Cat #410-MT-010). The plates were agitated 10 seconds to ensure that the collagen gels did not adhere to the well bottoms. After overnight culture at 37° C. and 5% CO₂, wells were adjusted to contain an additional 0.5 mL of DMEM containing 0.05% BSA and Compound-α at 4× the final desired concentration (final culture volumes were 2 mL). The plates were cultured an additional 48 hrs, at which time 1 mL of conditioned media were harvested into fresh eppendorf tubes containing 40 μL/mL of a 50% slurry of gelatin-conjugated sepharose (GE Healthcare Cat #17-0956-01). Samples were rotated for 2 hrs at 4° C. before centrifugation 1 min×200 g. Supernatants were discarded. The gelatin-sepharose pellets were washed once with 1 mL of ice cold DMEM, resuspended in 50 μL of 2× reducing Leamli buffer and heated 5 min at 95° C. Fifteen μL of eluted proteins were resolved on 4-12% NuPAGE gels and transferred to 0.45 μm pore-sized nitrocellose blots. Next, blots were incubated in blocking buffer (5% milk in Tris-buffered saline containing 0.1% Tween-20) for 1 hr at RT and probed overnight (4° C.) with blocking buffer containing 1 μg/mL primary antibodies. Blots were next probed 1 hr at RT with 1/10,000 dilutions of goat anti-mouse IgG-HRP or goat anti-rabbit IgG-HRP (Santa Cruz) in blocking buffer and developed using SuperSignal® West Fempto Maximum Sensitivity Substrate. Chemiluminesence signal was analyzed using a ChemiDoc imaging system (BioRad Laboratories) and Quantity One® image software. Electrophoretic mobility was estimated based on the mobility of standards (Novex Sharp Pre-Stained Protein Standards P/N 57318).

Mouse mAb-L51/82 (UC Davis/NIH NeuroMab Facility, Antibody Incorporated) was used to detect pro and processed forms of MMP9. Synoviocyte-conditioned media contained an approximately 80 kD form of MMP9 (FIG. 1A, lane 2). In the presence of 0.37-10 μM Compound-α (FIG. 1A, lanes 3-6), the 80 kD active MMP9 form was reduced in a dose dependent fashion, and a form of approximately 86 kD appeared. The 86 kD form was predominant in the presence of 10 μM Compound-α (FIG. 1A, lane 6). Lane 1 was loaded with a standard containing 3 ng of full-length rat proMMP9(1-708) (SEQ ID NO:11) and 3 ng of full-length rat proMMP9(1-708) (SEQ ID NO:11) converted to catalytic rat MMP9 by catalytic MMP3. The electrophoretic mobility of the 80 kD form present in synoviocyte conditioned medium was the same as the active MMP9 standard. The 86 kD form produced by synoviocytes in the presence of Compound-α demonstrated greater mobility than the full-length rat proMMP9(1-708) (SEQ ID NO:11) standard which ran with a mobility of approximately 100 kD. The 86 kD form demonstrated a mobility similar to an incompletely processed intermediate form described previously that retains the cysteine switch and lacks catalytic activity (J Biol Chem; 1992; 267:3581-4).

ProMMP9 is activated when cleaved between R106 and F107 (J Biol Chem; 1992; 267:3581-4). A rabbit polyclonal antibody (pAb-1246) was generated to the active MMP9 N-terminal neoepitope using an approach similar to that reported previously (Eur J Biochem; 1998; 258:37-43). Rabbits were immunized and boosted with a peptide, human MMP9(107-113) (SEQ ID NO:9) conjugated to keyhole limpet hemocyanin, and antibodies were affinity purified from serum using FQTFEGD-conjugated agarose affinity resin and 100 mM glycine (pH 2.5) elution. To resolve N-terminal neoepitope antibodies from antibodies directed to other epitopes within the sequence, eluted antibody was dialyzed in PBS and cross-absorbed by mixing with a peptide, human proMMP9(99-113) (SEQ ID NO:10), that was conjugated to agarose. The unbound fraction containing N-terminal neoepitope antibodies was recovered and was designated pAb-1246.

FIG. 1B, lane 1 demonstrated that pAb-1246 bound the 80 kD active MMP9 standard, but did not recognize the 100 kD proMMP9 standard. pAb-1246 detected 80 kD active MMP9 in synoviocyte conditioned medium, and Compound-α caused a dose-dependent reduction in active MMP9 (FIG. 1B, lanes 2-6). Band chemiluminescence intensities were measured directly and reported in Table 3. The production of active MMP9 was inhibited by Compound-α with an IC₅₀ of approximately 1.1 μM. pAb-1246 did not recognize the 86 kD form, providing further evidence that this likely represented an intermediate form whose further maturation was blocked by Compound-α.

TABLE 3
Compound-α blocked production of active MMP9 by rat
synoviocytes^{a}

| | Signal of 80 kD band | |
|---|---|---|
| Compound-α, μM | (INT * mm²)^{b} | % Inhibition^{c} |
| 0 | 84384 | 0 |
| 0.37 μM | 74381 | 12 |
| 1.1 μM | 45381 | 46 |
| 3.3 μM | 11554 | 86 |
| 10 μM | 2578 | 97 |

^{a}Rat synoviocytes embedded in collagen gels were stimulated 72 hrs with TNFα. Cultures were supplemented with the indicated concentrations of Compound-α for the final 48 hrs and conditioned media were assessed for the 80 kD active form of MMP9 by Western blotting with pAb-1246 developed against the N-terminal activation neoepitope.
^{b}Chemiluminesence captured during a 30 s exposure was analyzed using a ChemiDoc imaging system (BioRad Laboratories) and Quantity One ® image software. Signals were measured within uniform sized boxes drawn to circumscribe the 80 kD bands and were the product of the average intensity (INT) and the box area (mm²). Values given have been corrected for background signal.
^{c}Percent signal reduction relative to the signal generated by synoviocytes cultured in the absence of Compound-α.

Activation of proMMP 9 by Human Fetal Lung Fibroblast Cultures

Compound-α was assessed additionally for ability to block the maturation of proMMP9 to active MMP9 in cultures of human fetal lung fibroblasts (HFL-1, American Type Culture Collection # CCL-153). Unlike rat synoviocytes, HFL-1 cells were unable to process proMMP9 to the active form without addition of neutrophil elastase. Elastase did not directly cause processing of recombinant proMMP9 (data not shown). Rather, the function of elastase in this assay may be to inactivate tissue inhibitors of matrix metalloproteinases (TIMPs) that repress endogenous pathways of MMP9 activation (Am J Respir Crit. Care Med; 1999; 159:1138-46).

HLF-1 were maintained in monolayer culture in DMEM with 10% FCS and were used between passage numbers 5-15. HLF-1 were embedded in collagen gels as described for rat SCW synoviocytes (vida supra). Half mL gels containing 0.4 million cells were dislodged into wells of 12 well Costar plates containing 1 mL/well of DMEM adjusted to contain 0.05% BSA and 100 ng/mL human TNFα (R&D Systems Cat #210-TA/CF). After overnight culture (37° C. and 5% CO₂) wells were adjusted to contain an additional 0.5 mL of DMEM containing 0.05% BSA and with or without 13.2 μM Compound-α (final concentration was 3.3 μM Compound-α). Next, cultures were adjusted to contain 30 nM human elastase (Innovative Research). The plates were cultured an additional 72 hrs, at which time MMP9 secreted into the conditioned media was bound to gelatin-sepharose and evaluated by Western blot analysis as described for the rat synoviocyte cultures (vida supra). mAb-51/82 detected three forms of MMP9 in HFL-1 cultures.

These included a form of approximately 100 kD with mobility similar to recombinant rat proMMP9, an approximately 80 kD form with mobility similar to rat active MMP9, and an approximately 86 kD intermediate form. The band intensities are provided in Table 4. In the absence of Compound-α, most of the MMP9 was present as the 80 kD form. In the presence of Compound-α, the 80 kD form was a minor fraction of the total signal while nearly half of the signal were contributed each by the 100 kD and 86 kD forms. The total signal of the three bands was similar with or without Compound-α. These data indicate that the 100 kD and 86 kD forms of MMP9 were effectively stabilized by Compound-α and the formation of the 80 kD form was suppressed.

TABLE 4
Compound-α blocked processing of MMP9 by HFL-1 cells ^{a}

| Compound-α, | Signal (INT * mm²) ^{b} | | | | Percent of total signal | | |
|---|---|---|---|---|---|---|---|
| 3.3 μM | 100 kD | 86 kD | 80 kD | Total | 100 kD | 86 kD | 80 kD |
| − | 17190 | 24858 | 61925 | 103973 | 16 | 24 | 60 |
| + | 42107 | 43147 | 6092 | 91346 | 46 | 47 | 7 |

^{a} Human fetal lung fibroblasts (HFL-1) embedded in collagen gels were stimulated 90 hrs with TNFα. Cultures were supplemented with or without 3.3 μM Compound-α and with 30 nM elastase for the final 72 hrs and conditioned media were assessed for the MMP9 forms by Western blotting with mAb-L51/82.
^{b} Chemiluminesence captured during a 150 s exposure was analyzed using a ChemiDoc imaging system (BioRad Laboratories) and Quantity One ® image software. Signals were measured within uniform sized boxes drawn to circumscribe the bands and were the product of the average intensity (INT) and the box area (mm²). Values given have been corrected for background signal.

A second experiment was performed to determine if the 80 kD form was mature active MMP9 and to determine the potency of Compound-α as an inhibitor of MMP9 maturation in this assay. HFL-1 cells embedded in collagen gels were cultured as described above in the presence of TNFα overnight and the cultures were then adjusted to contain 30 nM elastase and graded concentrations of Compound-α for an additional 72 hrs at which time MMP9 secreted into the conditioned media was bound to gelatin-sepharose and evaluated by Western blot analysis for active MMP9 using pAb-1246 raised against the N-terminal neoepitope of active MMP9 (Table 5). In the absence of Compound-α, pAb-1246 readily detected MMP9 with an electrophoretic mobility of approximately 80 kD. Compound-α effectively inhibited the ability of HFL-1 cultures to process proMMP9 to active MMP9. Inhibition occurred over a dose range with an IC₅₀ of approximately 0.3 μM Compound-α.

TABLE 5
Compound-α blocked production of active MMP9 by human fetal
lung fibroblasts^{a}

| | Signal of 80 kD band | |
|---|---|---|
| Compound-α, μM | (INT * mm²)^{b} | % Inhibition^{c} |
| 0 | 168781 | 0 |
| 0.12 μM | 168211 | 0 |
| 0.37 μM | 45996 | 73 |
| 1.1 μM | 1747 | 99 |
| 3.3 μM | 152 | 100 |
| 10 μM | 0 | 100 |

^{a}Human fetal lung fibroblasts (HFL-1) embedded in collagen gels were stimulated 90 hrs with TNFα. Cultures were supplemented with the indicated concentrations of Compound-α and 30 nM elastase for the final 72 hrs and conditioned media were assessed for active MMP9 by Western blotting with pAb-1246 developed against the N-terminal activation neoepitope.
^{b}Chemiluminesence captured during a 10 s exposure was analyzed using a ChemiDoc imaging system (BioRad Laboratories) and Quantity One ® image software. Signals were measured within uniform sized boxes drawn to circumscribe the 80 kD bands and were the product of the average intensity (INT) and the box area (mm²). Values given have been corrected for background signal.
^{c}Percent signal reduction relative to the signal generated by HFL-1 cells cultured in the absence of Compound-α.

### In Vivo Studies

Expression and Activation of ProMMP9 In Vivo is Associated with Rat SCW-Arthritis

MMP9 protein expression was reportedly increased in the synovial fluid of patients with rheumatoid arthritis (Clinical Immunology and Immunopathology; 1996; 78:161-71). A preliminary study was performed to assess MMP9 expression and activation in a rat model of arthritis.

A polyarthritis can be induced in female Lewis rats following i.p. administration of streptococcal cell wall (SCW) proteoglycan-polysaccharides (PG-PS) (J Exp Med 1977; 146:1585-1602). The model has an acute phase (days 3-7) that is complement and neutrophil-dependent and that resolves. A chronic erosive phase begins at about day ten and is dependent on the development of specific T cell immunity to the PG-GS, which resists digestion and remains present in synovial macrophages for months. Like rheumatoid arthritis, SCW-induced arthritis is reduced by TNF inhibitors, and the dependence of SCW-induced arthritis on macrophages (Rheumatology; 2001; 40:978-987) and the strong association of rheumatoid arthritis severity with synovial-tissue macrophage counts (Ann Rheum Dis; 2005; 64:834-838) makes SCW-arthritis an attractive model for testing potential therapeutic agents. SCW PG-PS 10S (Beckton Dickinson Cat#210866) suspended in saline was vortexed for 30 seconds and sonicated for 3 min with a probe type sonicator prior to injection. Female Lewis (LEW/N) rats, 5-6 weeks of age (80-100 g) were injected (i.p.) with SCW PG-PS (15 μg of rhamnose/gram BW) in the lower left quadrant of the abdomen using a 1 mL syringe fitted with a 23-gauge needle. Control (disease-free) rats were treated in a similar manner with sterile saline. Control rats were sacrificed on day 5 and groups of SCW-injected rats were sacrificed on day 5 when acute inflammation was maximal or on day 18 when chronic inflammation was established.

Hind-limbs were skinned, severed just above the tibia-tarsus joint and below the metatarsals, and the tibia-tarsus joints (ankles) were weighed, snap frozen and pulverized on dry ice using a hammer and anvil. The pulverized tissue was suspended in 3 volumes (w:v) of ice-cold homogenization buffer containing 50 mM Tris pH 7.5, 150 mM NaCl, 5 mM EDTA, 1% Triton X100, 0.05% Brij 30, 10% dimethylsulfoxide and Complete EDTA-free Protease Inhibitor Cocktail (Roche Diagnostics). The suspended tissue was homogenized sequentially with a Kinematica AG Polytron and a Dounce homogenizer. Homogenates were centrifuged at 16,000×g for 10 min at 4° C. and the soluble fractions were saved. Dimethylsulfoxide was removed from a portion of each soluble fraction using PD MiniTrap™ G-25 desalting columns (GE Healthcare). Homogenates (0.25 mL), free of DMSO, were diluted with an equal volume of binding buffer (i.e., homogenization buffer without dimethylsufoxide) and adjusted to contain 50 μL of a 50% slurry of gelatin-conjugated sepharose. Following 2 hours of rotation at 4° C. the beads were washed twice in binding buffer and eluted in 100 μL 2×-reducing Laemmli buffer with heating to 95° C. for 5 minutes. Eluates (20 μL) were resolved on 4-12% NuPAGE gels, transferred to 0.45 μm pore-sized nitrocellose and immunoblotted for detection of proMMP9, active MMP9, and other processed forms using mAb-L51/82 and pAb-1246 as described above for detection of MMP9 forms in synoviocyte and HFL-1 cell conditioned media.

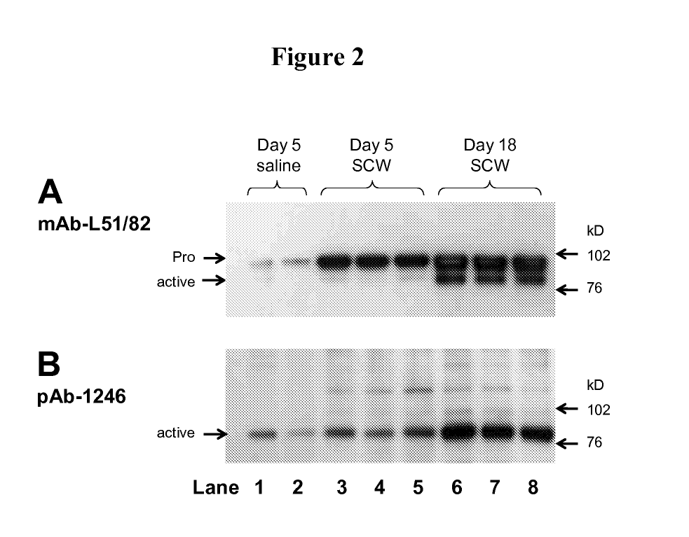

In healthy ankles of rats administered saline, mAb-L51/82 detected small amounts of an approximately 100 kD (proMMP9) and an approximately 80 kD form of MMP9 (FIG. 2A, lanes 1 and 2). proMMP9 was increased markedly in ankle homogenates 5 and 18 days after SCW-administration (FIG. 2A, lanes 3-5 and 6-8, respectively). The 80 kD MMP9 was increased mildly 5 days after SCW-administration (FIG. 2A, lanes 3-5) and was increased markedly 18 days after SCW-administration (FIG. 2A, lanes 6-8). In healthy ankles of rats administered saline, mAb-1246 detected small amounts active MMP9 at 80 kD (FIG. 2B, lanes 1 and 2). The 80 kD active MMP9 was increased mildly 5 days after SCW-administration (FIG. 2A, lanes 3-5) and was increased markedly 18 days after SCW-administration (FIG. 2A, lanes 6-8).

Efficacy of Compound-α in Rats with SCW Arthritis

Having shown that active MMP9 is increased in rats with SCW-induced arthritis, we next sought to determine the ability of Compound-α to reduce disease severity and to reduce active MMP9.

Compound-α Reduced Ankle Swelling of Rats with SCW-Induced Arthritis

To induce arthritis, Female Lewis (LEW/N) rats, 5-6 weeks of age (80-100 g) were injected (i.p.) with SCW PG-PS as described above. Eighteen days later, arthritis was well established. Calipers were used to measure the width (anterior to posterior surface) of the left and right hind ankles of each rat. Each ankle was measured 3 times and averaged, and treatment groups were randomized based on ankle thickness (Table 6). Commencing on day 18, randomized groups of arthritic rats (n=5 rats/group) received vehicle or 5, 20, or 50 mg/kg Compound-α BID by oral gavage. Vehicle consisted of an aqueous mixture containing 2% (v:v) N-methylpyrrolidone, 5% (v:v) glycerine, and 20% (w:v) captisol. Treatment continued daily through the morning of day 26.

By day 18 mean ankle thickness was increased an average of >4.4 mm compared to disease free rats. Rats treated with vehicle alone continued to gradually develop a more severe arthritis based on ankle thickness measurements over the eight-day treatment period (Table 6). Treatment with Compound-α induced a dose-dependent decrease in ankle thickness measurements. By day 26, the disease associated increase in ankle thickness had been reduced 27, 37, and 46 percent by 5, 20, and 50 mg/kg Compound-α, respectively.

TABLE 6
Ankle thickness of rats with SCW-arthritis dosed with
vehicle vs. Compound-α

| | | Ankle | | | |
|---|---|---|---|---|---|
| | | thickness | | | |
| | | (mm)^{a} | | Day 26 Δ mm | |
| Treatment | | Day 18 | Day 26 | (vs. group 1) | % Inh |
| Group 1: | mean (n = 4) | 7.20 | 7.26 | 0 | 100 |
| Sterile Saline | SD | 0.043 | 0.012 | | |
| Vehicle | p-value^{b} | 0.0000 | 0.0001 | | |
| Day 18-26 | | | | | |
| Group 2: | mean (n = 5) | 11.86 | 12.31 | 5.04 | 0 |
| PG-PS | SD | 0.77 | 1.26 | | |
| (15 μg/ | | | | | |
| gramBW) | | | | | |
| Vehicle | p-value* | na | na | | |
| Day 18-26 | | | | | |
| Group 3: | mean (n = 5) | 11.79 | 10.93 | 3.67 | 27 |
| PG-PS | SD | 0.56 | 0.21 | | |
| (15 μg/ | | | | | |
| gramBW) | | | | | |
| Compound- | p value* | 0.88 | 0.043 | | |
| α (5 mg/kg) | | | | | |
| Day 18-26 | | | | | |
| Group 4: | mean (n = 5) | 11.76 | 10.42 | 3.15 | 37 |
| PG-PS | SD | 0.73 | 0.93 | | |
| (15 μg/ | | | | | |
| gramBW) | | | | | |
| Compound- | p-value* | 0.85 | 0.028 | | |
| α | | | | | |
| (20 mg/kg) | | | | | |
| Day 18-26 | | | | | |
| Group 5: | mean (n = 5) | 11.68 | 9.99 | 2.73 | 46 |
| PG-PS | SD | 0.62 | 0.73 | | |
| (15 μg/ | | | | | |
| gramBW) | | | | | |
| Compound- | p-value* | 0.71 | 0.0075 | | |
| α | | | | | |
| (50 mg/kg) | | | | | |
| Day 18-26 | | | | | |

^{a}Calipers were used to measure the width (anterior to posterior surface) of the left and right hind ankles of each rat. Each ankle was measured 3 times and averaged.
^{b}Student's t-test vs. group 2

Hind paw inflammation clinical scores were assigned based on swelling and erythema. By day 18, nearly all rats induced with SCW PG-PS had a clinical score of 8 based on an 8-point scale (Table 7). Treatment with Compound-α induced a dose dependent decrease in clinical score measurements with significant effects emerging at the 20 mg/kg dose (Table 7).

TABLE 7
Clinical Scores of rats with SCW-arthritis dosed with
vehicle vs. Compound-α

| | | Clinical | | |
|---|---|---|---|---|
| | | Scores (0-8)^{a} | | Δ Day 18 vs. |
| Treatment | | Day 18 | Day 26 | day 26 |
| Group 1: | mean (n = 4) | 0 | 0 | 0 |
| Sterile Saline | SD | 0 | 0 | |
| Vehicle | p-value^{b} | <0.0001 | | |
| Day 18-26 | | | | |
| Group 2: | mean (n = 5) | 7.80 | 7.80 | 0 |
| PG-PS (15 μg/gramBW) | SD | 0.45 | 0.45 | |
| Vehicle | p-value | | na | |
| Day 18-26 | | | | |
| Group 3: | mean (n = 5) | 8.00 | 6.80 | −1.20 |
| PG-PS (15 ug/gramBW) | SD | 0.00 | 1.09 | |
| Compound-α (5 mg/kg) | p-value | 0.095 | | |
| Day 18-26 | | | | |
| Group 4: | mean (n = 5) | 8.00 | 5.20 | −2.80 |
| PG-PS (15 μg/gramBW) | SD | 0.00 | 1.79 | |
| Compound-α (20 mg/kg) | p-value | 0.014 | | |
| Day 18-26 | | | | |
| Group 5: | mean (n = 5) | 7.80 | 4.40 | −3.40 |
| PG-PS (15 μg/gramBW) | SD | 0.45 | 1.67 | |
| Compound-α (50 mg/kg) | p-value | 0.0023 | | |
| Day 18-26 | | | | |

^{a}Hind paw inflammation clinical scores were assigned based on swelling and erythema as follows: 1 = ankle involvement only; 2 = involvement of ankle and proximal ½ of tarsal joint; 3 = involvement of the ankle and entire tarsal joint down to the metatarsal joints; and 4 = involvement of the entire paw including the digits. Scores of both hind-paws were summed for a maximal score of 8.
^{b}Student's t-test vs. group 2

Compound-α Reduced Active MMP9 in Ankles of Rats with SCW-Induced Arthritis Demonstrated by Western Blot Analysis

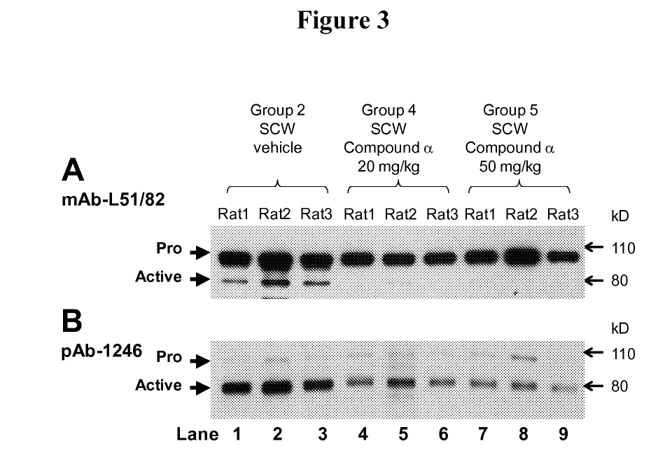

Rats in the study reported in Tables 4 and 5 were sacrificed on Day 26 four hours after the AM dose. Ankles harvested from the right-hind-limbs were processed by the method described above. Pro and active MMP9 were abundantly present in ankles of SCW-induced vehicle-treated rats (FIGS. 3A and 3B, lanes 1-3). Treatment of rats with Compound-α did not reduce the abundance of proMMP9 (FIG. 3A, lanes 4-9). However, treatment of rats with Compound-α resulted in a notable reduction in the active 80 kD form of MMP9 detected with pAb-1246 (FIG. 3B, lanes 4-9 vs. 1-3) and with mAb-L51/82 (FIG. 3A, lanes 4-9 vs. 1-3).

Compound-α Reduced MMP9 Mediated Gelatinase Activity in the Livers of Rats with SCW Arthritis

In situ zymography provides an alternative approach to assess active MMP9 in tissues (Frederiks). Tissue sections are overlain with fluorescene-conjugated gelatin wherein the conjugation is sufficiently dense to cause the fluorescene to be dye-quenched (DQ). Proteolytic degradation of the DQ-gelatin releases the fluorescene from the quenching effect giving rise to bright green fluorescence at the site of degradation. Because in situ zymography requires the use of frozen sections, calcified tissues are problematic. However, an additional feature of the SCW arthritis model is the development of hepatic granulomatous disease (J Immunol; 1986; 137:2199-2209), and MMP9 reportedly plays a role in macrophage recruitment in the granulomas response to mycobacteria (Infect Immun; 2006; 74:6135-6144). Consequently, granulomatous livers from SCW-treated rats were assessed for active MMP9 by in situ zymography.

As described above, Female Lewis (LEW/N) rats, 5-6 weeks of age (80-100 g) were injected (i.p.) with saline or SCW PG-PS. On day 28, when the granulomatous response was well established, animals were sacrificed and livers were frozen in OCT cryo-sectioning medium and 10 μm sections were cut on a Cryome HM 500 M cryotome and mounted on glass microscope slides. Sections were air dried briefly. MMP9 was confirmed as the source of the gelatinase activity in the liver by treating liver sections with monoclonal antibodies directed against the active site of the two major gelatinases MMP9 and MMP2. Liver sections overlain with 50 μL of 100 μg/mL neutralizing mouse monoclonal antibodies directed against MMP9 (Calbiochem, clone 6-6B), or MMP2 (Millipore, clone CA-4001), or with PBS for 1 hr at room temperature. Tissues were rinsed once with PBS, blotted, and briefly air dried and then overlain with DQ-gelatin (Invitrogen) dissolved to 1 mg/mL in deionized water and then diluted 1:10 in 1% wt/vol low gelling point agarose type VII (Sigma) in PBS. The sections were covered with coverslips, incubated in the dark at room temperature for 20 min, and imaged on an Olympus IX80 inverted microscope fitted with fluorescence optics, using SlideBook™ imaging software (Intelligent Imaging Innovations, Inc., Philadelphia, Pa.; version 5.0). Fluorescence intensity was determined (Table 8). When compared to a saline-treated rat, gelatinase activity was abundantly expressed in granulomatous liver sections obtained from a rat with SCW arthritis. The activity in the granulomatous liver sections was almost completely inhibited by treatment with anti-MMP9 monoclonal antibody but not by treatment with anti-MMP2 monoclonal antibody.

TABLE 8
Indentification of MMP9 as the gelatinase responsible for signals
detected by in situ zymography in SCW-granulomatous livers

| | |
|---|---|
| | Intensity |
| Disease | (RLU × 10⁶) |

| induction | Section treatment | Mean | SD |
|---|---|---|---|
| Saline-healthy | PBS | 11.4 | 2.91 |
| SCW- | PBS | 109 | 19.3 |
| granulomatous | Anti-MMP9 | 1.02 | 0.17 |
| | Anti-MMP2 | 128 | 36.2 |

Key:
RLU = relative light units;
SCW = Streptococcal cell wall peptidoglycan-polysaccharide equivalent to 15 μg rhamnose/gram BW.

Next, liver in situ zymography was used to assess the relative presence of active MMP9 in rats dosed with vehicle vs. Compound-α. Female Lewis (LEW/N) rats, 5-6 weeks of age (80-100 g) were injected (i.p.) with saline or SCW PG-PS. Commencing on day 25, randomized groups of rats (n=3 rats/group) received vehicle or 20 or 50 mg/kg Compound-α BID by oral gavage. Vehicle consisted of an aqueous mixture containing 2% (v:v) N-methylpyrrolidone, 5% (v:v) glycerine, and 20% (w:v) captisol. Treatment continued daily through the morning of day 28. Four hrs after the AM dose on day 28, rats were sacrificed and livers assessed for active MMP9 by in situ zymography (Table 9). Gelatinase activity was increased markedly in SCW-induced rats, but activity was reduced by approximately 80% in animals treated with 50 mg/kg Compound-α.

TABLE 9
In situ zymography determination of gelatinase activity in livers
of SCW-induced rats dosed with vehicle vs. Compound-α

| | | | | | | t-test vs. |
|---|---|---|---|---|---|---|
| | Intensity (RLU × 10⁶) | | | | | SCW- |
| Treatment | Rat 1 | Rat 2 | Rat 3 | Mean | SD | vehicle |
| Saline | 3.3 | 1.1 | 1.6 | 2.0 | 1.15 | 0.001 |
| Vehicle | | | | | | |
| Day 25-28 | | | | | | |
| SCW | 65.1 | 43.4 | 58.9 | 55.8 | 11.17 | 1 |
| Vehicle | | | | | | |
| Day 25-28 | | | | | | |
| SCW | 43.0 | 69.0 | 53.7 | 55.2 | 13.06 | 0.96 |
| Compound-α | | | | | | |
| (20 mg/kg) | | | | | | |
| Day 25-28 | | | | | | |
| SCW | 3.2 | 25.6 | 4.5 | 11.1 | 12.57 | 0.010 |
| Compound-α | | | | | | |
| (50 mg/kg) | | | | | | |
| Day 25-28 | | | | | | |

Key: RLU = relative light units; SCW = Streptococcal cell wall peptidoglycan-polysaccharide equivalent to 15 μg rhamnose/gram BW.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

All publications disclosed in the above specification are hereby incorporated by reference in full.

We claim:

1. The compounds of Formula I 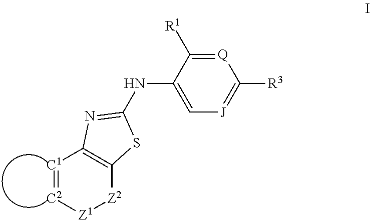
wherein:
R¹ is OC₍₁₋₅₎alkyl, O—C₍₃₋₅₎cycloalkyl, SCF₃, CF₃, CH₂CF₃, OCH₂—C₍₃₋₅₎cycloalkyl, OCF₃, OCH₂CF₃, SCH₂CF₃, NO₂, —CN, C₍₁₋₅₎alkyl, Cl, F, SC₍₁₋₄₎alkyl, SCH₂—C₍₃₋₅₎cycloalkyl, S—C₍₃₋₅₎cycloalkyl, or H;
Q is N or C—R²;
R² is F, Cl, Br, CF₃, CH₃, or H; or R² and R¹ may be taken together with the ring to which they are attached, to form a fused ring system selected from the group consisting of: quinolinyl, isoquinolinyl, quinazolinyl, quinoxalinyl, benzimidazolyl, napthalyl, benzofuranyl, 2,3-dihydro-benzofuranyl, benzothiophenyl, benzothiazolyl, benzotriazolyl, indolyl, indolinyl, and indazolyl, wherein said quinolinyl, isoquinolinyl, quinazolinyl, quinoxalinyl, benzimidazolyl, benzothiazolyl, napthalyl, benzofuranyl, 2,3-dihydro-benzofuranyl, benzothiophenyl, benzotriazolyl, indolyl, indolinyl, and indazolyl are optionally substituted with one methyl group or up to two fluorine atoms;
R³ is Cl, SO₂NH₂, SO₂CH₃, CO₂H, F, CONH₂, OCH₃, NO₂, —CN, CH₃, CF₃, or H; provided that R³ is not F if R⁴ is H;
J is N, or C—R⁴;
R⁴ is H, NH₂, NHC₍₁₋₃₎alkyl, N(C₍₁₋₃₎alkyl)₂, CONH₂, C₍₁₋₅₎alkyl, CO₂C₍₁₋₅₎alkyl, OC₍₁₋₅₎alkyl, CO₂H, SO₂NH₂, Cl, NHC(O)C₍₁₋₅₎alkyl, CF₃, CH₂CF₃, SO₂C₍₁₋₅₎alkyl, SO₂NHC₍₁₋₅₎alkyl, SO₂N(C₍₁₋₅₎alkyl)₂, SO₂NL¹L², CONL¹L², F, P(O)(OCH₂CH₃)₂, NO₂, —CN, P(O)(OH)₂, Br, —CH═CH₂, CONHC₍₁₋₃₎alkyl, CON(C₍₁₋₃₎alkyl)₂, CH₂CONH₂, —CONHCH₂CH₂-piperidinyl, —CONHCH₂Ph, or NHC(O)NH₂; provided that R⁴ is not OCH₃, if R¹ is C₍₁₋₄₎alkyl or H; or R⁴ is selected from the group consisting of: phenyl, pyridyl, pyrazolyl, imidazolyl, oxazolyl, isoxazolyl, thiazolyl, thiophenyl, pyrimidyl, pyrazyl, and furyl wherein said phenyl, pyridyl, pyrazolyl, imidazolyl, oxazolyl, isoxazolyl, thiazolyl, thiophenyl, pyrimidyl, pyrazyl, and furyl are optionally substituted with one substituent selected from the group consisting of C(O)C₍₁₋₄₎alkyl, C₍₁₋₄₎alkyl, F, Br, Cl, —CN, OC₍₁₋₄₎alkyl; or R⁴ and R³ may be taken together with the ring to which they are attached, to form the fused ring system 2,3-dihydroisoindolin-1-one;
L¹ and L² are taken together with their attached nitrogen to form a ring, selected from the group consisting of: 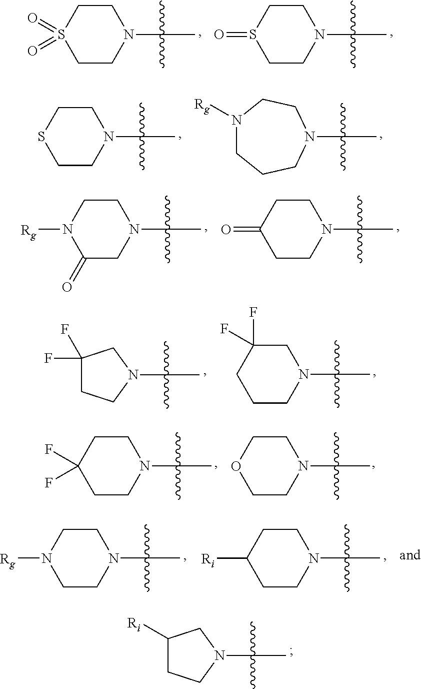
R_{g} is selected from the group consisting of H, CF₃, CH₂CF₃, CH₂CH₂CF₃, C₍₁₋₃₎alkyl, COC₍₁₋₃₎alkyl;
Rᵢ is H, CH₂OH, N(C₍₁₋₄₎alkyl)₂, C₍₁₋₆₎alkyl, CH₂OH, F, or OH;
wherein any said L¹ and L² ring may be optionally substituted with up to four methyl groups on two or more ring carbon atoms or optionally substituted with up to two CF₃ groups on any two ring carbon atoms;
Z¹—Z² is —CH═CH—, —(CH₂)₂—, or —(CH₂)₃—, provided that if Z¹—Z² is CH₂—CH₂ then R⁴ is not H;
C¹ and C² are carbon atoms which are further substituted to make a ring selected from the group consisting of: 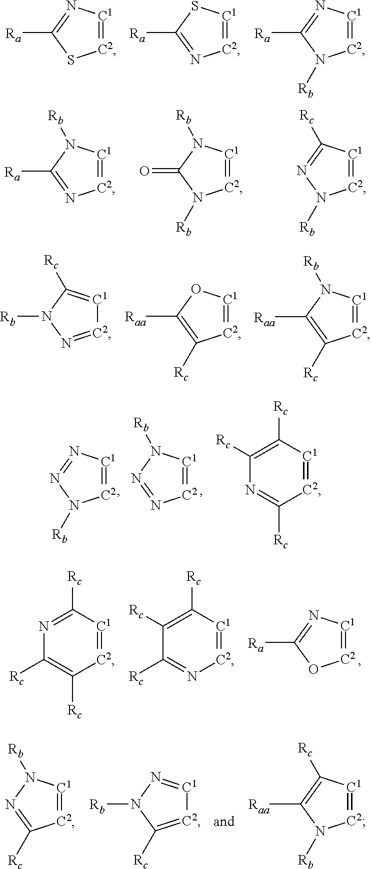
Rₐ is H, CF₃, CH₂CF₃, CH₂OH, Cl, Br, or C₍₁₋₆₎alkyl; or Rₐ may also be 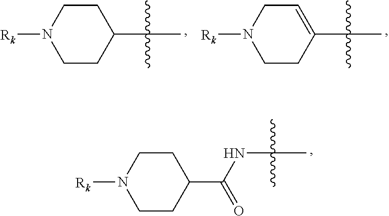
CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, pyridinyl, pyrimidinyl, pyrazinyl, NHPhOCH₂CH₃, NA¹A², C(O)NA¹A², SO₂NA¹A², SONA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², or C₍₁₋₆₎alkylNA¹A², provided that R_{b} is H, CF₃, CH₂CF₃, C₍₁₋₆₎alkyl, or C₍₃₋₆₎cycloalkyl; wherein said 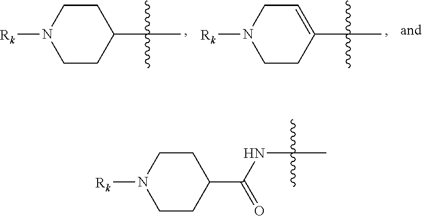
are optionally substituted with up to four methyl groups on two or more ring carbon atoms or optionally substituted with up to two CF₃ groups on any two ring carbon atoms;
A¹ is H, or C₍₁₋₃₎alkyl;
A² is H, C₍₁₋₆₎alkyl, C₍₃₋₆₎cycloalkyl, 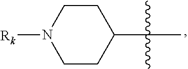
C₍₂₋₆₎alkylOH, C₍₂₋₆₎alkylOCH₃, SO₂C₍₁₋₄₎alkyl, C(O)Ph, C(O)C₍₁₋₄₎alkyl, pyrazinyl, or pyridyl, wherein said cycloalkyl, alkyl, pyrazinyl, pyridyl, or Ph groups may be optionally be substituted with two substituents selected from the group consisting of F, C₍₁₋₆₎alkyl, CF₃, pyrrolidinyl, CO₂H, C(O)NH₂, SO₂NH₂, OC₍₁₋₄₎alkyl, —CN, NO₂, OH, NH₂, NHC₍₁₋₄₎alkyl, N(C₍₁₋₄₎alkyl)₂; and said pyridyl, or Ph may be additionally be substituted with up to two halogens independently selected from the group consisting of: Cl, and Br; or A¹ and A² are taken together with their attached nitrogen to form a ring selected from the group consisting of: 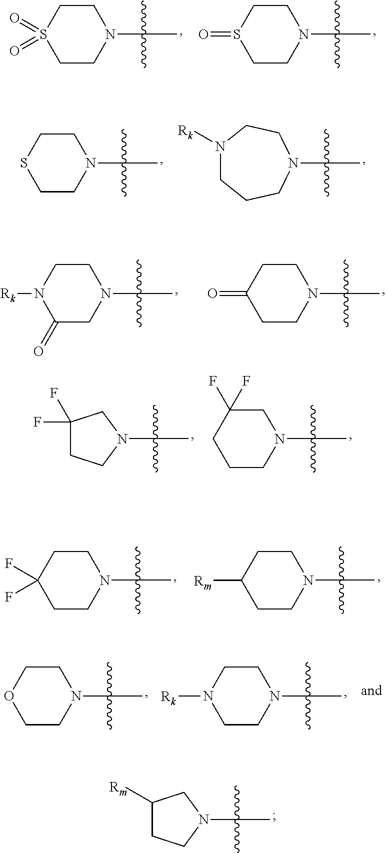
wherein any said A¹ and A² ring may be optionally substituted with up to four methyl groups on two or more ring carbon atoms or optionally substituted with up to two CF₃ groups on any two ring carbon atoms;
Rₖ is selected from the group consisting of H, CH₂CF₃, CH₂CH₂CF₃, C₍₁₋₆₎alkyl, COC₍₁₋₄₎alkyl, SO₂C₍₁₋₄₎alkyl, trifluoromethylpyridyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, C₍₁₋₆₎alkyl, F, or OH;
Rₐₐ is H, CF₃, CH₂CF₃, Cl, Br, C₍₁₋₆₎alkyl, CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, SO₂NA¹A², SONA¹A², C(O)NA¹A², C(O)N(C₍₁₋₃₎alkyl)C₍₂₋₄₎alkylNA¹A², C(O)NHC₍₂₋₄₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², or C₍₁₋₆₎alkylNA¹A²;
R_{b} is H, CF₃, CH₂CF₃, or C₍₁₋₆₎alkyl, or C₍₃₋₆₎cycloalkyl; or R_{b} may also be 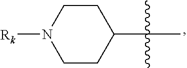
C(O)C₍₁₋₄₎alkyl, C(O)Ph, SO₂C₍₁₋₄₎alkyl, C₍₂₋₆₎alkylOC₍₁₋₆₎alkyl, C₍₂₋₆₎alkylOC₍₃₋₆₎cycloalkyl, C₍₂₋₆₎alkylOC₍₂₋₆₎alkylNA¹A², C₍₂₋₆₎alkylNHC₍₂₋₆₎alkylNA¹A², C₍₂₋₆₎alkylN(C₍₁₋₃₎alkyl)C₍₂₋₆₎alkylNA¹A², or C₍₂₋₆₎alkylNA¹A², provided that Rₐ is H, Cl, Br, CH₂OH, NH₂, CF₃, CH₂CF₃,
or C₍₁₋₆₎alkyl; wherein said 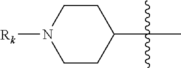
is optionally substituted with up to four methyl groups on two or more ring carbon atoms or optionally substituted with up to two CF₃ groups on any two ring carbon atoms;
R_{c} is H, C₍₁₋₃₎alkyl, or CF₃;
and solvates, hydrates, tautomers, and pharmaceutically acceptable salts thereof.

2. A Compound of claim 1, wherein:
R¹ is OC₍₁₋₅₎alkyl, O—C₍₃₋₅₎cycloalkyl, CF₃, CH₂CF₃, OCH₂—C₍₃₋₅₎cycloalkyl, OCF₃, OCH₂CF₃, NO₂, C₍₁₋₅₎alkyl, Cl, F, SC₍₁₋₄₎alkyl, SCH₂-cyclopentyl, S-cyclopropyl, or H;
R² is F, Cl, Br, CF₃, CH₃, or H; or R² and R¹ may be taken together with the ring to which they are attached, to form a fused ring system selected from the group consisting of: quinolinyl, isoquinolinyl, quinazolinyl, quinoxalinyl, benzimidazolyl, benzofuranyl, 2,3-dihydro-benzofuranyl, benzothiophenyl, benzothiazolyl, and indazolyl, wherein said quinolinyl, isoquinolinyl, quinazolinyl, quinoxalinyl, benzimidazolyl, benzothiazolyl, benzofuranyl, 2,3-dihydro-benzofuranyl, benzothiophenyl, and indazolyl are optionally substituted with one methyl group or up to two fluorine atoms;
R⁴ is H, NH₂, CONH₂, C₍₁₋₅₎alkyl, CO₂C₍₁₋₅₎alkyl, OC₍₁₋₅₎alkyl, CO₂H, SO₂NH₂, Cl, NHC(O)C₍₁₋₅₎alkyl, CF₃, CH₂CF₃, SO₂C₍₁₋₅₎alkyl, SO₂NHC₍₁₋₅₎alkyl, SO₂N(C₍₁₋₅₎alkyl)₂, SO₂NL¹L², F, P(O)(OCH₂CH₃)₂, NO₂, —CN, P(O)(OH)₂, Br, —CH═CH₂, —CONHCH₃, CH₂CONH₂, —CONHCH₂CH₂-piperidinyl, —CONHCH₂Ph, or NHC(O)NH₂; provided that R⁴ is not OCH₃, if R¹ is C₍₁₋₄₎alkyl or H; or R⁴ is selected from the group consisting of: phenyl, pyridyl, pyrazolyl, imidazolyl, oxazolyl, isoxazolyl, thiazolyl, and thiophenyl wherein said phenyl, pyridyl, pyrazolyl, imidazolyl, oxazolyl, isoxazolyl, thiazolyl, and thiophenyl are optionally substituted with one substituent selected from the group consisting of C(O)CH₃, F, Cl, —CN, OCH₃, and CH₃; L¹ and L² are taken together with their attached nitrogen to form a ring, selected from the group consisting of: 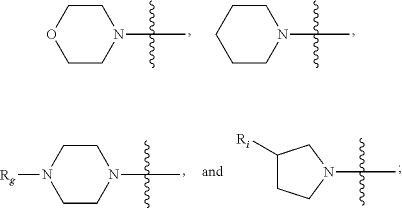
R_{g} is selected from the group consisting of H, COCH₃, and CH₃;
Rᵢ is H, CH₃, or CH₂OH;
C¹ and C² are carbon atoms which are further substituted to make a ring selected from the group consisting of: 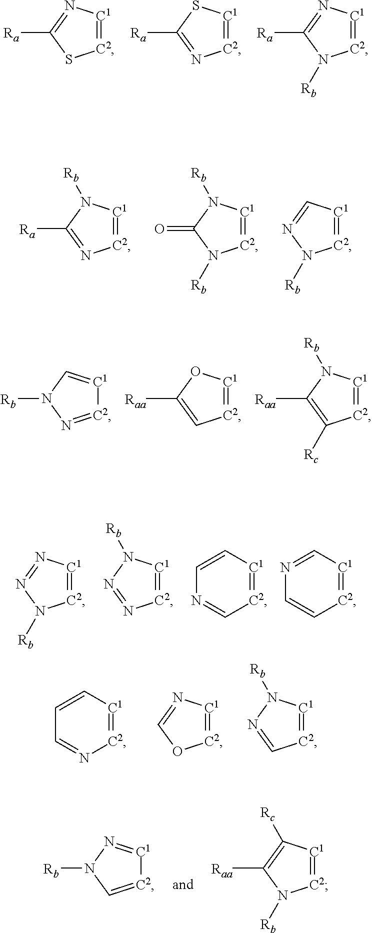
Rₐ is H, CF₃, CH₂CF₃, CH₂OH, Cl, Br, or C₍₁₋₆₎alkyl; or Rₐ may also be 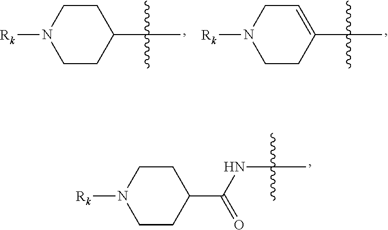
NHPhOCH₂CH₃, NA¹A², C(O)NA¹A², SO₂NA¹A², SONA¹A², C(O)N(CH₃)C₍₂₋₆₎alkylNA¹A², C(O)NHC₍₂₋₆₎alkylNA¹A², NHC(O)C₍₁₋₆₎alkylNA¹A², N(CH₃)C(O)C₍₁₋₆₎alkylNA¹A², CH₂OC₍₁₋₆₎alkyl, CH₂OC₍₃₋₆₎cycloalkyl, CH₂OC₍₂₋₆₎alkylNA¹A², CH₂NHC₍₂₋₆₎alkylNA¹A², CH₂N(CH₃)C₍₂₋₆₎alkylNA¹A², NHC₍₂₋₆₎alkylNA¹A², N(CH₃)C₍₂₋₆₎alkylNA¹A², or CH₂NA¹A², provided that R_{b} is H, CF₃, CH₂CF₃, C₍₁₋₆₎alkyl, or C₍₃₋₆₎cycloalkyl;
A¹ is H, or C₍₁₋₃₎alkyl;
A² is H, C₍₁₋₆₎alkyl, C₍₃₋₆₎cycloalkyl, 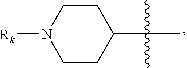
C₍₂₋₆₎alkylOH, C₍₂₋₆₎alkylOCH₃, SO₂C₍₁₋₄₎alkyl, C(O)Ph, C(O)C₍₁₋₄₎alkyl, pyrazinyl, or pyridyl; or A¹ and A² are taken together with their attached nitrogen to form a ring selected from the group consisting of: 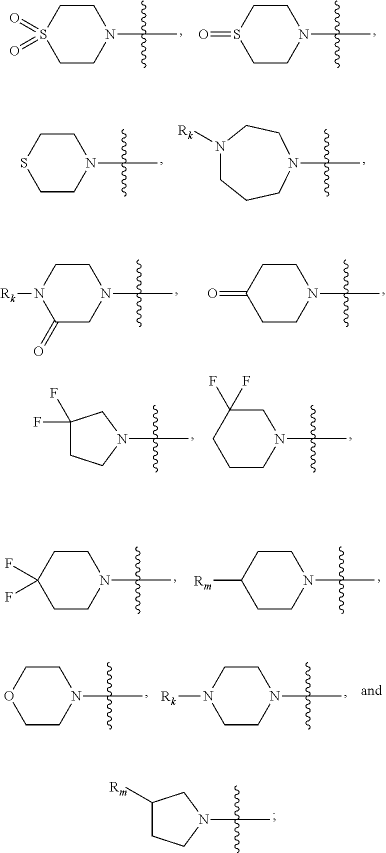
wherein any said A¹ and A² ring may be optionally substituted with up to four methyl groups on two or more ring carbon atoms or optionally substituted with up to two CF₃ groups on any two ring carbon atoms;
Rₖ is selected from the group consisting of H, CH₂CF₃, CH₂CH₂CF₃, C₍₁₋₃₎alkyl, COC₍₁₋₄₎alkyl, SO₂C₍₁₋₄₎alkyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, CH₃, F, or OH;
Rₐₐ is H, CF₃, CH₂CF₃, Cl, Br, C₍₁₋₆₎alkyl, SO₂NA¹A², SONA¹A², C(O)NA¹A², C(O)N(CH₃)C₍₂₋₄₎alkylNA¹A², C(O)NHC₍₂₋₄₎alkylNA¹A², CH₂OC₍₁₋₆₎alkyl, CH₂OC₍₃₋₆₎cycloalkyl, CH₂OC₍₂₋₆₎alkylNA¹A², CH₂NHC₍₂₋₆₎)alkylNA¹A², CH₂N(CH₃)C₍₂₋₆₎alkylNA¹A², or CH₂NA¹A²;
R_{b} is H, CF₃, CH₂CF₃, or C₍₁₋₆₎alkyl, or C₍₃₋₆₎cycloalkyl; or R_{b} may also be 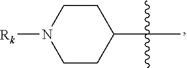
CH₂CH₂OC₍₁₋₆₎alkyl, CH₂CH₂OC₍₃₋₆₎cycloalkyl, CH₂CH₂OC₍₂₋₆₎alkylNA¹A², CH₂CH₂NHC₍₂₋₆₎alkylNA¹A², CH₂CH₂N(CH₃)C₍₂₋₆₎alkylNA¹A², or CH₂CH₂NA¹A², provided that Rₐ is H, Cl, Br, CH₂OH, NH₂, CF₃, CH₂CF₃, or C₍₁₋₆₎alkyl;
and solvates, hydrates, tautomers, and pharmaceutically acceptable salts thereof.

3. A compound of claim 2, wherein:
R¹ is OC₍₁₋₅₎alkyl, O-cyclopentyl, CF₃, CH₂CF₃, SCH₃, OCH₂-cyclopropyl, OCF₃, OCH₂CF₃, NO₂, C₍₁₋₅₎alkyl or H;
R² is F, CF₃, CH₃, or H; or R² and R¹ may be taken together with the ring to which they are attached, to form a fused ring system selected from the group consisting of: benzimidazolyl, quinolinyl, benzofuranyl, and 2,3-dihydro-benzofuranyl, wherein said benzimidazolyl, quinolinyl, benzofuranyl, and 2,3-dihydro-benzofuranyl are optionally substituted with one methyl group or up to two fluorine atoms;
R³ is F, CONH₂, OCH₃, NO₂, —CN, CH₃, CF₃, or H; provided that R³ is not F if R⁴ is H;
R⁴ is H, NH₂, CONH₂, C₍₁₋₅₎alkyl, CO₂C₍₁₋₅₎alkyl, OC₍₁₋₅₎alkyl, CO₂H, SO₂NH₂, Cl, NHC(O)C₍₁₋₅₎alkyl, CF₃, CH₂CF₃, SO₂C₍₁₋₅₎alkyl, SO₂NHC₍₁₋₅₎alkyl, SO₂N(C₍₁₋₅₎alkyl)₂, SO₂NL¹L², F, P(O)(OCH₂CH₃)₂, NO₂, —CN, P(O)(OH)₂, Br, pyrazolyl, —CH═CH₂, —CONHCH₃, CH₂CONH₂, —CONHCH₂CH₂-piperidinyl, —CONHCH₂Ph, or NHC(O)NH₂; provided that R⁴ is not OCH₃, if R¹ is C₍₁₋₄₎alkyl or H;
C¹ and C² are carbon atoms which are further substituted to make a ring selected from the group consisting of: 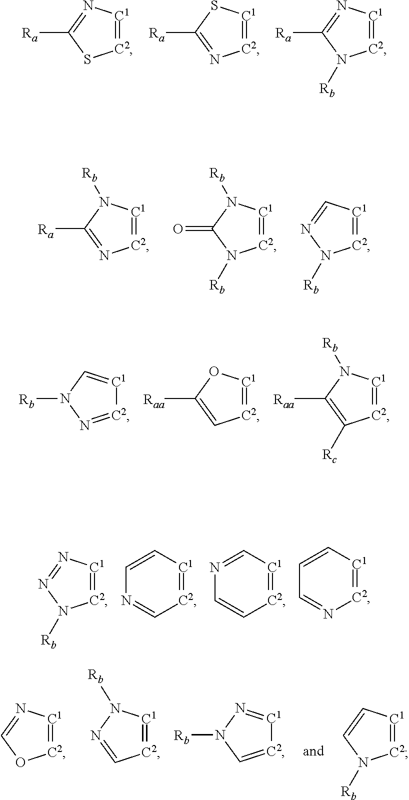
Rₐ is H, CF₃, Cl, CH₂OH, CH₂CF₃, or C₍₁₋₅₎alkyl; or Rₐ may also be 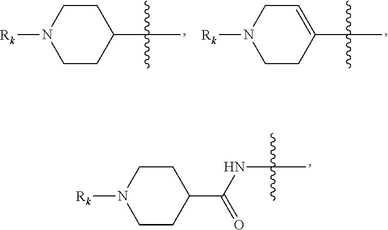
NHPhOCH₂CH₃, NA¹A², C(O)NA¹A², SO₂NA¹A², SONA¹A², C(O)N(CH₃)C₍₂₋₃₎alkylNA¹A², C(O)NHC₍₂₋₃₎alkylNA¹A², NHC(O)C₍₁₋₃₎alkylNA¹A², N(CH₃)C(O)C₍₁₋₃₎alkylNA¹A², CH₂OC₍₂₋₃₎alkylNA¹A², CH₂NHC₍₂₋₃₎alkylNA¹A², CH₂N(CH₃)C₍₂₋₃₎alkylNA¹A², NHC₍₂₋₃₎alkylNA¹A², N(CH₃)C₍₂₋₃₎alkylNA¹A², or CH₂NA¹A², provided that R_{b} is H, CF₃, CH₂CF₃, C₍₁₋₃₎alkyl, or C₍₃₋₆₎cycloalkyl;
A¹ is H, or C₍₁₋₃₎alkyl;
A² is H, C₍₁₋₃₎alkyl, 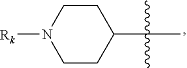
or C(O)C₍₁₋₄₎alkyl; or A¹ and A² are taken together with their attached nitrogen to form a ring selected from the group consisting of: 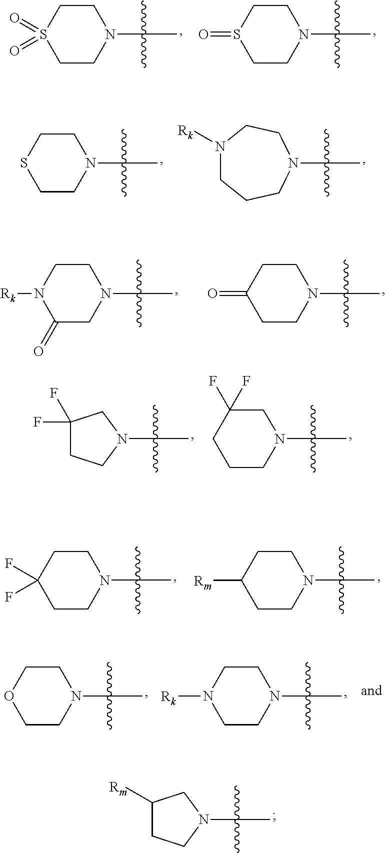
Rₖ is selected from the group consisting of H, C₍₁₋₃₎alkyl, COC₍₁₋₄₎alkyl, SO₂C₍₁₋₄₎alkyl, and C₍₃₋₆₎cycloalkyl;
Rₘ is H, OCH₃, CH₂OH, NHCH₃, N(CH₃)₂, NH₂, F, or OH;
Rₐₐ is H, CF₃, CH₂CF₃, C₍₁₋₅₎alkyl, SO₂NA¹A², SONA¹A², C(O)NA¹A², C(O)N(CH₃)C₍₂₋₃₎alkylNA¹A², C(O)NHC₍₂₋₃₎alkylNA¹A², CH₂OC₍₂₋₃₎alkylNA¹A², CH₂NHC₍₂₋₃₎alkylNA¹A², CH₂N(CH₃)C₍₂₋₃₎alkylNA¹A², or CH₂NA¹A²;
R_{b} is H, CF₃, CH₂CF₃, or C₍₁₋₅₎alkyl, or C₍₃₋₆₎cycloalkyl; or R_{b} may also be 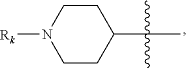
CH₂CH₂OC₍₂₋₃₎alkylNA¹A², CH₂CH₂NHC₍₂₋₃₎alkylNA¹A², CH₂CH₂N(CH₃)C₍₂₋₃₎alkylNA¹A², or CH₂CH₂NA¹A², provided that Rₐ is H, NH₂, CF₃, CH₂CF₃, Cl, CH₂OH, or C₍₁₋₅₎alkyl;
R_{c} is H, C₍₁₋₃₎alkyl, or CF₃;
and solvates, hydrates, tautomers, and pharmaceutically acceptable salts thereof.

4. A compound of claim 3, wherein:
R¹ is OC₍₁₋₄₎alkyl, O-cyclopentyl, SCH₃, OCH₂-cyclopropyl, OCF₃, OCH₂CF₃, NO₂, C₍₁₋₅₎alkyl or H;
R² is F, CF₃, CH₃, or H; or R² and R¹ may be taken together with the ring to which they are attached, to form a fused ring system selected from the group consisting of: 1-methyl benzoimidazol-4-yl;
R⁴ is H, NH₂, CONH₂, C₍₁₋₅₎alkyl, CO₂C₍₁₋₅₎alkyl, OC₍₁₋₅₎alkyl, CO₂H, SO₂NH₂, Cl, NHC(O)C₍₁₋₅₎alkyl, CF₃, CH₂CF₃, SO₂C₍₁₋₅₎alkyl, SO₂NHC₍₁₋₅₎alkyl, SO₂N(C₍₁₋₅₎alkyl)₂, SO₂-pyrrolidinyl, SO₂-piperidinyl, SO₂-morpholinyl, SO₂-piperazinyl, F, P(O)(OCH₂CH₃)₂, NO₂, —CN, P(O)(OH)₂, Br, pyrazolyl, or NHC(O)NH₂; provided that R⁴ is not OCH₃, if R¹ is C₍₁₋₄₎alkyl or H;
C¹ and C² are carbon atoms which are further substituted to make a ring selected from the group consisting of: 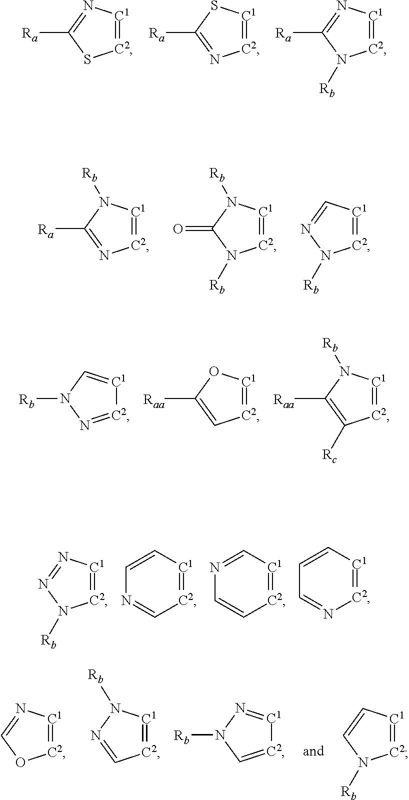
Rₐ is H, CF₃, CH₂CF₃, C₍₁₋₅₎alkyl, C₁, NH₂, CH₂OH, NHC(O)C₍₁₋₄₎alkyl, NHC₍₁₋₃₎alkyl, N(C₍₁₋₃₎alkyl)₂, NHPhOCH₂CH₃, C(O)NA¹A², or NA¹A²;
A¹ and A² are taken together with their attached nitrogen to form a ring selected from the group consisting of: 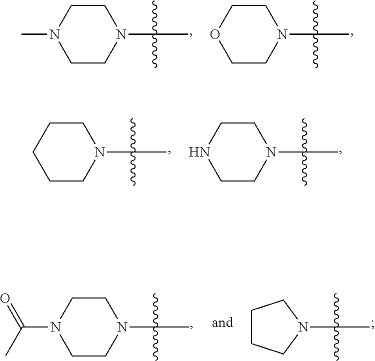
Rₐₐ is H, or C₍₁₋₅₎alkyl;
R_{b} is H, CH₂CF₃, or C₍₁₋₅₎alkyl;
R_{c} is H, or C₍₁₋₃₎alkyl;
and solvates, hydrates, tautomers, and pharmaceutically acceptable salts thereof.

5. A compound of claim 4, wherein:
R₁ is OC₍₁₋₄₎alkyl, OCF₃, OCH₂CF₃, NO₂, C₍₁₋₄₎alkyl or H;
R² is F, CF₃, or H; or R² and R¹ may be taken together with the ring to which they are attached, to form a fused ring system selected from the group consisting of: 1-methyl benzoimidazol-4-yl;
R³ is F, CONH₂, OCH₃, NO₂, —CN, CH₃, or H; provided that R³ is not F if R⁴ is H;
R⁴ is H, NH₂, CONH₂, CH₃, CO₂CH₃, OCH₃, CO₂H, SO₂NH₂, Cl, NHC(O)CH₃, CF₃, SO₂CH₃, SO₂CH₂CH₃, SO₂NHCH₃, SO₂N(CH₃)₂, SO₂N(CH₂CH₃)₂, SO₂-pyrrolidinyl, SO₂-piperidinyl, SO₂-morpholinyl, F, P(O)(OCH₂CH₃)₂, NO₂, —CN, P(O)(OH)₂, Br, pyrazolyl, or NHC(O)NH₂; provided that R⁴ is not OCH₃, if R¹ is C₍₁₋₄₎alkyl or H;
C¹ and C² are carbon atoms which are further substituted to make a ring selected from the group consisting of: 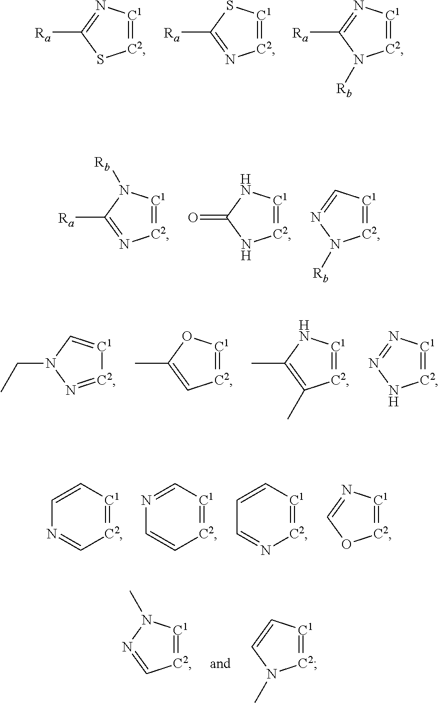
Rₐ is H, CH₃, C₁, NH₂, CH₂OH, NHC(O)CH₃, NHPhOCH₂CH₃, C(O)NA¹A², or NA¹A²;
A¹ and A² are taken together with their attached nitrogen to form a ring selected from the group consisting of 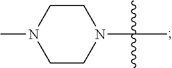
R_{b} is H, CH₂CF₃, or C₍₁₋₅₎alkyl;
and solvates, hydrates, tautomers, and pharmaceutically acceptable salts thereof.

6. A compound of claim 1, wherein:
R¹ is OCH(CH₃)₂;
Q is C—R²;
R² is H;
R³ is H;
J is C—R⁴; and
R⁴ is —CONH₂, —CO₂H, or —SO₂NH₂;
and solvates, hydrates, tautomers, and pharmaceutically acceptable salts thereof.

7. A compound which is selected from the group consisting of: 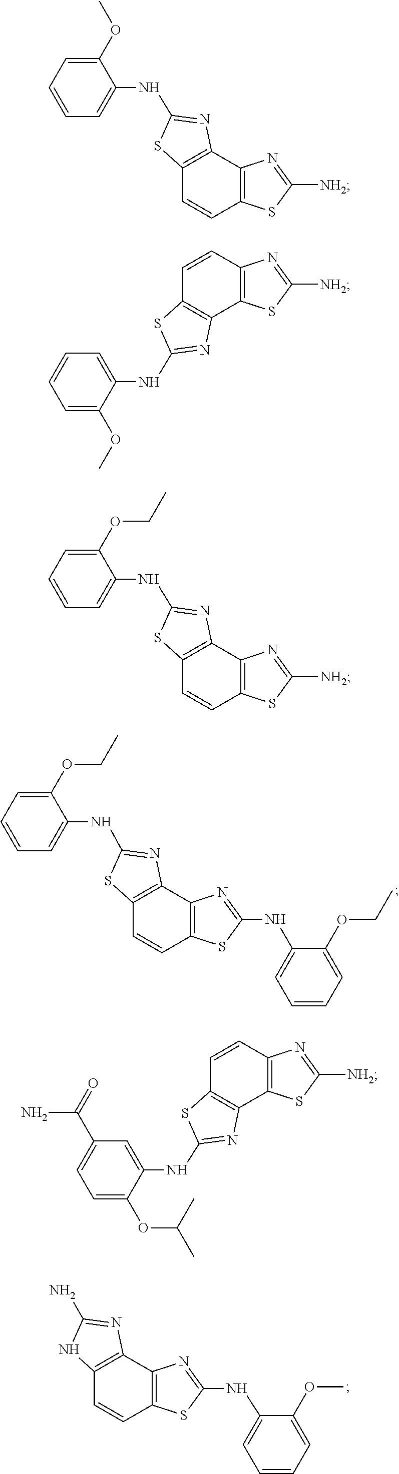 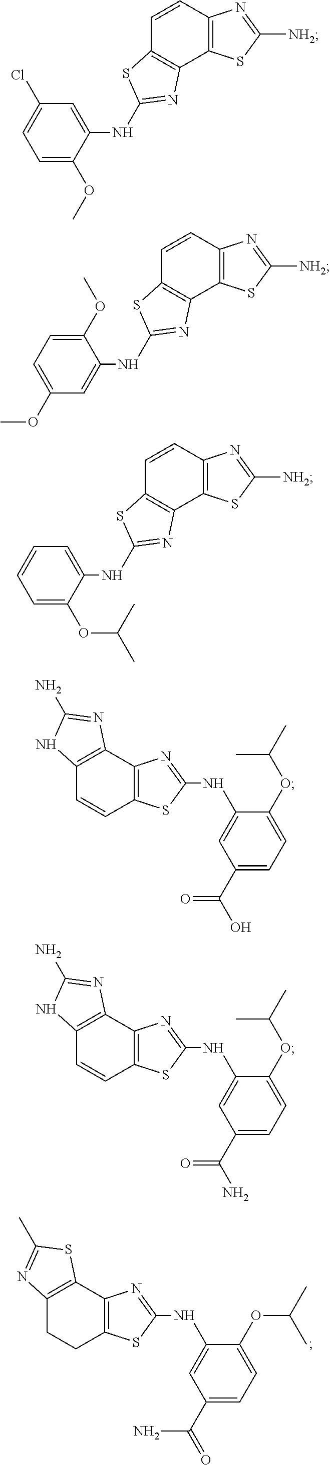 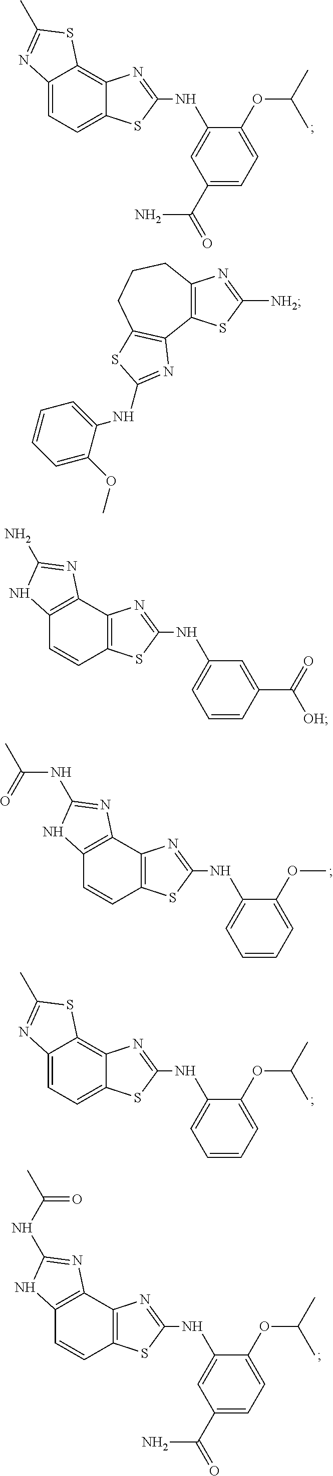 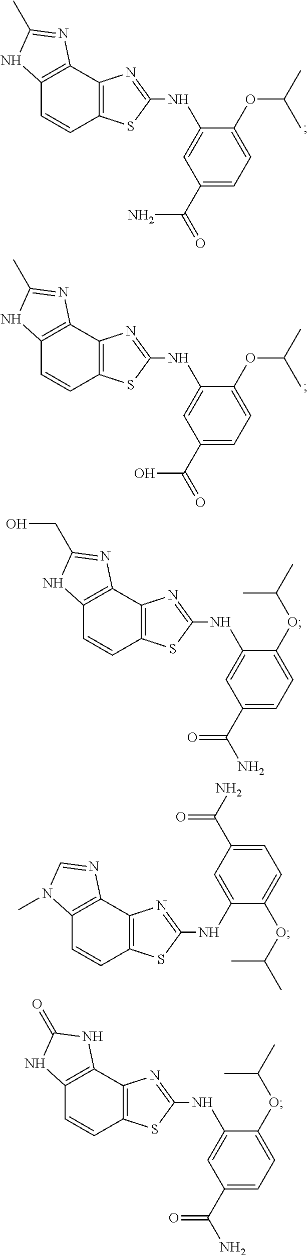 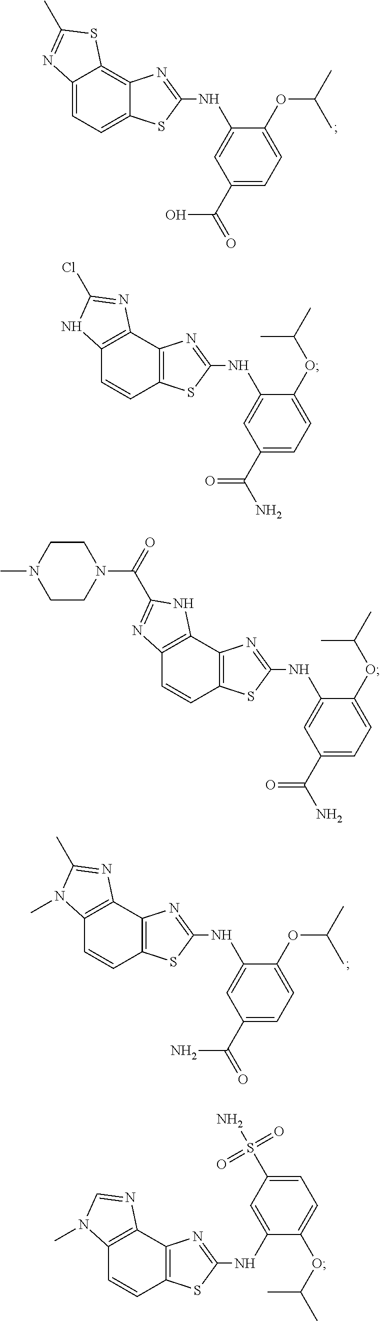 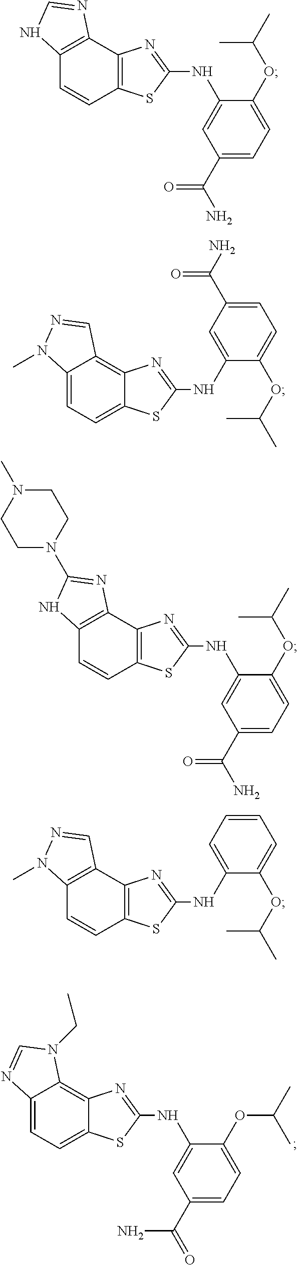 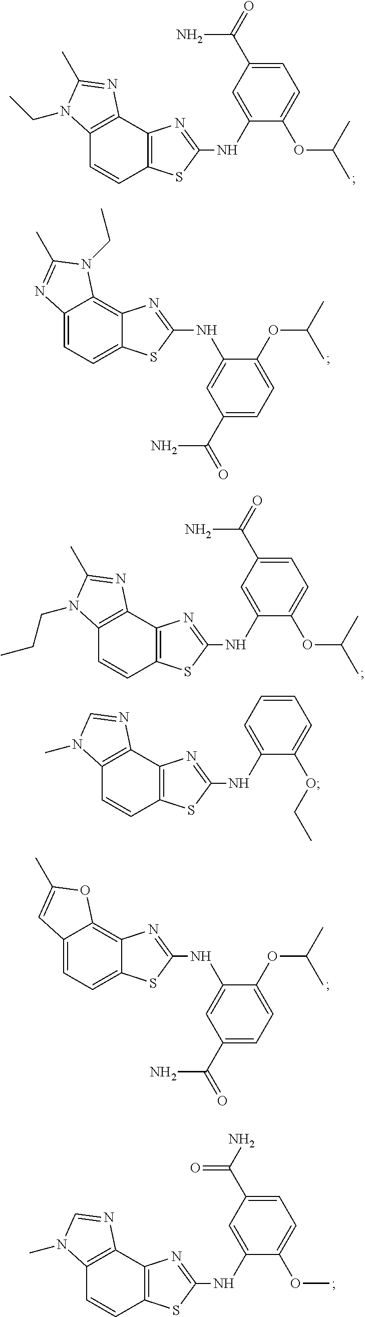 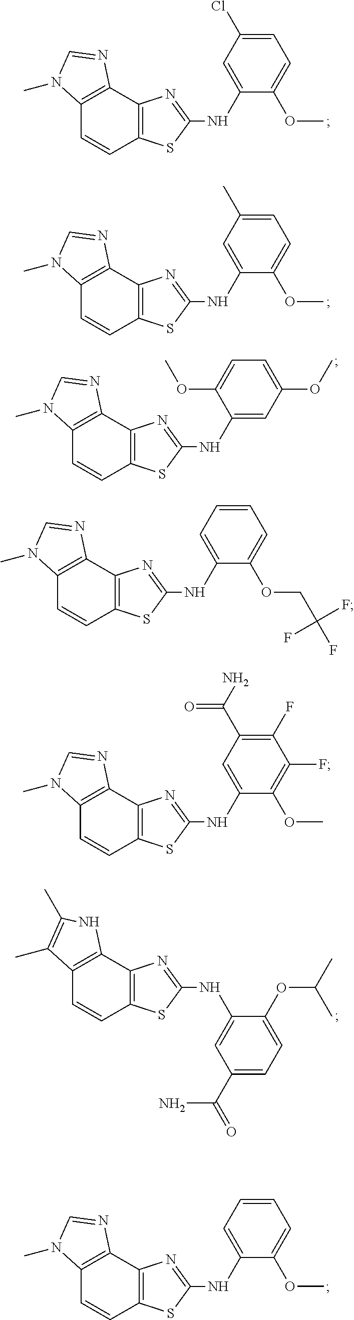 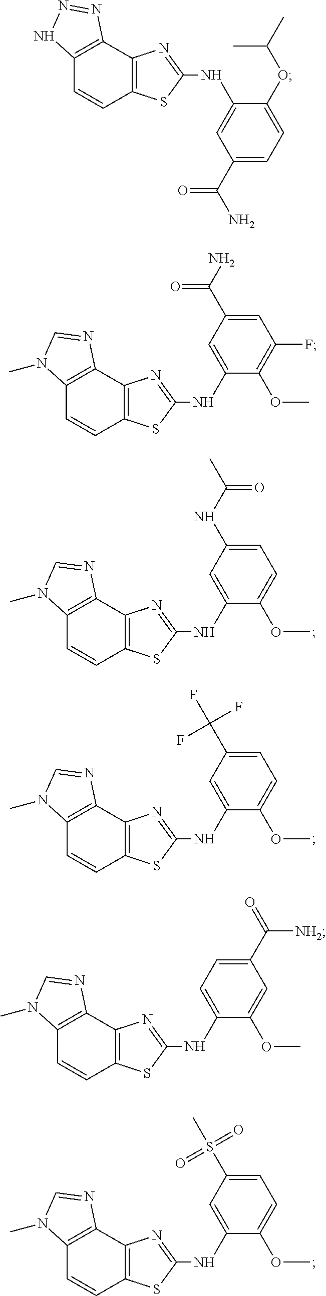 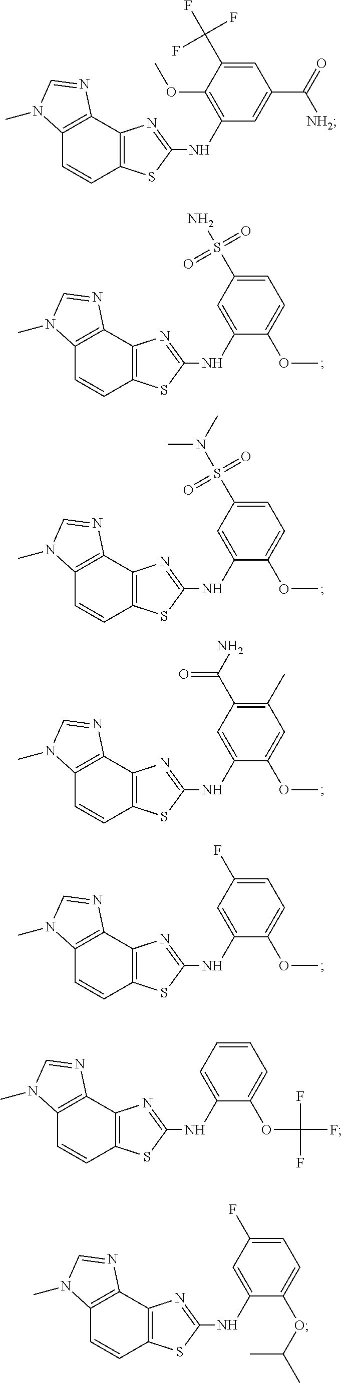 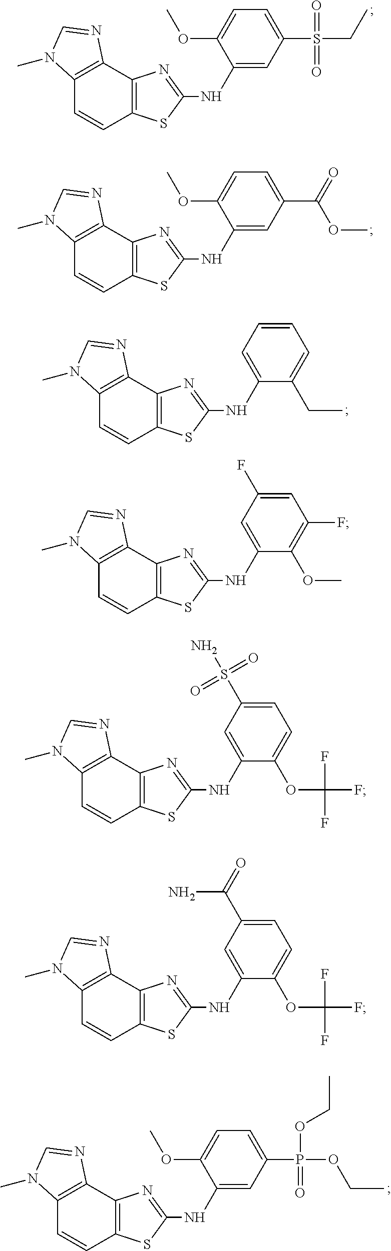 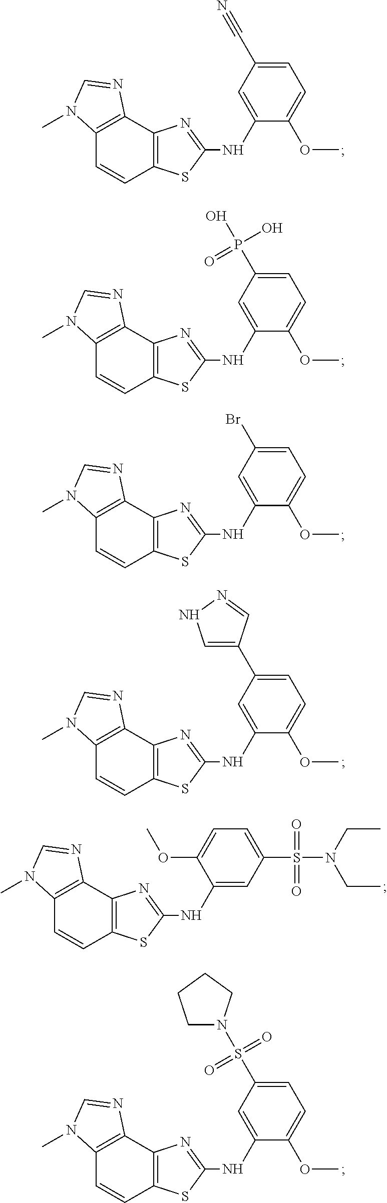 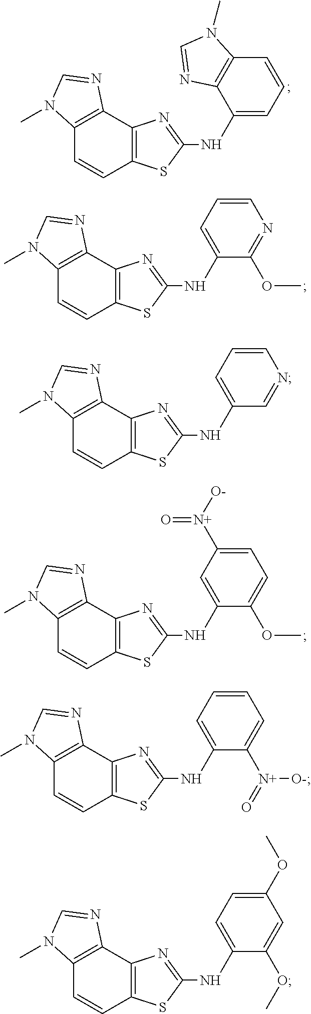 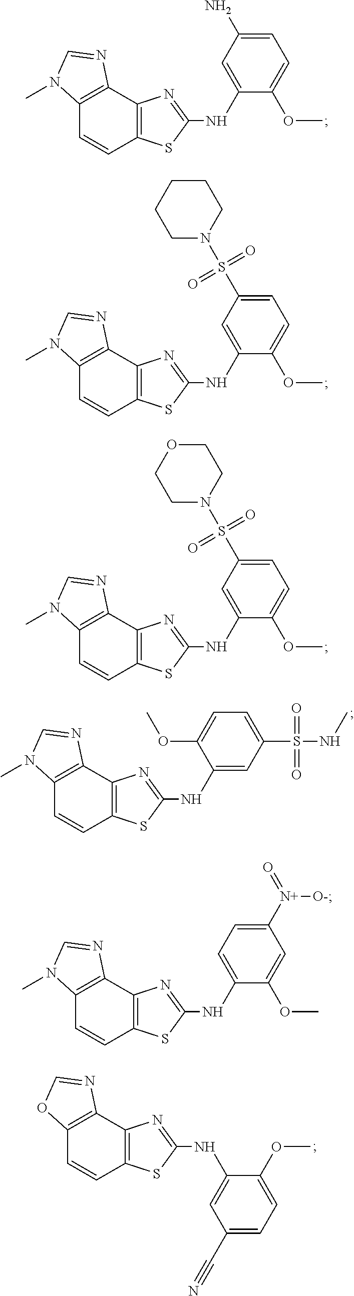 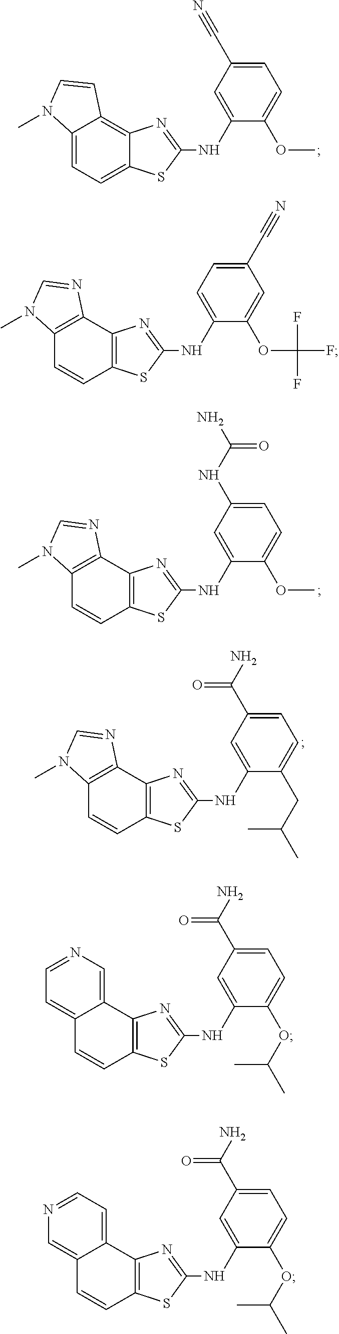 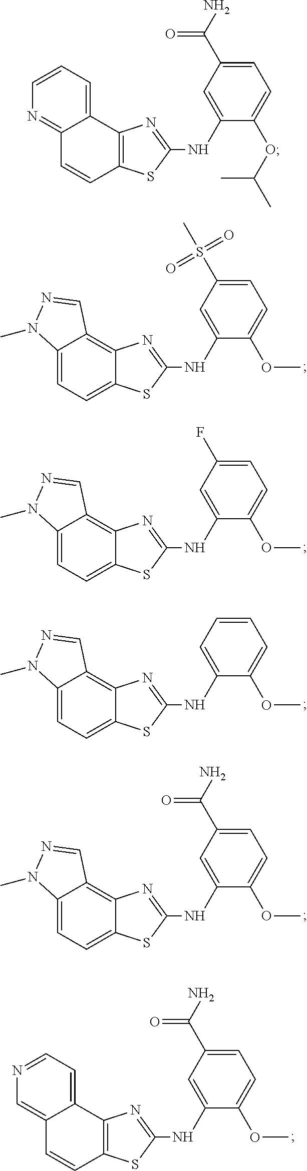 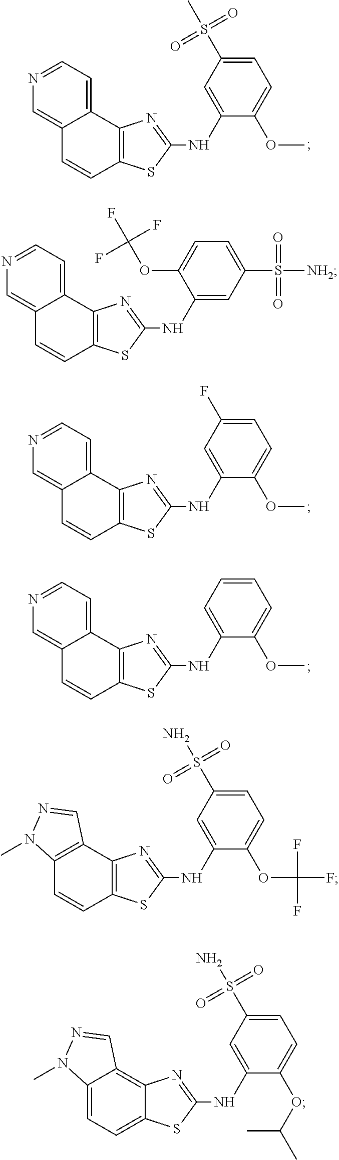 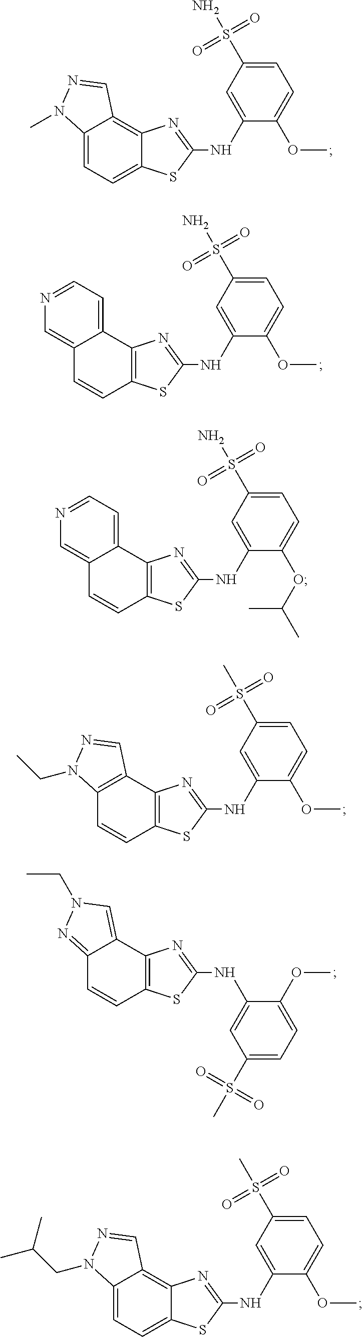 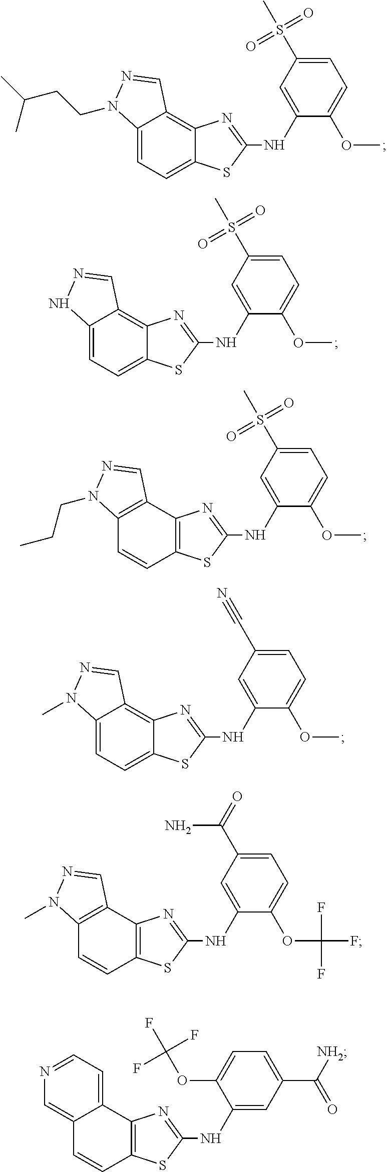 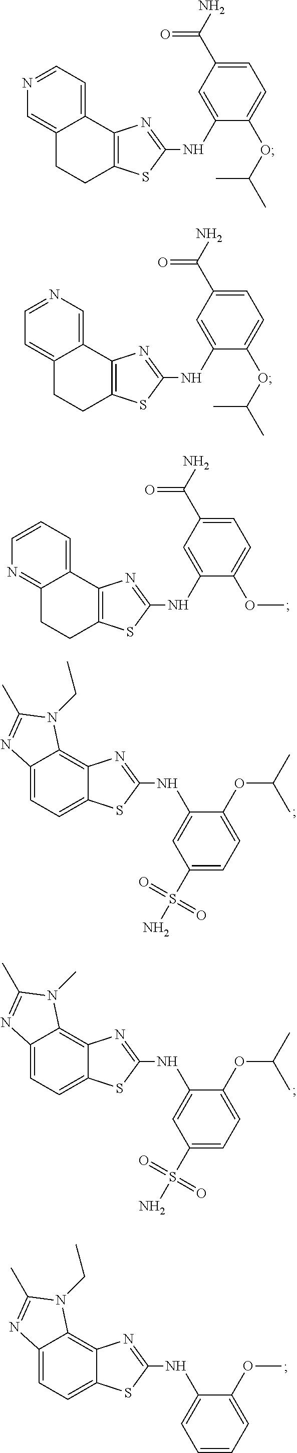 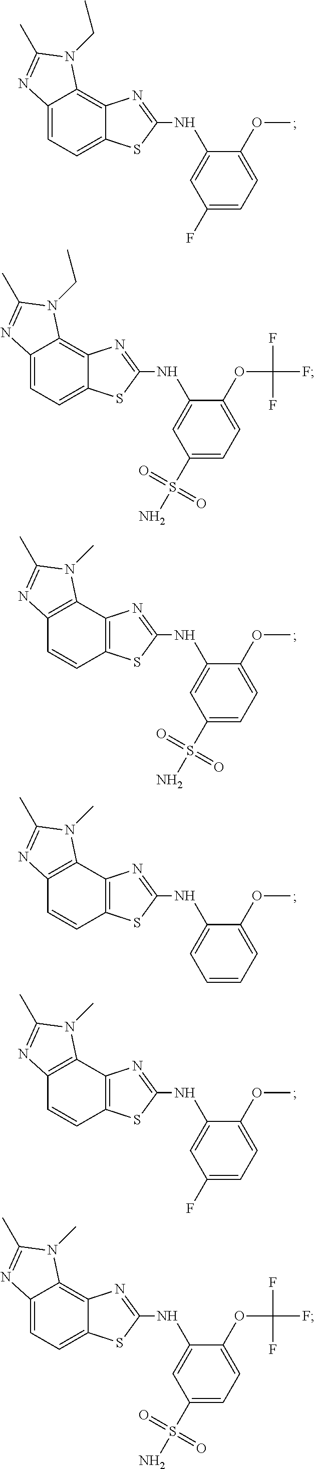 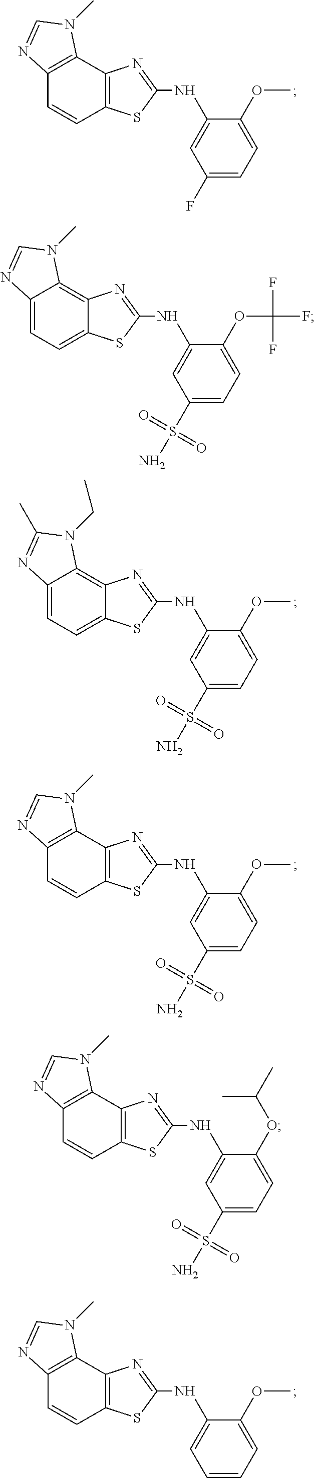 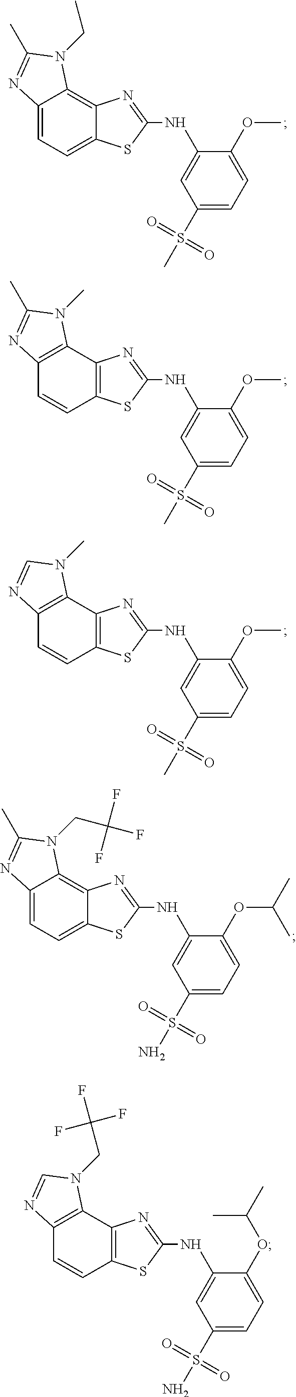 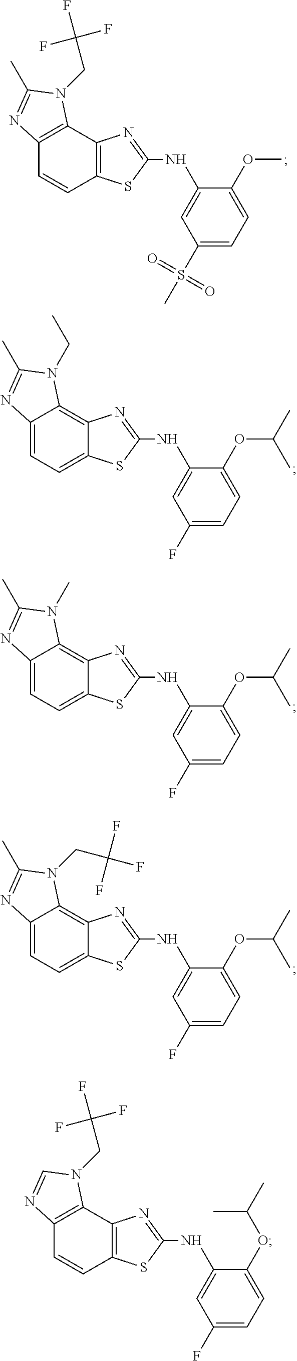 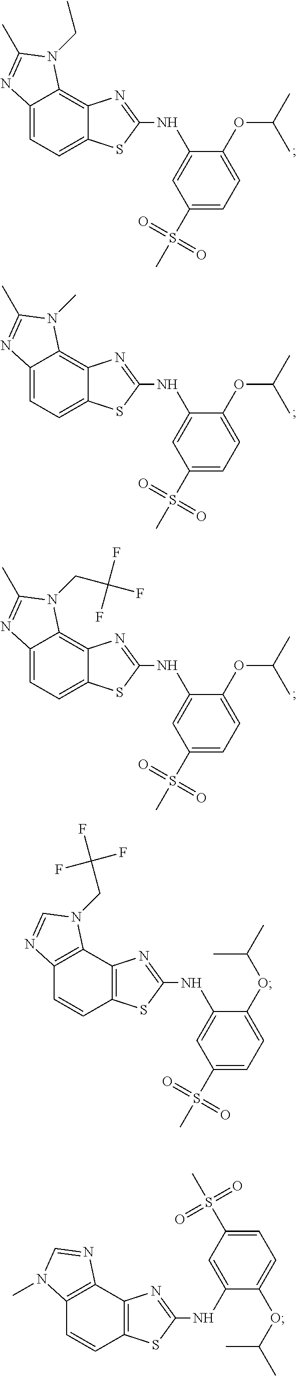 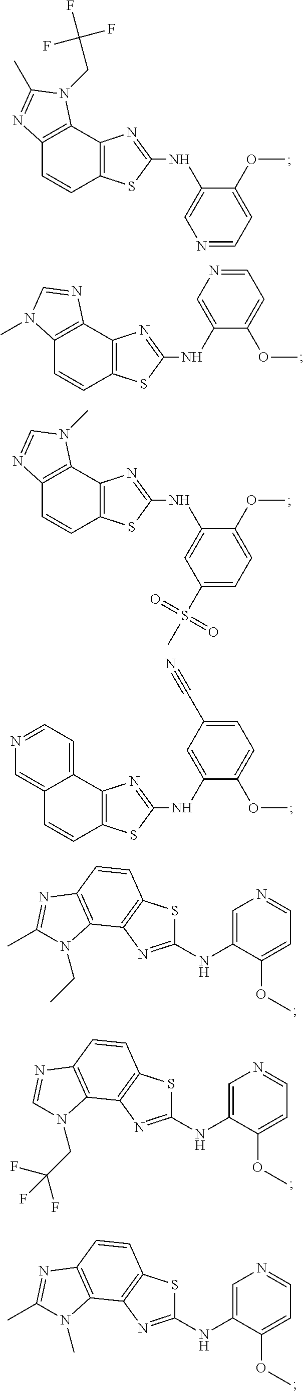 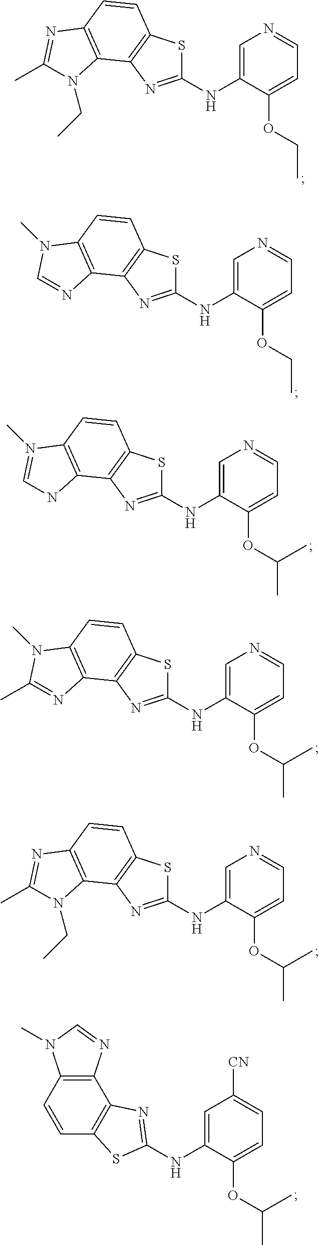 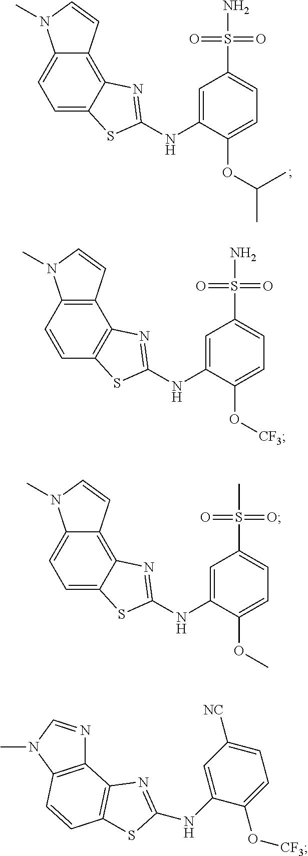
and solvates, hydrates, tautomers, and pharmaceutically acceptable salts thereof.

8. A pharmaceutical composition, comprising a compound of Claim 1 and a pharmaceutically acceptable carrier.

9. A pharmaceutical composition, comprising a compound listed in the Examples section of this specification and a pharmaceutically acceptable carrier.

10. A method for preventing, treating or ameliorating an MMP9 mediated syndrome, disorder or disease comprising administering to a subject in need thereof an effective amount of a compound of claim 1 or a form, composition or medicament thereof.

11. A method for preventing, treating or ameliorating an MMP9 mediated syndrome, disorder or disease wherein said syndrome, disorder or disease is associated with elevated MMP9 expression or MMP9 overexpression, or is a condition that accompanies syndromes, disorders or diseases associated with elevated MMP9 expression or MMP9 overexpression comprising administering to a subject in need thereof an effective amount of a compound of Formula I or a form, composition or medicament thereof.

12. A method of preventing, treating or ameliorating a syndrome, disorder or disease, wherein said syndrome, disorder or disease is selected from the group consisting of: neoplastic disorders, osteoarthritis, rheumatoid arthritis, cardiovascular diseases, gastric ulcer, pulmonary hypertension, chronic obstructive pulmonary disease, inflammatory bowel syndrome, periodontal disease, skin ulcers, liver fibrosis, emphysema, Marfan syndrome, stroke, multiple sclerosis, asthma, abdominal aortic aneurysm, coronary artery disease, idiopathic pulmonary fibrosis, renal fibrosis, and migraine, comprising administering to a subject in need thereof an effective amount of a compound of Formula I or a form, composition or medicament thereof.

13. The method of claim 12, wherein said syndrome, disorder or disease is a neoplastic disorder, which is ovarian cancer.

14. The method of claim 12, wherein said syndrome, disorder or disease is a cardiovascular disease, wherein said cardiovascular disease is selected from the group consisting of: atherosclerotic plaque rupture, aneurysm, vascular tissue morphogenesis, coronary artery disease, and myocardial tissue morphogenesis.

15. The method of claim 14, wherein said cardiovascular disease is atherosclerotic plaque rupture.

16. The method of claim 12, wherein said syndrome, disorder or disease is rheumatoid arthritis.

17. The method of claim 12, wherein said syndrome, disorder or disease is asthma.

18. The method of claim 12, wherein said syndrome, disorder or disease is chronic obstructive pulmonary disease.

19. The method of claim 12, wherein said syndrome, disorder or disease is inflammatory bowel syndrome.

20. The method of claim 12, wherein said syndrome, disorder or disease is abdominal aortic aneurism.

21. The method of claim 12, wherein said syndrome, disorder or disease is osteoarthritis.

22. The method of claim 12, wherein said syndrome, disorder or disease is idiopathic pulmonary fibrosis.

23. A method of inhibiting MMP9 activity in a mammal by administration of an effective amount of at least one compound of claim 1.

24. A method for preventing, treating or ameliorating an MMP13 mediated syndrome, disorder or disease comprising administering to a subject in need thereof an effective amount of a compound of claim 1 or a form, composition or medicament thereof.

25. A method for preventing, treating or ameliorating an MMP13 mediated syndrome, disorder or disease wherein said syndrome, disorder or disease is associated with elevated MMP13 expression or MMP13 overexpression, or is a condition that accompanies syndromes, disorders or diseases associated with elevated MMP13 expression or MMP13 overexpression comprising administering to a subject in need thereof an effective amount of a compound of Formula I or a form, composition or medicament thereof.

26. A method of inhibiting MMP13 activity in a mammal by administration of an effective amount of at least one compound of claim 1.
